(19) United States
(12) Patent Application Publication
(10) Pub. No.: US 2020/0157237 A1
(43) Pub. Date: May 21, 2020

(54) LYMPHOCYTE ANTIGEN CD5LIKE (CD5L) MONOMER, HOMODIMER, AND INTERLEUKIN 12B (P40) HETERODIMER ANTAGONISTS AND METHODS OF USE THEREOF

(71) Applicants: The Broad Institute, Inc., Cambridge, MA (US); Massachusetts Institute of Technology, Cambridge, MA (US); The Brigham and Women's Hospital, Inc., Boston, MA (US)

(72) Inventors: Aviv Regev, Cambridge, MA (US); Vijay K. Kuchroo, Boston, MA (US); Chao Wang, Boston, MA (US)

(21) Appl. No.: 16/616,536

(22) PCT Filed: May 25, 2018
(86) PCT No.: PCT/US2018/034782

§ 371 Date: Nov. 25, 2019

Related U.S. Application Data
(60) Provisional application No. 62/563,469, filed on Sep. 26, 2017.
(60) Provisional application No. 62/511,202, filed on May 25, 2017.

(51) Int. Cl. C07K 16/28; C07K 16/30; C12N 15/113; G01N 33/50; G01N 33/574; G16B 50/30; G16H 50/70

(52) U.S. Cl. CPC C07K 16/2896; C07K 16/30; C07K 16/2866; C12N 15/1138; A61K 38/00; G01N 33/57484; G16B 50/30; G16H 50/70; G01N 33/5011

(57) ABSTRACT

Described herein are antagonists of CD5L monomer, CD5L:CD5L homodimer, and CD5L:p40 heterodimer and compositions and methods for modulating or enhancing an immune response in a subject, e.g. a subject with cancer or chronic infection, involving said antagonists.

### CROSS-REFERENCE TO RELATED APPLICATIONS

This application claims the benefit of U.S. Provisional Application Nos. 62/511,202, filed May 25, 2017 and 62/563,469, filed Sep. 26, 2017. The entire contents of the above-identified applications are hereby fully incorporated herein by reference.

### STATEMENT REGARDING FEDERALLY SPONSORED RESEARCH

This invention was made with government support under Grant No.(s) AI056299, AI039671, AI073748, and AI045757 awarded by the National Institutes of Health. The government has certain rights in the invention.

### TECHNICAL FIELD

The subject matter disclosed herein is generally directed to compositions and methods for modulating an immune response in a subject by targeting CD5L:p40 heterodimers, downstream targets of CD5L:p40 heterodimers and/or the receptor for CD5L:p40 heterodimers.

### BACKGROUND

The cytokine environment influences immune cell differentiation, function and plasticity. Interleukin 23 (IL-23) has been identified as key player in inflammatory diseases, contributing largely to mucosal inflammation. It was discovered as a susceptibility gene in GWAS and is widely implicated in autoimmune diseases and cancer such as melanoma and colorectal carcinoma (Burkett et al., 2015; Cho and Feldman, 2015; Teng et al., 2015; Wang and Karin, 2015).

### SUMMARY

The present invention is based, at least in part, on the discovery that CD5L and p40 form heterodimers in vivo, and that these heterodimers modulate the immune response. CD5L exists as a monomer, and is also able to form dimers; both forms may also serve as immunomodulators.

In one aspect, the present invention provides for an antagonist against the function or signaling of one or more of a CD5L monomer, a CD5L:CD5L homodimer, and a CD5L:p40 heterodimer. In certain embodiments, the antagonist is an antibody, or an antigen binding fragment or equivalent thereof, that interacts with (e.g., specifically binds with) one or more of the CD5L monomer, the CD5L:CD5L homodimer, and the CD5L:p40 heterodimer. In certain embodiments, the antagonist is an antibody, or an antigen binding fragment or equivalent thereof, that interacts with (e.g., specifically binds with) Il12rb1. In certain embodiments, the antibody is a polyclonal antibody, a monoclonal antibody, a chimeric antibody, a human antibody, a veneered antibody, a diabody, a humanized antibody, an antibody derivative, a recombinant humanized antibody. In certain embodiments, the equivalent is an aptamer, affimer, non-immunoglobulin scaffold, small molecule, or fragment or derivative thereof.

In certain embodiments, the antibody specifically binds the CD5L monomer. In certain embodiments, the antibody specifically binds the CD5L:CD5L homodimer. The antibody may be produced by a cell line selected from the group of cell lines listed in Table 1.

In certain embodiments, the antibody specifically binds a CD5L:p40 heterodimer. The antibody may be produced by a cell line selected from the group of cell lines in Table 2.

In certain embodiments, the antagonist is an antibody, an antigen binding fragment or equivalent thereof, small molecule, or genetic modifying agent, said antagonist targeting a downstream target of a CD5L:p40 heterodimer, a CD5L monomer, or a CD5L:CD5L homodimer. The downstream target may be selected from the group consisting of Dusp2, Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Snx20, Lamp1, Faf1, Gpatch3, Dapk3, 1110065P20Rik, Vaultrc5, Il17f, Il17a, Ildr1, Il1r1, Lgr4, Ptpnl4, Paqr8, Timp1, Il1rn, Smim3, Gap43, Tigit, Mmp10, Il22, Enpp2, Iltifb, Ido1, I123r, Stom, Bcl2l11, 5031414D18Rik, Il24, Itga7, Il6, Epha2, Mt2, Upp1, Snord104, 5730577I03Rik, Slcl8b1, Ptprj, Clip3, Mir5104, Ppifos, Rab13, Hist1h2bn, Ass1, Cd200r1, E130112N10Rik, Mxd4, Casp6, Gatm, Tnfrsf8, Gp49a, Gadd45g, Ccr5, Tgm2, Lilrb4, Ecm1, Arhgap18, Serpinb5, Cysltr1, Enpp1, Selp, Slc38a4, Gm14005, Epb4.1l4b, Moxd1, Klra7, Igfbp4, Tnip3, Gstt1, Pglyrp2, Il12rb2, Ctla2a, Plac8, Ly6c1, Sell, Ncf1, Trp53i11, B3gnt3, Kremen2, Matk, Ltb4r1, Ets1, Tnfrsf26, Cd28, Rybp, Ppp1r3c, Thy1, Trib2, Sema3b, Pros1, 1133, Gm5483, Myh11, Cntd1, Ms4a4b, Treml2, 3110009E18Rik, Pglyrp1, Amd1, Slc24a5, Snhg9, Ifi27l1, Irf7, Mx1, Snhg10, 114, Snora43, H2-L, Myl4, Insl3, Tgoln2, BC022687, C230035I16Rik, Hvcn1, Myh10, Dhrs3, Acsl6, Rgs2, Ccl20, Ccl3, Dlg2, Ccr6, Ccl4, Dusp14, Apol9b, Cd72, Ispd, Cd70, S100a1, Lgals3, Slc15a3, Nkg7, Serpinc1, Olfr175-ps1, 119, Pdlim4, Il3, Insl6, Perp, Cd51, Serpine2, Galnt14, Tff1, Ppfibp2, Bdh2, Mlf1, Il1a, Osr2, Gm5779, Ebf1, Spink2, Egfr and Ccdc155.

In another aspect, the present invention provides for a composition comprising the antagonist of any one of claims 1 to 12 and a pharmaceutically acceptable carrier. The composition may further comprise an additional active agent used to treat a cancer. The cancer may not be inflammation related. The additional active agent may be one or more checkpoint inhibitors, anti-PD-1, anti-PDL-1, anti-CTLA4, anti-cancer vaccines, adoptive T cell therapy, and/or inhibitory nucleic acids that target CD5L and/or p40. The inhibitory nucleic acids may be genetic modifying agents, small interfering RNAs (e.g., shRNA), antisense oligonucleotides, and/or CRISPR system.

In another aspect, the present invention provides for a method of treating a cancer in a subject comprising administering to the subject a therapeutically effective amount of an antagonist of any one of claims 1 to 12 or a composition of any one of claims 13 to 17. The method may further comprise sequentially or simultaneously administering an additional active agent used to treat the cancer. The additional active agent may be a standard treatment for the cancer. The cancer treatment may be an immunotherapy treatment. The immunotherapy treatment may be checkpoint blockade therapy. The checkpoint blockade therapy may comprise anti-CTLA4, anti-PD1, anti-PDL1 or combination thereof. The cancer may be adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer.

In another aspect, the present invention provides for a method for enhancing an immune response in a subject, comprising administering to the subject a therapeutically effective amount of an antagonist of any one of claims 1 to 12 or a composition of any one of claims 13 to 17. The subject may have an immune deficiency. The immune deficiency may be a primary or secondary immune deficiency. The subject may have an infection with a pathogen. The pathogen may be a viral, bacterial, or fungal pathogen.

In another aspect, the present invention provides for a method of modulating CD8⁺ T cell exhaustion in a subject in need thereof, the method comprising administering to the subject a therapeutically effective amount of an antagonist antibody to one or more of a CD5L monomer, a CD5L:CD5L homodimer, and a CD5L:p40 heterodimer.

In another aspect, the present invention provides for an antagonistic antibody that associates with an epitope of one or more of a CD5L monomer, a CD5L:CD5L homodimer, and a CD5L:p40 heterodimer.

In another aspect, the present invention provides for a method of screening for an antagonist of one or more of a CD5L monomer, a CD5L:CD5L homodimer, and a CD5L:p40 heterodimer, the method comprising: exposing a cell or a population of cells to an agent that interacts with one or more of a CD5L monomer, a CD5L:CD5L homodimer, and a CD5L:p40 heterodimer; determining expression of a gene or set of genes up and/or down-regulated upon exposure to one or more of a CD5L monomer, a CD5L:CD5L homodimer, a CD5L:p40 heterodimer or antagonist thereof in the cell or population of cells; and determining that the agent is an antagonist based on the gene or set of genes up and/or down-regulated in the cell or population of cells. The antagonist may be an antibody.

In another aspect, the present invention provides for a method of screening for an antagonistic agent comprising: identifying an epitope on one or more of a CD5L monomer, a CD5L:CD5L homodimer, and a CD5L:p40 heterodimer that interacts with an antagonist of one or more of a CD5L monomer, a CD5L:CD5L homodimer, and a CD5L:p40 heterodimer; and screening against a library of candidate antagonistic agents for an antagonistic agent that interacts with the epitope. The antagonist may be an antibody. The antagonistic agent may be an antibody, a small molecule, a peptide, an aptamer, an affimer, a non-immunoglobulin scaffold, or fragment or derivative thereof. The library may comprise a computer database and the screening may comprise a virtual screening. The screening may comprise evaluating the three-dimensional structure of one or more of the CD5L monomer, the CD5L:CD5L homodimer, and the CD5L:p40 heterodimer.

In another aspect, the present invention provides for a method of identifying an agent for treating a cancer that is not inflammation related in a subject, comprising contacting the agent with a T cell, wherein decreased expression of CD5L monomer, CD5L:CD5L homodimer, and/or CD5L:p40 heterodimer indicates that the agent is effective for treating the cancer that is not inflammation related in the subject.

In another aspect, the present invention provides for a method of identifying an agent for enhancing an immune response in a subject, comprising contacting a myeloid cell with the agent, wherein decreased expression of CD5L monomer, CD5L:CD5L homodimer, and/or CD5L:p40 heterodimer indicates that the agent is effective for enhancing the immune response in the subject. The subject may have an immune deficiency. The immune deficiency may be a primary or secondary immune deficiency. The subject may have an infection with a pathogen. The pathogen may be a viral, bacterial, or fungal pathogen.

In another aspect, the present invention provides for a method of treating cancer in a subject, comprising administering to the subject a therapeutically effective amount of an antagonist of any one of claims 1 to 12 or a composition of any one of claims 13 to 17, wherein the cancer is promoted by complement. The antagonist may be an antibody. The antibody may specifically bind CD5L monomer. The antibody may specifically bind CD5L:CD5L homodimer. The antibody may specifically bind CD5L:p40 heterodimer.

In certain embodiments, the antagonist according to any embodiment herein is an antibody that binds to the fibronectin domain 2 of p40.

Aspects of the disclosure relate to a CD5L monomer, CD5L:CD5L homodimer, and/or CD5L:p40 heterodimer antagonist or and/one or more nucleic acids encoding the same. In some embodiments, the antagonist is an antibody or an antigen binding fragment thereof. In some embodiments, the antagonist is an aptamer, affimer, non-immunoglobulin scaffold, small molecule, or fragment or derivative thereof.

Further aspects of the disclosure relate to methods for enhancing an immune response in a subject, the method comprising administering to the subject a therapeutically effective amount of an antagonist and/or one or more nucleic acids encoding the same. In some embodiments, the subject has cancer, such as a non-inflammation related/non-inflammatory cancer.

Some embodiments comprise administering an anti-cancer immunotherapy to the subject, such as checkpoint inhibitors, PD-1/PDL-1, anti-cancer vaccines, adoptive T cell therapy, and/or combination of two or more thereof.

In some embodiments, the subject has an immune deficiency, e.g., a primary or secondary immune deficiency. In some embodiments, the subject has an infection with a pathogen, e.g., viral, bacterial, or fungal pathogen.

In embodiments that comprise administering inhibitory nucleic acids, the nucleic acids can include small interfering RNAs (e.g., shRNA), antisense oligonucleotides (e.g. antisense RNAs), and/or CRISPR-Cas.

In some embodiments, the subject has an immune deficiency, e.g., a primary or secondary immune deficiency. In some embodiments, the subject has an infection with a pathogen, e.g., viral, bacterial, or fungal pathogen.

Some aspects related to an antagonist against one or more of a CD5L monomer, a CD5L:CD5L homodimer, and a CD5L:p40 heterodimer. In some embodiments, the antagonist is an antibody, or an antigen binding fragment or equivalent thereof, that interacts with (e.g., specifically binds with) one or more of the CD5L monomer, the CD5L:CD5L homodimer, and the CD5L:p40 heterodimer. In some embodiments, the antibody is a polyclonal antibody, a monoclonal antibody, a chimeric antibody, a human antibody, a veneered antibody, a diabody, a humanized antibody, an antibody derivative, a recombinant humanized antibody. In some embodiments, the equivalent is an aptamer, affimer, non-immunoglobulin scaffold, small molecule, or fragment or derivative thereof.

In some embodiments, the antibody specifically binds the CD5L monomer.

In some embodiments, the antibody specifically binds the CD5L:CD5L homodimer. In some embodiments, the antibody is produced by a cell line selected from the group of cell lines listed in Table 1.

In some embodiments, the antibody specifically binds a CD5L:p40 heterodimer. In some embodiments, the antibody is produced by a cell line selected from the group of cell lines in Table 2.

Some aspects relate to compositions comprising an antagonist against one or more of a CD5L monomer, a CD5L:CD5L homodimer, and a CD5L:p40 heterodimer and a pharmaceutically acceptable carrier. Some embodiments further comprise an additional active agent used to treat a cancer that is not inflammation related. In some embodiments, the additional active agent is one or more checkpoint inhibitors, PD-1/PDL-1, anti-cancer vaccines, adoptive T cell therapy, and/or inhibitory nucleic acids that target CD5L and/or p40. In some embodiments, the inhibitory nucleic acids are small interfering RNAs (e.g., shRNA), antisense oligonucleotides, and/or CRISPR-Cas.

Some aspects relate to methods of treating a cancer that is not inflammation related in a subject comprising administering to the subject a therapeutically effective amount of an antagonist against one or more of a CD5L monomer, a CD5L:CD5L homodimer, and a CD5L:p40 heterodimer. Some embodiments further comprise sequentially or simultaneously administering an additional active agent used to treat the cancer. In some embodiments, the additional active agent is a standard treatment for the cancer.

In some embodiments, the cancer is adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer.

Some aspects relate to methods for enhancing an immune response in a subject, comprising administering to the subject a therapeutically effective amount of an antagonist against one or more of a CD5L monomer, a CD5L:CD5L homodimer, and a CD5L:p40 heterodimer, or a composition comprising the antagonist. In some embodiments, the subject has an immune deficiency. In some embodiments, the immune deficiency is a primary or secondary immune deficiency.

In some embodiments, the subject has an infection with a pathogen. In some embodiments, the pathogen is a viral, bacterial, or fungal pathogen.

Some aspects relate to methods of modulating CD8⁺ T cell exhaustion in a subject in need thereof, the method comprising administering to the subject a therapeutically effective amount of an antagonist antibody to one or more of a CD5L monomer, a CD5L:CD5L homodimer, and a CD5L:p40 heterodimer.

Some aspects relate to antagonistic antibodies that associate with an epitope of one or more of a CD5L monomer, a CD5L:CD5L homodimer, and a CD5L:p40 heterodimer.

Some aspects relate to methods of identifying a gene or a set of genes up and/or downregulated in response to an agonistic antibody, the method comprising: exposing a cell or population of cells to the antagonist of one or more of a CD5L monomer, a CD5L:CD5L homodimer, and a CD5L:p40 heterodimer, and introducing one or more guide RNAs that target one or more endogenous genes into the cell or population of cells, wherein the cell or population of cells express a CRISPR-Cas9 protein or a CRISPR-Cas9 protein or a nucleic acid encoding the CRISPR-Cas9 protein has been introduced into the cell or population of cells simultaneously or sequentially with the guide RNAs, assaying for a phenotype indicative of enhanced or suppressed immune response, and identifying a gene or set of genes up and/or down regulated in the cell or population of cells with the enhanced or suppressed immune response. In some embodiments, the cell or population of cells are cancer cell(s). In some embodiments, the cancer is adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer. In some embodiments, the cancer cell(s) are human cells. In some embodiments, the human cancer cell(s) have been transplanted into a mouse.

Some aspects relate to methods of treating a cancer that is not inflammation related comprising administering to a subject in need thereof (i) an antagonist of one or more of a CD5L monomer, a CD5L:CD5L homodimer, and a CD5L:p40 heterodimerO and (ii) an agent that targets a gene or set of genes identified as provided herein.

Some aspects relate to methods of screening for an antagonist of one or more of a CD5L monomer, a CD5L:CD5L homodimer, and a CD5L:p40 heterodimer, the method comprising: exposing a cell or a population of cells to an agent that interacts with one or more of a CD5L monomer, a CD5L:CD5L homodimer, and a CD5L:p40 heterodimer; identifying a gene or set of genes up and/or down-regulated in the cell or population of cells; and determining that the agent is an antagonist based on the gene or set of genes up and/or down-regulated in the cell or population of cells. In some embodiments, the antagonist is an antibody. Some embodiments further comprise comparing the identified gene or set of genes to a previously-identified gene or set of genes up and/or down-regulated upon exposure to an antagonist of one or more of a CD5L monomer, a CD5L:CD5L homodimer, and a CD5L:p40 heterodimer.

Some aspects relate to methods of screening for an antagonistic agent comprising: identifying an epitope on one or more of a CD5L monomer, a CD5L:CD5L homodimer, and a CD5L:p40 heterodimer that interacts with an antagonist of one or more of a CD5L monomer, a CD5L:CD5L homodimer, and a CD5L:p40 heterodimer; and screening against a library of candidate antagonistic agents for an antagonistic agent that interacts with the epitope. In some embodiments, the antagonist is an antibody. In some embodiments, the antagonistic agent is an antibody, a small molecule, a peptide, an aptamer, an affimer, a non-immunoglobulin scaffold, or fragment or derivative thereof. In some embodiments, the library comprises a computer database and the screening comprises a virtual screening. In some embodiments, the screening comprises evaluating the three-dimensional structure of one or more of the CD5L monomer, the CD5L:CD5L homodimer, and the CD5L:p40 heterodimer.

Some aspects relate to methods of treating cancer in a subject, comprising administering to the subject a therapeutically effective amount of any of the antagonists described herein or any of the compositions described herein, wherein cancer is promoted by complement.

The invention relates to an antagonist of a CD5L:p40 heterodimer, a CD5L: CD5L homodimer, or a CD5L monomer, wherein the antagonist is capable of inhibiting growth of a MC38 colon carcinoma tumor xenograft in a mouse, e.g. compared to control. The MC38 colon carcinoma tumor xenograft may comprise about 1×10⁶ MC38 colon carcinoma cells injected subcutaneously in mice at day 0, and wherein tumor size is measured up to 14 days or more post-injection.

The antagonist may be capable of increasing the amount of tumor infiltrating CD8⁺ T cells in a MC38 colon carcinoma tumor xenograft in a mouse, e.g. compared to control. The MC38 colon carcinoma tumor xenograft may comprise about 1×10⁶ MC38 colon carcinoma cells injected subcutaneously in mice at day 0, and CD8⁺ tumor infiltrating lymphocytes (TILs) measured up to 14 days or more post-injection.

The antagonist may be capable of increasing the amount of tumor infiltrating CD8⁺ T cells which are positive for interleukin-2 (IL-2) in a MC38 colon carcinoma tumor xenograft in a mouse, e.g. compared to control. The antagonist may be capable of increasing the amount of tumor infiltrating CD8⁺ T cells which are positive for interferon gamma (IFNγ) in a MC38 colon carcinoma tumor xenograft in a mouse, e.g. compared to control. The antagonist may be capable of increasing the amount of tumor infiltrating CD8⁺ T cells which are positive for tumor necrosis factor alpha (TNFα) in a MC38 colon carcinoma tumor xenograft in a mouse, e.g. compared to control. The amount of tumor infiltrating CD8⁺ T cells which are positive for IL-2, IFNγ or TNFα may be assessed following isolation of T cells from the tumor at day 14 and following treatment of T cells with PMA/ionomycin for about 6 hours.

The antagonist may be capable of increasing the amount of tumor infiltrating CD4⁺ T cells in a MC38 colon carcinoma tumor xenograft in a mouse, e.g. compared to control. The MC38 colon carcinoma tumor xenograft may comprise about 1×10⁶ MC38 colon carcinoma cells injected subcutaneously in mice at day 0, and CD8⁺ TILs measured up to 14 days or more post-injection.

The antagonist may be capable of increasing the amount of tumor infiltrating CD4⁺ T cells which are positive for IL-2 in a MC38 colon carcinoma tumor xenograft in a mouse, e.g. compared to control. The antagonist may be capable of increasing the amount of tumor infiltrating CD4⁺ T cells which are positive for TNFα in a MC38 colon carcinoma tumor xenograft in a mouse, e.g. compared to control. The amount of tumor infiltrating CD4⁺ T cells which are positive for IL-2 or TNFα may be assessed following isolation of T cells from the tumor at day 14 and following treatment of T cells with PMA/ionomycin for about 6 hours.

The antagonist may be capable of reducing the amount of myeloid-derived suppressor cells (MDSCs) infiltrating into a MC38 colon carcinoma tumor xenograft in a mouse, e.g. compared to control, including the number of MDSCs which are positive for TNFα. The MC38 colon carcinoma tumor xenograft may comprise about 1×10⁶ MC38 colon carcinoma cells injected subcutaneously in mice at day 0, and MDSCs measured up to 14 days or more post-injection. The amount of infiltrating MDSCs, including the number of MDSCs which are positive for TNFα, may be assessed following isolation of MDSCs from the tumor at day 14 and following treatment of MDSCs with LPS for about 24 hours, with golgi stop/plug added in about the last four hours.

The antagonist may be capable of inhibiting the suppression of the production of IL-17 from pathogenic Th17 (Th17p) cells in vitro mediated by a CD5L:p40 heterodimer or agonist thereof, a CD5L: CD5L homodimer or agonist thereof, or a CD5L monomer or agonist thereof, e.g. compared to control.

The Th17p cells may be differentiated in vitro from naïve T cells under pathogenic Th17 conditions, e.g. using IL-1b, IL-6 and IL-23, and wherein IL-23 may be provided at 0.8 ng/ml or more, 4 ng/ml or more, or 20 ng/ml or more, optionally wherein IL-17 expression is measured in cell supernatant after 3 days of culture. The naïve T cells may be CD44^{low}CD62L⁺CD25-CD4⁺.

The antagonist may be capable of inhibiting the suppression of the production of IFN-γ from Th1 cells in vitro mediated by a CD5L:p40 heterodimer or agonist thereof, a CD5L: CD5L homodimer or agonist thereof, or a CD5L monomer or agonist thereof, e.g. compared to control.

The Th1 cells may be differentiated in vitro from naïve T cells under Th1 conditions, e.g. using IL-12, and wherein IL-12 may be provided at 0.16 ng/ml or more, 0.8 ng/ml or more, 4 ng/ml or more, or 20 ng/ml or more, optionally wherein IFN-γ expression is measured in cell supernatant after 3 days of culture. The naïve T cells may be CD44^{low}CD62L+CD25-CD4⁺.

The antagonist may be capable of promoting one or more of IFNγ production from CD8 T cells. The antagonist may be capable of promoting suppression on IL-12 from BMDC-T cells, and/or suppression on IL-23 from BMDC-T cells. The antagonist may be capable of promoting induction of Tim-3, PD-1 or TIGIT expression on T cells from BMDC-T cells coculture. The antagonist may be capable of promoting the induction of MCP-1 from DSS-colitis mouse. The antagonist may inhibit the induction of one or more of Dusp2, Anp32b, 1110065P20Rik, Atad3a, BC022687, Cyth2, Dapk2, Faf1, Fance, Gpatch3, Hccs, Il4, Itsn2, Lamp1, Marcksl1, Nol9, Nop9, Nubp1, Pithd1, Plk3, Ppp4c, Prkca, Snx20, Smnf1, Thap11, Tusc2, and Utp18.

The antagonist may be capable of inhibiting the reduction of neuroinflammation mediated by a CD5L:p40 heterodimer or agonist thereof, a CD5L: CD5L homodimer or agonist thereof, or a CD5L monomer or agonist thereof in a mouse model of experimental autoimmune encephalomyelitis (EAE), e.g. compared to control.

The antagonist may be capable of inhibiting the reduction of the EAE score mediated by a CD5L:p40 heterodimer or agonist thereof, a CD5L: CD5L homodimer or agonist thereof, or a CD5L monomer or agonist thereof in a mouse model of EAE, e.g. compared to control.

Inhibition of the reduction of neuroinflammation and/or EAE score may be observed from 20 days or more following induction of EAE.

The antagonist may be capable of inhibiting the reduction of the amount of CD4 T cells expressing interleukin-17 (IL-17) in CNS mediated by a CD5L:p40 heterodimer or agonist thereof, a CD5L: CD5L homodimer or agonist thereof, or a CD5L monomer or agonist thereof in a mouse model of EAE, e.g. compared to control. Inhibition may be observed from 20 days or more following induction of EAE.

The antagonist may be capable of inhibiting the reduction of the amount of CD4 T cells expressing interferon gamma (IFN-γ) mediated by a CD5L:p40 heterodimer or agonist thereof, a CD5L: CD5L homodimer or agonist thereof, or a CD5L monomer or agonist thereof in a mouse model of EAE, e.g. compared to control. Inhibition may be observed from 20 days or more following induction of EAE.

The mouse model of EAE may comprise immunization of mice with myelin oligodendrocyte glycoprotein (MOG) followed by injection with pertussis toxin (PT) prior to intraperitoneal administration of heterodimer, homodimer, monomer or agonist thereof.

The antagonist may be capable of inhibiting the reduction in colitis mediated by a CD5L:p40 heterodimer or agonist thereof, a CD5L: CD5L homodimer or agonist thereof, or a CD5L monomer or agonist thereof in a mouse model of colitis, e.g. compared to control.

The antagonist may be capable of inhibiting the reduction of weight loss mediated by a CD5L:p40 heterodimer or agonist thereof, a CD5L: CD5L homodimer or agonist thereof in a mouse model of colitis, e.g. compared to control.

Body weight may be measured over a period of 8 days or more following induction of colitis.

The antagonist may be capable of inhibiting the reduction of the amount of CD4 T cells expressing interleukin-17 (IL-17) mediated by a CD5L:p40 heterodimer or agonist thereof, a CD5L: CD5L homodimer or agonist thereof in a mouse model of colitis, e.g. compared to control.

The antagonist may be capable of inhibiting the reduction of the amount of CD4 T cells expressing interferon gamma (IFN-γ) mediated by a CD5L:p40 heterodimer or agonist thereof, a CD5L: CD5L homodimer or agonist thereof in a mouse model of colitis, e.g. compared to control.

The antagonist may be capable of inhibiting the reduction of the amount of group 3 innate lymphoid cells (ILC3s) in colon mediated by a CD5L:p40 heterodimer or agonist thereof, a CD5L: CD5L homodimer or agonist thereof in a mouse model of colitis, e.g. compared to control.

The mouse model of colitis may comprise induction of colitis by administration of 2% dextran sulfate sodium (DSS) in drinking water prior to administration of heterodimer, homodimer, monomer or agonist thereof.

The invention relates to any antagonist of a CD5L:p40 heterodimer, a CD5L: CD5L homodimer, or a CD5L monomer described above and herein for use as a medicament.

The invention relates to the use of any antagonist of a CD5L:p40 heterodimer, a CD5L: CD5L homodimer, or a CD5L monomer described above and herein in the manufacture of a medicament.

The invention relates to a pharmaceutical composition comprising any antagonist of a CD5L:p40 heterodimer, a CD5L: CD5L homodimer, or a CD5L monomer described above and herein and a pharmaceutically acceptable carrier or excipient.

With regard to any of the medical uses, medicaments or pharmaceutical uses, the associated medical treatment may be a method of treating a disease by enhancing the immune response, as described herein. The associated medical treatment may be a method of treating cancer as described herein, such as a non-inflammatory cancer as described herein. The cancer may be any cancer as described herein. The associated medical treatment may be a method of treating a subject that has an immune deficiency, e.g. a primary or secondary immune deficiency as described herein. The associated medical treatment may be a method of treating a subject that has an infection with a pathogen as described herein, e.g. a viral, bacterial or fungal pathogen as described herein. The associated medical treatment may be a method of treating any of the diseases described herein by modulating T cells as described herein.

Any of the CD5L:p40 heterodimer antagonists, CD5L: CD5L homodimer antagonists, CD5L monomer antagonists as described above and herein may be isolated antagonists.

Any of the antagonistic agents described above and herein may be an aptamer, affimer, non-immunoglobulin scaffold, small molecule, or binding portion or fragment or derivative thereof.

Any of the antagonistic agents described above and herein may be an antagonistic antibody or an antagonistic antigen-binding portion, fragment or equivalent thereof as described herein.

An antagonistic agent, such as an antibody or an antagonistic antigen-binding portion, fragment or equivalent thereof, may bind to and antagonize any function of a CD5L:p40 heterodimer, a CD5L: CD5L homodimer and/or a CD5L monomer e.g. as described herein, and wherein the antagonistic agent may possess any of the functional characteristics described above and herein. An antagonistic agent may bind to CD5L, p40, or both CD5L and p40 and antagonize any function of a CD5L:p40 heterodimer. An antagonistic agent may bind to CD5L and antagonize any function of a CD5L: CD5L homodimer or a CD5L monomer. An antagonistic agent may bind to an endogenous CD5L:p40 heterodimer, CD5L: CD5L homodimer and/or CD5L monomer. The antagonistic agent may bind to a recombinant soluble CD5L:p40 heterodimer, CD5L: CD5L homodimer and/or CD5L monomer.

The invention also relates to a cell line producing an antagonistic antibody or an antagonistic antigen-binding portion, fragment or equivalent thereof as described herein. The cell line may be a hybridoma. The cell line may be a transfectoma.

The invention also relates to a nucleic acid molecule encoding an antagonistic antibody or an antagonistic antigen-binding portion, fragment or equivalent thereof as described above and herein.

The invention also relates to any of the methods of screening for an antagonistic agent as described herein, such as an agonistic antibody or an antagonistic antigen-binding portion, fragment or equivalent thereof, wherein the agonistic agent may possess any of the functional characteristics described above and herein.

Any of the antagonistic agents described herein may possess any of the functional characteristics as described above.

With regard to any of the functional characteristics described above, a “control” may be the absence of the heterodimer, homodimer, monomer or agonist as appropriate.

Unless otherwise defined, all technical and scientific terms used herein have the same meaning as commonly understood by one of ordinary skill in the art to which this invention belongs. Nonlimiting methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Soluble CD5L can regulate T cell function, largely reversing CD5L deficiency-induced gene expression pattern in T cells. WT or CD5L−/− naïve T cells were sorted and activated under Th0 condition and treated with either PBS or soluble CD5L (50 nM). RNA was extracted at 96h and analyzed using nanostring platform using Th17 codesets of 312 genes (only those showing a difference between any of the tested conditions were included in further analysis).

FIG. 2—Soluble CD5L (CD5Lm) and CD5L/p40 premix can have unique functions on T cells. Similar to FIG. 1, Th0 cells were incubated with soluble CD5L, CD5L/p40 mixture (premixed for 4 hours), p40 or control PBS.

FIG. 3A-C—The impact of soluble CD5L or CD5L/p40 can be dependent on IL-23R expression. CD5L−/− or CD5L−/− IL-23R−/− Th0 cells were incubated with soluble CD5L, CD5L/p40 mixture (premixed for 4 hours), p40 or control PBS.

FIG. 4A-G—CD5L regulates ILC function at steady state and during inflammation. A-D) Naïve 6-month old mice that are either wildtype or CD5L−/− were sacrificed and cells from tissues as indicated are analyzed by flow cytometry or quantitative real time PCR. (A) IL-23R.GFP+/− reporter mice that are otherwise wildtype or CD5L−/− were used and cells were stained directly ex vivo; (B-C) Cells were incubated with IL-7 or IL-7/CD5L overnight and restimulated with PMA/ionomycine in the presence of brefaldin A for four hours. Cells were subsequently stained and analyzed by flow cytometry; (D) Cells were analyzed directly ex vivo by flow cytometry or sorted, RNA-extracted and analyzed by real time qPCR; E-G. 6-8 wk old WT or CD5L−/− IL-17CreRosa26Td-tomato mice were treated with 2.5% DSS in drinking water for 6 days followed by 5 days of regular water. Mice were then sacrificed and cells isolated from respective tissues for PMA/ionomycine restimulation and flow cytometry analysis. FIG. 4F shows that, using a DSS-induced acute colitis model, a similar percent of Rosa26⁺ ILC comparing 8-wk old WT.Il17a^{Cre}Rosa26^{Td-tomato} and Cd51^{-/-} Il17a^{Cre}Rosa26^{Td-tomato} mice at day 11 since DSS treatment. FIG. 4E shows that the percent of ILC that expresses Rorgt is not significantly altered. FIG. 4G shows that ILC from WT.Il17a^{Cre}Rosa26^{Td-tomato} make little IL-17 and turned on IL-10 expression in striking contrast to those from Cd5^{-/-}Il17a^{Cre}Rosa26^{Td-tomato} mice, which continue to produce much higher expression of IL-17 and are IL-10 negative.

FIG. 5—CD5L and CD5L:p40 regulate CD11c+DC function. CD11c+ cells were enriched and sorted from spleen of WT, CD36−/− and IL-23R−/− naïve mice. CD11c+ cells were stimulated with 100 ng/ml LPS in the presence of either control, sCD5L, p40 or CD5L:p40 at 5 uM. Cells were harvested at 24 hours.

FIG. 6A-D—CD5L−/− mice have more severe colitis in response to DSS-induced injury. 6-8 wk old WT or CD5L−/− mice were treated with 2.5% DSS in drinking water for 7 days followed by 7 days of regular water. Weight (A), colitis score (B) and colon length (C) and representative histology (D) were shown.

FIG. 7A-C—Recombinant CD5L can bind to Th1 and Th17p (pathogenic Th17) cells and alleviate diseases severity of EAE and DSS induced colitis. Recombinant CD5L was generated with a His tag. A) Th0, Th1 (IL-12) and Th17p (IL-1b, IL-6, IL-23) are differentiated from naïve CD4 T cells in vitro for 4 days and cells were harvested for staining with recombinant CD5L followed by anti-His APC antibodies and flow cytometry analysis. B) Wildtype (WT) mice were immunized with MOG/CFA followed by PT injection to induce EAE. Mice at peak of disease (score=3) were injected with either PBS (solid circles) or recombinant CD5L (empty circles, CD5Lm) intraperitoneally daily for five consecutive days and mice were followed for disease progression. C) WT mice were induced with colitis with 2.5% DSS in drinking water for a consecutive of 6 days followed by normal water for 8 days. Mice were given either control (PBS) or recombinant CD5L (CD5Lm) intraperitoneally on day 4, 6 and 8. Colon length and colitis score are recorded on day 14.

FIG. 8A-B—A) Recombinant CD5L and CD5L:p40 (genetically linked) were custom ordered from Biolegend. CD5L monomer formed a homodimer and CD5L:CD5L homodimer, which was further purified and was used in subsequent experiments to test its function separately; B) Serum was collected kinetically from WT and Cd51−/− mice with DSS-induced colitis (2% DSS in drinking water for 6 days followed by 7 days of normal water) and the level of CD5L:p40 was measured using an ELISA developed in house using anti-p40 antibody for capturing, biotinylated anti-CD5L antibody for detection and recombinant CD5L:p40 as a positive control.

FIG. 9A-B—FIG. 9A sets forth results of a screening assay showing that TLR ligands can induce secretion of CD5L:p40. FIG. 9B sets forth flow cytometry experiments showing that IL-27 induces expression of CD5L.

FIG. 10A-D—FIG. 10A sets forth results of FACS experiments showing that CD5L homodimers and CD5L:p40 heterodimers inhibit IL-17 expression in pathogenic Th17 cells; FIG. 10(B) shows results of an serum ELISA measurements showing that both forms of CD5L inhibit IL-17 expression; FIGS. 10C and D show cell signatures for pathogenic Th17 cells treated with CD5L homodimers and CD5L:p40 heterodimers, respectively.

FIG. 11A-C—FIG. 11A shows inhibited IL-27 expression in pathogenic Th17 cells treated with CD5L homodimers and CD5L:p40 heterodimers, as measured by ELISA and qPCR; FIG. 11B shows that IFNg expression in Th1 cells is inhibited by CD5L:CD5L homodimer and CD5L:p40 heterodimer, as measured by flow cytometry analysis. FIG. 11C shows reversal of the effect in FIG. 11A in an IL12rb1 knockout demonstrating that the effects of CD5L:p40 heterodimer and CD5L:CD5L homodimer on Th17 cells are IL12rb1 dependent.

FIG. 12A-B—FIGS. 12A and B show heat maps and GSEA analysis for Th17 cells and Th1 cells, respectively, following treatment with CD5L homodimers and CD5L:p40 heterodimers.

FIG. 13A-B—FIG. 13A compares EAE disease severity measurements in wildtype mice and CD5L knockout mice; FIG. 13B compares CD5L expression levels in Th17 and macrophage cells in the spleen and CNS.

FIG. 14A-B—FIG. 14A shows a construct used to generate CD5L conditional knockout mice; FIG. 14B shows that mice CD5L deletion mice were produced in myeloid lineage cells, T cells, and IL-17 producing cells.

FIG. 15A-B—FIG. 15A sets forth a plot demonstrating tumor growth in CD5Lflox/floxLymzCre+ mice injected with colon carcinoma; FIG. 15B sets forth pictures showing tumor size in CD5Lflox/flox mice and CD5L knockout mice 19 days after tumor injection.

FIG. 16—FIG. 16 depicts the lipodome of wildtype and CD5L−/− Th17 cells differentiated under pathogenic and non-pathogenic conditions.

FIG. 17—FIG. 17 is a plot showing that metabolic transcriptome expression covaries with Th17 cell pathogenicity.

FIG. 18A-D—FIG. 18 sets forth plots showing suppression of tumor progression in CD5L−/− mice injected with MC38 (FIG. 19A) and MC38-OVA (FIG. 19B) colon carcinoma; FIGS. 18C and D set forth flow cytometry diagrams assessing tumor infiltrating lymphocytes and cytokines, respectively, in CD5L−/− mice and control mice.

FIG. 19A-B—FIG. 19 sets forth graphs showing CD5L:CD5L homodimer expression (FIG. 19A) and CD5L:p40 heterodimer expression (FIG. 19B) in serum during tumor progression, as measured using ELISA assays.

FIG. 20—FIG. 20 sets forth a heat map showing differentially expressed genes in CD5L:CD5L and CD5L:p40 experiments as compared to the control (differentially expressed genes are defined by p<0.5 as compared to control).

FIG. 21A-B—FIGS. 21A-B set forth data showing the impact of CD5L:p40 and CD5L:CD5L on Tregs in vivo in DSS-induced colitis; FIG. 21A shows frequency of Foxp3+CD4 T cells in cells from mesenteric lymph node (mLN), peyer's patches (pp), lamina propria of colon (LP), and intraepithelial lymphocytes (IEL); FIG. 21B sets forth data showing that CD5L:p40 decreased ILC3 in lamina propria cells but that there was an increase of % total ILC cells in the gut.

FIG. 22A-B—FIG. 22A sets forth data showing serum concentrations of CD5L:p40 and CD5L:CD5L in mice immunized with CD5L:p40 and CD5L:CD5L, respectively; FIG. 22B sets forth data showing pools of antibodies specific to either CD5L:p40 or CD5L:CD5L, and which were obtained from mice immunized with CD5L:p40 and CD5L:CD5L, respectively.

FIG. 23A-D—FIG. 23 demonstrates homology between mice and human protein sequences for CD5L (FIG. 23A (SEQ ID NOS 3 and 4, respectively, in order of appearance)), p19 (FIG. 23B (SEQ ID NOS 5 and 6, respectively, in order of appearance)), p40 (FIG. 23C (SEQ ID NOS 7 and 1, respectively, in order of appearance)), and p35 (FIG. 23D (SEQ ID NOS 8 and 9, respectively, in order of appearance)).

FIG. 24 A-C—FIG. 24A demonstrates that CD5L expression in vivo Th17 cells (Th17), innate lymphoid cells (ILC), γΔ T cells (TCRgd), myeloid cells (CD11c+ and F4/80+) but not in IL17− T cells isolated from the intestines of wildtype mouse and a lack of CD5L expression in myeloid cells (F4/80+) from a CD5L knockout mouse. FIG. 24B depicts data from an EAE mouse model showing high CD5L expression in IL17+ cells but not IL17− cells in the spleen or IL17+ or IL17− cells in the CNS. FIG. 24C shows CD5L expression in various in vivo tumoral cells and in vitro tumor cell lines.

FIG. 25—FIG. 25 shows that while administration of soluble CD5L monomer and CD5L:CD5L homodimer to cell populations also comprising dendritic cells and Th0 or Th17 cells, CD5L:p40 heterodimer demonstrated a regulatory effect on dendritic cells. Not to be bound by theory, it is believed that CD5L:p40 heterodimer may have a regulatory mechanism that is unique relative to CD5L monomer and CD5L:CD5L homodimer.

FIG. 26A-B—FIG. 26A shows the results of an assay carried out along the lines of FIG. 7 to assess CD5L:p40 heterodimer binding to Th17, Th1, and naive T cells (Th0) in IL-23r, il12rb1, il12rb2, and CD36 knockout mice. The results demonstrate that CD5L:p40 binding to Th17 and Th1 cells depends on IL-23r, il12rb1, il12rb2 but not CD36. FIG. 26B shows the results of an assay carried out along the lines of FIG. 7 to assess CD5L:CD5L homodimer binding to Th17, Th1, and naive T cells (Th0) in IL-23r, il12rb1, il12rb2, and CD36 knockout mice. The results demonstrate that CD5L:CD5L binding to Th17 cells depends on IL-23r, il12rb1, il12rb2 but not CD36.

FIG. 27—FIG. 27 shows a FACs plot and a dot plot with each dot representing a TIL, both demonstrating that CD5L deficiency promotes antigen specific CD8 T cell frequencies.

FIG. 28A-B—FIG. 28A shows the percentage of CD8 cells expressing IL-12, TNFa, IFNg, and IL-10 in CD5L flox/flox and CD5L conditional knockout mice, with and without Bre/Mon (control). Where CD5L is conditionally silenced, CD8 function was promoted. FIG. 28B shows the percentage of CD4 cells positive for IL-12, TNFa, IFNg, and IL-10 in CD5L flox/flox and CD5L conditional knockout mice, with and without Bre/Mon (control). Where CD5L is conditionally silenced, CD4 function was promoted.

FIG. 29—FIG. 29 shows the percentage of MDSC and CD11C+ cells and those expressing TNFα in CD5L flox/flox and CD5L conditional knockout mice sitmulated with LPS. Where CD5L is conditionally silenced, CD8 function was promoted.

FIG. 30A-B—FIG. 30A shows the optical density results for an ELISA performed with CD5L, CD5L:p40, p40:p40, CD5L:CD5L, IL-12, and IL-23 (0.5 micrograms/mL of protein) for antibodies from the listed cell lines; the selected antibodies are CD5L:p40 specific.

FIG. 30B shows the results for the same assay, where the selected antibodies are CD5L and/or CD5L:CD5L specific.

FIG. 31—FIG. 31 shows mRNA expression levels for CD5L, p35, p40, and p19 in bone marrow derived dendritic cells/macrophages.

FIG. 32A-C—FIG. 32A shows CD5L alterations in a variety of human tumors from the Cancer Genome Atlas (“TCGA”) and/or other sources. FIG. 32B shows the result of RNA sequencing in human tumors (TCGA). CD5L is highly expressed in the listed tumors from adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), and liver cancer. FIG. 32C shows the association between CD5L mutation and overall survival in patients with liver hepatocellular carcinoma.

FIG. 33A-B—FIG. 33A depicts the results a binding assay similar to FIGS. 30A and B using CD5L:CD5L and CD5L:p40 to select CD5L:p40 specific antibodies. FIG. 33B is a functional readout showing the impact of various CD5L:p40 antibodies on IFN-γ production in Th1 cells.

FIG. 34—FIG. 34 shows the optical density results of an ELISA performed for IL-17 (in Th17 cells) and IFNg (in Th1 cells) production.

FIG. 35—FIG. 35 shows that the therapeutic effects of CD5L:p40 heterodimer in DSS colitis and EAE. At day −1, wildtype (WT) mice were injected intravenously with 10,000 naïve 2D2 CD4 T cells for analysis of antigen specific cells. WT mice were immunized with MOG/CFA followed by PT injection to induce EAE. Mice at onset of disease (score=1) were injected with either PBS (solid circles) or recombinant CD5L:p40 (triangles), or CD5L (rectangles) intraperitoneally daily for six consecutive days and mice were followed for disease progression. FIG. 35A shows that CD5L:p40 heterodimer alleviates established neuroinflammation in the EAE model. WT mice were also induced with colitis with 2% DSS in drinking water for a consecutive of 7 days followed by normal water. Mice were given either control (PBS) or recombinant CD5L:p40 (triangles), CD5L (squares) or CD5L:CD5L homodimer (triangles) intraperitoneally on day 4, 6 and 8. FIG. 35B shows that CD5L:p40 heterodimer alleviates acute colitis. FIG. 35C shows cell analysis of antigen specific cells on day 23 of the experiment in FIG. 35A. Va3.2 is used as a surrogate to track 2D2 antigen-specific cells transferred. FIG. 35D shows cell analysis on day 9 of mice from experiment described in FIG. 35B. L=CD5L; L:L=CD5L:CD5L; L:4=CD5L:p40.

FIG. 36—FIG. 36A-E shows that CD5L:p40 induces unique signature genes on pathogenic Th17 cell transcriptome as compared to CD5L monomer, CD5L:CD5L homodimer and p40:p40 homodimer. Pathogenic Th17 cells were differentiated from naïve CD4 T cells (CD44lowCD62L+CD25-CD4+) from wildtype mice with IL-1b, IL-6 and IL-23 in the presence of control, CD5L monomer (L), CD5L homodimer (L:L), CD5L:p40 heterodimer (L:4) or p40:p40 homodimer (4:4) for 48 hours. RNA were extracted and analyzed by NextSeq for DE genes comparing each treatment with control is shown here in the binary plot (FIG. 36A). Volcano plots showing DE genes from L:4, L:L, L, and 4:4 treatment are shown in FIG. 36B-E.

FIG. 37—FIG. 37 shows that CD5L and p40 can be secreted as heterodimer. Two constructs containing either CD5L or p40 are used to cotransfact 293T cells. Flow cytometry (A) or ELISA (B) are used to assess CD5L and p40 expression intracellularly in cell (A) or in supernatant (B). Golgi stop and Golgi plug were used in (A) for 4 hours prior to harvesting cells for staining and flow cytometry. FIG. 37A shows that cells that stained positive for CD5L also stained positive for p40. FIG. 37B shows immunoprecipitation of CD5L and p40.

FIG. 38—FIG. 38 shows generation of CD5L and p40 mutant constructs. FIG. 38A shows wild-type CD5L and CD5L mutants. CD5L.Mul is a CD5L mutant with the SRCR I domain truncated, thus contains amino acid 128-352 of the wild type CD5L. CD5L.Mu2 is a CD5L mutant with the SRCRII domain truncated, and with the SRCRI domain (amino acid 23-140) directly joined to the SRCRIII domain (amino acid 241-352). CD5L.Mu3 is a CD5L mutant with the SRCRIII domain truncated, and contains amino acid 23-241 of the wild type CD5L. FIG. 38B shows wild-type p40 and p40 mutants. p40.D2D3 is a p40 mutant with D1 domain truncated, and contains amino acid 105-335 of the wild type p40. p40.D1D3 is a p40 mutant with D2 domain truncated and with the D1 domain (amino acid 1-109) directly joined to the D3 domain (amino acid 232-335). p40.D1D2 is a p40 mutant with the D3 domain truncated and contains amino acid 1-232 of the wild type p40. p40.D316E is a p40 mutant with a single amino acid substitution D316E. p40.Y318A is a p40 mutant with a single amino acid substitution Y318A.

FIG. 39—FIG. 39 shows that the therapeutic effects of CD5L:p40 heterodimer in DSS colitis and EAE. WT mice were induced with colitis with 2% DSS in drinking water for a consecutive of 7 days followed by normal water. Mice were given either control (PBS) or recombinant CD5L:p40 (down triangle), CD5L (closed circle) or CD5L:CD5L homodimer (up triangle) intraperitoneally on day 4, 6 and 8. FIG. 39A shows that CD5L:p40 heterodimer alleviates acute colitis, as shown by less weight loss and longer colon length. L=CD5L; L:L=CD5L:CD5L; L:4=CD5L:p40.

FIG. 40—FIG. 40 shows that myeloid cells are the major generator of CD5L:p40 heterodimer in DSS colitis in vivo and conditional deletion of CD5L in myeloid cells (Lyz2cre) resulted in more severe weight loss and shorter colon length in acute colitis model. Wild type, CD5L^{fl/+}Lyz2^{Mu/+}, CD5L^{fl/fl}Lyz2^{mu/+} and CD5L^{-/-} are induced with colitis by adding 2% DDS in drinking water for 7 days followed by 7 days of water. Plasma of respective mice were collected on day 12 and analyzed by sandwich ELISA using anti-IL-12b as coating antibody and bio-anti-CD5L as detection antibody. Recombinant CD5L:p40 was used as standard. Colon length was measured on day 12. FIG. 40A shows that mice with CD5L knockout in myeloid cells (CD5L^{fl/fl}Lyz2^{mu/+}) have lower body weight and shorter colon length compared to the wild type mice. FIG. 40B shows that IL-12 and IL-23 expression level in serum of CD5L knockout mice are higher compared to wild-type mice.

FIG. 41—FIG. 41A shows that CD5L:P40, but not CD5L monomer or CD5L:CD5L homodimer can rescue CD5L deficiency in myeloid cells in female mice undergoing DSS-colitis. No rescue was observed in male mice that are CD5L global knockout. WT, CD5L^{fl/+}Lyz2^{Mu/+} and CD5L^{fl/fl}Lyz2^{Mu/+} mice are induced with colitis by adding 2% DSS in drinking water for 7 days followed by 7 days of normal water. 1 pmol/g of recombinant CD5L, CD5L:CD5L homodimer or CD5L:p40 heterodimer were injected intraperitoneally on day 7,9 and 11. FIG. 41B shows that recombinant CD5L:p40 promoted MCP-1 during recovery phase of DSS-colitis. Splenocytes from respective mice were isolated from day 12 and incubated ex vivo for 4 hours in the presence of Monensin and Brefeldin A. Supernatent was harvested for analysis of MCP-1. MCP-1 was shown to contribute to gut homeostasis and is important in recruiting M2 macrophase (Takada et al., Journal of Immunology (2010) 184(5):2671-2676). MCP-1 drives TH2 differentiation (Gu et al., Nature (2000) 404 (6776):407-411) and its expressin is significantly correlated with infiltration of tumor-associated macrophase, angiogenesis and poor survival in breast cancer patients (reviewed in Lim et al., Oncotarget (2016) 7(19):28697-710); and Deshmane et al., J. Interferon Cytokine Res. (2009) 29(6):313-326).

FIG. 42—FIG. 42 shows that CD5L:p40 suppresses IFNγ expression from CD8 T cells. Total CD8 T cells were isolated from naïve mice and activated with anti-CD3 (1 μg/ml) and anti-CD28 (2 μg/ml) in the presence of control, CD5L, CD5L:CD5L or CD5L:p40 (140 nM) fro 4 days. Supernatant were analyzed for expression of IFNγ or TNF using legendplex (Biolegend, Calif.).

FIG. 43—FIG. 43 shows that CD5L:p40 has limited direct effect on CD8 T cell proliferation or PD-1 expression. L=CD5L; L:L=CD5L:CD5L; L:4=CD5L:p40.

FIG. 44—FIG. 44 shows that in addition to suppressive effect on IFNγ and IL-17, CD5L:p40 and CD5L suppress IL-12 and IL-23, but not IL-6, IL-1 from BMDC/T cell culture. BMCD were differentiated with GM-CSF from bone marrow of WT and CD5LKO mice for 9 days following standard protocol. CD11c+ live BMDC were sorted and plated at 20,000 cells per well with 100,000 naïve 2D2 cells in the presence of 5 μM MOG peptide. Supernatant were harvested from BMDC-T cell coculture after 3 days and measured for cytokines using Legendplex. L=CD5L; LL=CD5L:CD5L; L4=CD5L:p40; 44=p40:p40.

FIG. 45—FIG. 45 confirms the generation of anti-human CD5L:p40 and CD5L antibodies. Recombinant human CD5L:p40 were prepared with CFA and injected intraperitoneally into CD5L knockout mice. Mice were boosted on day 22, 38 with recombinant human CD5L:p40/IFA and recombinant human CD5L:p40 on day 55. Spleens were then fused to generate hybridoma. Serum titer from immunized and unimmunized mice were tested in ELISA against recombinant protein of mouse CD5L(L), CD5L:CD5L(LL), CD5L:p40(L:4), human CD5L (L), CD5L:p40 (L) and CTLA-4. Serum were taken on day 49 post first immunization.

FIG. 46—FIG. 46 shows that CD5L deficiency in BMDC promoted T cell proliferation and expression of coinhibitory molecules on T cells under tolerogenic condition. BMDC were differentiated with GM-CSF from bone marrow of WT and CD5LKO mice for 9 days following standard protocol. CD11c+ live BMDC were sorted and plated at 20,000 cells per well with 100,000 naïve 2D2 cells (pulsed with CFSE) in the presence of MOG peptide. T cells were analyzed 4 days after coculture by flow cytometry.

FIG. 47—FIG. 47 shows that CD5L deficiency in BMDC promoted IL-2 expression, and decreased IL-10 expression in T cells under tolerogenic condition.

FIG. 48A-G—CD5L and p40 can form a heterodimer. A, B) screen of binders for CD5L; A) heat map; B) representative result from sandwich ELISA; C) Immunoprecipitation. Anti-CD5L antibody or isotype control was used to pull down CD5L complex from supernatant generated from B) and blotted for CD5L and p40 as indicated under both reducing and non-reducing conditions. D) Generation of mutant p40 constructs and analysis of their bindings to CD5L using the same system as in B). Results show that p40.D1D2 fail to bind to CD5L suggest that the Fibronectin domain 2 (D3) is required for CD5L binding. E, F) Generation of recombinant CD5L:p40 (L4) E) sequence (SEQ ID NO 24); F) Coomassie stain of the recombinant CD5L:p40 and CD5L under reducing and non-reducing conditions. G) Schematic showing binding location of p35, p19 and CD5L on p40.

FIG. 49A-H—CD5L:p40 is secreted during inflammation and myeloid cells are a major producer. A, B) Expression (red line) of CD5L:p40 in serum of mice at specified time during disease course as indicated; black lines indicate disease score (A) or weight change (B); C) Secreted total CD5L (left) and CD5L:p40 (right) by Th17 cells differentiated from naïve T cells under TGFb+IL-6 (Th17n) or IL-1b+IL-6+IL-23 (Th17p) conditions. D-G) CD5L:p40 is secreted under certain stimulation conditions by BMDM macrophage. D, E) mRNA expression of CD5L and p40 under specific conditions; F, G) ELISA detection of total CD5L or CD5L:p40 heterodimer. H) Myeloid cells are a major producer of CD5L:p40 during DSS colitis. Detection of CD5L:p40 using sandwich ELISA from serum of respective mice during DSS colitis.

FIG. 50A-B—Expression of p19 and p35 in myeloid cells and their regulation by Cd51. A) screen of CD5L:p40 secretion from BMDC/BMM mixed cultures stimulated by different TLR ligands; B) qPCR of mRNA extracted from BMDM cells from CD5L+/− and CD5L−/− mice.

FIG. 51A-B—Generation and validation of conditional CD5L knockout mice in myeloid cells A) construct used to generate CD5L conditional ready mice (CD5Lcw−/−). CD5Lflox/flox mice were generated by breeding the CD5L conditional ready mice to Flp recombinase transgenic mice; B) Validation of CD5Lcw−/− as a total knockout mice and CD5Lfl/flLyz2cre mice as conditional knockout mice. Upper left panel: BMM generated from bone marrow cells using M-CSF from respective mice; Lower left panel: Th17n cells differentiated from naïve T cells under TGFb+IL-6 condition; right panel, summary of upper left panel.

FIG. 52A-D—Recombinant CD5L:p40 alters antigen specific responses. Wildtype B6 mice (A,C) were immunized with MOG/CFA and recombinant CD5L:p40 were given at 1 pmol/g of body weight on day 2, 4 and 7 post immunization by intraperitoneal injection. A) Representative analysis of cytokine production from antigen-specific T cells from a similar experiment where naïve 2D2 T cells were transferred 2 days prior to immunization; B) Ex vivo MOG recall response: cytokines are measured by legendplex from supernatant of cells isolated from inguinal/draining lymph node in response to MOG peptide for 3 days; C) Thymidine incorporation assay from same condition as in B). D) CD5L+/− or CD5L−/− mice were immunized by MOG/CFA and inguinal lymph nodes were isolated for MOG recall assay in the presence of control or recombinant CD5L:p40 followed by thymidine incorporation assay as in C.

FIG. 53—Recombinant CD5L:p40 suppresses IFNg production but promotes Th2 cytokines from Th1 cells in vitro. Naïve T cells were differentiated under Th1 condition in the presence of different dose of CD5L:p40. IFNg, IL-4, IL-5 and IL-13 are measured using legendplex using a flow-based assay on day 3 of T cell culture.

FIG. 54—Recombinant CD5L:p40 effect on Th17 cells. CD5L−/− and CD5L+/− Th17 cells in the presence of different doses of CD5L:p40.

FIG. 55A-D—Recombinant CD5L:p40 suppresses Th17 responses and promotes type 2 responses directly in vitro. A) Naïve T cells were differentiated under pathogenic Th17 condition (IL-1b+IL-6+IL-23) in the presence of either control (BSA) or CD5L:p40 (L4). Flow cytometry analysis of intracellular IL-17 production from 6 biological replicates are shown. B) Th17p cells were differentiated as in A), and are further expanded in IL-23 without addition of other cytokines (e.g. L4). Flow cytometry analysis of intracellular IL-17 production from 6 biological replicates are shown. C) Cytokine secretion detected in supernatant of Th17p differentiation culture as in A). The bracket indicating either 1:10, 1:100 or 1:400 dilution correspond to the concentration of IL-1b, IL-6 and IL-23 used. Original concentration is 20 ng/ml of IL-1b, IL-6 and IL-23. D) qPCR result of mRNA isolated from Th17p cells as in A).

FIG. 56A-F—Recombinant CD5L:p40 can bind to Th17 cells directly and alters T cell signaling pathways and metabolism. A) recombinant CD5L:p40 (his-tagged) were used to stain pathogenic Th17 cells (IL-1b+IL-6+IL-23),Th1 cells (IL-12) or Th0 cells differentiated from naïve T cells isolated from wildtype or Il12rb1KO mice followed by anti-His antibodies and analysis by flow cytometry. B-D) As Stat3 and Stat4 regulate IL-17 responses, we analyzed pStat3 and pStat4 expression. B-C) CD5L:p40 suppresses pStat3. 28 nM recombinant CD5L:p40 were used to stimulate naïve T cells in the presence of 10 ug/ml of anti-CD3 antibodies (TCR) with or without other indicated cytokines (B) or to stimulate Th17p cells (C) and cells were harvested for phosphoflow preparation and analysis of pStat3 at the indicated time points. D) CD5L:p40 suppresses pStat4 but not pTyk2. Western blotting analysis of pStat4 and pTyk2 expression by Th17p cells (equal protein loaded per lane) differentiated from naïve T cells isolated from wildtype mice and subjected to stimulation by either control (C), CD5L monomer (L), CD5L homodimer (LL), CD5L:p40 (L4) or p40:p40 homodimer (44) for the indicated time. E) CD5L:p40 suppresses pRictorY, pS6 and p38. To study whether CD5L:p40 influence other signaling pathways, we analysed several other phospho-proteins as indicated. Th17 cells were differentiated from naive under either IL-lb+IL-6+IL-23 or IL-1b+IL-6 conditions for 6 hours (left panels) or 24 hours (right panels) and are stimulated by either by BSA (C), CD5L monomer (L), CD5L homodimer (LL), CD5L:p40 (L4) or p40:p40 homodimer (44) for the indicated time. Equal protein were loaded per lane. F) CD5L:p40 alters T cell metabolism in response to glutamate. Seahorse assay were performed on Th17p cells (differentiated under IL-1b+IL-6+IL-23) or Th1 cells (IL-12) in the presence of either control (C), CD5L monomer (L), CD5L homodimer (LL), CD5L:p40 (L4) or p40:p40 homodimer (44) for 3 days and cells were harvested for seahorse assay in media containing only L-gluatmate.

FIG. 57A-B—Effect of CD5L:p40 on pStat3 as compared to other related cytokines. A and B correspond to the same experiment as shown in FIGS. 56B and 56C respectively.

FIG. 58A-F—Th17p cells treated with CD5L:p40 showed reduced pathogenicity in vivo in transfer EAE model. Th17p cells were differentiated (IL-1b+IL-6+IL-23) in the presence of either BSA or CD5L:p40 from naïve T cells isolated from 2D2 transgenic mice. Th17 cells were then transferred into wildtype host and mice were followed for EAE clinical scores and CNS infiltrating cells and splenocytes were analyzed for cell surface markers and cytokine production. A-C) flow cytometry or legendplex analysis of CNS or spleen infiltrating cells. A) number of CNS-infiltrating antigen-specific CD4 T cells is reduced in mice transferred with L4 treated Th17 cells; B) coinhibitory receptor expression on antigen-specific CD4 T cells is enhanced in mice with L4 treated Th17 cell transfer; C) frequency of induced antigen-specific Treg cells is unchanged; D-E) flow cytometry or legendplex analysis of CNS or spleen infiltrating cells. D, E) Antigen-specific T cells make more IL-10 and less IFNg (D, flow cytometry) and make more type2 cytokines in response to antigen (E, legendplex analysis of supernatant from CNS lymphocytes restimulated with MOG peptide or control for 3 days). F) EAE score.

FIG. 59A-D—Recombinant CD5L:p40 induces a unique transcriptome in Th17 cells. A) Heatmap showing differentially expressed genes in Th17 cells treated with control, CD5L, CD5L:p40, CD5L:CD5L and p40:p40. B) Schematic showing significant differentially expressed genes of Th17 cells treated with CD5L, CD5L:p40, CD5L:CD5L and p40:p40. C) Plot showing differentially expressed genes after treating Th17 cells with CD5L:p40. D) Graphs showing the relative expression of Il17f, Il17, Dusp2 and Rxra after the indicated treatments.

FIG. 60A-C—Dusp2 is a downstream signaling molecule of CD5L:p40 and deleting Dusp2 rescues the effect of rCD5L:p40. A) Experimental scheme; B-C) Dusp2 is deleted using CRISPR/Cas9 system and the effect of CD5L:p40 on Th17 cells is re-evaluated under control or Dusp2 deletion conditions.

FIG. 61—Generation of anti-human-CD5L:p40 antibody. ELISA is shown using antibody clones specific for human CD5L:p40, CD5L and p40.

FIG. 62A-B—The effect of CD5L:p40 on Th17p does not depend on CD36, but is dependent on IL-12RB1. A-B) Heatmaps showing gene expression on Th17 cells treated with control or CD5L:p40 in either wildtype cells, CD36−/− cells or Il12rb1−/− cells.

FIG. 63—Screening of cell lines that bind to recombinant CD5L:p40. Cell lines were first screened through expression of potential receptor subunits such as Il112rb and then used for testing binding to HIS-tagged CD5L:p40. Anti-his APC antibody is used as secondary and cells were analyzed using flow cytometry.

FIG. 64—CD5L deficiency has additive or synergistic effect with PD-1 blockade in mice implanted with B16-F10 melanoma. Control or CD5L−/− mice were implanted with B16-F10 melanoma subcutaneously. PD-1 blocking antibody (RMP1-14) or isotype control antibodies were given intraperitoneally to control or CD5L−/− mice at 200 ug/mice on day 5, 8 and 11. Whereas PD-1 blockade or CD5L deficiency alone did not show significant effect on b16 tumor growth under the tested condition, combining PD-1 blockade and CD5L deficiency resulted in enhance tumor control.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International publication numbers WO2016138488, WO2015130968 and WO2014134351.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide antagonists of CD5L, specifically antagonists of CD5L monomers, CD5L:CD5L homodimers, and CD5L:p40 heterodimers.

As disclosed in PCT/US2016/062592, published as WO2017087708, IL-23 is formed of a heterodimer by p19 and p40. p40, also known as interleukin 12B, can form heterodimers with two other cytokines: p35 to make IL-12 and CD5 Antigen Like protein (CD5L) (also known as apoptosis inhibitor of macrophage (AIM), SP-a, and Api6) to make CD5L:p40. It has not previously been demonstrated that the CD5L:p40 dimer has any function. Th17-cell intrinsic CD5L can regulate Th17 cell pathogenicity and regulate IL-23R expression (see WO2015130968). CD5L is a secreted protein and it may form a heterodimer with p40 (Abdi et al., 2014). Applicants tested the hypothesis that soluble CD5L, as a monomer, homodimer, or heterodimer with p40, can function as a cytokine regulating T cell function. Surprisingly, Applicants found that soluble CD5L monomer, CD5L:CD5L homodimer, and CD5L:p40 heterodimer share a distinct ability to regulate T cell function. Specifically, Applicants discovered downstream targets upregulated and downregulated specific for each of CD5L monomer, CD5L:CD5L homodimer, CD5L:p40 and p40:p40.

Differentially expressed genes in Th17 cells treated with control, CD5L, CD5L:p40, CD5L:CD5L and p40:p40 that may be downstream targets of each molecule include 1117f, 1117a, Ildr1, Il1r1, Lgr4, Ptpnl4, Paqr8, Timp1, Il1rn, Smim3, Gap43, Tigit, Mmp10, Il22, Enpp2, Iltifb, Ido1, 1123r, Stom, Bcl2l11, 5031414D18Rik, Il24, Itga7, Il6, Epha2, Mt2, Upp1, Snord104, 5730577I03Rik, Slcl8b1, Ptprj, Clip3, Mir5104, Ppifos, Rab13, Hist1h2bn, Ass1, Cd200r1, E130112N10Rik, Mxd4, Casp6, Gatm, Tnfrsf8, Gp49a, Gadd45g, Ccr5, Tgm2, Lilrb4, Ecm1, Arhgap18, Serpinb5, Cysltr1, Enpp1, Selp, Slc38a4, Gm14005, Epb4.1l4b, Moxd1, Klra7, Igfbp4, Tnip3, Gstt1, Pglyrp2, Il12rb2, Ctla2a, Plac8, Ly6c1, Sell, Ncf1, Trp53i11, B3gnt3, Kremen2, Matk, Ltb41, Ets1, Tnfrsf26, Cd28, Rybp, Ppp1r3c, Thy1, Trib2, Sema3b, Pros1, Il33, Gm5483, Myh11, Cntd1, Ms4a4b, Trem12, 3110009E18Rik, Pglyrp1, Amd1, Slc24a5, Snhg9, Ifi27l1, Irf7, Mx1, Snhg10, 114, Snora43, H2-L, Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Dusp2, Snx20, Lamp1, Faf1, Gpatch3, Dapk3, 1110065P20Rik, Vaultrc5, Myl4, Insl3, Tgoln2, BC022687, C230035I16Rik, Hvcn1, Myh10, Dhrs3, Acsl6, Rgs2, Ccl20, Cc13, Dlg2, Ccr6, Cc14, Dusp14, Apo19b, Cd72, Ispd, Cd70, S100al, Lgals3, Slc15a3, Nkg7, Serpinc1, Olfr175-ps1, 119, Pdlim4, Il3, Insl6, Perp, Cd51, Serpine2, Galnt14, Tff1, Ppfibp2, Bdh2, Mlf1, Il1a, Osr2, Gm5779, Ebf1, Spink2, Egfr and Ccdc155. Specific genes upregulated by CD5L:p40 include Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Dusp2, Snx20, Lamp, Faf1, Gpatch3, Dapk3, 1110065P20Rik and Vaultrc5. Applicants identified and characterized Dusp2 as a downstream signaling molecule of CD5L:p40. Applicants show herein that deleting Dusp2 rescues the effect of rCD5L:p40. Dusp2 has previously been reported to control the activity of the transcription activator STAT3 and regulate TH17 differentiation (see, e.g., Lu et al., Nat Immunol. 2015 December; 16(12):1263-73. doi: 10.1038/ni.3278).

Not to be bound by theory, CD5L, either as a monomer, homodimer, or a heterodimer, is suspected to interfere with the pathogenic and non-pathogenic program of Th17 cells. Such findings have therapeutic implications with respect to neuroinflammation, autoimmune disorders, inflammatory cancers, and non-inflammatory cancers and disorders, inter alia.

CD5L function is largely dependent not on CD36, the known receptor for CD5L, but IL-23R expression on T cells. Further, CD5L:p40 appears to be less dependent on IL-23R and may require a different receptor for signaling. Moreover, CD5L can regulate not only T cells, but also other IL-23R expressing cells such as innate lymphoid cells and dendritic cells. CD5L plays a critical role in protecting host from acute inflammation and potentially tumor progression. Applicants have determined for the first time that Il12rb1 is a subunit of a receptor for CD5L:p40. Thus, CD5L can regulate not only T cells, but also other Il12rb1 expressing cells. Not being bound by a theory the IL-12 receptor may be the receptor for CD5L:p40. The findings characterizing CD5L function in vitro and in vivo, including the effects of CD5L proteins on immune cell function as disclosed herein has allowed for the discovery of novel agonists and antagonists of CD5L signaling. Applicants have further discovered novel uses for agonists and antagonists in the treatment of disease. Finally, Applicants have identified an additive or synergistic effect of CD5L deficiency with checkpoint blockade therapy to enhance tumor control.

As used herein, a CD5L agonist includes CD5L monomers, CD5L:CD5L homodimers, and CD5L:p40 heterodimers (including fusion proteins), as well as antibodies or small molecules having agonist activity. As used herein, a CD5L antagonist includes CD5L monomers, CD5L:CD5L homodimers, and CD5L:p40 heterodimers that have been modified (e.g., by mutation) to be antagonistic, as well as antibodies or small molecules having antagonist activity. Agonists or antagonists may be antibodies, proteins, small molecules or nucleic acids that bind to, block or activate Il12rb containing receptors (e.g., IL-12 receptor). Agonists or antagonists may also be genetic modifying agents as described herein. Agonists or antagonists may target any downstream target described herein (e.g., antibody, small molecule, genetic modifying agent).

### CD5L Monomer, CD5L:CD5L Homodimer, and CD5L:p40 Heterodimer

Aspects of this disclosure relate to CD5L monomers, CD5L:CD5L homodimers, and/or CD5L:p40 heterodimers.

The homodimers include CD5L complexed to another CD5L, preferably complexed together in a homodimeric form. The heterodimers include p40 protein and CD5L protein, preferably complexed together in a heterodimeric form. The protein sequences will preferably be chosen based on the species of the recipient; thus, for example, human p40 and/or human CD5L can be used to treat a human subject. The sequences of human p40 and CD5L are as follows:

| Human p40 (interleukin-12 subunit beta) precursor | | | | | | |
|---|---|---|---|---|---|---|
| (SEQ ID NO: 1) | | | | | | |
| 1 | mchqqlvisw | fslvflaspl | vaiwelkkdv | yvveldwypd | apgemvvltc | dtpeedgitw |
| 61 | tldqssevlg | sgktltiqvk | efgdaggytc | hkggevlshs | llllhkkedg | iwstdilkdq |
| 121 | kepknktflr | ceaknysgrf | tcwwlttist | dltfsvkssr | gssdpqgvtc | gaatlsaery |
| 181 | rgdnkeyeys | vecqedsacp | aaeeslpiev | mvdavhklky | enytssffir | diikpdppkn |
| 241 | lqlkplknsr | qvevsweypd | twstphsyfs | ltfcvqvqgk | skrekkdrvf | tdktsatvic |
| 301 | rknasisvra | qdryysssws | ewasvpcs | | | |

In some embodiments, amino acids 23-328 of SEQ ID NO: 1 (leaving off the signal sequence) are used. An exemplary mRNA sequence encoding p40 is accessible in GenBank at No. NM 002187.2.

| CD5 molecule-like (CD5L) | | | | | | |
|---|---|---|---|---|---|---|
| (SEQ ID NO: 2) | | | | | | |
| 1 | mallfslila | ictrpgflas | psgvrlvggl | hrcegrveve | qkgqwgtvcd | dgwdikdvav |
| 61 | lcrelgcgaa | sgtpsgilye | ppaekeqkvl | igsysctgte | dtlagcegee | vydcshdeda |
| 121 | gascenpess | fspvpegvrl | adgpghckgr | vevkhqnqwy | tvcqtgwslr | aakvvcrqlg |
| 181 | cgravltqkr | cnkhaygrkp | iwlsqmscsg | reatlqdcps | gpwgkntcnh | dedtwveced |
| 241 | pfdlrlvggd | nlcsgrlevl | hkgvwgsvcd | dnwgekedqv | vckqlgcgks | lspsfrdrkc |
| 301 | ygpgvgriwl | dnvrcsgeeq | sleqcqhrfw | gfhdcthqed | vavicsg | |

In some embodiments, amino acids 20-347 of SEQ ID NO:2 (leaving off the signal sequence) are used. An exemplary mRNA sequence encoding CD5L is accessible in GenBank at No. NM 005894.2.

### Recombinant Production

Methods for making recombinant proteins are well known in the art, including in vitro translation and expression in a suitable host cell from nucleic acid encoding the variant protein. A number of methods are known in the art for producing proteins. For example, the proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In some embodiments, recombinant p40 and CD5L proteins are obtained and mixed in roughly equimolar amounts of p40 with CD5L and incubated, e.g., at 37° C. Immunoprecipitation and purification can be used to confirm formation of heterodimers, as can size exclusion chromatography or other purification methods, to obtain a substantially pure population of heterodimers. In some embodiments, nucleic acid encoding a p40 or CD5L polypeptides is incorporated into a gene construct that is used to co-transfect cell lines to obtain a substantially pure composition of heterodimers secreted into media. In some embodiments, p40 and CD5L are simply mixed together under conditions sufficient for heterodimerization, and optionally purified to obtain a substantially pure composition of heterodimers; alternatively, the heterodimers can be cross-linked and then purified. In some embodiments, an agent such as TLR9 can be used to increase heterodimer formation, e.g., in vitro or in vivo.

In some embodiments, the methods include administering nucleic acids encoding a p40 and/or CD5L polypeptides or active fragment thereof. In some embodiments, the nucleic acids are incorporated into a gene construct to be used as a part of a gene therapy or cell therapy protocol. In some embodiments, the methods include targeted expression vectors for transfection and expression of polynucleotides that encode p40 and/or CD5L polypeptides, in particular cell types, especially in T cells and myeloid cells such as dendritic cells or macrophage. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized conjugates (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include Psi-Crip, Psi-Cre, Psi-2 and Psi-Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986)).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992)). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993)).

In addition to viral transfer methods, such as those illustrated above, non-viral methods can also be employed to cause expression of a nucleic acid compound (e.g., nucleic acids encoding p40 and/or CD5L polypeptides) in the tissue of a subject. Typically, non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22): 1896-905 (2000); or Tam et al., Gene Ther. 7(21): 1867-74 (2000).

In some embodiments, genes encoding p40 and/or CD5L polypeptides are entrapped in liposomes bearing positive charges on their surface (e.g., lipofectins), which can be tagged with antibodies against cell surface antigens of the target tissue (see, e.g., Mizuno et al., No Shinkei Geka 20:547-551 (1992); PCT publication WO91/06309; Japanese patent application 1047381; and European patent publication EP-A-43075)).

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Agonists

Aspects of the disclosure relate to a CD5L monomer, CD5L:CD5L homodimer, and/or CD5L:p40 heterodimer agonist or and/one or more nucleic acids encoding the same.

Without being bound by theory, CD5L monomers, homodimers and heterodimers with p40 are believed to regulate T cells and alter immune function, and can promote suppression of pathogenic Th17 and Th1 phenotypes and CD8⁺ T cell exhaustion. Additional effects are disclosed in the description of the figures provided above, the examples provided below, and throughout this disclosure.

Agonists of CD5L monomers, CD5L:CD5L homodimers, and/or CD5L:p40 heterodimers are thus contemplated herein as modulators or suppressors of the immune response in a subject.

As used herein, the term “agonist” refers to an agent that activates a target (e.g. CD5L monomer, CD5L:CD5L homodimer, or CD5L:p40 heterodimer) to produce its biological response. In some embodiments, the present invention provides agonist specific for CD5L monomer, which specifically activates CD5L monomer to produce its biological response, and does not activate CD5L:CD5L homodimer or CD5L:p40 heterodimer. In some embodiments, the present invention provides agonist specific for CD5L:CD5L homodimer, which specifically activates CD5L:CD5L homodimer to produce its biological response, and does not activate CD5L monomer, or CD5L:p40 heterodimer. In some embodiments, the present invention provides agonist specific for CD5L:p40 heterodimer, which specifically activates CD5L:p40 heterodimer, and does not activate CD5L monomer, or CD5L homodimer.

A variety of assays are known in the art for demonstrating agonistic effect. For example, any ligand binding assay may be used to determine whether a candidate agent, such as the proteins or polypeptides, antibodies, equivalents, and/or compositions disclosed herein, has an agonistic effect on CD5L. Further, comparative analysis of candidate agents can be performed with an untreated negative control and a soluble target (e.g. CD5L monomer, CD5L:CD5L homodimer, or CD5L:p40 heterodimer) treated positive control according to the methods disclosed in the examples herein below to determine if treatment with a candidate agent recapitulates or enhances the endogenous effects of the target. Suitable methods employing any one of the model CRISPR-Cas systems disclosed herein may also be employed to conduct gain of function or loss of function analysis where appropriate.

In some embodiments, agonistic effect can be determined by assessing the downstream biological effects of the antibody, e.g. the impact on production of one or more cytokines implicated in the CD5L monomer, CD5L:CD5L homodimer, and/or CD5L:p40 heterodimer mediated signal cascade or pathway. It is appreciated that while the results of these types of assays may indicate an agonistic result for some aspects but not others, e.g. an antibody may have agonistic effects with respect to one cytokine but not another.

In some embodiments, the agonist of the present disclosure includes small molecules, peptides, and antibodies that bind to and occupy a binding site of CD5L monomer, CD5L:CD5L homodimer, and/or CD5L:p40 heterodimer, or a binding partner thereof, promoting their normal biological activity or response. Small molecule agonists are usually less than 10K molecular weight, e.g. 100 to about 20,000 daltons (Da), about 500 to about 15,000 Da, about 1000 to about 10,000 Da, and may possess a number of physicochemical and pharmacological properties which enhance cell penetration, resist degradation and prolong their physiological half-lives (Gibbs, J. Pharmaceutical Research in Molecular Oncology, Cell, Vol. 79 (1994)).

The present invention also provides methods for identifying agonists for CD5L monomer, CD5L:CD5L homodimer, and/or CD5L:p40 heterodimer. In one aspect, the invention contemplates screening libraries of small molecules to identify agonists, for example, by high-throughput screening (HTS). “High-throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonably short period of time (e.g., days). Preferably, the process is amenable to automation, such as robotic simultaneous handling of 96 samples, 384 samples, 1536 samples or more. Atypical IHT-IS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology. Thus “high-throughput screening” as used herein does not include handling large quantities of radioactive materials, slow and complicated operator-dependent screening steps, and/or prohibitively expensive reagent costs, etc.

Diverse sets of chemical libraries, containing more than 200,000 unique small molecules, as well as natural product libraries, can be screened. This includes, for example, the Prestwick library (1, 120 chemicals) of off-patent compounds selected for structural diversity, collective coverage of multiple therapeutic areas, and known safety and bioavailability in humans, as well as the NINDS Custom Collection 2 consisting of a 1,040 compound-library of mostly FDA-approved drugs (see, e.g., U.S. Pat. No. 8,557,746) are also contemplated. The NIH's Molecular Libraries Probe Production Centers Network (MLPCN) offers access to thousands of small molecules or chemical compounds that can be used as tools to probe basic biology and advance our understanding of disease. The Broad Institute's Probe Development Center (BIPDeC) is part of the MLPCN and offers access to a growing library of over 330,000 compounds for large scale screening and medicinal chemistry. In some embodiments, agonists can be screened using the NIB Clinical Collections (see, http://www.nihclinicalcoilection.com,”). The Clinical Collection and NIH Clinical Collection 2 are plated arrays of 446 and 281, respectively, small molecules that have a history of use in human clinical trials. In another embodiment collections of FDA approved drugs are assayed. Advantages of these collections are that the clinically tested compounds are highly drug-like with known safety profiles. Any of these compounds may be utilized for screening compounds to identify agonists of the present invention.

Additionally, libraries can be selected, constructed, or designed specifically for an agonist. In some embodiments, agonists can be modified based the structure of the binding site of the CD5L monomer, CD5L:CD5L homodimer, and/or CD5L:p40 heterodimer.

In another aspect, the present invention provides agonists of the CD5L monomer, CD5L:CD5L homodimer, and/or CD5L:p40 heterodimer, which are genetic modifying agents capable of activating as described further herein.

### Antagonists

Aspects of the disclosure relate to a CD5L monomer, CD5L:CD5L homodimer, and/or CD5L:p40 heterodimer antagonist or and/one or more nucleic acids encoding the same.

Without being bound by theory, CD5L monomers, homodimers and heterodimers with p40 are believed to regulate T cells and alter immune function, and can promote suppression of pathogenic Th17 and Th1 phenotypes and CD8⁺ T cell exhaustion. Additional effects are disclosed in the description of the figures provided above, the examples provided below, and throughout this disclosure.

Antagonists of CD5L monomers, CD5L:CD5L homodimers, and/or CD5L:p40 heterodimers are thus contemplated herein as enhancers of the immune response in a subject.

As used herein, the term “antagonist” refers to an agent that inhibits a target (e.g. CD5L monomer, CD5L:CD5L homodimer, or CD5L:p40 heterodimer) from producing its biological response. In some embodiments, the present invention provides antagonist specific for CD5L:CD5L homodimer, which specifically inhibits CD5L:CD5L homodimer from producing its biological response, and does not inhibit CD5L monomer, or CD5L:p40 heterodimer. In some embodiments, the present invention provides antagonist specific for CD5L:p40 heterodimer, which specifically inhibits CD5L:p40 heterodimer, and does not inhibit CD5L monomer, or CD5L homodimer.

A variety of assays are known in the art for demonstrating antagonistic effect. For example, any ligand binding assay may be used to determine whether a candidate agent, such as the proteins or polypeptides, antibodies, equivalents, and/or compositions, has an antagonistic effect on CD5L. Further, comparative analysis of candidate agents can be performed with an untreated negative control and a known inhibitor treated positive control according to the methods in the examples below to determine if treatment with a candidate agent inhibits the endogenous effects of the target. Suitable methods employing any one of the model CRISPR-Cas systems may also be employed to conduct gain of function or loss of function analysis where appropriate.

In some embodiments, antagonistic effect can be determined by assessing the downstream biological effects of the antibody, e.g. the impact on production of one or more cytokines implicated in the CD5L monomer, CD5L:CD5L homodimer, and/or CD5L:p40 heterodimer mediated signal cascade or pathway. It is appreciated that while the results of these types of assays may indicate an antognistic result for some aspects but not others, e.g. an antibody may have antagonistic effects with respect to one cytokine but not another.

In some embodiments, the antagonist of the present disclosure includes small molecules, peptides, and antibodies that bind to and occupy a binding site of CD5L monomer, CD5L:CD5L homodimer, and/or CD5L:p40 heterodimer, or a binding partner thereof, inhibiting their normal biological activity or response. Applicants show that p40.D1D2 fails to bind to CD5L suggesting that the fibronectin domain 2 (D3) is required for CD5L binding. In one embodiment, the antagonist blocks formation of CD5L:p40 heterodimers. In one embodiment, the antagonist blocks CD5L:p40 heterodimer formation by modification of or binding to a fibronectin domain on p40. In certain embodiments, p40 will bind to p19 and p35 when the fibronectin domain is blocked by an antagonist and generate an inflammatory immune state or inhibit a suppressive immune state.

In certain embodiments, the antibodies are directed against the fibronectin domain 2 of p40. In certain embodiments, the antagonistic antibodies bind an epitope in the fibronectin 2 domain. In certain embodiments, antibodies directed to the fibronection domain 2 blocks CD5L binding to p40. In certain embodiments, p40 will bind to p19 and p35 when the fibronectin domain is blocked by an antagonist antibody and generate an inflammatory immune state or inhibit a suppressive immune state. The fibronectin domain may be the fibronectin domain from wildtype p40 as exemplified in SEQ ID NOS 1 and 7.

Small molecule antagonist are usually less than 10K molecular weight, e.g. 100 to about 20,000 daltons (Da), about 500 to about 15,000 Da, about 1000 to about 10,000 Da, and may possess a number of physicochemical and pharmacological properties which enhance cell penetration, resist degradation and prolong their physiological half-lives (Gibbs, J. Pharmaceutical Research in Molecular Oncology, Cell, Vol. 79 (1994)).

The present invention also provides methods for identifying antagonists for CD5L monomer, CD5L:CD5L homodimer, and/or CD5L:p40 heterodimer. In one aspect, the invention contemplates screening libraries of small molecules to identify antagonists, for example, by high-throughput screening (HTS). “High-throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonably short period of time (e.g., days). Preferably, the process is amenable to automation, such as robotic simultaneous handling of 96 samples, 384 samples, 1536 samples or more. Atypical IHT-IS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology. Thus “high-throughput screening” as used herein does not include handling large quantities of radioactive materials, slow and complicated operator-dependent screening steps, and/or prohibitively expensive reagent costs, etc.

Diverse sets of chemical libraries, containing more than 200,000 unique small molecules, as well as natural product libraries, can be screened. This includes, for example, the Prestwick library (1, 120 chemicals) of off-patent compounds selected for structural diversity, collective coverage of multiple therapeutic areas, and known safety and bioavailability in humans, as well as the NINDS Custom Collection 2 consisting of a 1,040 compound-library of mostly FDA-approved drugs (see, e.g., U.S. Pat. No. 8,557,746) are also contemplated. The NIH's Molecular Libraries Probe Production Centers Network (MLPCN) offers access to thousands of small molecules or chemical compounds that can be used as tools to probe basic biology and advance our understanding of disease. The Broad Institute's Probe Development Center (BIPDeC) is part of the MLPCN and offers access to a growing library of over 330,000 compounds for large scale screening and medicinal chemistry. In some embodiments, antagonist can be screened using the NIB Clinical Collections (see, www.nihclinicalcoilection.com,”). The Clinical Collection and NIH Clinical Collection 2 are plated arrays of 446 and 281, respectively, small molecules that have a history of use in human clinical trials. In another embodiment collections of FDA approved drugs are assayed. Advantages of these collections are that the clinically tested compounds are highly drug-like with known safety profiles. Any of these compounds may be utilized for screening compounds to identify antagonists of the present invention.

Additionally, libraries can be selected, constructed, or designed specifically for an antagonist. In some embodiments, antagonists can be modified based the structure of the binding site of the CD5L monomer, CD5L:CD5L homodimer, and/or CD5L:p40 heterodimer.

In another aspect, the present invention provides aptamers antagonists of the CD5L monomer, CD5L:CD5L homodimer, and/or CD5L:p40 heterodimer. Aptamers are usually created by selection of a large random sequence pool, but natural aptamers also exist. Inhibition of the target molecule by an aptamer may occur by binding to the target, by catalytically altering the target, by reacting with the target in a way that modifies/alters the target or the functional activity of the target, by covalently attaching to the target as a suicide inhibitor, by facilitating the reaction between the target and another inhibitory molecule. Oligonucleotide aptamers may be comprised of multiple ribonucleotide units, deoxyribonucleotide units, or a mixture of those units. Oligonucleotide aptamers may further comprise one or more modified bases, sugars, phosphate backbone units. Peptide aptamers are small, highly stable proteins that provide a high affinity binding surface for a specific target protein. They usually consist of a protein scaffold with variable peptide loops attached at both ends. The variable loop is typically composed of ten to twenty amino acids, and the scaffold can be any protein that has good solubility and compacity properties. This double structural constraint greatly increases the binding affinity of the peptide aptamer to its target protein. Aptamers can be designed to target the CD5L monomer, CD5L:CD5L homodimer, and/or CD5L:p40 heterodimer.

In another aspect, the present invention provides antagonists of the CD5L monomer, CD5L:CD5L homodimer, and/or CD5L:p40 heterodimer, which are anti-sense oligonucleotides. Antisense oligonucleotides can be DNA, RNA, a DNA-RNA chimera, or a derivative thereof. Upon hybridizing with complementary bases in an RNA or DNA molecule of interest, antisense oligonucleotides can interfere with the transcription or translation of the target gene, e.g., by inhibiting or enhancing mRNA transcription, mRNA splicing, mRNA transport, or mRNA translation or by decreasing mRNA stability. As presently used, “antisense” broadly includes RNA-RNA interactions, RNA-DNA interactions, and RNaseH mediated arrest. Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607).

In another aspect, the present invention provides antagonists of the CD5L monomer, CD5L:CD5L homodimer, and/or CD5L:p40 heterodimer, which are RNAi agents. A “RNAi agent” can be an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target gene, and which mediates the targeted cleavage of another RNA transcript via an RNA-induced silencing complex (RISC) pathway. In some embodiments, the RNAi agent is an oligonucleotide composition that activates the RISC complex/pathway. In some embodiments, the RNAi agent comprises an antisense strand sequence (antisense oligonucleotide). In some embodiments, the RNAi comprises a single strand. This single-stranded RNAi agent oligonucleotide or polynucleotide can comprise the sense or antisense strand, as described by Sioud 2005 J. Mol. Bio. 348: 1079-1090, and references therein. Thus the disclosure encompasses RNAi agents with a single strand comprising either the sense or the antisense strand of an RNAi agent described herein. The use of the RNAi agent to a target gene results in a decrease of target activity, level and/or expression, e.g., a “knockdown” or “knock-out” of the target gene or target sequence.

In another aspect, the present invention provides antagonists of the CD5L monomer, CD5L:CD5L homodimer, and/or CD5L:p40 heterodimer, which are genetic modifying agents as described further herein.

### Antibodies

Some aspects provide an isolated or substantially purified antibody or antigen binding fragment which may be capable of specific binding to a CD5L monomer, a CD5L:CD5L homodimer, and/or a CD5L:p40 heterodimer. Such antibodies or antigen-binding fragments or derivatives thereof may be in the form of a polyclonal antibody, a monoclonal antibody, a chimeric antibody, a human antibody, a veneered antibody, a diabody, a humanized antibody, an antibody derivative, a recombinant humanized antibody, or an antigen-binding fragment or derivative of any of these. Antibodies or antigen binding fragments or derivatives encompassing permutations of the light and/or heavy chains between a monoclonal, chimeric, humanized or human antibody are also encompassed herewith.

The term “antibody” refers to an intact antibody, including monoclonal or polyclonal antibodies. The term “antibody” also encompasses multispecific antibodies such as bispecific antibodies. The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain. The term “antibody” also is intended to include antibodies of all immunoglobulin isotypes and subclasses.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus, once a set of CDR sequences (i.e., the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term“purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

It should be understood that proteins, including antibodies of the invention may associate with a specified region through various interactions to form ligand-receptor complexes. These interactions include but are not limited to electrostatic forces, such as hydrogen-bonding and Van der Waal forces, dipole-dipole interactions, hydrophobic interactions, pi-pi stacking, and so on. Other associations which describe more specific types of interactions include covalent bonds, electronic and conformational rearrangements, steric interactions, and so on. Thus, as used herein the term “associate” generally relates to any type of force which connects an antibody to a specified region. As used herein the term “interacts” generally relates to a more specific and stronger connection of an antibody to a specified region. As used herein the term “sterically blocks” is a specific type of association which describes an antibody interacting with a specific region and preventing other ligands from associating with that region through steric interactions. The terms “binds” or “specifically binds” as used throughout this application may be interpreted to relate to the terms “associates”, “interacts” or “sterically blocks” as required. By “specifically binds” is meant a compound or antibody that recognizes and binds a polypeptide of the invention, but which does not substantially recognize and bind other molecules in a sample, for example, a biological sample, which naturally includes a polypeptide of the invention.

The antibody specifically binding to CD5L monomer, or CD5L:CD5L homodimer, or CD5L:p40 heterodimer, or the antigen binding fragments thereof, may include variants of amino acid sequences disclosed herein within a range retaining the ability to specifically recognize the CD5L monomer, or CD5L:CD5L homodimer, or CD5L:p40 heterodimer. For example, to enhance the binding affinity and/or other biological properties of the antibody, the amino acid sequences of the antibody may be mutated. For example, such mutations include deletion, insertion, and/or substitution of amino acid sequence residues of the antibody. An amino acid mutation is made based on the relative similarity of the amino acid side chain substituents, for example, with respect to hydrophobic properties, hydrophilic properties, charges, or sizes. For example, arginine, lysine, and histidine are each a positively charged residue; alanine, glycine, and serine have a similar size; and phenylalanine, tryptophan, and tyrosine have a similar shape. Therefore, based on the considerations described above, arginine, lysine, and histidine may be biological functional equivalents; alanine, glycine, and serine may be biological functional equivalents; and phenylalanine, tryptophan, and tyrosine may be biological functional equivalents. Amino acid substitution in a protein in which the activity of the molecule is not completely changed is well known in the art. Typical substitutions include Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly substitutions. Considering mutations with biologically equivalent activity, an antibody specifically binding to CD5L monomer, or CD5L:CD5L homodimer, or CD5L:p40 heterodimer or the antigen-binding fragments thereof may also include sequences substantially identical to sequences disclosed herein. In this regard, a substantially identical amino acid sequence may be a sequence with at least 60% homology, at least 70% homology, at least 80% homology, at least 90%, at least 95% homology or 100% homology to a sequence disclosed herein, when the amino acid sequences are aligned to correspond to each other as much as possible. The aligned amino acid sequences are analyzed using an algorithm known in the art. Alignment methods for sequence comparison are well known to one of ordinary skill in the art. For example, a sequence analysis program available on the Internet at the NCBI Basic Local Alignment Search Tool (BLAST) home page, such as blastp, blastx, tblastn, or tblastx, may be used.

“Specific binding” of an antibody means that the antibody exhibits appreciable affinity for a particular antigen or epitope and, generally, does not exhibit significant crossreactivity. Specific binding includes binding with an affinity of at least 25 pM. Antibodies with affinities greater than 1×10⁷ M⁻¹ (or a dissociation coefficient of 1 pm or less or a dissociation coefficient of 1 nm or less) typically bind with correspondingly greater specificity. Values intermediate of those set forth herein are also intended to be within the scope of the present invention and antibodies of the invention bind to antigen with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to the antigen will not significantly react with non-antigen proteins or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

In some embodiments, the present invention provides antibodies specific for CD5L monomer, which specifically binds and/or activates CD5L monomer to produce its biological response, and does not bind or activate CD5L:CD5L homodimer, or CD5L:p40 heterodimer. In some embodiments, the present invention provides antibodies specific for CD5L:CD5L homodimer, which specifically binds and/or activates CD5L:CD5L homodimer to produce its biological response, and does not bind or activate CD5L monomer, or CD5L:p40 heterodimer. In some embodiments, the present invention provides antibodies specific for CD5L:p40 heterodimer, which speicifically binds and/or activates CD5L:p40 heterodimer, and does not bind or activate CD5L monomer, or CD5L homodimer.

A typical antigen binding site is comprised of the variable regions formed by the pairing of a light chain immunoglobulin and a heavy chain immunoglobulin. The structure of the antibody variable regions is consistent and exhibits similar structures. These variable regions are typically comprised of relatively homologous framework regions (FR) interspaced with three hypervariable regions termed Complementarity Determining Regions (CDRs). The overall binding activity of the antigen binding fragment is often dictated by the sequence of the CDRs. The FRs often play a role in the proper positioning and alignment in three dimensions of the CDRs for optimal antigen binding. However, in general, the CDR residues are directly and most substantially involved in influencing antigen binding.

A number of antibody production systems are described in Birch & Radner (2006) Adv. Drug Delivery Rev. 58:671-685, Rodrigues et al. (2010) Biotechnol. Prog. 26(2):332-351; Shukla and Thommes (2010) Trends Biotechnol. 28(5):253-261; Whaley et al. (2014) Curr. Top. Microbiol. Immunol. 375:107-126; Chon and Zarbis-Papastoitsis (2011) N. Biotechnol. 28(5):458-463; Li et al. (2010) MAbs 2(5):466-479; Grisowold and Bailey-Kellogg (2016) Cur. Opin. Struct. Biol. 39:79-88.

The term “humanized antibody” encompasses fully humanized antibody (i.e., frameworks are 100% humanized) and partially humanized antibody (e.g., at least one variable domain contains one or more amino acids from a human antibody, while other amino acids are amino acids of a non-human parent antibody). Typically, a “humanized antibody” contains CDRs of a non-human parent antibody (e.g., mouse, rat, rabbit, non-human primate, etc.) and frameworks that are identical to those of a natural human antibody or of a human antibody consensus. In such instance, those “humanized antibodies” are characterized as fully humanized. A “humanized antibody” may also contain one or more amino acid substitutions that have no correspondence to those of the human antibody or human antibody consensus. Such substitutions include, for example, back-mutations (e.g., re-introduction of non-human amino acids) that may preserve the antibody characteristics (e.g., affinity, specificity etc.). Such substitutions are usually in the framework region. A “humanized antibody” optionally also comprises at least a portion of a constant region (Fc) which is typically that of a human antibody. Typically, the constant region of a “humanized antibody” is identical to that of a human antibody.

The term “natural human antibody” refers to an antibody that is encoded (encodable) by the human antibody repertoire, i.e., germline sequence.

0001The term “chimeric antibody” refers to an antibody having non-human variable region(s) and human constant region.

The term “hybrid antibody” refers to an antibody comprising one of its heavy or light chain variable region (its heavy or light chain) from a certain type of antibody (e.g., humanized) while the other of the heavy or light chain variable region (the heavy or light chain) is from another type (e.g., murine, chimeric).

In some embodiments, the heavy chain and/or light chain framework region of the humanized antibody may comprise from one to thirty amino acids from the non-human antibody which is sought to be humanized and the remaining portion being from a natural human antibody or a human antibody consensus. In some instances, the humanized antibody may comprise from 1 to 6 non-human CDRs, e.g., wherein the six CDRs are non-human.

The natural human antibody selected for humanization of the non-human parent antibody may comprise a variable region having a three-dimensional structure similar to that of (superimposable to) a (modeled) variable region of the non-human parent antibody. As such, the humanized antibody has a greater chance of having a three-dimensional structure similar to that of the non-human parent antibody.

The light chain variable region of the natural human antibody selected for humanization purposes, may have, for example an overall (over the entire light chain variable region) identity of at least 70%, 75%, 80%, etc. identity with that of the non-human parent antibody. Alternatively, the light chain framework region of the natural human antibody selected for humanization purposes, may have, for example, at least 70% 75%, 80%, 85% etc. sequence identity with the light chain framework region of the non-human parent antibody. In some embodiments, the natural human antibody selected for humanization purposes may have the same or substantially the same number of amino acids in its light chain complementarity determining region to that of a light chain complementarity determining region of the non-human parent antibody.

The heavy chain variable region of the natural human antibody selected for humanization purposes, may have, for example an overall (over the entire heavy chain variable region) identity of at least 60%, 70%, 75%, 80%, etc. identity with that of the non-human parent antibody. In some embodiments, the human framework region amino acid residues of the humanized antibody heavy chain may be from a natural human antibody heavy chain framework region having at least 70%, 75%, 89% etc. identity with a heavy chain framework region of the non-human parent antibody. In some embodiments, the natural human antibody selected for humanization purposes may have the same or substantially the same number of amino acids in its heavy chain complementarity determining region to that of a heavy chain complementarity determining region of the non-human parent antibody.

The natural human antibody that is selected for humanization of the non-human parent antibody may comprise a variable region having a three-dimensional structure similar to that of (superimposable to) a (modeled) variable region of the non-human parent antibody. As such, the humanized or hybrid antibody has a greater chance of having a three-dimensional structure similar to that of the non-human parent antibody.

For example, the natural human antibody heavy chain variable region which may be selected for humanization purposes may have the following characteristics: a) a three-dimensional structure similar to or identical (superimposable) to that of a heavy chain of the non-human antibody and/or b) a framework region having an amino acid sequence at least 70% identical to a heavy chain framework region of the non-human antibody. Optionally, (a number of) amino acid residues in a heavy chain CDR (e.g., all three CDRs) is the same or substantially the same as that of the non-human heavy chain CDR amino acid residues.

Alternatively, the natural human antibody light chain variable region which may be selected for humanization purposes may have the following characteristics: a) a three-dimensional structure similar to or identical (superimposable) to that of a light chain of the non-human antibody, and/or b) a framework region having an amino acid sequence at least 70% identical to a light chain framework region of the non-human antibody. Optionally, (a number of) amino acid residues in a light chain CDR (e.g., all three CDRs) that is the same or substantially the same as that of the non-human light chain CDR amino acid residues.

Chimeric, humanized or primatized antibodies can be prepared based on the sequence of a reference monoclonal antibody prepared using standard molecular biology techniques. DNA encoding the heavy and light chain immunoglobulins can be obtained from the hybridoma of interest and engineered to contain non-reference (e.g., human) immunoglobulin sequences using standard molecular biology techniques. For example, to create a chimeric antibody, the murine variable regions can be linked to human constant regions using methods known in the art (U.S. Pat. Nos. 4,816,567, 5,565,332; Morrison (1984) PNAS 81(21):6851-6855; LoBuglio (1989) PNAS 86(11):4220-4224). To create a humanized antibody, the murine CDR regions can be inserted into a human framework using methods known in the art (U.S. Pat. Nos. 5,225,539; 5,530,101; 5,585,089; 5,693,762; 6,180,370; Lo (2014) “Antibody humanization by CDR grafting.” Antibody Engineering: Methods and Protocols. 135-159; Kettleborough et al. (1991) Protein Eng. 4(7):773-783.). Similarly, to create a primatized antibody the murine CDR regions can be inserted into a primate framework using methods known in the art (WO 93/02108; WO 99/55369). Further approaches to “species”-ization of antibodies are known in the art and include structure-guided methods and computational design.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al, (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; U.S. Pat. No. 6,075,181).

The antibodies also can be modified to create chimeric antibodies. Chimeric antibodies are those in which the various domains of the antibodies' heavy and light chains are coded for by DNA from more than one species. See, e.g., U.S. Pat. Nos. 4,816,567; 5,202,238; 5,565,332; 5,482,856; 6,808,901; 6,965,024; 9,346,873.

Alternatively, the antibodies can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological interest, Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; described in Padlan et al. (1991) Mol. Immunol. 28(4-5):489-498.

The variable region of the antibodies can be modified by mutating amino acid residues within the VH and/or VL CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. In certain embodiments, conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

Antibodies and/or antigen binding fragments may originate, for example, from a mouse, a rat or any other mammal or from other sources such as through recombinant DNA technologies.

The antibodies can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies include naturally purified products, products of chemical synthetic procedures, and products produced by recombinant techniques from a eukaryotic host, including, for example, yeast, higher plant, insect and mammalian cells, or alternatively from a prokaryotic host as described above.

Some embodiments comprise polynucleotides that encode the amino acid sequence of the antibody and/or antigen-binding fragment thereof, as well as methods to produce recombinantly or chemically synthesize the antibody polypeptides and/or antigen-binding fragments thereof. The antibody polypeptides can be produced in a eukaryotic or prokaryotic cell, or by other methods known in the art.

Antibodies also can be generated using conventional techniques known in the art and are well-described in the literature. For example, polyclonal antibodies can be produced by immunization of a suitable mammal such as, but not limited to, chickens, goats, guinea pigs, hamsters, horses, mice, rats, and rabbits. In some embodiments, an antigen injected into the mammal induces B-lymphocytes to produce immunoglobulins (e.g., antibodies) that bind to the antigen, which may be purified from the mammal's serum. Antibodies specific to a CD5L monomer, a CD5L:CD5L homodimer, or a CD5L:p40 heterodimer can thus be generated by injection of a CD5L monomer, CD5L:CD5L homodimer, or CD5L:p40 heterodimer, respectively, or a fragment thereof.

Variations of antibody production methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a stow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods known in the art some of which are described in Leenars and Hendriksen, ILAR J (2005) 46 (3): 269-279; Stevens et al. (2012). The laboratory rabbit, guinea pig, hamster, and other rodents. Oxford: Academic; U.S. Pat. Nos. 7,279,559; 7,119,179; 7,060,800; 6,709,659; 6,656,746; 6,322,788; 5,686,073; 5,670,153; and Newcombe and Newcombe (2007), J Chromatogr B Analyt Technol Biomed Life Sci. 848(1):2-7.

Monoclonal antibodies can be generated using conventional hybridoma techniques known in the art and described in the literature (e.g. Zhang. “Hybridoma technology for the generation of monoclonal antibodies.” Antibody methods and protocols (2012): 117-135) or hybridoma-free methods (e.g. Pasqualini et al. (2004) Hybridoma-free generation of monoclonal antibodies. PNAS 101(1):257-259). For example, a hybridoma can be produced by fusing a suitable immortal cell line or any other suitable cell line as known in the art (see, those at the following web addresses e.g., atcc.org, lifetech.com, and other suitable databases), with antibody producing cells. Examples of immortal cell lines include, but are not limited to, a myeloma cell line such as, but not limited to, Sp2/0, Sp2/0-AG14, NSO, NS1, NS2, AE-1, L.5, P3X63Ag8,653, Sp2 SA3, Sp2 MAI, Sp2 SS1, Sp2 SA5, U397, MIA 144, ACT IV, MOLT4, DA-1, JURKAT, WEHI, K-562, COS, RAJI, NIH 313, HL-60, MLA 144, NAMAIWA, NEURO 2A, CHO, PerC.6, YB2/O) or the like, or heteromyelomas, fusion products thereof, or any cell or fusion cell derived there from. Examples of suitable antibody producing cells include, but are not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof.

Antibody producing cells can also be obtained from the peripheral blood or, in particular embodiments, the spleen or lymph nodes, of humans or other suitable animals that have been immunized with the antigen of interest. Any other suitable host cell can also be used for expressing-heterologous or endogenous nucleic acid encoding an antibody, specified fragment or variant thereof. The fused cells (hybridomas) or recombinant cells can be isolated using selective culture conditions or other suitable known methods, and cloned by limiting dilution or cell sorting, or other known methods.

Particular isotypes of a monoclonal antibody can be prepared either directly by selecting from an initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653, Spira et al. (1984) J. Immunol. Methods 74:307. Alternatively, recombinant DNA techniques may be used, e.g. the CRISPR-Cas method for switching provided in Cheong et al. (2016) supra.

The isolation of other monoclonal antibodies with the specificity of the monoclonal antibodies can also be accomplished by one of ordinary skill in the art by producing anti-idiotypic antibodies. See Herlyn et al. (1986) Science 232:100 and/or commercially available protocols. An anti-idiotypic antibody is an antibody which recognizes unique determinants present on the monoclonal antibody of interest.

Other methods of producing or isolating antibodies can be used, including, but not limited to, methods that select recombinant antibody from a peptide or protein library (e.g., but not limited to, a bacteriophage, ribosome, oligonucleotide, cDNA, or the like, or a display library, e.g., as available from various commercial vendors such as MorphoSys Creative Biolabs, Biolnvent, or Affitech) using methods known in the art. Art known methods are described in the patent literature (e.g. U.S. Pat. Nos. 4,704,692; 5,723,323; 5,763,192; 5,814,476; 5,817,483; 5,824,514; 5,976,862; 6,849,425; 7,175,996; 8,691,730; 8,877,688) and more generally in, for example, Hoogenboom (2005) Nature Biotechnol. 23:1105-1116. Alternative methods rely upon immunization of transgenic animals (e.g., SCID mice; Nguyen et al. (1977) Microbiol. Immunol. 41:901-907 (1997); Sandhu et al. (1996) Crit. Rev. Biotechnol. 16:95-118; Eren et al. (1998) Immunol. 93(2):154-16; Bruggemann et al. (2015) Arch Immunol Ther Exp (Warsz). 63(2): 101-108) that are capable of producing a repertoire of human antibodies, as known in the art. Such techniques, include, but are not limited to, ribosome display (e.g., Hanes et al. (1997) PNAS 94:4937-4942; Hanes et al. (1998) Proc. Natl. Acad. Sci. USA 95:14130-14135), Edwards and He (2012) Methods Mol. Biol. 907:281-292, He and Khan (2005) Expert Rev. Proteomics 2(3):421-430, Kanamori et al. (2014) BBA Proteins and Proteomics. 1844(11):1925-1932); single cell antibody producing technologies (e.g., selected lymphocyte antibody method (“SLAM”); U.S. Pat. No. 5,627,052, Wen et al. (1987) J. Immunol 17:887-892; Babcook et al. (1996) Proc. Natl. Acad. Sci. USA 93:7843-7848, Tilled et al. (2008) J Immunol Methods. 329(1-2): 112-124; Ouisse et al. (2017) BMC Biotechnol. 17:3); gel microdroplet and flow cytometry (e.g. Powell et al. (1990) Biotechnol. 8:333-337; One Cell Systems, (Cambridge, Mass.); Gray et al. (1995) J. Imm. Meth. 182:155-163; and Kenny et al. (1995) Bio. Technol. 13:787-790); B-cell selection (e.g. Steenbakkers et al. (1994) Molec. Biol. Reports 19:125-134); and the use of CRISPR-Cas system to edit immunoglobulin genes and obtain a desired antibody (e.g. Cheong et al. (2016) Nature Comm. 7:10934).

Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; 8,937,162; 9,090,994; 9,550,986; 9,593,161; 8,296,079; and WO 2014/99542; WO2012/092374; and Safdari et al. (2013) Biotechnol. Genet. Eng. Rev. 29:175-86; Harrison (2014) Nature Rev. Drug Discover 13:336; Ahmadzadeh et al. (2014) Monoclon. Antib. Immunodiagn. Immunother. 33(2):67-73; Hanf et al. (2014) Methods 65(1):68-76; Gonzales et al. (2005) Tumor Biol. 26(1):31-43.

The term “antigen-binding fragment”, as used herein, refers to one or more fragments of an antibody that retain the ability to bind to an antigen (e.g., a CD5L monomer, a CD5L:CD5L homodimer, and/or a CD5L:p40 heterodimer). It has been shown that the antigen-binding function of an antibody can be performed by fragments of an intact antibody. Examples of binding fragments encompassed within the term “antigen-binding fragment” of an antibody include (i) a Fab fragment, a monovalent fragment consisting of the VL, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR), e.g., V_{H} CDR3. Furthermore, although the two domains of the Fv fragment, V_{L} and V_{H}, are coded for by separate genes, they can be joined, using recombinant methods, by a synthetic linker that enables them to be made as a single polypeptide chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region may be modified by replacing one or more cysteine residues with serine residues so as to prevent dimerization. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

Antibody derivatives can also be prepared by delivering a polynucleotide encoding an antibody or fragment thereof to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; 5,304,489; and those references mentioned herein above.

The term “antibody derivative” includes post-translational modification to a linear polypeptide sequence of the antibody or fragment. For example, U.S. Pat. No. 6,602,684 describes a method for the generation of modified glycol-forms of antibodies, including whole antibody molecules, antibody fragments, or fusion proteins that include a region equivalent to the Fc region of an immunoglobulin, having enhanced Fe-mediated cellular toxicity, and glycoproteins so generated.

The term “antibody derivative” also includes “diabodies” which are small antibody fragments with two antigen-binding sites, wherein fragments comprise a heavy chain variable domain connected to a light chain variable domain in the same polypeptide chain. (e.g. EP 404,097; WO 93/11161; and Hollinger et al. (1993) Proc. Natl. Acad. Sci. USA 90:6444-6448.) Without being bound by theory, it is believed that by using a linker that is too short to allow pairing between the two domains on the same chain, the domains are forced to pair with the complementary domains of another chain and create two antigen-binding sites. (e.g., U.S. Pat. No. 6,632,926).

The term “antibody derivative” further includes engineered antibody molecules, fragments and single domains such as scFv, dAbs, nanobodies, minibodies, unibodies, and affibodies. See, e.g., Hudson (2005) Nature Biotech 23(9):1126-36; U.S. Patent Application Publication No. 2006/0211088; WO 2007/059782; U.S. Pat. No. 5,831,012).

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Ed segments (V_{H}-C_{H}1-V_{H}-C_{H}1) which form a pair of antigen binding regions. Linear antibodies can be bispecific or monospecific.

The antibodies also include derivatives that are modified by the covalent attachment of any type of molecule to the antibody such that covalent attachment does not prevent the antibody from generating an anti-idiotypic response. Antibody derivatives include, but are not limited to, antibodies that have been modified by glycosylation, acetylation, pegylation, phosphorylation, amidation, derivatization by known protecting/blocking groups, proteolytic cleavage, linkage to a cellular ligand or other protein, etc. Additionally, the derivatives may contain one or more non-classical amino acids.

Antibody derivatives also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118, and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147, and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and references cited therein. Thus, antibodies can also be produced using transgenic plants, according to know methods. See Ko et al. (2009) Curr. Top Microbiol. Immunol. 332:55-78; Buyel et al. (2017) Biotecnol. Adv. S0734-9750(17)30029-0. doi: 10.1016/j.biotechadv.2017.03.011.

Antibody derivatives also can be produced, for example, by adding exogenous sequences to modify immunogenicity or to reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

In addition, the antibodies may be engineered to include modifications within the Fc region to alter one or more functional properties of the antibody, such as serum half-life, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (e.g. U.S. Pat. No. 5,677,425) and amino acid mutations in the Fc hinge region to decrease biological half-life of the antibody (e.g. U.S. Pat. No. 6,165,745).

Additionally, the antibodies may be chemically modified. Glycosylation of an antibody can be altered, for example, by modifying one or more sites of glycosylation within the antibody sequence to increase the affinity of the antibody for antigen (e.g. U.S. Pat. Nos. 5,714,350, 6,350,861, Jefferis (2009) Nature Rev. Drug Discovery 8:226-234; Abes (2010)). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (e.g. Shields, R. L. et al. (2002) J. Biol. Chem. 277:26733-26740; Umana et al. (1999) Nat. Biotech. 17:176-180).

The antibodies can be pegylated to increase biological half-life by reacting the antibody or fragment thereof with polyethylene glycol (PEG) or a reactive ester or aldehyde derivative of PEG, under conditions in which one or more PEG groups become attached to the antibody or antibody fragment. Antibody pegylation may be carried out by an acylation reaction or an alkylation reaction with a reactive PEG molecule (or an analogous reactive water soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. The antibody to be pegylated can be an aglycosylated antibody. Methods for pegylating proteins are known in the art (e.g. EP 0154316, EP 0401384).

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See Harlow and Lane (1988) supra.

Additionally, antibodies may be chemically modified by conjugating or fusing the antigen-binding region of the antibody to serum protein, such as human serum albumin, to increase half-life of the resulting molecule. Such approach is for example described in EP 0322094, EP 0486525, Chapman (2002) Adv. Drug Delivery Rev. 54(4):531-545.

The antibodies or fragments thereof may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include¹²⁵I, ¹³¹I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydral groups (Koyama 1994 Chem. Abstr. 120: 217262t) of amino acid residues and carbohydrate groups (Rodwell et al. (1986) PNAS USA 83:2632-2636; Quadri et al. (1993) Nucl. Med. Biol. 20:559-570).

Further, the antibodies or fragments thereof may be conjugated to a therapeutic agent. Suitable therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabinc, cladribine), alkylating agents (such as mechlorethamine, thioepa, chloramhucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrietocin, phenomycin, enomycin toxins and mixed toxins. Other therapeutics suitable for use in the methods of treatment may be optionally conjugated with the antibodies along the lines described. It is appreciated that a therapeutic should be conjugated to an antibody only if suited to treat the disease, disorder, or condition for which the immunoconjugate will target.

Additional suitable conjugated molecules include ribonuclease (RNase), DNase, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (e.g. U.S. Pat. No. 5,631,146) and theophiline (e.g. U.S. Pat. No. 5,580,737), as well as large molecules, such as reverse transcriptase (e.g. U.S. Pat. No. 5,786,462) and thrombin (e.g. U.S. Pat. No. 5,543,293). Ribozymes are nucleic acid molecules that are capable of catalyzing a chemical reaction, either intra-molecularly or inter-molecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen basepairing. Triplex molecules can bind target regions with high affinity and specificity. Suitable conjugated molecules may further include any protein that binds to DNA provided that it does not create or stabilize biofilm architecture; it is envisioned that at least a subset of such proteins may facilitate the kinetics of binding for the interfering agents.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (e.g. Yu et al. (1994) Int. J. Cancer 56: 244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, Engineering and Clinical Application, Ritter et al. (eds.), pages 60-84 (Cambridge University Press 1995)).

Techniques for conjugating therapeutic agents to antibodies are well known (e.g. Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al, (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy”, in Monoclonal Antibodies For Cancer Detection And Therapy, Baldwin et al. (eds.), pp. 303-16 (Academic Press 1985), and Thorpe et al. (1982) Immunol. Rev. 62:119-58; Sievers and Senter (2013) Annual Rev. Med. 64:15-29; Wu and Senter (2005) Nature Biotechnol. 23(9):1137-1146; Sassoon and Blanc (2013) “Antibody-drug conjugate (ADC) clinical pipeline: a review.” Antibody-Drug Conjugates: 1-27.).

The antibodies or antigen-binding regions thereof can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or non-covalent association. Multi-specific molecules can further include a third binding specificity, in addition to the first and second target epitope.

Bi-specific and multi-specific molecules can be prepared using methods known in the art. For example, each binding unit of the bi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

In some aspects, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble. Those skilled in the art will know of other suitable carriers for binding monoclonal antibodies, or will be able to ascertain such, using routine experimentation.

In certain aspects, the disclosure relates to an antibody or antigen-binding fragments or derivatives that specifically recognize or binds CD5L and/or a CD5L:CD5L homodimer. Non-limiting exemplary antibodies are produced by the clones disclosed in Table 1.

TABLE 1

| Specificity | Clone Name |
|---|---|
| CD5L/CD5L:CD5L | 2B9-10-10-3 |
| CD5L/CD5L:CD5L | 2B9-10-10-4 |
| CD5L/CD5L:CD5L | 2B9-10-10-5 |
| CD5L/CD5L:CD5L | 2B9-10-10-6B |
| CD5L/CD5L:CD5L | 3F11-3-10-1 |

In certain aspects, the disclosure relates to an antibody or antigen binding fragment that specifically recognizes or binds CD5L:p40 heterodimer. Non-limiting exemplary antibodies are produced by the clones disclosed in Table 2.

TABLE 2

| Specificity | Clone Name |
|---|---|
| CD5L:p40 | 2B9-10-10-6A |
| CD5L:p40 | 2B9-10-10-2A |
| CD5L:p40 | 2B9-12-1-2 |
| CD5L:p40 | 2B9-10-10-15 |
| CD5L:p40 | 2B9-12-1-2 |
| CD5L:p40 | 2B9-12-3 |
| CD5L:p40 | 2B9-10-10-16 |
| CD5L:p40 | 2B9-10-10-4? |
| CD5L:p40 | 2B9-10-10-3 |
| CD5L:p40 | 2B9-10-10-5 |

Hybridoma cell lines derived from the clones in Tables 1 and 2 that produce monoclonal antibodies that specifically recognize and bind CD5L monomer, CD5L:CD5L homodimer, and/or CD5L:p40 heterodimer are generated. These hybridomas are assigned an Accession Number upon deposit with American Type Culture Collection (ATCC) pursuant to the provisions of the Budapest Treaty.

Some aspects relate to an isolated antibody that is at least 85% identical to an antibody selected from the group consisting of the clones listed in Table 1 and the clones listed in Table 2.

Some aspects relate to an isolated antibody comprising one or more CDRs of the heavy and/or light chain of an antibody selected from the group consisting of the clones listed in Table 1 and the clones listed in Table 2.

In some aspects, the heavy chain variable domain comprises the heavy chain variable domain sequence of an antibody selected from the group consisting of the clones listed in Table 1 and the clones listed in Table 2, or a sequence at least 60%, 65%, 70%, 75%, 80%, 85%, 90%, 95%, or 99% identical thereto.

In some aspects, the light chain variable domain comprises the light chain variable domain sequence of an antibody selected from the group consisting of the clones listed in Table 1 and the clones listed in Table 2, or a sequence 60%, 65%, 70%, 75%, 80%, 85%, 90%, 95%, or 99% identical thereto.

In some aspects, the antibody binds CD5L monomer, CD5L:CD5L homodimer, and/or CD5L:p40 heterodimer with a dissociation constant (K_{D}) of less than 10⁻⁴ M, 10⁻⁵ M, 10⁻⁶ M, 10⁻⁷ M, 10⁻⁸ M, 10⁻⁹ M, 10^{31 10} M, 10⁻¹¹ M, or 10⁻¹² M.

In some of the aspects, the antibody is a full-length antibody.

In some of the aspects, the heavy and light chain variable domain sequences are components of the same polypeptide chain. In some of the aspects, the heavy and light chain variable domain sequences are components of different polypeptide chains.

In some of the aspects, the antibody is a monoclonal antibody. In some of the aspects, the antibody is a chimeric antibody.

In some of the aspects, the antibody is selected from the group consisting of Fab, F(ab)′2, Fab′, scFv, and Fv. In some of the aspects, the antibody is soluble Fab. In some of the aspects, the antibody comprises an Fc domain.

In some of the aspects, the antibody is a mouse, rat, or rabbit antibody. In some of the aspects, the antibody is a human or humanized antibody and/or is non-immunogenic in a human. In some of the aspects, the antibody comprises a human antibody framework region.

In some aspects, one or more amino acid residues in a CDR of the antibodies are substituted with another amino acid. The substitution may be “conservative” in the sense of being a substitution within the same family of amino acids. The naturally occurring amino acids may be divided into the following four families and conservative substitutions will take place within those families.
1) Amino acids with basic side chains: lysine, arginine, histidine.
2) Amino acids with acidic side chains: aspartic acid, glutamic acid.
3) Amino acids with uncharged polar side chains: asparagine, glutamine, serine, threonine, tyrosine.
4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies can comprise such varied CDR sequences that still bind CD5L monomer, CD5L:CD5L homodimer, and/or CD5L:p40 heterodimer with similar specificity and sensitivity profiles as the disclosed antibodies. This may be tested by way of the binding assays.

The constant regions of antibodies may also be varied. For example, antibodies may be provided with Fc regions of any isotype: IgA (IgA1, IgA2), IgD, IgE, IgG (IgG1, IgG2, IgG3, IgG4) or IgM. Non-limiting examples of constant region sequences include:

Human IgD constant region, Uniprot: P01880
(SEQ ID NO 10)
APTKAPDVFPIISGCRHPKDNSPVVLACLITGYHPTSVTVTWYMGTQSQPQRTFPEIQRRDSYYMTSSQLSTPLQ
QWRQGEYKCVVQHTASKSKKEIFRWPESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDAHLTWEVAGKVPTGGVEEGLLERHSN
GSQSQHSRLTLPRSLWNAGTSVTCTLNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQPATYTCVVSHEDSRTLLNASRSLEVS
YVTDHGPMK
Human IgG1 constant region, Uniprot: P01857
(SEQ ID NO 11)
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLEPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG2 constant region, Uniprot: P01859
(SEQ ID NO 12)
ASTKGPSVFPLAPCSRSTSESTAALGCLVKDYFPEPVTVSWNSGALTSGVHTFPAVLQSSGLYSLSSVVTVPSSN
FGTQTYTCNVDHKPSNTKVDKTVERKCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKCKVSNKGLPAPIEKTISKTKGQPREP
QVYTLPPSREEMTKNQVSLTCLVKGFYPSDISVEWESNGQPENNYKTTPPMLDSDGSFFLYSKLTVDKSRWQQGN
VFSCSVMHEALHNHYTQKSLSLSPGK
Human IgG3 constant region, Uniprot: P01860
(SEQ ID NO 13)
ASTKGPSVFPLAPCSRSTSGGTAALGCLVKDYFPEPVTVSWNSGALTSGVHTFPAVLQSSGLYSLSSVVTVPSSS
LGTQTYTCNVNHKPSNTKVDKRVELKTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHEDPEVQFKWYVDGVEVHNAKTKPREEQ
YNSTFRVVSVLTVLHQDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHEALHNRFTQKSLSLSP
GK
Human IgM constant region, Uniprot: P01871
(SEQ ID NO 14)
GSASAPTLFPLVSCENSPSDTSSVAVGCLAQDFLPDSITLSWKYKNNSDISSTRGFPSVLRGGKYAATSQVLLPS
KDVMQGTDEHVVCKVQHPNGNKEKNVPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVDHRGLTFQQNASSMCVPDQDTAIRVF
AIPPSFASIFLTKSTKLTCLVTDLTTYDSVTISWTRQNGEAVKTHTNISESHPNATFSAVGEASICEDDWNSGER
FTCTVTHTDLPSPLKQTISRPKGVALHRPDVYLLPPAREQLNLRESATITCLVTGFSPADVFVQWMQRGQPLSPE
KYVTSAPMPEPQAPGRYFAHSILTVSEEEWNTGETYTCVAHEALPNRVTERTVDKSTGKPTLYNVSLVMSDTAGT
CY
Human IgG4 constant region, Uniprot: P01861
(SEQ ID NO 15)
ASTKGPSVFPLAPCSRSTSESTAALGCLVKDYFPEPVTVSWNSGALTSGVHTFPAVLQSSGLYSLSSVVTVPSSS
LGTKTYTCNVDHKPSNTKVDKRVESKYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
Human IgA1 constant region Uniprot: P01876
(SEQ ID NO 16)
ASPTSPKVFPLSLCSTQPDGNVVIACLVQGFFPQEPLSVTWSESGQGVTARNFPPSQDASGDLYTTSSQLTLPAT
QCLAGKSVTCHVKHYTNPSQDVTVPCPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGKTFTCTAAYPESKTPLTATLSKSGNT
FRPEVHLLPPPSEELALNELVTLTCLARGFSPKDVLVRWLQGSQELPREKYLTWASRQEPSQGTTTFAVTSILRV
AAEDWKKGDTESCMVGHEALPLAFTQKTIDRLAGKPTHVNVSVVMAEVDGTCY
Human IgA2 constant region, Uniprot: P01877
(SEQ ID NO 17)
ASPTSPKVFPLSLDSTPQDGNVVVACLVQGFFPQEPLSVTWSESGQNVTARNFPPSQDASGDLYTTSSQLTLPAT
QCPDGKSVTCHVKHYTNPSQDVTVPCPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKTPLTANITKSGNTFRPEVHLLPPPSE
ELALNELVTLTCLARGFSPKDVLVRWLQGSQELPREKYLTWASRQEPSQGTTTFAVTSILRVAAEDWKKGDTFSC
MVGHEALPLAFTQKTIDRMAGKPTHVNVSVVMAEVDGTCY
Human Ig kappa constant region, Uniprot: P01834
(SEQ ID NO 18)
TVAAPSVFIFPPSDEQLKSGTASVVCLLNNEYPREAKVQWKVDNALQSGNSQESVTEQDSKDSTYSLSSTLTLSK
ADYEKHKVYACEVTHQGLSSPVTKSFNRGEC

In some aspects, the antibody binds to the epitope bound by an antibody selected from the group consisting of the clones listed in Table 1 and the clones listed in Table 2.

In some aspects, the antibody contains structural modifications to facilitate rapid binding and cell uptake and/or slow release. In some aspects, the antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

In some embodiments, the antibody or derivative or fragment thereof is conjugated to a diagnostic, therapeutic, and/or detectable agent. In some embodiments, the antibody or derivative or fragment thereof is used to detect CD5L monomer, CD5L:CD5L homodimer, and/or CD5L:p40 heterdimer, using an immunodetection method. In some embodiments, the immunodetection method is enzyme-linked immunosorbent assay (ELISA), histology, fluorescence-activated cell sorting, radioimmunoassay (RIA), immunoradiometric assay, immunohistochemistry, fluoroimmunoassay, chemiluminescent assay, bioluminescent assay, Western blotting, or dot blotting. In an ELISA assay, two different antibodies recognizing two different epitopes of a given protein can be used to detect the protein through the detection of a substrate linked to one of the antibodies in a colorimetric assay. In Histology, a labeled antibody can be used to detect a protein in a tissue sample either in fresh frozen tissue or in formalin-fixed, paraffin embedded samples. In fluorescence-activated cell sorting, a fluorochrome-labeled antibody can be used to detect cells that express a particular protein. In the case of a secreted protein there are techniques available that allow the intracellular staining of said proteins by procedures known to those skilled in the art. In a radioimmunoassay a radioactively labeled protein can be used to measure the amount of protein present in a given sample by measuring the amount of radioactivity present in a competition assay (for example, by using a specific antibody). Variations of these assays involve the use of antibody/labeling compounds to measure the amount of a particular protein in a given sample through competition assays that depend on the affinity/avidity of the specific antibody. In a Western blot, a given protein can be detected by the use of a specific antibody following a gel transfer, a method that also allows the technician to know the molecular weight of the protein detected.

Compositions comprising or alternatively consisting essentially of or yet further, consisting of one or more of the above embodiments are further provided herein.

The antibodies, fragments, and equivalents thereof can be combined with a carrier, e.g., a pharmaceutically acceptable carrier or other agents to provide a formulation for use and/or storage.

### EQUIVALENTS

In certain embodiments, antibodies may be used to screened for equivalents. As used herein, the term “equivalent” when used in reference to an antibody intends any molecule which achieves the same biological effect as the reference antibody, e.g. an agonistic or antagonistic effect on CD5L monomer, CD5L:CD5L homodimer, and/or CD5L:p40 heterodimer.

Non-limiting examples included within the scope of equivalents include aptamers, affimers, non-immunoglobulin scaffolds, small molecules, fragments and derivatives thereof, and genetic modifying agents.

If a molecule being tested binds with the same protein or polypeptide as an antibody contemplated by this disclosure, it should be considered a possible equivalent. If a genetic modifying agent being tested provides similar or improved agonist or antagonist activity as compared to an antibody contemplated by this disclosure, it should be considered a possible equivalent. Candidate equivalents can be tested for equivalence to the reference antibody.

It also is possible to determine without undue experimentation, whether the molecule has the same specificity as an antibody by determining whether the molecule being tested prevents an antibody from binding the protein or polypeptide with which the antibody is normally reactive.

If the molecule being tested competes with the antibody as shown by a decrease in binding by the antibody, then it is likely that the molecule and the reference antibody bind to the same or a closely related epitope.

Alternatively, one can pre-incubate the antibody with a protein with which it is normally reactive, and determine if the molecule is inhibited in its ability to bind the antigen. If the molecule being tested is inhibited then, in all likelihood, it has the same, or a closely related, epitopic specificity as the antibody.

### Method of Identifying CD5L Receptor

In one aspect, the present invention provides methods for identifying a receptor for CD5L including a receptor for the CD5L monomer, a receptor for CD5L homodimer, and/or a receptor for CD5L:p40 heterodimer. Methods can be utilized as described herein where the CD5L monomer, CD5L homodimer, and/or CD5L:p40 heterodimer is labeled with either a tag (such as HA, MYC, FLAG or HIS-tag) or radioactive label. If labeled with an amino acid based label (such as HA, MYC, FLAG or HIS-tag) the successful binding of CD5L, including the CD5L monomer, the CD5L homodimer, and/or the CD5L:p40 heterodimer to their respective receptors can be detected by using a secondary anti-HA, anti-MYC, anti-FLAG or anti-HIS antibody labeled with a fluorochrome and detected in a fluorescence-activated cell sorter (FACS). If labeled with radioactivity, the binding can be monitored by measuring the radioactive counts bound to a cell expressing the receptor.

In some embodiments, the antibodies described herein for the CD5L monomer, CD5L homodimer, and/or CD5L:p40 heterodimer can be used to identify a receptor for CD5L. The method includes using the antibody or antigen binding fragment thereof as a ligand for binding to the CD5L receptor. The CD5L receptor can be identified by using labeled CD5L that can be used to bind to its receptor. The ligand/receptor complex can then be immunoprecipitated using an anti-CD5L or anti-label antibody. Examples of such labels include His-Tag, Flag-tag, and the like. CD5L can also be radiolabeled to first detect via radioimmunoassay cells that express the receptor. Different cells are incubated with radiolabeled CD5L, and following incubation the cells are washed or passed through gradients that separate by viscosity and centrifugation free versus bound radiolabeled CD5L. Cells that retain radioactivity should express the specific CD5L receptor.

### Method of Identifying Functional Domain of CD5L

The present invention also provides functional domain or fragment of CD5L, and nucleic acid molecules encoding such functional fragments. A “functional” CD5L or fragment thereof defined herein is characterized by its biological activity to regulate T cell function, its ability to bind to its partner p40 in forming a heterodimer CD5L:p40, or by its ability to bind specifically to an anti-CD5L antibody or other molecules (either agonist or antagonist). Moreover, functional fragments also include the signal peptide, intracellular signaling domain, and the like. Routine deletion analyses of nucleic acid molecules can be performed to obtain functional fragments of a nucleic acid molecule that encodes a CD5L polypeptide. As an illustration, DNA molecules having the nucleotide sequence of CD5L or fragments thereof, can be digested with nuclease to obtain a series of nested deletions. These DNA fragments are then inserted into expression vectors in proper reading frame, and the expressed polypeptides are isolated and tested for activity, or for the ability to bind anti-CD5L antibodies or other ligands. One alternative to exonuclease digestion is to use oligonucleotide-directed mutagenesis to introduce deletions or stop codons to specify production of a desired CD5L fragment. Alternatively, particular fragments of a CD5L polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5A synthetase induced by human interferon,” in Biological Interferon Systems, Proceedings of ISTR-TNO Meeting on Interferon Systems, Cantell (ed.), pages 65-72 (Nijhoff 1987); Herschman, “The EGF Receptor,” in Control of Animal Cell Proliferation L_Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50: 1295 (1995); and Meisel et al, Plant Molec. Biol. 30: 1 (1996).

Multiple amino acid substitutions can be made and tested using known methods of mutagenesis and screening, such as those disclosed by Reidhaar-Olson and Sauer (Science 241:53-57, 1988) or Bowie and Sauer (Proc. Natl. Acad. Sci. USA 86:2152-2156, 1989). Other methods that can be used include phage display (e.g., Lowman et al., Biochem. 30:10832-10837, 1991; Ladner et al, U.S. Pat. No. 5,223,409; Huse, WIPO Publication WO 92/062045) and region-directed mutagenesis (Derbyshire et al., Gene 46:145, 1986; Ner et al., DNA 7:127, 1988). Variants of the CD5L DNA and polypeptide sequences can be generated through DNA shuffling as disclosed by Stemmer, Nature 370:389-91, 1994, Stemmer, Proc. Natl. Acad. Sci. USA 91:10747-51, 1994 and WHO Publication WO 97/20078.

Mutagenesis methods as disclosed herein can be combined with high-throughput, automated screening methods to detect activity of cloned, mutagenized CD5L receptor polypeptides in host cells. Preferred assays in this regard include cell proliferation assays and biosensor-based ligand-binding assays, which are described below. Mutagenized DNA molecules that encode active receptors or portions thereof (e.g., ligand-binding fragments, signaling domains, and the like) can be recovered from the host cells and rapidly sequenced using modern equipment. These methods allow the routine and rapid determination of the importance of individual amino acid residues in a polypeptide of interest.

The CD5L polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al, Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology. John Wiley and Sons, Inc., NY, 1987.

Generally, a DNA sequence encoding a CD5L polypeptide is operably linked to other genetic elements required for its expression, including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

### Method of Screening Agents

In one aspect, the present invention provides methods for characterizing an agent for the ability to regulate T cell function. Such agent may be useful for treating autoimmune disease inflammatory response or hyperimmune resposne. Such agent may be useful for treating a cancer that is not inflammation related, inflammation related (e.g., after the cancer has progressed following inflammation) or enhancing an immune response in a subject. The method generally involves exposing a target cell to a test agent, and characterizing the effect of the agent on the target cell relative to a control target cell not exposed to the test agent, for example, by measuring the activity of a target gene or analyzing the transcriptional profile of the cell.

As used herein, the term “test compound” or “candidate agent” refers to an agent or collection of agents (e.g., compounds) that are to be screened for their ability to have an effect on the cell. Test compounds can include a wide variety of different compounds, including chemical compounds, mixtures of chemical compounds, e.g., polysaccharides, small organic or inorganic molecules (e.g. molecules having a molecular weight less than 2000 Daltons, less than 1000 Daltons, less than 1500 Dalton, less than 1000 Daltons, or less than 500 Daltons), biological macromolecules, e.g., peptides, proteins, peptide analogs, and analogs and derivatives thereof, peptidomimetics, nucleic acids, nucleic acid analogs and derivatives, an extract made from biological materials such as bacteria, plants, fungi, or animal cells or tissues, naturally occurring or synthetic compositions.

Depending upon the particular embodiment being practiced, the test compounds can be provided free in solution, or can be attached to a carrier, or a solid support, e.g., beads. A number of suitable solid supports can be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

A number of small molecule libraries are known in the art and commercially available. These small molecule libraries can be screened using the screening methods described herein. A chemical library or compound library is a collection of stored chemicals that can be used in conjunction with the methods described herein to screen candidate agents for a particular effect. A chemical library comprises information regarding the chemical structure, purity, quantity, and physiochemical characteristics of each compound. Compound libraries can be obtained commercially, for example, from Enzo Life Sciences™, Aurora Fine Chemicals™, Exclusive Chemistry Ltd.™, ChemDiv, ChemBridge™, TimTec Inc.™, AsisChem™, and Princeton Biomolecular Research™, among others.

Without limitation, the compounds can be tested at any concentration that can exert an effect on the cells relative to a control over an appropriate time period. In some embodiments, compounds are tested at concentrations in the range of about 0.01 nM to about 100 mM, about 0.1 nM to about 500 μM, about 0.1 μM to about 20 μM, about 0.1 μM to about 10 μM, or about 0.1 μM to about 5 μM.

The compound screening assay can be used in a high through-put screen. High throughput screening is a process in which libraries of compounds are tested for a given activity. High through-put screening seeks to screen large numbers of compounds rapidly and in parallel. For example, using microtiter plates and automated assay equipment, a laboratory can perform as many as 100,000 assays per day in parallel.

The compound screening assays described herein can involve more than one measurement of the cell or reporter function (e.g., measurement of more than one parameter and/or measurement of one or more parameters at multiple points over the course of the assay). Multiple measurements can allow for following the biological activity over incubation time with the test compound. In one embodiment, the reporter function is measured at a plurality of times to allow monitoring of the effects of the test compound at different incubation times.

The screening assay can be followed by a subsequent assay to further identify whether the identified test compound has properties desirable for the intended use. For example, the screening assay can be followed by a second assay selected from the group consisting of measurement of any of: bioavailability, toxicity, or pharmacokinetics, but is not limited to these methods.

Preferably, the screening assays measure, either directly or indirectly, the effect of the test compounds on T cell function. In some embodiments, the screening assays measure the effect of the test compounds on the expression of CD5L monomer, CD5L homodimer, and/or CD5L:p40 heterodimer. In certain embodiments, test compounds that increase the expression or activity of CD5L monomer, CD5L homodimer, and/or CD5L:p40 heterodimer are useful for treating an autoimmune disease, inflammation or hyperimmune response in a subject. In certain embodiments, test compounds that decrease the expression or activity of CD5L monomer, CD5L homodimer, and/or CD5L:p40 heterodimer are useful for treating a cancer that is not inflammation related, inflammation related after cancer progression or enhancing an immune response in a subject.

In another aspect, the present invention provides a method for predicting the effect of a test agent on a target cell of a patient in vivo, comprising culturing a target cell obtained from a patient in the system of the invention, exposing it to the test agent, and assaying for a pharmacological effect of the test agent on the target cell relative to a control target cell not treated with the test agent. In certain embodiments, the effect is selected from proliferation, viability, and differentiation, or combinations thereof. In certain embodiments, the effect is detected by assessing a change in gene expression profile between the target cell and the control target cell.

In some embodiments, the test agent is an agonist for CD5L monomer. In specific embodiments, the agonist is an antibody for CD5L monomer. In some embodiments, the test agent is an agonist for CD5L:CD5L homodimer. In specific embodiments, the agonist is an antibody for CD5L:CD5L homodimer. In some embodiments, the test agent is an agonist for CD5L:p40 heterodimer. In specific embodiments, the agonist is an antibody for CD5L:p40 heterodimer.

In another aspect, the present invention provides a method for screening a candidate pharmaceutical compounds, comprising culturing a target cell obtained from a patient in the system of the invention, expositing it to the candidate compound, and assaying for a pharmacological effect of the candidate compound on the target cell relative to a control target cell exposed to a CD5L monomer, CD5L:CD5L homodimer, and/or CD5L:p40 heterodimer. In certain embodiments, the effect is selected from proliferation, viability, and differentiation, or combinations thereof. In certain embodiments, the effect is detected by assessing a change in gene expression profile between the target cell and the control target cell.

In some embodiments, these methods can be used to screen for test agents (such as solvents, small molecule drugs, peptides, and polynucleotides) or environmental conditions (such as culture conditions or manipulation) that affect the characteristics of cells. Two or more agents can be tested in combination (by exposing to the cells either simultaneously or sequentially), to detect possible drug-drug interactions and/or rescue effects (e.g., by testing a toxin and a potential anti-toxin). Agent(s) and environmental condition(s) can be tested in combination (by treating the cells with a drug either simultaneously or sequentially relative to an environmental condition), to detect possible agent-environment interaction effects.

In certain embodiments, the assay to determine the characteristics of cells is selected in a manner appropriate to the cell type and agent and/or environmental factor being studied as disclosed in WO 2002/04113, which is hereby incorporated by reference in its entirely. For example, changes in cell morphology may be assayed by standard light, or electron microscopy. Alternatively, the effects of treatments or compounds potentially affecting the expression of cell surface proteins may be assayed by exposing the cells to either fluorescently labeled ligands of the proteins or antibodies to the proteins and then measuring the fluorescent emissions associated with each cell on the plate. As another example, the effects of treatments or compounds which potentially alter the pH or levels of various ions within cells may be assayed using various dyes which change in color at determined pH values or in the presence of particular ions. The use of such dyes is well known in the art. For cells, which have been transformed or transfected with a genetic marker, such as the β-galactosidase, alkaline phosphatase, or luciferase genes, the effects of treatments or compounds may be assessed by assays for expression of that marker. In particular, the marker may be chosen so as to cause spectrophotometrically assayable changes associated with its expression.

### Pharmaceutical Compositions

The methods include the manufacture and use of pharmaceutical compositions, which include any one or more of the agents described herein as active ingredient(s). Also included are the pharmaceutical compositions themselves. Further contemplated are compositions comprising one or more of the agents described herein alone or in combination with an agent useful in one or more of the diagnostic or treatment methods disclosed below.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, and combinations of two or more thereof, compatible with pharmaceutical administration. Supplementary active compounds can also be incorporated into the compositions.

Pharmaceutical compositions are typically formulated to be compatible with the intended route of administration. Examples of routes of administration include parenteral (e.g., intravenous), intrathecal, oral, and nasal or intranasal (e.g., by administration as drops or inhalation) administration. In some embodiments, such as for compounds that don't cross the blood brain barrier, delivery directly into the CNS or CSF can be used, e.g., using implanted intrathecal pumps (see, e,g., Borrini et al., Archives of Physical Medicine and Rehabilitation 2014; 95:1032-8; Penn et al., N. Eng. J. Med. 320:1517-21 (1989); and Rezai et al., Pain Physician 2013; 16:415-417) or nanoparticles, e.g., gold nanoparticles (e.g., glucose-coated gold nanoparticles, see, e.g., Gromnicova et al. (2013) PLoS ONE 8(12): e81043). Methods of formulating and delivering suitable pharmaceutical compositions are known in the art, see, e.g., the books in the series Drugs and the Pharmaceutical Sciences: a Series of Textbooks and Monographs (Dekker, N.Y.); and Allen et al., Ansel's Pharmaceutical Dosage Forms and Drug Delivery Systems, Lippincott Williams & Wilkins; 8th edition (2004).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

For oral administration, the compositions can be formulated with an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998).

Liposomes (e.g., as described in U.S. Pat. No. 6,472,375) and microencapsulation can also be used to deliver a compound. Biodegradable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser, e.g., single-dose dispenser together with instructions for administration. The container, pack, or dispenser can also be included as part of a kit that can include, for example, sufficient single-dose dispensers for one day, one week, or one month of treatment.

### Methods of Treatment

The term “treating” is art-recognized and includes administration to the host or patient or subject of one or more of the subject compositions, e.g., to diminish, ameliorate, or stabilize the existing unwanted condition or side effects thereof. In aspects of the invention, treatment is for the patient or subject in need thereof. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. By “ameliorate” is meant decrease, suppress, attenuate, diminish, arrest, or stabilize the development or progression of a disease.

As used herein, a subject means a human or animal (in the case of an animal, more typically a mammal, and can be, but is not limited to, a non-human animal or mammal). In one aspect, the subject is a human. A “subject” mammal can include, but is not limited to, a human or non-human mammal, such as a primate, bovine, equine, canine, ovine, feline, or rodent; and, it is understood that an adult human is typically about 70 kg, and a mouse is about 20 g, and that dosing from a mouse or other non-human mammal can be adjusted to a 70 kg human by a skilled person without undue experimentation.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably more than a 30% change, a 35% change, a 40% change, and most preferably a 50% or greater change in expression levels. In a more preferred embodiment of the invention, the upregulation or increase in biomarker levels is at least greater than a 30% increase over baseline or normal population reference standards.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “marker”, “biomarker” or “biological marker” is meant any clinical indicator, protein, metabolite, or polynucleotide having an alteration associated with a disease or disorder or a measurable indicator of some biological state or condition. Biomarkers are often measured and evaluated (e.g. whether their levels are increased or decreased or remain unchanged) to examine normal biological processes, pathogenic processes, or pharmacologic responses to a therapeutic intervention. By “reference” is meant a standard or control condition.

Without being bound by theory, CD5L monomer, CD5L:CD5L homodimers and CD5L:p40 heterodimers are believed to regulate T cells and alter immune function, and can promote suppression of pathogenic Th17 and Th1 phenotypes. Agonists of CD5L monomer, CD5L:CD5L homodimers, and/or CD5L:p40 heterodimers (e.g., CD5L:p40 heterodimer polypeptides), can be administered to modulate or suppress an immune response. Antagonists of CD5L monomer, CD5L:CD5L homodimers, and/or CD5L:p40 heterodimers (e.g., CD5L:p40 heterodimer polypeptides), can be administered to enhance immune response.

Aspects of disclosure relate to the use of one or more of the proteins or polypeptides, antibodies, equivalents, or compositions for use in the treatment of conditions associated with overactive inflammation or immunity, e.g., autoimmune diseases, e.g., in which pathogenic T cells are present at increased levels and/or have increased activity, such as multiple sclerosis (MS). Autoimmune conditions that may benefit from treatment using the compositions and methods include, but are not limited to, for example, MS, Addison's Disease, alopecia, ankylosing spondylitis, antiphospholipid syndrome, autoimmune hemolytic anemia, autoimmune hepatitis, autoimmune oophoritis, Bechet's disease, bullous pemphigoid, celiac disease, chronic fatigue immune dysfunction syndrome (CFIDS), chronic inflammatory demyelinating polyneuropathy, Churg-Strauss syndrome, cicatricial pemphigoid, cold agglutinin disease, CREST Syndrome, Crohn's disease, diabetes (e.g., type I), dysautonomia, endometriosis, eosinophilia-myalgia syndrome, essential mixed cryoglobulinemia, fibromyalgia, syndrome/fibromyositis, Graves' disease, Guillain Barre syndrome, Hashimoto's thyroiditis, idiopathic pulmonary fibrosis, idiopathic thrombocytopenia purpura (ITP), inflammatory bowel disease (IBD), lichen planus, lupus, Meniere's disease, mixed connective tissue disease (MCTD), multiple sclerosis, myasthenia gravis, pemphigus, spernicious anemia, polyarteritis nodosa, polychondritis, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sj ogren's syndrome, spondyloarthropathy (spondyloarthritides), stiff-man syndrome, Takayasu arteritis, temporal arteritis/giant cell arteritis, autoimmune thyroid disease, ulcerative colitis, autoimmune uveitis, autoimmune vasculitis, vitiligo, and Wegener's granulomatosis. In some embodiments, the autoimmune disease is MS, IBD, Crohn's disease, spondyloarthritides, Systemic Lupus Erythematosus, Vitiligo, rheumatoid arthritis, psoriasis, Sjögren's syndrome, or diabetes, e.g., Type I diabetes, all of which have been linked to Th17 cell dysfunction (see, e.g., Korn et al., Annu Rev Immunol. 2009; 27:485-517Dong, Cell Research (2014) 24:901-903; Zambrano-Zaragoza et al., Int J Inflam. 2014; 2014: 651503; Waite and Skokos, International Journal of Inflammation; Volume 2012 (2012), Article ID 819467, 10 pages, dx.doi.org/10.1155/2012/819467; Han et al., Frontiers of Medicine 9(1):10-19 (2015).

Some embodiments include treatment of autoimmune diseases, such as multiple sclerosis (MS) or IBD, using one or more of the agonists. In some embodiments, once it has been determined that a person has an autoimmune disease, e.g., MS or IBD, then a treatment comprising administration of a therapeutically effective amount of one or more of the agonists. In some embodiments, soluble CD5L monomers, CD5L homodimers and/or CD5L:p40 heterodimers can be administered in combination with the one or more agonists.

Generally, the methods include administering a therapeutically effective amount of one or more of the agents, to a subject who is in need of, or who has been determined to be in need of, such treatment. As used in this context, to “treat” means to ameliorate or reduce the severity of at least one symptom of a disease or condition. For instance, a treatment can result in a reduction in one or more symptoms of an autoimmune disease, e.g., for MS, e.g., depression and fatigue, bladder dysfunction, spasticity, pain, ataxia, and intention tremor. A therapeutically effective amount can be an amount sufficient to prevent the onset of an acute episode or to shorten the duration of an acute episode, or to decrease the severity of one or more symptoms, e.g., heat sensitivity, internuclear ophthalmoplegia, optic neuritis, and Lhermitte symptom. In some embodiments, a therapeutically effective amount is an amount sufficient to prevent the appearance of, delay or prevent the growth (i.e., increase in size) of, or promote the healing of a demyelinated lesion in one or more of the brain, optic nerves, and spinal cord of the subject, e.g., as demonstrated on MRI.

Alternatively or in addition, the methods can be used to treat other conditions associated with hyperimmune responses, e.g., cancers associated with inflammation such as colorectal cancers. In certain inflammation-related cancers the IL-23 pathway has been shown to promote tumorigenesis (e.g., in colorectal cancer, carcinogen-induced skin papilloma, fibrosarcomas, mammary carcinomas and certain cancer metastasis; these studies have suggested that IL-23 and Th17 cells play a role in some cancers, such as, by way of non-limiting example, colorectal cancers. See e.g., Ye J, Livergood R S, Peng G. “The role and regulation of human Th17 ceils in tumor immunity.” Am J Pathol 2013 January; 182(1): 10-20. doi: 10.1016/j.ajpath.2012.08.041. Epub 2012 Nov. 14). In such cancer types, CD5L and CD5L:p40 and agents that promote their function can have anti-tumor effects. (Teng et al., 2015 Nat Med 21; Wang and Karin, Clin Exp Rheumatol 2015; 33). Thus CD5L monomers, CD5L homodimers and/or CD5L:p40 heterodimers, or nucleic acids encoding CD5L monomers, CD5L homodimers and/or CD5L:p40 heterodimers, can be used to treat or reduce risk of developing these cancers.

Some embodiments relate to the use of one or more of the proteins or polypeptides, antibodies, equivalents, or compositions for use in the treatment of cancers that would benefit from immunotherapy (e.g., cancers that are not inflammation related); subjects who have a primary or secondary immune deficiency; or subjects who have an infection with a pathogen, e.g., viral, bacterial, or fungal pathogen.

As used in this context, to “treat” means to ameliorate or reduce the severity of at least one clinical parameter of a condition (e.g., cancer). In some embodiments, the parameter is tumor size, tumor growth rate, recurrence, or metastasis, and an improvement would be a reduction in tumor size or no change in a normally fast growing tumor; a reduction or cessation of tumor growth; a reduction in, delayed, or no recurrence, or a reduction in, delayed, or no metastasis. Administration of a therapeutically effective amount of a compound for the treatment of a cancer would result in one or more of a reduction in tumor size or no change in a normally fast growing tumor; a reduction or cessation of tumor growth; or a reduction in, delayed, or no metastasis. In some embodiments, e.g., a treatment designed to prevent recurrence of cancer, the treatment would be given after a localized tumor has been removed, e.g., surgically, or treated with radiation therapy or with targeted therapy with or without other therapies such as standard chemotherapy. Without wishing to be bound by theory, such a treatment may work by keeping micrometastases dormant, e.g., by preventing them from being released from dormancy.

As used herein, the term “hyperproliferative” refer to cells having the capacity for autonomous growth, i.e., an abnormal state or condition characterized by rapidly proliferating cell growth. Hyperproliferative disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A “tumor” is an abnormal growth of hyperproliferative cells. “Cancer” refers to pathologic disease states, e.g., characterized by malignant tumor growth. The methods can be used to treat cancer, e.g., solid tumors of epithelial origin, e.g., as defined by the ICD-O (International Classification of Diseases—Oncology) code (revision 3), section (8010-8790), e.g., early stage cancer, is associated with the presence of a massive levels of satellite due to increase in transcription and processing of satellite repeats in epithelial cancer cells. Thus the methods can include the interference of satellite repeats in a sample comprising cells known or suspected of being tumor cells, e.g., cells from solid tumors of epithelial origin, e.g adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), liver cancer (e.g. liver hepatocellular carcinoma), neuroendocrine prostate cancer (NEPC), non-small cell lung cancer (NSCLC), stomach and/or esophageal cancer, desmoplastic small-round-cell tumor (DESM) cells.

Cancers of epithelial origin can include pancreatic cancer (e.g., pancreatic adenocarcinoma), lung cancer (e.g., non-small cell lung carcinoma or small cell lung carcinoma), prostate cancer, breast cancer, renal cancer, ovarian cancer, melanoma or colon cancer. Leukemia may include AML, CML or CLL and in some embodiments comprises cancerous MDSC. The methods can also be used to treat early preneoplastic cancers as a means to prevent the development of invasive cancer.

Aspects of disclosure also relate to the use of one or more of the proteins or polypeptides, antibodies, equivalents, or compositions in the treatment of cancer, wherein the cancer is inhibited by complement. Complement is a central part of the immune system that has developed as a first defense against non-self cells. Neoplastic transformation is accompanied by an increased capacity of the malignant cells to activate complement. In fact, clinical data demonstrate complement activation in cancer patients. Complement has two pathways, the classical pathway associated with specific defense, and the alternative pathway that is activated in the absence of specific antibody, and is thus non-specific. In the classical pathway, antigen-antibody complexes are recognized when Cl interacts with the Fc of the antibody, such as IgM and to some extent, IgG, ultimately causing mast cells to release chemotactic factors, vascular mediators and a respiratory burst in phagocytes, as one of many mechanisms. The key complement factors include C3a and C5a, which cause mast cells to release chemotactic factors such as histamine and serotonin that attract phagocytes, antibodies and complement, etc. Other key complement factors are C3b and C5b, which enhance phagocytosis of foreign cells, and C8 and C9, which induce lysis of foreign cells (membrane attack complex). Recent research showed that complement elements can promote tumor growth in the context of chronic inflammation. Roben Pio et al. Adv. Exp. Med. Biol. (2014) 772:229-262. On the basis of the use of protective mechanisms by malignant cells, complement activation is considered part of the body's immunosurveillance against cancer. Research showed that in hepatocellular carcinoma cells, CD5L accumulates on the cell surface and specifically provokes cell death through activation of complement cascade (Maehara et al., Cell Reports, (2014) 9:61-74). Therefore, the present disclosure encompasses methods of treating cancer that is inhibited by complement cascade, by administering an agonist of CD5L, CD5L homodimer, and/or CD5L:p40 heterodimer. In some embodiments, the cancer is hepatocellular carcinoma (HCC). Therefore, the present disclosure encompasses methods of treating cancer that is promoted by complement, by administering an antagonist of CD5L, CD5L homodimer, and/or CD5L:p40 heterodimer. In some embodiments, complement activation is increased in the cancer patient. In specific embodiments, the cancer is selected from the group consisting of non-small cell lung cancer, ovarian cancer, colorectal cancer, carcinomas of the digested tract, brain tumor, chronic lymphatic leukemia, cervical cancer, papillary thyroid carcinoma, follicular lymphoma, mucosa-associated lymphoid tissue lymphoma, multiple myeloma.

In some embodiments, CD5L, CD5L homodimer, and/or CD5L:p40 heterodimer may be used as a biomarker for disease progression. For example, serum CD5L, CD5L homodimer, and/or CD5L:p40 concentration can be measured and compared against a control concentration. In some embodiments, serum CD5L, CD5L homodimer, and/or CD5L:p40 concentration in a subject is measured at multiple time points, and the change in concentration is used to indicate disease progression or effectiveness of treatment.

### Combination Therapy

In some embodiments, a treatment is administered in combination with a treatment for an autoimmune disease, inflammation and/or a hyperimmune response.

In some embodiments, the treatment used in combination with one or more agonist is a standard treatment for autoimmune disease, inflammation and/or a hyperimmune response, e.g. an FDA approved therapeutic for any one of the aforementioned autoimmune diseases and/or a hyperimmune responses.

For example, in the case of MS, treatment can include administration of corticosteroid therapy, interferon beta-1b, Glatiramer acetate, mitoxantrone, Fingolimod, teriflunomide, dimethyl fumarate, natalizumab, cannabis, or a combination thereof. In some embodiments, the treatment is administered in combination with a treatment for one or more symptoms of MS, e.g., depression and/or fatigue, bladder dysfunction, spasticity, pain, ataxia, and intention tremor. Such treatments can include pharmacological agents, exercise, and/or appropriate orthotics. Additional information on the diagnosis and treatment of MS can be found at the National MS Society website (nationalmssociety.org).

In certain embodiments, the treatment used in combination with one or more agonists is a standard treatment for cancer. Standards of care for cancer generally include surgery, lymph node removal, radiation, chemotherapy, targeted therapies, antibodies targeting the tumor, and immunotherapy. Glucocorticoids are often administered to help patients tolerate treatment, rather than as a chemotherapeutic that targets the cancer itself (see, e.g., Pufall, Glucocorticoids and Cancer, Adv Exp Med Biol. 2015; 872: 315-333. doi:10.1007/978-1-4939-2895-8_14). In certain embodiments, one or more agonists are used for their anti-inflammatory properties or to prevent hypersensitivity caused by a standard treatment. Immunotherapy can include checkpoint blockers (CBP), chimeric antigen receptors (CARs), and adoptive T-cell therapy. In certain embodiments, immunotherapy leads to immune-related adverse events (irAEs) and the standard of care includes treatment with glucocorticoids to generally suppress immune responses (see, e.g., Gelao et al., Immune Checkpoint Blockade in Cancer Treatment: A Double-Edged Sword Cross-Targeting the Host as an “Innocent Bystander”, Toxins 2014, 6, 914-933; doi:10.3390/toxins6030914). In certain embodiments, one or more agonists are used to more specifically prevent immune-related adverse events (irAEs) in a combination treatment with one or more checkpoint inhibitors. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Specific check point inhibitors include, but are not limited to anti-CTLA4 antibodies (e.g., Ipilimumab), anti-PD-1 antibodies (e.g., Nivolumab, Pembrolizumab), and anti-PD-L1 antibodies (e.g., Atezolizumab). In certain embodiments, one or more agonists are used to relieve bone pain other discomfort that may arise from metastatic disease and CNS compression due to metastatic disease.

In some embodiments, the treatment used in combination with one or more antagonist is a standard treatment for cancer, e.g. an FDA approved therapeutic for any one of the aforementioned cancers. In some embodiments, the methods include administering a standard anti-cancer therapy to a subject. Cancer treatments include those known in the art, e.g., surgical resection with cold instruments or lasers, radiotherapy, phototherapy, biologic therapy (e.g., with tyrosine kinase inhibitors), radiofrequency ablation (RFA), radioembolisation (e.g., with 90Y spheres), chemotherapy, and immunotherapy.

Immunotherapies can also include administering one or more of: adoptive cell transfer (ACT) involving transfer of ex vivo expanded autologous or allogeneic tumor-reactive lymphocytes, e.g., dendritic cells or peptides with adjuvant; chimeric antigen receptors (CARs); cancer vaccines such as DNA-based vaccines, cytokines (e.g., IL-2), cyclophosphamide, anti-interleukin-2R immunotoxins, Prostaglandin E2 Inhibitors (e.g., using SC-50) and/or checkpoint inhibitors including antibodies such as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Kruger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4):342-7. In some embodiments, the methods include administering a composition comprising tumor-pulsed dendritic cells, e.g., as described in WO2009/114547 and references cited therein. See also Shiao et al., Genes & Dev. 2011. 25: 2559-2572.

In certain embodiments, the treatment used in combination with one or more antagonists is a check point blockade therapy to enhance an immune response. In certain embodiments, the one or more antagonists are co-administered with, administered before or administered after a check point blockade therapy. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Specific check point inhibitors include, but are not limited to anti-CTLA4 antibodies (e.g., Ipilimumab), anti-PD-1 antibodies (e.g., Nivolumab, Pembrolizumab), and anti-PD-L1 antibodies (e.g., Atezolizumab).

In some embodiments, the treatment used in combination with one or more agonists is adoptive cell therapy. In certain embodiments, an agonist of CD5L is used to prevent an autoimmune reaction. In certain embodiments, the one or more agonists are administered with or after adoptive cell transfer.

In some embodiments, the treatment used in combination with one or more antagonists is adoptive cell therapy. In some embodiments, the treatment used in combination with one or more antagonists is adoptive cell therapy using engineered immune cells, such as T-cells (e.g., CAR T cells or tumor infiltrating lymphocytes). In certain embodiments, an antagonist of CD5L is used to enhance an immune response. In certain embodiments, the one or more antagonists are administered before, with or after adoptive cell transfer.

### Adoptive Cell Transfer

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 September 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); K-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member lA (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p5³; p⁵³ mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and 3 chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L23 5E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3t or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3; see U.S. Pat. Nos. 8,911,993; 8,916,381; 8,975,071; 9,101,584; 9,102,760; 9,102,761). Third-generation CARs include a combination of costimulatory endodomains, such a CD3i-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3t or scFv-CD28-OX40-CD3; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3t or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD1 id, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3I chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS (SEQ ID NO 19)). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3 chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO 20) and continuing all the way to the carboxy-terminus of the protein. To encode the anti-CD 19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3 chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO 20) and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS (SEQ ID NO 19). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD 19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am.J.Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-13) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3t and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+Th1 cells and CD8+ CTLs to induce a synergistic antitumour response (see, e.g., Li et al., Adoptive cell therapy with CD4+T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr. 12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.33 89/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each a and 3 chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRa or TCRP3 can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block a downstream target of CD5L monomers, CD5L:CD5L homodimer, CD5L:p40 heterodimers and p40:p40 homodimers. In certain embodiments, inhibiting or blocking, or inducing or enhancing a downstream target in an immune cell (e.g., T cell, Th17 cell) may enhance and immune response or suppress inflammation or an autoimmune response upon transfer. The downstream targets may include Il17f, Il17a, Ildr1, Il1r1, Lgr4, Ptpnl4, Paqr8, Timp1, Il1rn, Smim3, Gap43, Tigit, Mmp10, Il22, Enpp2, Iltifb, Ido1, 1123r, Stom, Bc12111, 5031414D18Rik, Il24, Itga7, Il6, Epha2, Mt2, Upp1, Snord104, 5730577I03Rik, Slcl8b1, Ptprj, Clip3, Mir5104, Ppifos, Rab13, Hist1h2bn, Ass1, Cd200r1, E130112N10Rik, Mxd4, Casp6, Gatm, Tnfrsf8, Gp49a, Gadd45g, Ccr5, Tgm2, Lilrb4, Ecm1, Arhgap18, Serpinb5, Cysltr1, Enpp1, Selp, Slc38a4, Gm14005, Epb4.1l4b, Moxd1, Klra7, Igfbp4, Tnip3, Gstt1, Pglyrp2, Il12rb2, Ctla2a, Plac8, Ly6c1, Sell, Ncf1, Trp53i11, B3gnt3, Kremen2, Matk, Ltb4r1, Ets1, Tnfrsf26, Cd28, Rybp, Ppp1r3c, Thy1, Trib2, Sema3b, Pros1, 1133, Gm5483, Myh11, Cntd1, Ms4a4b, Treml2, 3110009E18Rik, Pglyrp1, Amd1, Slc24a5, Snhg9, Ifi27l1, Irf7, Mx1, Snhgl0, Il4, Snora43, H2-L, Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Dusp2, Snx20, Lamp1, Faf1, Gpatch3, Dapk3, 1110065P20Rik, Vaultrc5, Myl4, Insl3, Tgoln2, BC022687, C230035I16Rik, Hvcn1, Myh10, Dhrs3, Acsl6, Rgs2, Cc120, Cc13, Dlg2, Ccr6, Cc14, Dusp14, Apol9b, Cd72, Ispd, Cd70, S100al, Lgals3, Slc15a3, Nkg7, Serpinc1, Olfr175-ps1, 119, Pdlim4, Il3, Insl6, Perp, Cd51, Serpine2, Galnt14, Tff1, Ppfibp2, Bdh2, Mlf1, Il1a, Osr2, Gm5779, Ebf1, Spink2, Egfr and Ccdc155. Specific genes upregulated by CD5L:p40 may include Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Dusp2, Snx20, Lamp1, Faf1, Gpatch3, Dapk3, 1110065P20Rik and Vaultrc5. In certain embodiments, Dusp2 is inhibited or deleted in T cells to enhance an immune response (e.g., CD8 T cells, Th17 cells).

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAMIVSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, 0-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, 0-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled 32-microglobulin (132m) into MHC class I/02m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

In some embodiments, relevant candidates to be used in the combination with one or more agonists or antagonists may be screened according to a variety of approaches. In certain embodiments, genetic modifying agents may be used (e.g. those involving CRISPR-Cas or other gene editing or gene therapy based approaches).

As mentioned above, some embodiments comprise methods gene targeting and/or genome editing. Such methods are useful, e.g., in the context of decreasing protein expression in vivo and/or modifying cells in vitro (e.g., in the context of adoptive cell therapies). In some embodiments, genes are targeting and/or edited using DNA binding proteins.

### Genetic Modifying Agents

In certain embodiments, the one or more modulating agents may be a genetic modifying agent. The genetic modifying agent may comprise a CRISPR system, a zinc finger nuclease system, a TALEN, or a meganuclease.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10. 1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US 13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the (3-actin promoter, the phosphoglycerol kinase (PGK) promoter, and the EF 1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoide cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemicially modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemicially modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (T), N1-methylpseudouridine (melΨ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas proten (Chen et al. Cell. (2013); 155(7): 1479-1491). In particular embodiments the stem of the stemloop is extended by at least 1, 2, 3, 4, 5 or more complementary basepairs (i.e. corresponding to the addition of 2,4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the compelementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/j ournal/v8/n5/full/nchembio. 922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone HEREEdinburgh, London & NY, 1977HERE).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extented length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide moleucle, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi: 10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCasI3d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366): 1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure™, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/naturel5521. Epub 2015 September 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpfl Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.

Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.

Hsu et al. (2014) is a review article that discusses generally CRISPR-Cas9 history from yogurt to genome editing, including genetic screening of cells.

Wang et al. (2014) relates to a pooled, loss-of-function genetic screening approach suitable for both positive and negative selection that uses a genome-scale lentiviral single guide RNA (sgRNA) library.

Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

Swiech et al. demonstrate that AAV-mediated SpCas9 genome editing can enable reverse genetic studies of gene function in the brain.

Konermann et al. (2015) discusses the ability to attach multiple effector domains, e.g., transcriptional activator, functional and epigenomic regulators at appropriate positions on the guide such as stem or tetraloop with and without linkers.

Zetsche et al. demonstrates that the Cas9 enzyme can be split into two and hence the assembly of Cas9 for activation can be controlled.

Chen et al. relates to multiplex screening by demonstrating that a genome-wide in vivo CRISPR-Cas9 screen in mice reveals genes regulating lung metastasis.

Ran et al. (2015) relates to SaCas9 and its ability to edit genomes and demonstrates that one cannot extrapolate from biochemical assays.

Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.

Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.

Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.

Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.

Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.

Zetsche et al. (2015) reported characterization of Cpfl, a class 2 CRISPR nuclease from Francisella novicida U112 having features distinct from Cas9. Cpfl is a single RNA-guided endonuclease lacking tracrRNA, utilizes a T-rich protospacer-adjacent motif, and cleaves DNA via a staggered DNA double-stranded break.

Shmakov et al. (2015) reported three distinct Class 2 CRISPR-Cas systems. Two system CRISPR enzymes (C2c1 and C2c3) contain RuvC-like endonuclease domains distantly related to Cpfl. Unlike Cpfl, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 June. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 14 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

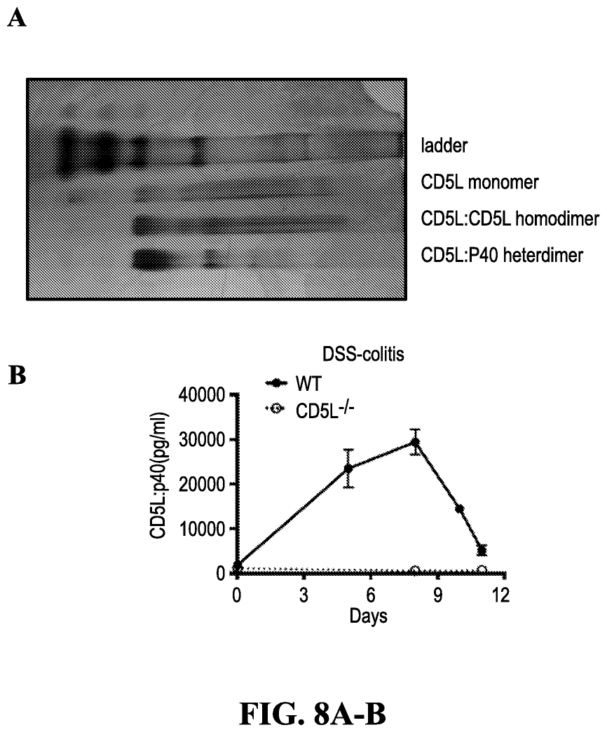

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ. I.D. No. 21)
M D P I R S R T P S P A R E L L S G P Q P D G V Q P T A D R G V S P
P A G G P L D G L P A R R T M S R T R L P S P P A P S P A F S A D S
F S D L L R Q F D P S L F N T S L F D S L P P F G A H H T E A A T G
E W D E V Q S G L R A A D A P P P T M R V A V T A A R P P R A K P A
P R R R A A Q P S D A S P A A Q V D L R T L G Y S Q Q Q Q E K I K P
K V R S T V A Q H H E A L V G H G F T H A H I V A L S Q H P A A L G
T V A V K Y Q D M I A A L P E A T H E A I V G V G K Q W S G A R A L
E A L L T V A G E L R G P P L Q L D T G Q L L K I A K R G G V T A V
E A V H A W R N A L T G A P L N

An exemplary amino acid sequence of a C-terminal capping region is:

(SEQ. I.D. No. 22)
R P A L E S I V A Q L S R P D P A L A A L T N D H L V A L A C L G
G R P A L D A V K K G L P H A P A L I K R T N R R I P E R T S H R
V A D H A Q V V R V L G F F Q C H S H P A Q A F D D A M T Q F G M
S R H G L L Q L F R R V G V T E L E A R S G T L P P A S Q R W D R
I L Q A S G M K R A K P S P T S T Q T P D Q A S L H A F A D S L E
R D L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleasesi characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

Some embodiments comprise decreasing protein expression (e.g., CD5L or p40 expression) with inhibitory nucleic acids. Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes, and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112; Burnett and Rossi (2012) Chem Biol. 19 (1):60-71; and WO2015130968, which is incorporated herein by reference in its entirety.

In some embodiments, the inhibitory nucleic acids are 10 to 50, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range there within. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies inhibitory nucleic acids having antisense portions of 12, 13, 14, 15, 16, 17, 18, 19, 20, 21, 22, 23, 24, 25, 26, 27, 28, 29 or 30 nucleotides in length, or any range there within.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 8,604,192; 8,697,663; 8,703,728; 8,796,437; 8,865,677; and 8,883,752 each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)-O—CH2 (known as a methylene(methylimino) or MMI backbone], CH2-O—N (CH3)-CH2, CH2-N(CH3)-N(CH3)-CH2 and O—N(CH3)-CH2-CH2 backbones, wherein the native phosphodiester backbone is represented as O—P—O—CH,); amide backbones (De Mesmaeker (1995) Ace. Chem. Res. 28:366-374); morpholino backbone structures (Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, Nielsen (1991) Science 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, phosphonoacetate phosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey (2002) Biochemistry 41(14), 4503-4510); Genesis, volume 30, issue 3, 2001; Heasman, (2002) Dev. Biol. 243, 209-214; Nasevicius (2000) Nat. Genet. 26, 216-220; Lacerra (2000) Proc. Natl. Acad. Sci. 97, 9591-9596; and U.S. Pat. No. 5,034,506, issued Jul. 23, 1991. Cyclohexenyl nucleic acid oligonucleotide mimetics are described in Wang (2000) Am. Chem. Soc. 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623, 070; 5,663,312; 5,633,360; 5,677,437; 5,677,439; and 8,927,513 each of which is herein incorporated by reference.

One or more substituted sugar moieties can also be included, e.g., one of the following at the 2′ position: OH, SH, SCH₃, F, OCN, OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)](Martin (1995) Helv. Chim. Acta 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, 2,6-diaminopurine; 5-ribosyluracil (Carlile (2014) Nature 515(7525): 143-6). Kornberg, A., DNA Replication, W. H. Freeman & Co., San Francisco, 1980, pp 75-77; Gebeyehu (1987) Nucl. Acids Res. 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide. In some embodiments, both the nucleobase and backbone may be modified to enhance stability and activity (El-Sagheer (2014) Chem Sci 5:253-259)

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen (1991) Science 254, 1497-1500; and Shi (2015).

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger (1989) Proc. Natl. Acad. Sci. USA 86, 6553-6556), cholic acid (Manoharan (1994) Bioorg. Med. Chem. Let. 4, 1053-1060), a thioether, e.g., hexyl-S-tritylthiol (Manoharan (1992) Ann. N. Y. Acad. Sci. 660, 306-309; Manoharan (1993) Bioorg. Med. Chem. Let. 3, 2765-2770), a thiocholesterol (Oberhauser (1992) Nucl. Acids Res. 20, 533-538), an aliphatic chain, e.g., dodecandiol or undecyl residues (Kabanov (1990) FEBS Lett. 259, 327-330; Svinarchuk (1993) Biochimie 75, 49-54), a phospholipid, e.g., di-hexadecyl-rac-glycerol or triethylammonium 1, 2-di-O-hexadecyl-rac-glycero-3-H-phosphonate (Manoharan (1995) Tetrahedron Lett. 36, 3651-3654; Shea (1990) Nucl. Acids Res.18, 3777-3783), a polyamine or a polyethylene glycol chain (Mancharan (1995) Nucleosides & Nucleotides 14, 969-973), or adamantane acetic acid (Manoharan (1995) Tetrahedron Lett. 36, 3651-3654), a palmityl moiety (Mishra (1995) Biochim. Biophys. Acta 1264, 229-237), or an octadecylamine or hexylamino-carbonyl-t oxycholesterol moiety (Crooke (1996) J. Pharmacol. Exp. Ther. 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928; 5,688,941, 8,865,677; 8,877,917 each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The inhibitory nucleic acids useful in the present methods are sufficiently complementary to the target lncRNA, i.e., hybridize sufficiently well and with sufficient specificity, to give the desired effect. “Complementary” in this context refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a lncRNA, then the bases are considered to be complementary to each other at that position. 100% complementarity is not required.

In some embodiments, the location on a target lncRNA to which an inhibitory nucleic acids hybridizes is defined as a target region to which a protein binding partner binds. These regions can be identified by reviewing the data submitted herewith in Appendix I and identifying regions that are enriched in the dataset; these regions are likely to include the protein binding sequences. Routine methods can be used to design an inhibitory nucleic acid that binds to this sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure. Target segments 5-500 nucleotides in length comprising a stretch of at least five (5) consecutive nucleotides within the protein binding region, or immediately adjacent thereto, are considered to be suitable for targeting as well. Target segments can include sequences that comprise at least the 5 consecutive nucleotides from the 5′-terminus of one of the protein binding regions (the remaining nucleotides being a consecutive stretch of the same RNA beginning immediately upstream of the 5′-terminus of the binding segment and continuing until the inhibitory nucleic acid contains about 5 to about 100 nucleotides). Similarly preferred target segments are represented by RNA sequences that comprise at least the 5 consecutive nucleotides from the 3′-terminus of one of the illustrative preferred target segments (the remaining nucleotides being a consecutive stretch of the same lncRNA beginning immediately downstream of the 3′-terminus of the target segment and continuing until the inhibitory nucleic acid contains about 5 to about 100 nucleotides). One having skill in the art armed with the sequences provided herein will be able, without undue experimentation, to identify further preferred protein binding regions to target.

Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target RNAs), to give the desired effect.

The inhibitory nucleic acids used to practice the methods described herein, whether RNA, cDNA, genomic DNA, vectors, viruses or hybrids thereof, can be isolated from a variety of sources, genetically engineered, amplified, and/or expressed, generated recombinantly or synthetically by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; Maier (2000) Org Lett 2(13):1819-1822; Egeland (2005) Nucleic Acids Res 33(14):e125; Krotz (2005) Pharm Dev Technol 10(2):283-90 U.S. Pat. No. 4,458,066. Recombinant nucleic acid sequences can be individually isolated or cloned and tested for a desired activity. Any recombinant expression system can be used, including e.g. in vitro bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion or “seamless cloning”, ligation, transformation, plasmid purification, and DNA sequencing, for example as described in Sambrook et al. “Molecular Cloning: A Laboratory Manual.” (1989)), Coffin et al. (Retroviruses. (1997)) and “RNA Viruses: A Practical Approach” (Alan J. Cann, Ed., Oxford University Press, (2000)). “Seamless cloning” allows joining of multiple fragments of nucleic acids in a single, isothermal reaction (Gibson (2009) Nat Methods 6:343-345; Werner (2012) Bioeng Bugs 3:38-43; Sanjana (2012) Nat Protoc 7:171-192). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus (Warnock (2011) Methods in Molecular Biology 737:1-25). The recombinant vectors capable of expressing the nucleic acids of the invention can be delivered as described herein, and persist in target cells (e.g., stable transformants).

This can be achieved, for example, by administering an inhibitory nucleic acid, e.g., antisense oligonucleotides complementary to p40 and/or CD5L. Other inhibitory nucleic acids for use in practicing the methods described herein and that are complementary to p40 and/or CD5L can be those which inhibit post-transcriptional processing of p40 or CD5L, such as inhibitors of mRNA translation (antisense), agents of RNA interference (RNAi), catalytically active RNA molecules (ribozymes), and RNAs that bind proteins and other molecular ligands (aptamers). Additional methods exist to inhibit endogenous microRNA (miRNA) activity through the use of antisense-miRNA oligonucleotides (antagomirs) and RNA competitive inhibitors or decoys (miRNA sponges).

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to p40 and/or CD5L. Thus, oligonucleotides are chosen that are sufficiently complementary to the target, i.e., that hybridize sufficiently well and with sufficient specificity, to give the desired effect, while striving to avoid significant off-target effects i.e. must not directly bind to, or directly significantly affect expression levels of, transcripts other than the intended target. The optimal length of the antisense oligonucleotide may very but it should be as short as possible while ensuring that its target sequence is unique in the transcriptome i.e. antisense oligonucleotides may be as short as 12-mers (Seth (2009) J Med Chem 52:10-13) to 18-22 nucleotides in length.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target.

It is understood in the art that a complementary nucleic acid sequence need not be 100% complementary to that of its target nucleic acid to be specifically hybridisable. A complementary nucleic acid sequence of the invention is specifically hybridisable when binding of the sequence to the target DNA or RNA molecule interferes with the normal function of the target DNA or RNA to cause a loss of activity, and there is a sufficient degree of complementarity to avoid non-specific binding of the sequence to non-target sequences under conditions in which specific binding is desired, i.e., under physiological conditions in the case of in vivo assays or therapeutic treatment, and in the case of in vitro assays, under conditions in which the assays are performed under suitable conditions of stringency. The antisense oligonucleotides useful in the methods described herein have at least 80% sequence complementarity to a target region within the target nucleic acid, e.g., 90%, 95%, or 100% sequence complementarity to the target region within p40 or CD5L (e.g., a target region comprising the seed sequence). Percent complementarity of an antisense compound with a region of a target nucleic acid can be determined routinely using basic local alignment search tools (BLAST programs) (Altschul (1990) J. Mol. Biol. 215, 403-410; Zhang and Madden (1997) Genome Res. 7, 649-656). The specificity of an antisense oligonucleotide can also be determined routinely using BLAST program against the entire genome of a given species

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York, Hilario (2007) Methods Mol Biol 353:27-38.

Inhibitory nucleic acids for use in the methods described herein can include one or more modifications, e.g., be stabilized against nucleolytic degradation such as by the incorporation of a modification, e.g., a nucleotide modification. For example, inhibitory nucleic acids can include a phosphorothioate at least the first, second, or third internucleotide linkage at the 5′ or 3′ end of the nucleotide sequence. As another example, inhibitory nucleic acids can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the inhibitory nucleic acids can include at least one 2′-O-methyl-modified nucleotide, and in some embodiments, all of the nucleotides include a 2′-O-methyl modification.

Chemical modifications, particularly the use of locked nucleic acids (LNAs) (Okiba (1997) Tetrahedron Lett 39:5401-5404; Singh (1998) Chem Commun 4:455-456), 2′-O-methoxyethyl (2′-O-MOE) (Martin (1995) Helv Chim Acta 78:486-504; You (2006) Nucleic Acids Res 34(8):e60; Owczarzy (2011) Biochem 50(43):9352-9367), constrained ethyl BNA (cET) (Murray (2012) Nucleic Acids Res 40: 6135-6143), and gapmer oligonucleotides, which contain 2-5 chemically modified nucleotides (LNA, 2′-O-MOE RNA or cET) at each terminus flanking a central 5-10 base “gap” of DNA (Monia (1993) J Biol Chem 268:14514-14522; Wahlestedt (2000) PNAS 97:5633-5638), improve antisense oligonucleotide binding affinity for the target RNA, which increases the steric block efficiency. Antisense oligos that hybridize to p40 or CD5L, can be identified through experimentation.

Techniques for the manipulation of inhibitory nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

In some embodiments, the inhibitory nucleic acids are “locked,” i.e., comprise nucleic acid analogues in which the ribose ring is “locked” by a methylene bridge connecting the 2′-O atom and the 4′-C atom (see, e.g., Kaupinnen (2005) Drug Disc. Today 2(3):287-290; Koshkin (1998) J. Am. Chem. Soc. 120(50):13252-13253). For additional modifications see US 20100004320, US 20090298916, and US 20090143326.

In some embodiments, the nucleic acid sequence that is complementary to p40 or CD5L can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference. RNA interference may cause translational repression and degradation of target mRNAs with imperfect complementarity or sequence-specific cleavage of perfectly complementary mRNAs.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. After the siRNA has cleaved its target, it is released from that RNA to search for another target and can repeatedly bind and cleave new targets (Brummelkamp (2002) Science 296:550-553; Lee (2002) Nature Biotechnol., 20, 500-505; Miyagishi and Taira (2002) Nature Biotechnol 20:497-500; Paddison (2002) Genes & Dev. 16:948-958; Paul (2002) Nature Biotechnol 20, 505-508; Sui (2002) Proc. Natl. Acad. Sd. USA 99(6), 5515-5520; Yu (2002) Proc Natl Acad Sci USA 99:6047-6052; Peer and Lieberman (2011) Gen Ther 18, 1127-1133).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target. shRNAs that are constitutively expressed form promoters can ensure long-term gene silencing. Most methods commonly used for delivery of siRNAs rely on commonly used techniques for introducing an exogenous nucleic acid into a cell including calcium phosphate or calcium chloride precipitation, microinjection, DEAE-dextrin-mediated transfection, lipofection, commercially available cationic polymers and lipids and cell-penetrating peptides, electroporation or stable nucleic acid-lipid particles (SNALPs), all of which are routine in the art. siRNAs can also be conjugated to small molecules to direct binding to cell-surface receptors, such as cholesterol (Wolfrum (2007) Nat Biotechnol 25:1149-1157), alpha-tocopherol (Nishina (2008) Mol Ther 16:734-40), lithocholic acid or lauric acid (Lorenz (2004) Bioorg Med Chem Lett 14:4975-4977), polyconjugates (Rozema (2007) PNAS 104:12982-12987). A variation of conjugated siRNAs are aptamer-siRNA chimeras (McNamara (2006) Nat Biotechnol 24:1005-1015; Dassie (2009) Nat Biotechnol 27:839-849) and siRNA-fusion protein complexes, which is composed of a targeting peptide, such as an antibody fragment that recognizes a cell-surface receptor or ligand, linked to an RNA-binding peptide that can be complexed to siRNAs for targeted systemic siRNA delivery (Yao (2011) Sci Transl Med 4(130): 130ra48.

Trans-cleaving enzymatic nucleic acid molecules can also be used; they have shown promise as therapeutic agents for human disease (Usman & McSwiggen, (1995) Ann. Rep. Med. Chem. 30, 285-294; Christoffersen and Marr (1995) J. Med. Chem. 38, 2023-2037; Weng (2005) Mol Cancer Ther 4, 948-955; Armado (2004) Hum Gene Ther 15, 251-262; Macpherson (2005) J Gene Med 7,552-564; Muhlbacher (2010) Curr Opin Pharamacol 10(5):551-6). Enzymatic nucleic acid molecules can be designed to cleave specific p40 and/or CD5L targets within the background of cellular RNA. Such a cleavage event renders the p40 and/or CD5L non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel (1979) Proc. R. Soc. London B 205, 435) have been used to evolve new nucleic acid catalysts with improved properties, new functions and capable of catalyzing a variety of reactions, such as cleavage and ligation of phosphodiester linkages and amide linkages, (Joyce (1989) Gene 82, 83-87; Beaudry (1992) Science 257, 635-641; Joyce (1992) Scientific American 267, 90-97; Breaker (1994) TIBTECH 12, 268; Bartel (1993) Science 261:1411-1418; Szostak (1993) TIBS 17, 89-93; Kumar (1995) FASEB J. 9, 1183; Breaker (1996) Curr. Op. Biotech. 1, 442; Scherer (2003) Nat Biotechnol 21, 1457-1465; Berens (2015) Curr. Op. Biotech. 31, 10-15). Ribozymes can also be engineered to be allosterically activated by effector molecules (riboswitches, Liang (2011) Mol Cell 43, 915-926; Wieland (2010) Chem Biol 17, 236-242; U.S. Pat. No. 8,440,810). The development of ribozymes that are optimal for catalytic activity would contribute significantly to any strategy that employs RNA-cleaving ribozymes for the purpose of regulating gene expression. The most common ribozyme therapeutics are derived from either hammerhead or hairpin/paperclip motifs. The hammerhead ribozyme, for example, functions with a catalytic rate (kcat) of about 1 min-1 in the presence of saturating (10 rnM) concentrations of Mg2+ cofactor. An artificial “RNA ligase” ribozyme has been shown to catalyze the corresponding self-modification reaction with a rate of about 100 min-1. In addition, it is known that certain modified hammerhead ribozymes that have substrate binding arms made of DNA catalyze RNA cleavage with multiple turn-over rates that approach 100 min-1. Ribozymes can be delivered to target cells in RNA form or can be transcribed from vectors. Due to poor stability of fully-RNA ribozymes, ribozymes often require chemical modification, such as, 5′-PS backbone linkage, 2′-O-Me, 2′-deoxy-2′-C-allyl uridine, and terminal inverted 3′-3′ deoxyabasic nucleotides (Kobayashi (2005) Cancer Chemother Pharmacol 56, 329-336).

### Perturbation Screening

In certain embodiments, genes or gene signatures relating to the autoimmune diseases, inflammation, and hyperimmune responses are screened by perturbation of target genes. In certain embodiments, genes or gene signatures relating to cancer and chronic infection are screened by perturbation of target genes. In certain embodiments, genes or gene signatures relating to CD5L monomers, CD5L:CD5L homodimers, CD5L:p40 heterodimers, or agonists or antagonists thereof are screened in perturbation studies. In certain embodiments, perturbation is performed in immune cells (e.g., Th1, Th17 cells). In certain embodiments, one or more genes selected from Dusp2, Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Snx20, Lamp1, Faf1, Gpatch3, Dapk3, 1110065P20Rik, Vaultrc5, Il17f, Il17a, Ildr1, Il1r1, Lgr4, Ptpnl4, Paqr8, Timp1, Il1rn, Smim3, Gap43, Tigit, Mmp10, Il22, Enpp2, Iltifb, Ido1, I123r, Stom, Bcl2l11, 5031414D18Rik, Il24, Itga7, Il6, Epha2, Mt2, Upp1, Snord104, 5730577I03Rik, Slcl8b1, Ptprj, Clip3, Mir5104, Ppifos, Rab13, Hist1h2bn, Ass1, Cd200r1, E130112N10Rik, Mxd4, Casp6, Gatm, Tnfrsf8, Gp49a, Gadd45g, Ccr5, Tgm2, Lilrb4, Ecm1, Arhgap18, Serpinb5, Cysltr1, Enpp1, Selp, Slc38a4, Gm14005, Epb4.1l4b, Moxd1, Klra7, Igfbp4, Tnip3, Gstt1, Pglyrp2, 112rb2, Ctla2a, Plac8, Ly6c1, Sell, Ncf1, Trp53i11, B3gnt3, Kremen2, Matk, Ltb4r1, Ets, Tnfrsf26, Cd28, Rybp, Ppp1r3c, Thy1, Trib2, Sema3b, Pros1, 1133, Gm5483, Myh11, Cntd1, Ms4a4b, Treml2, 3110009E18Rik, Pglyrp1, Amd1, Slc24a5, Snhg9, Ifi27l1, Irf7, Mx1, Snhg10, 114, Snora43, H2-L, Myl4, Insl3, Tgoln2, BC022687, C230035I16Rik, Hvcn1, Myh10, Dhrs3, Acsl6, Rgs2, Cc120, Cc13, Dlg2, Ccr6, Cc14, Dusp14, Apol9b, Cd72, Ispd, Cd70, S100al, Lgals3, Slc15a3, Nkg7, Serpinc1, Olfr175-ps1, 119, Pdlim4, Il3, Insl6, Perp, Cd51, Serpine2, Galnt14, Tff1, Ppfibp2, Bdh2, Mlf1, Il1a, Osr2, Gm5779, Ebf1, Spink2, Egfr and Ccdc155 are perturbed.

In certain embodiments, the invention involves plate based single cell RNA sequencing (see, e.g., Picelli, S. et al., 2014, “Full-length RNA-seq from single cells using Smart-seq2” Nature protocols 9, 171-181, doi: 10. 1038/nprot.2014.006).

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

Methods and tools for genome-scale screening of perturbations in single cells using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; and International publication serial number WO/2017/075294). The present invention is compatible with perturb-seq, such that signature genes may be perturbed and the perturbation may be identified and assigned to the proteomic and gene expression readouts of single cells. In certain embodiments, signature genes may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a cancer. Thus, in certain embodiments, perturb-seq is used to discover novel drug targets to allow treatment of specific cancer patients having the gene signature of the present invention.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a single cell is determined by performing single cell RNA sequencing (RNA-seq). In preferred embodiments, the single cell RNA-seq is performed by any method as described herein (e.g., Drop-seq, InDrop, 10× genomics). In certain embodiments, unique barcodes are used to perform Perturb-seq. In certain embodiments, a guide RNA is detected by RNA-seq using a transcript expressed from a vector encoding the guide RNA. The transcript may include a unique barcode specific to the guide RNA. Not being bound by a theory, a guide RNA and guide RNA barcode is expressed from the same vector and the barcode may be detected by RNA-seq. Not being bound by a theory, detection of a guide RNA barcode is more reliable than detecting a guide RNA sequence, reduces the chance of false guide RNA assignment and reduces the sequencing cost associated with executing these screens. Thus, a perturbation may be assigned to a single cell by detection of a guide RNA barcode in the cell. In certain embodiments, a cell barcode is added to the RNA in single cells, such that the RNA may be assigned to a single cell. Generating cell barcodes is described herein for single cell sequencing methods. In certain embodiments, a Unique Molecular Identifier (UMI) is added to each individual transcript and protein capture oligonucleotide. Not being bound by a theory, the UMI allows for determining the capture rate of measured signals, or preferably the binding events or the number of transcripts captured. Not being bound by a theory, the data is more significant if the signal observed is derived from more than one protein binding event or transcript. In preferred embodiments, Perturb-seq is performed using a guide RNA barcode expressed as a polyadenylated transcript, a cell barcode, and a UMI.

Perturb-seq combines emerging technologies in the field of genome engineering, single-cell analysis and immunology, in particular the CRISPR-Cas9 system and droplet single-cell sequencing analysis. In certain embodiments, a CRISPR system is used to create an INDEL at a target gene. In other embodiments, epigenetic screening is performed by applying CRISPRa/i/x technology (see, e.g., Konermann et al. “Genome-scale transcriptional activation by an engineered CRISPR-Cas9 complex” Nature. 2014 Dec. 10. doi: 10.1038/naturel4136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimising deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035). Numerous genetic variants associated with disease phenotypes are found to be in non-coding region of the genome, and frequently coincide with transcription factor (TF) binding sites and non-coding RNA genes. Not being bound by a theory, CRISPRa/i/x approaches may be used to achieve a more thorough and precise understanding of the implication of epigenetic regulation. In one embodiment, a CRISPR system may be used to activate gene transcription. A nuclease-dead RNA-guided DNA binding domain, dCas9, tethered to transcriptional repressor domains that promote epigenetic silencing (e.g., KRAB) may be used for “CRISPRi” that represses transcription. To use dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, other CRISPR-based perturbations are readily compatible with Perturb-seq, including alternative editors such as CRISPR/Cpfl. In certain embodiments, Perturb-seq uses Cpfl as the CRISPR enzyme for introducing perturbations. Not being bound by a theory, Cpfl does not require Tracr RNA and is a smaller enzyme, thus allowing higher combinatorial perturbations to be tested.

The cell(s) may comprise a cell in a model non-human organism, a model non-human mammal that expresses a Cas protein, a mouse that expresses a Cas protein, a mouse that expresses Cpfl, a cell in vivo or a cell ex vivo or a cell in vitro (see e.g., WO 2014/093622 (PCT/US13/074667); US Patent Publication Nos. 20120017290 and 20110265198 assigned to Sangamo BioSciences, Inc.; US Patent Publication No. 20130236946 assigned to Cellectis; Platt et al., “CRISPR-Cas9 Knockin Mice for Genome Editing and Cancer Modeling” Cell (2014), 159(2): 440-455; “Oncogenic models based on delivery and use of the crispr-cas systems, vectors and compositions” WO2014204723A1 “Delivery and use of the crispr-cas systems, vectors and compositions for hepatic targeting and therapy” WO2014204726A1; “Delivery, use and therapeutic applications of the crispr-cas systems and compositions for modeling mutations in leukocytes” WO2016049251; and Chen et al., “Genome-wide CRISPR Screen in a Mouse Model of Tumor Growth and Metastasis” 2015, Cell 160, 1246-1260). The cell(s) may also comprise a human cell. Mouse cell lines may include, but are not limited to neuro-2a cells and EL4 cell lines (ATCC TIB-39). Primary mouse T cells may be isolated from C57/BL6 mice. Primary mouse T cells may be isolated from Cas9-expressing mice.

In one embodiment, CRISPR/Cas9 may be used to perturb protein-coding genes or non-protein-coding DNA. CRISPR/Cas9 may be used to knockout protein-coding genes by frameshifts, point mutations, inserts, or deletions. An extensive toolbox may be used for efficient and specific CRISPR/Cas9 mediated knockout as described herein, including a double-nicking CRISPR to efficiently modify both alleles of a target gene or multiple target loci and a smaller Cas9 protein for delivery on smaller vectors (Ran, F. A., et al., In vivo genome editing using Staphylococcus aureus Cas9. Nature. 520, 186-191 (2015)). A genome-wide sgRNA mouse library (˜10 sgRNAs/gene) may also be used in a mouse that expresses a Cas9 protein (see, e.g., WO2014204727A1).

In one embodiment, perturbation is by deletion of regulatory elements. Non-coding elements may be targeted by using pairs of guide RNAs to delete regions of a defined size, and by tiling deletions covering sets of regions in pools.

In one embodiment, perturbation of genes is by RNAi. The RNAi may be shRNA's targeting genes. The shRNA's may be delivered by any methods known in the art. In one embodiment, the shRNA's may be delivered by a viral vector. The viral vector may be a lentivirus, adenovirus, or adeno associated virus (AAV).

A CRISPR system may be delivered to primary mouse T-cells. Over 80% transduction efficiency may be achieved with Lenti-CRISPR constructs in CD4 and CD8 T-cells. Despite success with lentiviral delivery, recent work by Hendel et al, (Nature Biotechnology 33, 985-989 (2015) doi:10.1038/nbt.3290) showed the efficiency of editing human T-cells with chemically modified RNA, and direct RNA delivery to T-cells via electroporation. In certain embodiments, perturbation in mouse primary T-cells may use these methods.

In certain embodiments, whole genome screens can be used for understanding the phenotypic readout of perturbing potential target genes. In preferred embodiments, perturbations target expressed genes as defined by a gene signature using a focused sgRNA library. Libraries may be focused on expressed genes in specific networks or pathways. In other preferred embodiments, regulatory drivers are perturbed. In certain embodiments, Applicants perform systematic perturbation of key genes that regulate T-cell function in a high-throughput fashion. In certain embodiments, Applicants perform systematic perturbation of key genes that regulate cancer cell function in a high-throughput fashion (e.g., immune resistance or immunotherapy resistance). Applicants can use gene expression profiling data to define the target of interest and perform follow-up single-cell and population RNA-seq analysis. Not being bound by a theory, this approach will accelerate the development of therapeutics for human disorders, in particular cancer. Not being bound by a theory, this approach will enhance the understanding of the biology of T-cells and tumor immunity, and accelerate the development of therapeutics for human disorders, in particular cancer, as described herein.

Not being bound by a theory, perturbation studies targeting the genes and gene signatures described herein could (1) generate new insights regarding regulation and interaction of molecules within the system that contribute to suppression of an immune response, such as in the case within the tumor microenvironment, and (2) establish potential therapeutic targets or pathways that could be translated into clinical application.

In certain embodiments, after determining Perturb-seq effects in cancer cells and/or primary T-cells, the cells are infused back to the tumor xenograft models (melanoma, such as B16F10 and colon cancer, such as CT26) to observe the phenotypic effects of genome editing. Not being bound by a theory, detailed characterization can be performed based on (1) the phenotypes related to tumor progression, tumor growth, immune response, etc. (2) the TILs that have been genetically perturbed by CRISPR-Cas9 can be isolated from tumor samples, subject to cytokine profiling, qPCR/RNA-seq, and single-cell analysis to understand the biological effects of perturbing the key driver genes within the tumor-immune cell contexts. Not being bound by a theory, this will lead to validation of TILs biology as well as lead to therapeutic targets.

### Mouse Models of CD5L

A “knock-out” of a gene means an alteration in the sequence of the gene that results in a decrease of function of the target gene, preferably such that target gene expression is undetectable or insignificant. A knock-out of an endogenous CD5L gene means that function of the CD5L gene has been substantially decreased so that expression is not detectable or only present at insignificant levels. “Knock-out” transgenics can be transgenic animals having a heterozygous knock-out of the CD5L gene or a homozygous knock-out of the CD5L gene. “Knock-outs” also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

A “knock-in” of a target gene means an alteration in a host cell genome that results in altered expression (e.g., increased (including ectopic)) of the target gene, e.g., by introduction of an additional copy of the target gene, or by operatively inserting a regulatory sequence that provides for enhanced expression of an endogenous copy of the target gene. “Knock-ins” also encompass conditional knock-ins.

In certain embodiments, the present invention provides for CD5L mouse models. In preferred embodiments, the CD5L mouse models can be used to study CD5L function, screen for therapeutics, generate specific antibodies, and to perform perturbation studies. In certain embodiments, the mouse model is a CD5L knock out mouse. In certain embodiments, the mouse model is a conditional CD5L knockout mouse. In certain embodiments, the mouse model is an inducible CD5L knockout mouse. In certain embodiments, the mouse model expresses a genetic modifying agent (e.g., CRISPR, TALE, Zn finger protein). In certain embodiments, the genetic modifying agent is inducibly expressed resulting in decreased or abolished expression of CD5L. In certain embodiments, the inducible knockout mouse may express a recombinase (e.g., Cre, Flp) under the control of an inducible promoter (e.g., Dox inducible). In certain embodiments, the mouse model may be a CD5L knockout mouse, such that the endogenous CD5L gene is knocked out and the mouse expresses a recombinant CD5L protein from a transgene. The term “transgene” is used herein to describe genetic material which has been or is about to be artificially inserted into the genome of a mammal, particularly a mammalian cell of a living animal. The transgene may be under the control of an inducible promoter, such that CD5L expression may be controlled. In certain embodiments, the endogenous CD5L gene is knocked out conditionally (e.g., in a specific cell type or tissue by expression of a tissue specific recombinase). In certain embodiments, the transgene expresses a non-mouse CD5L (e.g., human CD5L). In certain embodiments, the transgene comprises a mutation (e.g., a point mutation or a deletion in a domain). In certain embodiments, the transgene expresses a CD5L-p40 heterodimer fusion protein. In certain embodiments, the transgene is introduced to cells ex vivo by a method as described herein.

### Generating Mice

The generation of knockout mice is known in the art (see, e.g., Hall, B., Limaye, A. and Kulkarni, A. B. (2009), Overview: Generation of Gene Knockout Mice. Current Protocols in Cell Biology, 44: 19.12.1-19.12.17. doi:10.1002/0471143030.cb1912s44). In certain embodiments, a conditional knockout mouse is generated by crossing a mouse that expresses a tissue specific Cre recombinase to a CD5L flox/flox mouse. In certain embodiments, conditional ready mice may be used to generate a CD5L knockout or conditional knockout mouse. Conditional ready mice are available commercially (e.g., B6NTac; B6N-Cd5ltmla(KOMP)Mbp/H). The conditional ready mouse may be crossed to a flpo mouse (Jackson Labs) to generate the CD5L flox/flox mouse. The conditional ready mouse can also be crossed to any Cre mouse to generate a reporter deletional knockout. Tissue specific expression may be controlled by placing the Cre gene under control of a tissue specific promoter.

In some embodiments, one or more of the disclosed CRISPR-Cas systems of those known in the prior art may be used to generate transgenic animal models for the one or more diseases, e.g. the autoimmune diseases or hyperimmune responses, decribed herein by altering the gene expression profile of suitable model animal. See, e.g., Platt et al. (2014) Cell 159:440-455; Wang et al. (2013) Cell 153:910-918; Xue et al. (2014) Nature 514:380-384; Nelson et al. (2016) 351(6271):403-407; Chen et al. (2015) Cell 160:1246-1260; Tabebordbar et al. (2016) 351(6271):407-411; WO 2014/204726; WO 2014/204723; WO 2016/049251.

In certain embodiments, CD5L knockout mice may be generated by using a CRISPR system. In certain embodiments, Cre-dependent Cas9 knockin mice or any Cre-dependent CRISPR enzyme mouse (e.g., Cpfl) may be crossed with tissue-specific Cre transgenic or knockin mice to limit expression of the CRISPR enzyme to a specific cell type and limit CD5L knockout to specific cell types (see, e.g., Platt et al., “CRISPR-Cas9 Knockin Mice for Genome Editing and Cancer Modeling” Cell (2014), 159(2): 440-455). In certain embodiments, expression of Cre is limited to immune cells whereby the CRISPR enzyme is expressed exclusively in immune cells. In a specific embodiment, one or more CRISPR guide sequences targeting CD5L may be introduced to a CRISPR knockin mouse to generate a knockout. In certain embodiments, guide sequences are introduced to mouse ES cells from a CRISPR mouse for generating a CD5L knockout or conditional knockout mouse.

In certain embodiments, CD5L is knocked out in a mouse model of disease (e.g., autoimmune disease, cancer). Cancer models include, but are not limited to, melanoma, such as B16F10 and colon cancer, such as CT26. Models of colitis can be generated by treating mice with DSS. Models of autoimmunity can be generated by immunizing mice with MOG(35-55) (EAE model).

In certain embodiments, tissue-specific Cre transgenic or knockin mice are used to limit knockout of CD5L to a specific cell type (see, e.g., Sharma and Zhu, Immunologic Applications of Conditional Gene Modification Technology in the Mouse, Curr Protoc Immunol. 2014; 105: 10.34.1-10.34.13). Most of the existing Cre mouse lines can be found at the CREATE (Coordination of resources for conditional expression of mutated mouse alleles) consortium (creline.org/), which includes the Cre mouse database at Mouse Genome Informatics (MGI, loxP.creportal.org/). In certain embodiments, CD5L is knocked out specifically in immune cells.

Some commonly used Cre mice for studying the immune system and that are applicable for use in the present invention are summarized in the Table below (Tg refers to transgenic and KI refers to knock in).

| | | Expression in cell | | |
|---|---|---|---|---|
| Name | Tg/Kl | types | Note | Reference |
| ROSA26- | Kl | Most cells except | High deletion efficiency with | Seibler et al. (2003) |
| CreER^{T2} | | those in the brain | tamoxifen treatment both in | |
| | | | vitro and in vivo | |
| Vav-Cre | Tg | All hematopoietic | High deletion efficiency; may | de Boer et al. (2003) |
| | | lineages, testis and | cause germ line deletion in | |
| | | ovaries | some offspring | |
| CD2-Cre | Tg | Common lymphoid | High deletion efficiency; some | Zhumabekok et al. |
| | | progenitors (CLPs) | modified CD2-Cre lines may | (1995); de Boer et al. |
| | | | only delete genes in T cells but | (2003) |
| | | | not B cells | |
| Lck-Cre | Tg | Early DN stage in | Deletion efficiency varies | Lee et al. (2001) |
| | | the thymus | | |
| CD4-Cre | Tg | Late DN to DP stage, | High deletion efficiency | Lee et al. (2001) |
| | | deleting floxed | | |
| | | genes in both CD4 | | |
| | | and CD8 T cells | | |
| CD4-CreER^{T2} | Tg | Deleting floxed | Inducible by tamoxifen; | Aghajani et al. (2012) |
| | | genes only CD4 but | deletion efficiency up to 80% in | |
| | | not CD8 T cells in | vivo | |
| | | the periphery | | |
| dLck-Cre (line | Tg | Late DP to SP stage | −70% deletion efficiency in | Wang et al. (2001) |
| 3779) | | | CD4 T cells; higher efficiency | |
| | | | (80% to 90%) in CD8 T cells; very | |
| | | | low in Tregs | |
| OX40-Cre | Kl | Tregs and activated | Endogenous OX40 gene is | Yagi et al. (2010) |
| | | CD4⁺ T cells | disrupted by Cre; very low | |
| | | | efficiency in activated CD8 T | |
| | | | cells | |
| CD8a-Cre | Tg | Mature CD8⁺ but | Also known as E8I-Cre; Cre | Maekawa et al. |
| | | not CD4⁺ T cells | expression driven by the core | (2008) |
| | | | E8I enhancer and Cd8a | |
| | | | promoter | |
| Granzyme-B- | Tg | Activated CD4⁺ and | Cre driven by truncated | Jacob and Baltimore |
| Cre | | CD8⁺ T cells | granzyme B promoter | (1999) |
| Mb1-Cre | Kl | Starting from Pre- | Endogenous Mb1 gene | Hobeika et al. (2006) |
| | | Pro-B stage | encoding Igα signaling subunit | |
| | | | of the BCR is disrupted by Cre; | |
| | | | deletion efficiency is better | |
| | | | than CD19-Cre | |
| CD19-Cre | Kl | Starting Pro-B stage | Endogenous Cd19 gene is | Rickert et al. (1997) |
| | | | disrupted by Cre; deletion | |
| | | | efficiency is 75% to 95% | |
| CD19-CreER^{T2} | BAC | Similar to CD19-Cre, | Inducible by tamoxifen; | Boross et al. (2009) |
| | Tg | but its activity | deletion efficiency 25% to 60% | |
| | | requires tamoxifen | | |
| | | treatment | | |
| Foxp3-YFPCre | Kl | Only in Foxp3⁺ Tregs | YFP is dim; endogenous Foxp3 | Rubtsov et al. (2008) |
| | | | expression intact | |
| Foxp3- | Kl | Only in Foxp3⁺ Tregs | Inducible but with low deletion | Rubtsov et al. (2010) |
| GFPCreER^{T2} | | | efficiency (10% to 20%); | |
| | | | endogenous Foxp3 expression | |
| | | | intact | |
| Id2-CreER^{T2} | Kl | Id2-expressing cells: | Inducible but with low deletion | Rawlins et al. (2009) |
| | | epithelial cells in the | efficiency; endogenous Id2gene | |
| | | lung distal tips as | is disrupted by CreER^{T2} | |
| | | well as progenitor | | |
| | | of ILCs and T cells | | |

### Studying CD5L Function

A dynamic regulatory network controls Th17 differentiation (See e.g., Yosef et al., Dynamic regulatory network controlling Th17 cell differentiation, Nature, vol. 496: 461-468 (2013); Wang et al., CD5L/AIM Regulates Lipid Biosynthesis and Restrains Th17 Cell Pathogenicity, Cell Volume 163, Issue 6, p1413-1427, 3 Dec. 2015; Gaublomme et al., Single-Cell Genomics Unveils Critical Regulators of Th17 Cell Pathogenicity, Cell Volume 163, Issue 6, p1400-1412, 3 Dec. 2015; and Internationational publication numbers WO2016138488A2, WO2015130968, WO/2012/048265, WO/2014/145631 and WO/2014/134351, the contents of which are hereby incorporated by reference in their entirety.

CD5L has previously been identified as a novel molecule expressed by non-pathogenic Th17 cells using single-cell RNA sequencing and a cell intrinsic role was demonstrated for CD5L in Th17 cells. As described herein, CD5L, CD5L-p40 heterodimer as well as their molecular mimics such as antibodies and small molecules targeting the IL-23R-dependent pathway can be used to treat or alleviate symptoms of autoimmune diseases such as inflammatory bowel diseases, multiple sclerosis, psoriasis and other inflammation-based diseases such as colorectal cancer and other tumors. In addition, preventing interaction of CD5L, CD5L:p40 heterodimers with their receptors may promote their immune responses and enhance immune responses against tumors and chronic viral and bacterial infections. The CD5L knockout mouse and conditional knockout mouse generated and described herein can be used to study CD5L function. As CD5L has been shown to function in T cell balance and immunity, in certain embodiments, CD5L function may be studied in immune cells using the mouse model of the present invention.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T cells (including, e.g., thymocytes, Th or Tc; Th1, Th2, Th17, Thap3, CD4+, CD8+, effector Th, memory Th, regulatory Th, CD4+/CD8+ thymocytes, CD4-/CD8-thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, Ti B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

Studying CD5L in immune cells may be performed using conditional knockout mice as described herein. In certain embodiments, conditional knockout mice are generated by crossing floxed mice with mice expressing a tissue specific Cre recombinase. In certain embodiments, immune cells knocked out for CD5L can be isolated from the mouse model. In certain embodiments, immune cells knocked out for CD5L are used for study in other mouse models (e.g., mouse models of disease). In certain embodiments, immune cells isolated from the mouse model may be used to determine cell types where CD5L has a function in inflammatory responses.

In certain embodiments, the mouse is treated, such that the mouse has a disease phenotype (e.g., cancer, autoimmune disease). In one embodiment, the mouse expresses a CRISPR enzyme targeting CD5L exclusively in immune cells in a mouse having a disease phenotype.

### Screening for Therapeutics

The CD5L knockout mouse and conditional knockout mouse generated and described herein can be used for identifying intervention tools that can have diagnostic and therapeutic potential. For example, knockout of CD5L leads to increased EAE in mouse models. Thus, the mouse models of the present invention may be used to screen for therapeutics for treating autoimmunity. Of particular interest are screening assays for agents that have a low toxicity for human cells. Depending on the particular assay, whole animals may be used, or cells derived there from may be used. Cells may be freshly isolated from an animal, or may be immortalized in culture. Cells of particular interest are immune cells.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A number of assays are known in the art for determining the effect of a drug on an immune response and other phenomena associated with inflammatory diseases and cancer (e.g., experimental autoimmune encephalomyelitis (EAE), colitis, tumor growth assays). In certain embodiments, a CD5L knockout mouse according to the present invention is used to test drugs capable of treating EAE or colitis. In certain embodiments, mice are treated with DSS to induce colitis in the mouse model. Mice can be screened for body weight after induction of colitis. In certain embodiments, for active induction of EAE, mice can be immunized by subcutaneous injection MOG(35-55) in CFA, then receive 200 ng pertussis toxin intraperitoneally. Mice can be monitored and assigned scores daily for development of classical and atypical signs of EAE according to the following criteria (Jager et al., Th1, Th17, and Th9 effector cells induce experimental autoimmune encephalomyelitis with different pathological phenotypes. Journal of immunology 2009 183, 7169-7177) 0, no disease; 1, decreased tail tone or mild balance defects; 2, hind limb weakness, partial paralysis or severe balance defects that cause spontaneous falling over; 3, complete hind limb paralysis or very severe balance defects that prevent walking; 4, front and hind limb paralysis or inability to move body weight into a different position; 5, moribund state. In certain embodiments, the pathological phenotypes may be alleviated by a therapeutic identified in the screen. In certain embodiments, a tumor is transplanted to a mouse of the present invention and therapeutics are screened that enhance an immune response against the tumor. It will be understood by one of skill in the art that many other assays may also be used. The subject animals may be used by themselves, or in combination with control animals.

The screen using the animals of the present invention can employ any phenomena associated with an immune response (e.g., autoimmunity, inflammation, tumor immunity) that can be readily assessed in an animal model. The screening can include assessment of phenomena including, but not limited to analysis of molecular markers (e.g., levels of expression of signature gene products). In certain embodiments, the secretion of cytokines or expression of surface markers are screened.

The therapeutic agents may be administered in a variety of ways, orally, topically, aerosol, parenterally e.g. subcutaneously, intraperitoneally, by viral infection, intravascularly, etc. Oral treatments are of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### Generating CD5L Specific Antibodies

The CD5L knockout mouse and conditional knockout mouse generated and described herein can be used for generation of antibodies. In certain embodiments, the antibodies are CD5L antagonist antibodies. In certain embodiments, the antibodies are CD5L agonist antibodies. In certain embodiments, the mouse of the present invention includes immune cells that do not recognize CD5L as a self-protein. Thus, in certain embodiments, the CD5L knockout mouse can be used to generate highly specific antibodies because antibodies against CD5L are not eliminated as self-antibodies.

An antibody generated may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility (e.g., CD5L, CD5L:p40). By means of example and not limitation, monoclonal antibodies may be made by the hybridoma method first described by Kohler et al. 1975 (Nature 256: 495).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

In certain embodiments, antibodies generated against CD5L or an epitope thereof are sequenced and cloned. In certain embodiments, the sequence of the CD5L antibodies are modified. Naturally occurring residues may be divided into classes based on common side chain properties: 1) hydrophobic: norleucine, Met, Ala, Val, Leu, lie; 2) neutral hydrophilic: Cys, Ser, Thr, Asn, GIn; 3) acidic: Asp, GIu; 4) basic: His, Lys, Arg; 5) residues that influence chain orientation: Gly, Pro; and 6) aromatic: Trp, Tyr, Phe. In certain embodiments, non-conservative substitutions may involve the exchange of a member of one of these classes for a member from another class. Such substituted residues may be introduced into regions of a human antibody that are homologous with non-human antibodies, or into the non-homologous regions of the molecule.

In making substitutions, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, in certain instances, is understood in the art. Kyte et al., J. Mol. Biol., 157:105-131 (1982). It is known that in certain instances, certain amino acids may be substituted for other amino acids having a similar hydropathic index or score and still retain a similar biological activity. In making changes based upon the hydropathic index, in certain embodiments, the substitution of amino acids whose hydropathic indices are within +2 is included. In certain embodiments, those which are within +1 are included, and in certain embodiments, those within +0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (−+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One may also identify epitopes from primary amino acid sequences on the basis of hydrophilicity. These regions are also referred to as “epitopic core regions.”

In certain embodiments of humanized antibodies, one or more complementarity determining regions (CDRs) from the light and heavy chain variable regions of an antibody with the desired binding specificity (the “donor” antibody) are grafted onto human framework regions (FRs) in an “acceptor” antibody (e.g., CDR's from a monoclonal antibody developed in a CD5L knockout mouse). Exemplary CDR grafting is described, e.g., in U.S. Pat. Nos. 6,180,370, 5,693,762, 5,693,761, 5,585,089, and 5,530,101; Queen et al. (1989) Proc. Nat'l Acad. Sci. USA 86:10029-10033. In certain embodiments, one or more CDRs from the light and heavy chain variable regions are grafted onto consensus human FRs in an acceptor antibody. To create consensus human FRs, in certain embodiments, FRs from several human heavy chain or light chain amino acid sequences are aligned to identify a consensus amino acid sequence.

In certain embodiments, certain FR amino acids in the acceptor antibody are replaced with FR amino acids from the donor antibody. In certain such embodiments, FR amino acids from the donor antibody are amino acids that contribute to the affinity of the donor antibody for the target antigen (see, e.g., in U.S. Pat. Nos. 6,180,370, 5,693,762, 5,693,761, 5,585,089, and 5,530,101; Queen et al. (1989) Proc. Nat'l Acad. Sci. USA 86:10029-10033. In certain embodiments, computer programs are used for modeling donor and/or acceptor antibodies to identify residues that are likely to be involved in binding antigen and/or to contribute to the structure of the antigen binding site, thus assisting in the selection of residues, such as FR residues, to be replaced in the donor antibody.

In certain embodiments, CDRs from a donor antibody are grafted onto an acceptor antibody comprising a human constant region. In certain such embodiments, FRs are also grafted onto the acceptor. In certain embodiments, CDRs from a donor antibody are derived from a single chain Fv antibody. In certain embodiments, FRs from a donor antibody are derived from a single chain Fv antibody. In certain embodiments, grafted CDRs in a humanized antibody are further modified (e.g., by amino acid substitutions, deletions, or insertions) to increase the affinity of the humanized antibody for the target antigen. In certain embodiments, grafted FRs in a humanized antibody are further modified (e.g., by amino acid substitutions, deletions, or insertions) to increase the affinity of the humanized antibody for the target antigen.

In certain embodiments, non-human antibodies may be humanized using a “human engineering” method. See, e.g., U.S. Pat. Nos. 5,766,886 and 5,869,619. In certain embodiments of human engineering, information on the structure of antibody variable domains (e.g., information obtained from crystal structures and/or molecular modeling) is used to assess the likelihood that a given amino acid residue in a variable region is (a) involved in antigen binding, (b) exposed on the antibody surface (i.e., accessible to solvent), or (c) buried within the antibody variable region (i.e., involved in maintaining the structure of the variable region). Furthermore, in certain embodiments, human variable region consensus sequences are generated to identify residues that are conserved among human variable regions. In certain embodiments, that information provides guidance as to whether an amino acid residue in the variable region of a non-human antibody should be substituted.

In certain embodiments, a CD5L knockout mouse as described herein is immunized with an immunogen (e.g., CD5L, CD5L fragment, CD5L:p40). In certain embodiments, lymphatic cells (such as B-cells) from mice that express antibodies are obtained. In certain such embodiments, such recovered cells are fused with an “immortalized” cell line, such as a myeloid-type cell line, to produce hybridoma cells. In certain such embodiments, hybridoma cells are screened and selected to identify those that produce antibodies specific to the antigen of interest. In certain embodiments, human monoclonal antibodies against CD5L are suitable for use as therapeutic antibodies.

In certain embodiments, to generate antibodies, an animal is immunized with an immunogen. In certain embodiments, an immunogen is a polypeptide comprising CD5L. In certain embodiments, an immunogen is a polypeptide comprising a fragment of CD5L.

In certain embodiments, an immunogen comprises a human CD5L. In certain embodiments, an immunogen comprises a mouse CD5L. In certain such embodiments, a peptide is selected that is likely to be immunogenic. Exemplary guidance for selecting suitable immunogenic peptides is provided, for example, in Ausubel et al. (1989) Current Protocols in Molecular Biology Ch. 11.14 (John Wiley & Sons, NY); and Harlow and Lane (1988) Antibodies: A Laboratory Manual Ch. 5 (Cold Spring Harbor Laboratory, Cold Spring Harbor, N.Y.).

Certain exemplary algorithms are known to those skilled in the art for predicting whether a peptide segment of a protein is likely to be immunogenic. Certain such algorithms use the primary sequence information of a protein to make such predictions. Certain such algorithms are based on the method of, for example, Hopp and Woods (1981) Proc. Nat'l Acad. Sci. USA 78:3824-3828, or Kyte and Doolittle (1982) J. MoI. Biol. 157:105-132. Certain exemplary-algorithms are known to those skilled in the art for predicting the secondary structure of a protein based on the primary amino acid sequence of the protein. See, e.g., Corrigan et al. (1982) Comput. Programs Biomed. 3:163-168. Certain such algorithms are based on the method of, for example, Chou and Fasman (1978) Ann. Rev. Biochem. 47:25-276.

In certain embodiments, an animal is immunized with an immunogen and one or more adjuvants. In certain embodiments, an adjuvant is used to increase the immunological response, depending on the host species. Certain exemplary adjuvants include, but are not limited to, Freund's adjuvant (complete and incomplete), mineral salts such as aluminum hydroxide or aluminum phosphate, surface active substances, chitosan, lysolecithin, pluronic polyols, polyanions, peptides, oil emulsions, and potentially useful human adjuvants such as BCG (bacille Calmette-Guerin) and Corynehacterium parvum. In certain embodiments, the immune response to an irnmunogen, e.g., a peptide immunogen, is enhanced by coupling the immunogen to another immunogenic molecule or “carrier protein.” Certain exemplary carrier proteins include, but are not limited to, keyhole limpet hemocyanin (KLH), tetanus toxoid, diphtheria toxoid, ovalbumin, cholera toxoid, and immunogenic fragments thereof. For exemplary guidance in coupling peptide immunogens to carrier proteins, see, e.g., Ausubel et al. (1989) Current Protocols in Molecular Biology Ch. 11.15 (John Wiley & Sons, NY); and Harlow and Lane (1988) Antibodies: A Laboratory Manual Ch. 5 (Cold Spring Harbor Laboratory, Cold Spring Harbor, N.Y.).

In certain embodiments, any of the above immunogens can be produced using standard recombinant methods. For example, in certain embodiments, a polynucleotide encoding a mouse or human CD5L or fragment thereof or CD5L:p40 chimeric peptide may be cloned into a suitable expression vector. In certain embodiments, the recombinant vector is then introduced into a suitable host cell. In certain embodiments, the polypeptide is then isolated from the host cell by standard methods. For certain exemplary methods of recombinant protein expression, see, e.g., Ausubel et al. (1991) Current Protocols in Molecular Biology Ch. 16 (John Wiley & Sons, NY).

In certain embodiments, the mouse of the present invention may be used with perturbation methods and tools described herein to allow reconstruction of a cellular network or circuit. In one embodiment, the method comprises (1) introducing single-order or combinatorial perturbations to a population of cells (in vivo or ex vivo) from the mouse model, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a single cell is determined by performing single cell RNA sequencing (RNA-seq). In preferred embodiments, the single cell RNA-seq is performed by any method as described herein (e.g., Drop-seq, InDrop, 10× genomics). In certain embodiments, unique barcodes are used to perform Perturb-seq. In certain embodiments, a guide sequence is detected by RNA-seq using a transcript expressed from a vector encoding the guide RNA. The transcript may include a unique barcode specific to the guide sequence. Thus, a perturbation may be assigned to a single cell by detection of a guide sequence barcode in the cell. In certain embodiments, a cell barcode is added to the RNA in single cells, such that the RNA may be assigned to a single cell. Generating cell barcodes is described herein for single cell sequencing methods. In certain embodiments, a Unique Molecular Identifier (UMI) is added to each individual transcript and protein capture oligonucleotide. Not being bound by a theory, the UMI allows for determining the capture rate of measured signals, preferably the binding events or the number of transcripts captured. In preferred embodiments, perturbations are detected in single cells by detecting a guide sequence barcode expressed as a polyadenylated transcript, a cell barcode, and a UMI. In certain example embodiments, the guide sequence may further encode an optical barcode as described in WO/2016/149422 entitled “Encoding of DNA Vector Identity via Iterative Hybridization Detection of a Barcode Transcript” filed Mar. 16, 2016. Optical barcode allows for identification of delivery of guide sequences and association of such delivery with a particular cell phenotype.

The ability to generate high throughput in vivo single cell data provides transcriptional insight to the heterogeneity of cell states. However, the ability to perturb each candidate gene (e.g., regulatory candidate) in in vivo mouse models is laborious and time-consuming, and has become a limiting factor in the mapping and annotation of regulatory drivers. To enable the efficient testing of tens of candidate regulators Applicants can adapt the Pertub-seq system to screen for regulators in vivo (e.g., tumor mouse models). In vivo Perturb-seq may be performed with a set of perturbations. The set of perturbations may be selected based on targets in a specific pathway or determined by RNA-seq or determined by performing Perturb-seq in vitro. The perturbations may preferably include up to 10, 20, 30, 40, 50, 60, 70, 80, 100 perturbations. In certain embodiments, more than 100 perturbations are screened by in vivo Perturb-seq. In certain embodiments, target genes may be perturbed in cells ex vivo and introduced to an animal model in vivo. In certain embodiments, perturbed cells are extracted from an in vivo organism. For example, methods for isolating TILs are known in the art. Perturbed cells may be further isolated by sorting cells expressing a selectable marker, such as a fluorescent marker as described herein.

### Dosage

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions can include a single treatment or a series of treatments.

The invention is further described in the following examples, which do not limit the scope of the invention described in the claims.

### EXAMPLES

### Example 1—Materials and Methods

The CD5L monomer, CD5L dimer and CD5L:p40 heterodimer generations were out-sourced to Biolegend under CDA. Briefly, to generate the CD5L:p40 heterodimer, Cd51 and I112b (p40) were cloned into mammalian expression vector through a linker: P40-linker 2-3 (SGGG)-CD5L with His tag (“SGGG” disclosed as SEQ ID NO: 23). Similarly, CD5L monomer and dimer were generated by cloning CD5L with His tag at C-terminus into a mammalian expression vector. The plasmids are expressed in mammalian cell line and secreted CD5L:p40, CD5L (monomer and dimer) were purified and confirmed by gel electrophoresis and HPLC.

| CD5L sequence cloned: | | | | | | |
|---|---|---|---|---|---|---|
| (SEQ ID NO: 3) | | | | | | |
| 1 | (maplfnlmla | ilsifvgscf s) | *esptkvqlv | ggahrcegry | evehngqwgt | vcddgwdrrd |
| 61 | vavvcrelnc | gaviqtprga | syqppaseqr | vliggvdong | tedtlaqcel | nydvfdcshe |
| 121 | edagaqcenp | dsdllfiped | vrlvdgpghc | qgrvevlhqs | qwstvckagw | nlqvskvvcr |
| 181 | qlgcgrallt | ygscnkstqg | kgpiwmgkms | csggeanlrs | cllsrlennc | thgedtwmec |
| 241 | edpfelklvg | gdtpcsgrle | vlhkgswgsv | cddnwgeked | qvvckqlgcg | kslhpspktr |
| 301 | kiygpgagri | wlddvncsgk | eqslefcrhr | lwgyhdcthk | edvevictdf | dv |
| *the sinaling peptide was not included to better guide protein secretion | | | | | | |
| in the expression system | | | | | | |
| p40/1112b sequence cloned | | | | | | |
| (SEQ ID NO: 7) | | | | | | |
| 1 | mcpqkltisw | faivllvspl | mamwelekdv | yvvevdwtpd | apgetvnitc | dtpeedditw |
| 61 | tsdqrhgvig | sgktltitvk | efldaggytc | hkggetlshs | hlllhkkeng | iwsteilknf |
| 121 | knktflkcea | pnysgrftcs | wlvqrnmdlk | fniksssssp | dsravtcgma | slsaekvtld |
| 181 | qrdyekysys | cqedvtcpta | eetlpielal | earqqnkyen | ystsffirdi | ikpdppknlq |
| 241 | mkplknsqve | vsweypdsws | tphsyfslkf | fvriqrkkek | mketeegcnq | kgaflvekts |
| 301 | tevqckggnv | cvqaqdryyn | sscskwacvp | crvrs | | |

Recombinant protein CD5L monomer and homodimer was purified from the supernanant of 293E cells transfected with a CD5L expression vector. Recombinant mCD5L:p40 was recovered from the supernatant of 293E cells transfected with the CD5L:p40 expression vector. After harvesting transfected 293E cells by centrifugation, the protein was affinity purified from the supernatant using Ni Sepharose 6 Fast Flow resin (GE Healthcare). After binding the protein to resin, the resin was washed with 20 mM Tris, 0.3M NaCl, pH 8.0 and the protein eluted using 20 mM Tris, 0.3M NaCl, 0.4M Imidazole, pH 8.0. The protein was further polished by a Superdex S200 sizing exclusion column (GE Healthcare) in buffer 10 mM NaHPO4, 0.15M NaCl, pH 7.2. The S200 profile of the mCD5L:p40 showed a single peak. The S200 profile of the mCD5L transfection showed two overlapping peaks, corresponding to the homo-dimer fraction first and then monomer fraction

### Example 2—Soluble CD5L and CD5L/p40 can Regulate T Cell Function and have Overlapping as Well as Distinct Roles

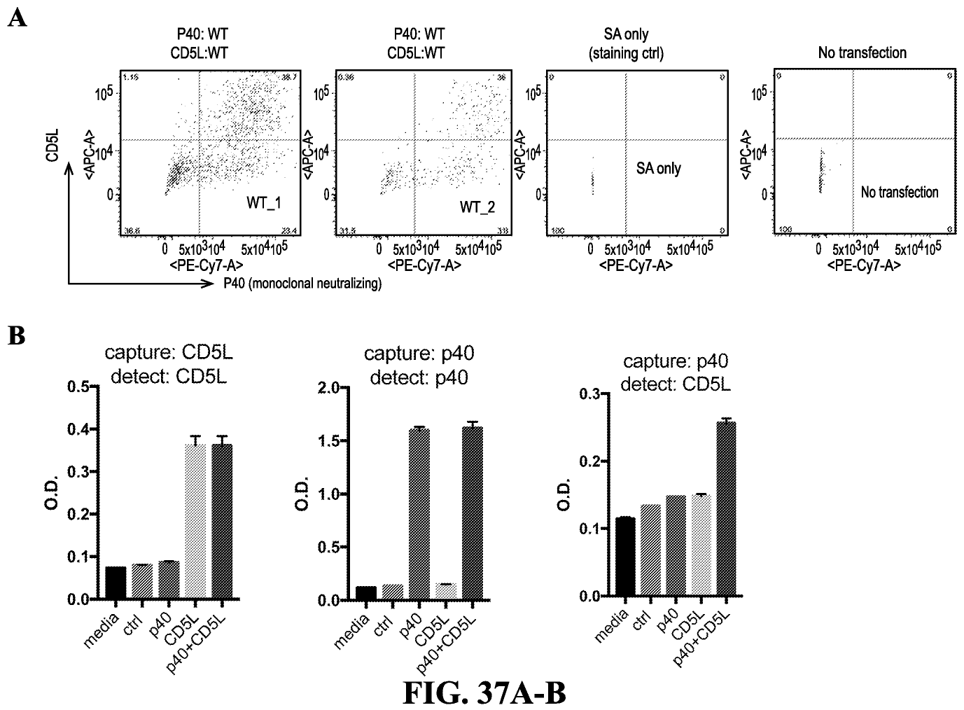

CD5L can be secreted by macrophages (Miyazaki et al., 1999) and given its T-cell intrinsic role, we tested the hypothesis that soluble CD5L can regulate T cell function directly in vitro. Although Abdi et al. reported that CD5L can form a heterodimer with p40, no specific function was attributed to this potential cytokine (Abdi et al., 2014). Immunoprecipitation experiments showed that CD5L and p40 can be secreted as heterodimer (FIG. 37). We hypothesized that both soluble CD5L and CD5L:p40 heterodimer can regulate T cell function directly.

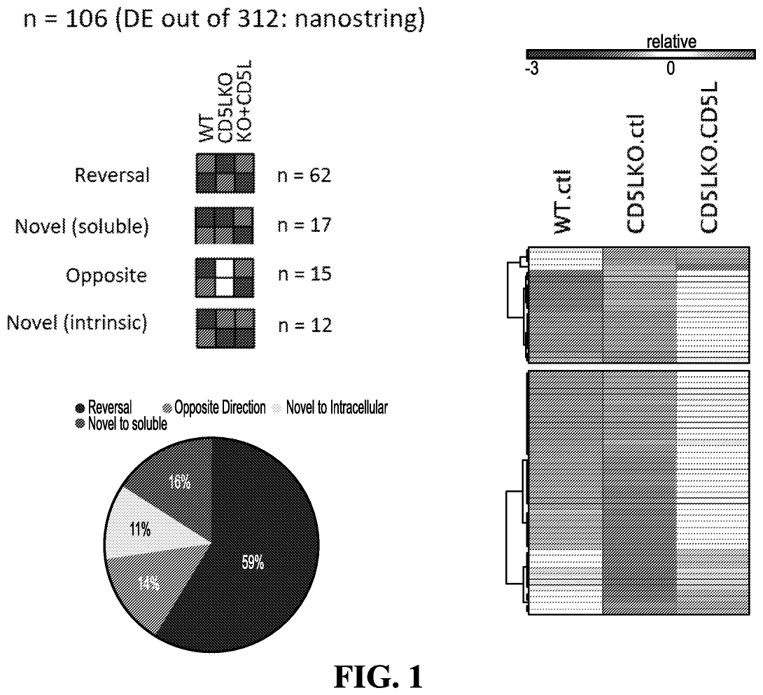

To this end, we used recombinant CD5L monomer either alone or with recombinant p40 monomer and analyzed the transcriptome of activated CD4 T cells, either WT or CD5L^{-/-}, co-incubated with these soluble factors. First, we analyzed the effect of soluble CD5L alone. We reasoned that if soluble CD5L (sCD5L) functions similarly to that of T-cell intrinsic CD5L, the addition of sCD5L can reverse the effects of CD5L deficiency on T cells. Indeed, we showed that sCD5L reversed the expression profile of majority of genes differentially regulated by any of the conditions tested (FIG. 1A). To exclude inference from T cell endogenous CD5L expression, we focused on the impact of sCD5L on Cd51^{-/-} T cells. Of interest, sCD5L also regulated expression profile of genes that were not changed comparing WT and Cd51^{-/-} T cells or opposed the T-cell intrinsic function of CD5L (FIG. 1A), suggesting potential novel role of the soluble CD5L.

Next, we performed pathway analysis of genes regulated by soluble CD5L and found sCD5L regulated gene profile contains both a regulatory and an inflammatory component. First, we observed that in sCD5L treated T cells there was a significant enrichment of signature genes of regulatory T cells from four different datasets using MSigDB (Table 3). Interestingly the key transcription factor of Treg, Foxp3, was downregulated by sCD5L (Table 3). This is consistent with sCD5L also promoting factors (such as 114, 119) that have been implicated in destabilizing Foxp3 expression antagonizing retinoic acid (Table 3 and (Hill et al., 2008)). These data suggest that soluble CD5L may promote a regulatory program but independent of Foxp3 expression and maybe an inducer of Th9 response. In addition to the regulatory component, we found that sCD5L regulated genes are significantly enriched for genes induced by IL-6/IL-1B but downregulated by IL-6/IL-1B/IL-23, suggesting soluble CD5L may antagonize IL-23 function (Table 3).

TABLE 3
Pathway analysis of soluble CD5L-dependent regulation of T cells.

| | |
|---|---|
| Enriched pathways | genes |

A. Reversal/Novel (soluble) UP

| | |
|---|---|
| Treg (4 independent | (PDL2, LIF, SOCS2, IKZF4, ICOS, |
| datasets) (FDR q-value | PROCR, NFIL3, CD200, TGM2, PRNP, |
| 1.63e−8) | CD70, XBP1, ATF4, LAD1, KLF9, CD83, |
| | Runx2, IRF8, IFNg etc) |
| RA treated memory CD4 | IER3, IL4, RAB33A, FZD7, NFIL3, |
| (FDR q-value 9.58e−10) | SLAMF7, TNFSF9, FAIM3, IL9, Foxp3 |
| IL-6/IL-1B | IL-22, GJA1, EGR2, IL1RN, CD200, ITGA3 |
| IL-4 | IL-4 |

B. Reversal/Novel (soluble) DOWN

| | |
|---|---|
| -IL-6/IL-1B/IL-23 | GMFG, MGLL, FRMD4B, MINA |

Soluble CD5L induces both a regulatory and proinflammatory program including 119 response. Differentially regulated genes were investigated using Msigdb and selected significant enrichment are listed in A and B showing those upregulated and downregulated by soluble CD5L respectively. Red and Green indicates directionality: Red pathway means soluble CD5L treatment goes with, green pathway means goes against such pathways (In the above tables, the “Treg,” “IL-6/IL-1B,” and “IL-4” rows are red pathways, and the “RA treated treated memory CD4” and “IL-6/IL-1B/IL-23” rows are green pathways).

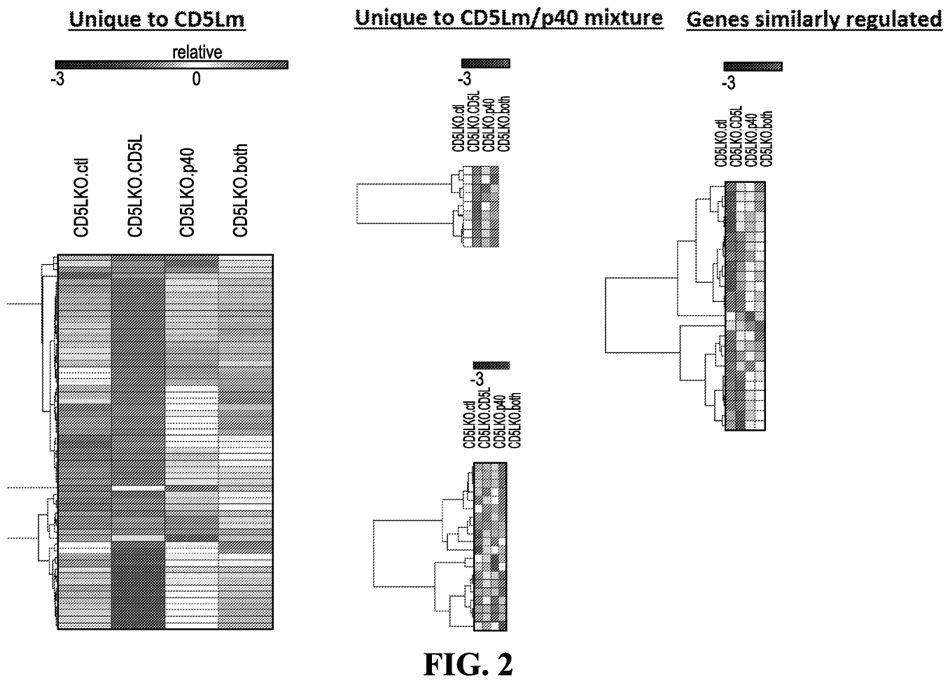

Finally, we compared the effect of sCD5L to that of sCD5L:p40 and found these two cytokines to regulate the expression profile of both similar and distinct set of genes (FIG. 2). Thus, these data collectively suggest sCD5L and sCD5L:p40 are novel cytokines that can regulate T cell function.

### Example 3—T Cell Regulation by sCD5L and CD5L:p40 Depends on IL-23R Signaling

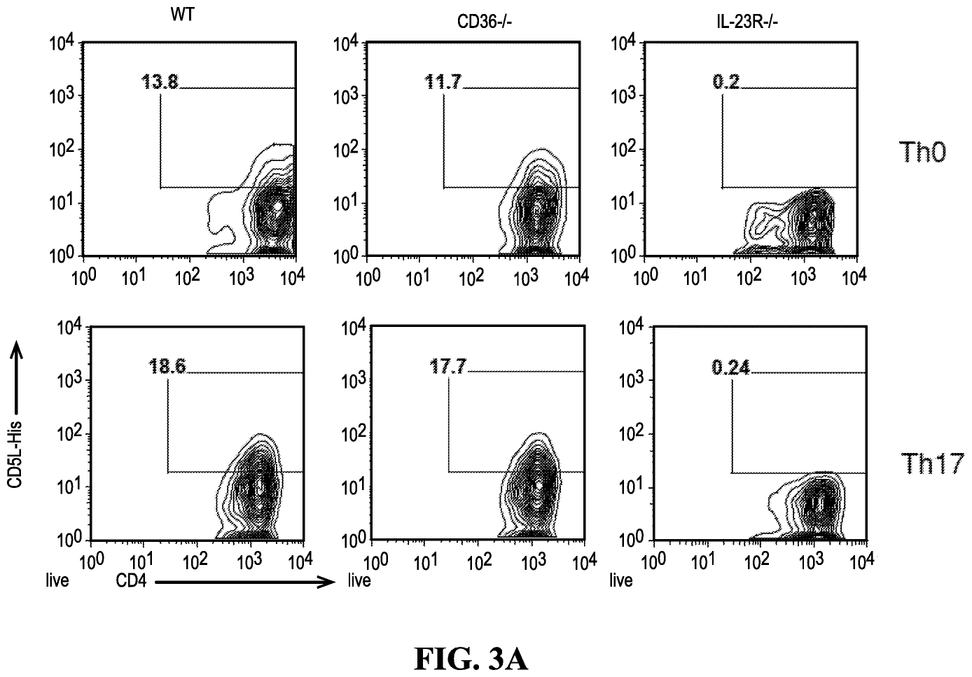

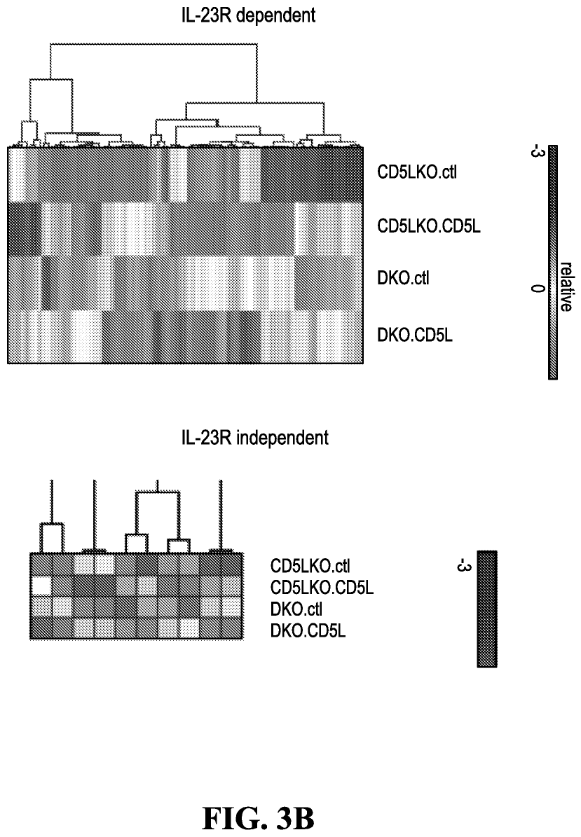

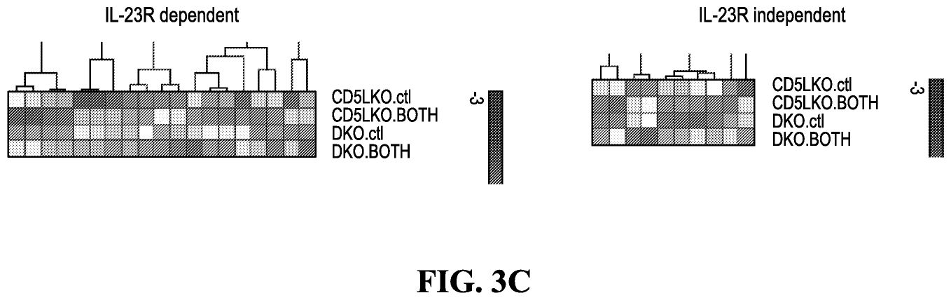

As sCD5L and CD5L:p40 can regulate gene expression in T cells, we investigated what receptor(s) might be responsible for their function. CD5L was reported to interact with CD36, a scavenger receptor, and thus can be internalized into adipocytes (Kurokawa et al., 2010). We investigated whether CD36 is required for signaling of sCD5L in T cells. We showed that His-tagged sCD5L can stain WT and CD36^{-/-} T cells equally well even at lower concentrations (FIG. 3A and data not shown). While this data is consistent with lower expression of CD36 on T cells compared to macrophage (ImmGen database), it also raises the question whether the sCD5L can bind to a different receptor on T cells.

CD5L can form a heterodimer with p40 and p40 can bind to either p19 or p35. We hypothesized that if sCD5L binds to a surface receptor it may be co-regulated/dependent on receptors for the other two cytokines: that is IL-12RB1, IL-12RB2 or IL-23R. We tested whether sCD5L can stain Il12rb1^{-/-}, Il2rb2^{-/-} or 1123r^{-/-} T cells as compared to WT (FIG. 3A and data not shown). Interestingly, the binding of sCD5L is abolished on 1123r^{-/-} T cells and partially reduced on 1l2rb1^{-/-}, 1l2rb2-T cells. These findings suggest that CD5L may interact with a receptor that depends on IL-23R signaling.

Next, we asked the question whether the function of sCD5L is also affected by the absence of IL-23R on T cells. To this end, we crossed Cd51^{-/-} mice with 1123r^{-/-} mice and found that in the absence of IL-23R, the expression of 89% of genes (84 out of 94 based on nanostring set) regulated by sCD5L were no longer affected (FIG. 3B). The effect of CD5L:p40 heterodimer could also be partially dependent on IL-23R expression (FIG. 3C). Thus sCD5L and CD5L:p40 may interact with different receptors on T cells.

### Example 4—CD5L Regulates not Only T Cells but Also Restrains Proinflammatory Function of Innate Lymphoid Cells (ILC) and is Expressed by ILC in Naïve Mouse

The discovery that soluble CD5L can regulate T cell function directly and that its impact may dependent on IL-23R expression prompted us to study whether CD5L can regulate other cells that may also express IL-23R. To this end, we investigated the impact of CD5L on two such populations that express IL-23R: innate lymphoid cells (ILC) and dendritic cells (DC).

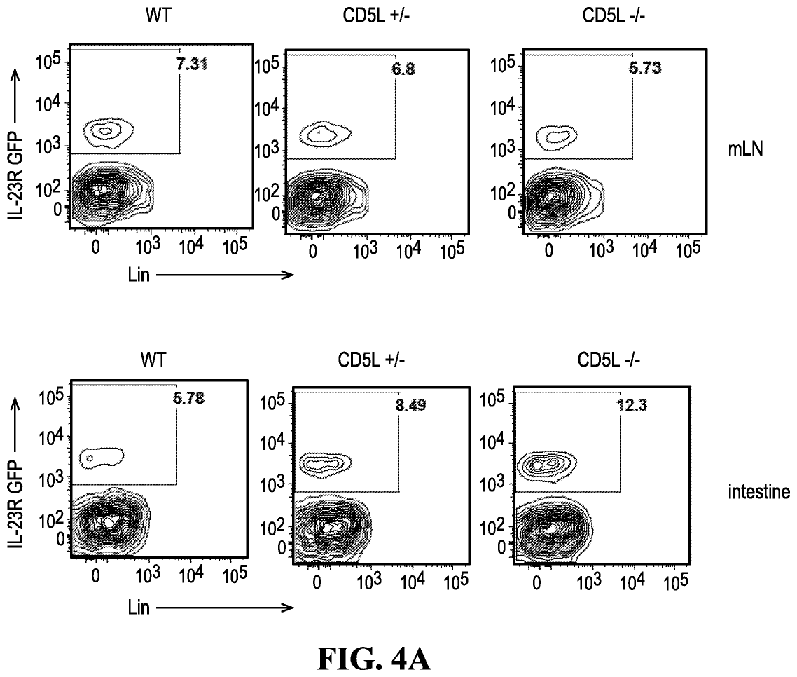

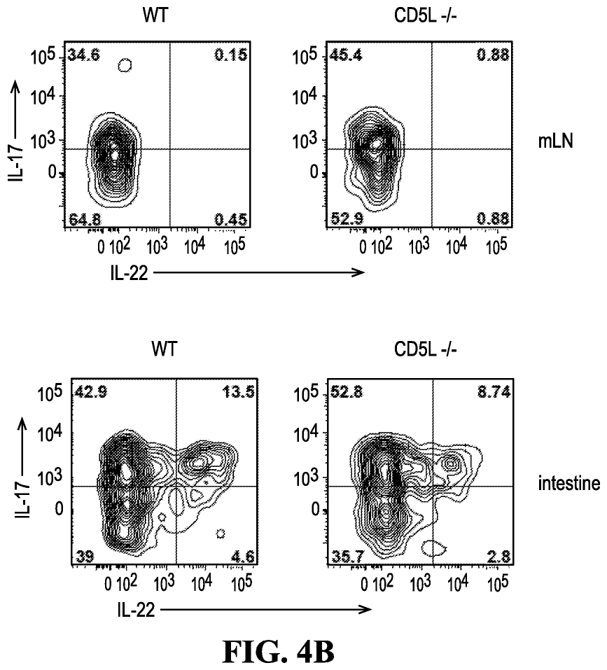

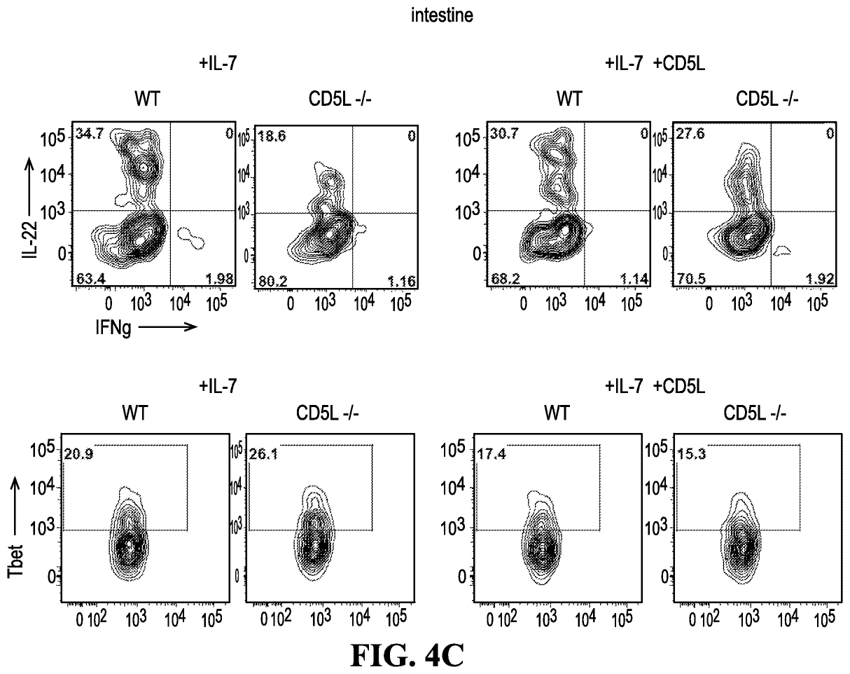

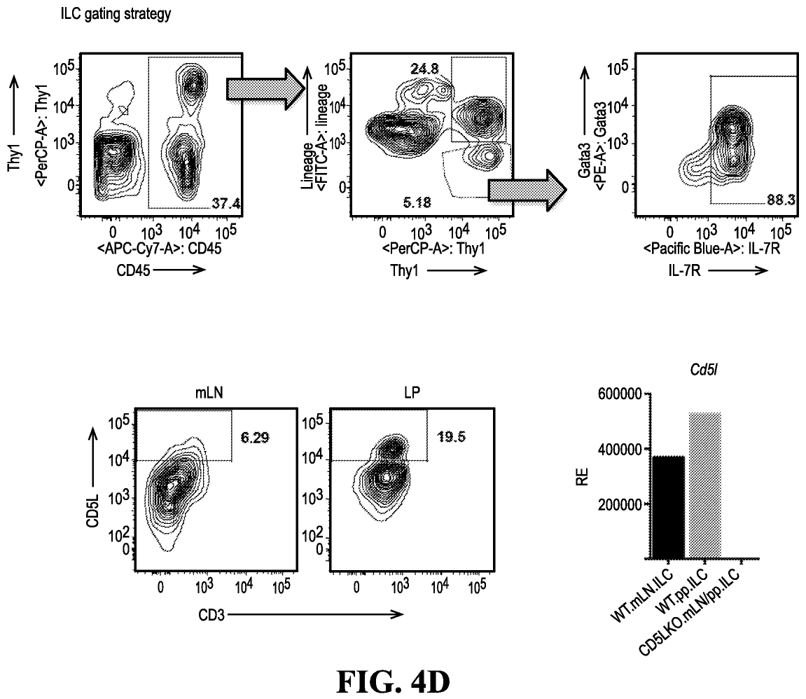

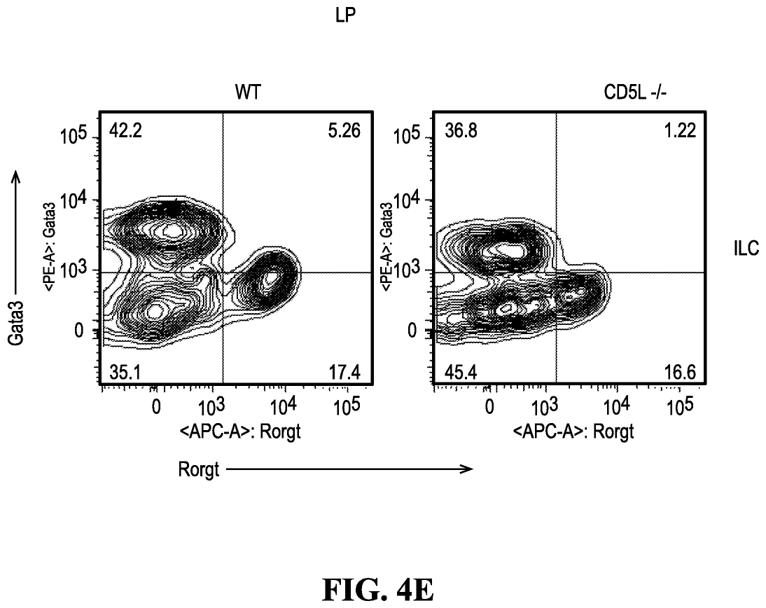

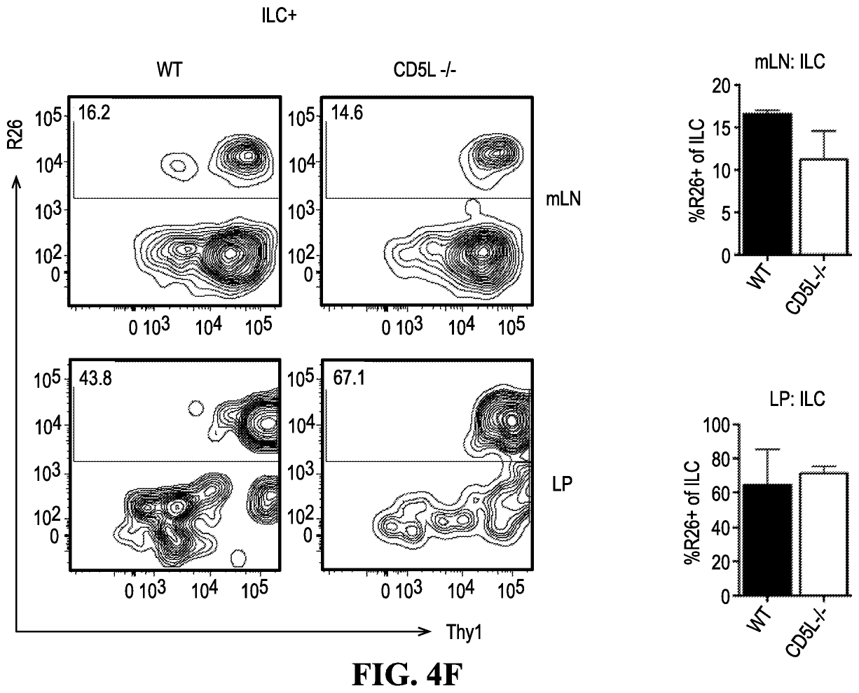

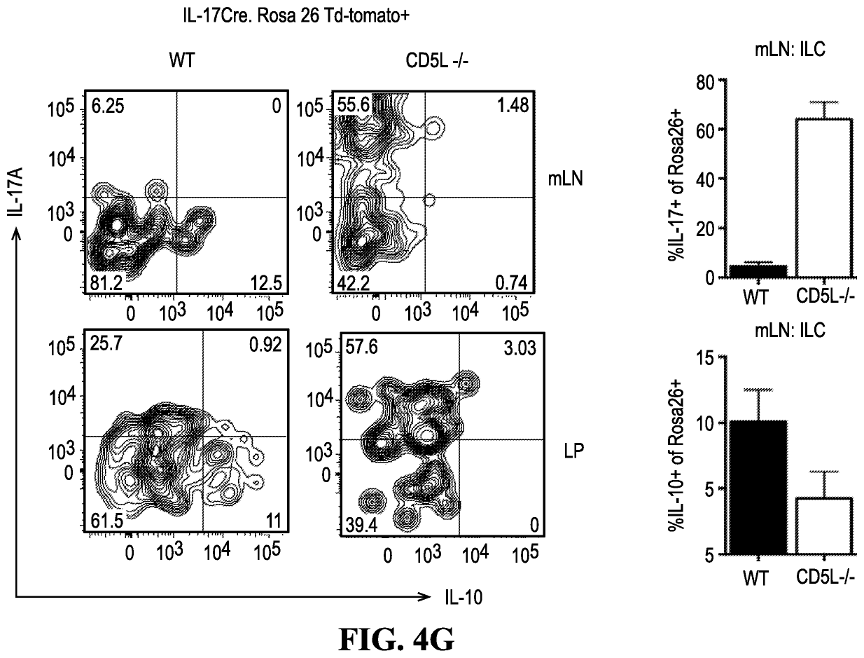

First, we analyzed the percent and function of ILC in naïve 6-month old WT versus Cd5^{-/-} mice. We observed that IL-23R expression on ILC from lamina propria is significantly increased in the absence of CD5L (FIG. 4A). This is accompanied with higher proportion of ILCs producing IL-17 and Tbet, but lower percent of IL-22 producers (FIG. 4BC). We further demonstrated that the reduced IL-22 expression and increased Tbet expression by ILC can be reverted by soluble CD5L ex vivo (FIG. 4C). These data suggest that CD5L can regulate ILC function at steady state. Of interest, we observed that ILC isolated from both mLN and lamina propria from naïve mice can express CD5L (FIG. 4D).

Next, we asked whether CD5L influence ILC during inflammation. As CD5L regulates IL-17 and IL-17 production is associated with ILC3, we crossed Cd51−/− mice with fate mapping reporter mice Il17a^{Cre}Rosa26^{Td-tomato} to better track ILC3 that has ever transcribed sufficient IL-17 to turn on the Cre. Using the DSS-induced acute colitis model, we showed that there is similar percent of Rosa26⁺ ILC comparing 8-wk old WT.Il17Ta^{Cre}Rosa26^{Td-tomato} and Cd5^{-/-}Il17a^{Cre}Rosa26^{Td-tomato} mice at day 11 since DSS treatment (FIG. 4F), suggesting CD5L does not influence the differentiation of ILCs initially. Consistently, the percent of ILC that expresses Rorgt is not significantly altered (FIG. 4E). In contrast to the Rosa26 expression, ILC from WT.Il7a^{Cre}Rosa26^{Td-tomato} make little IL-17 and turned on IL-10 expression in striking contrast to those from Cd51^{-/-}Il17a^{Cre}Rosa26^{Td-tomato} mice which continue to produce much higher expression of IL-17 and are IL-10 negative (FIG. 4G). Thus CD5L can restrain proinflammatory function of ILC during acute inflammation.

### Example 5—CD5L:p40 Promotes Regulatory Programs in CD11c+ Cells in an IL-23R but not CD36 Dependent Manner

It has been reported that CD5L can induce autophagy in the human macrophage cell line, THP, limiting TNFa and IL-1B expression and promoting IL-10 expression (Sanjurjo et al., 2015). The authors propose CD36 is the major recipient of CD5L in these cells. As we discovered that sCD5L (and CD5L:p40 heterodimer) could regulate T cells through an IL-23R-dependent alternative receptor, we tested the hypothesis that CD5L and CD5L:p40 may regulate myeloid cells in an IL-23R dependent pathway.

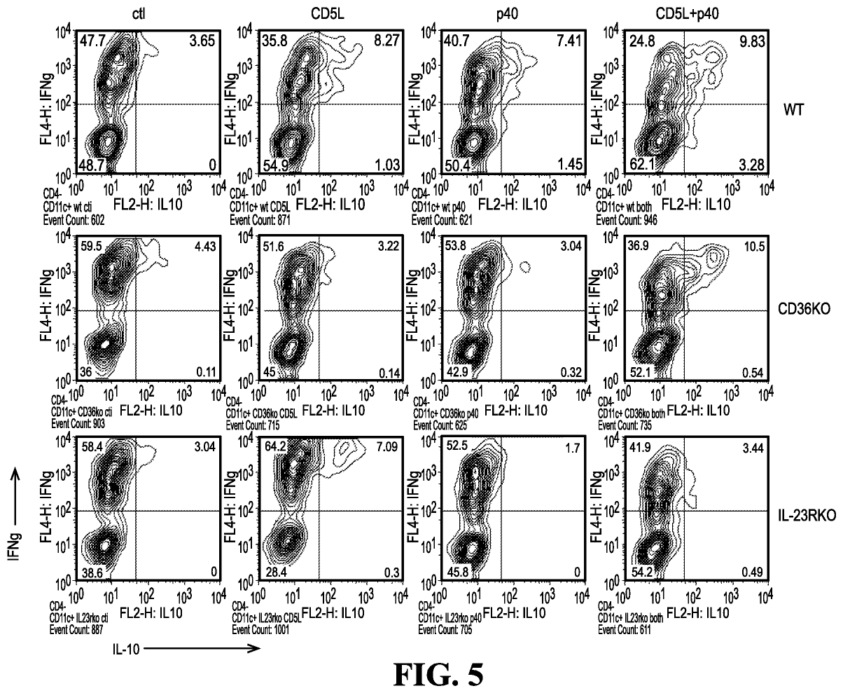

To test this hypothesis, we isolated WT, CD36^{-/-} and IL-23R^{-/-} CD11c⁺ cells from spleen of naïve mice and stimulated the cells with LPS in the presence of sCD5L, p40 or CD5L:p40. We showed that sCD4L, p40 and CD5L:p40 can all induce IL-10 expression from CD11c⁺ cells, however the effect of CD5L:p40 is dependent on IL-23R whereas the effect of sCD5L is dependent on CD36 (FIG. 5).

### Example 6—CD5L Plays a Protective Role in Acute Colitis and Cancer

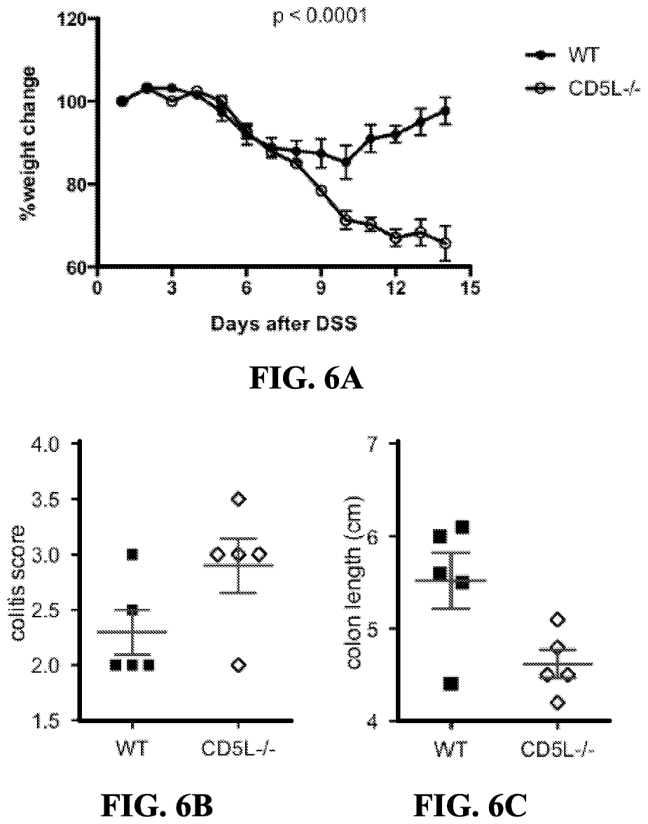

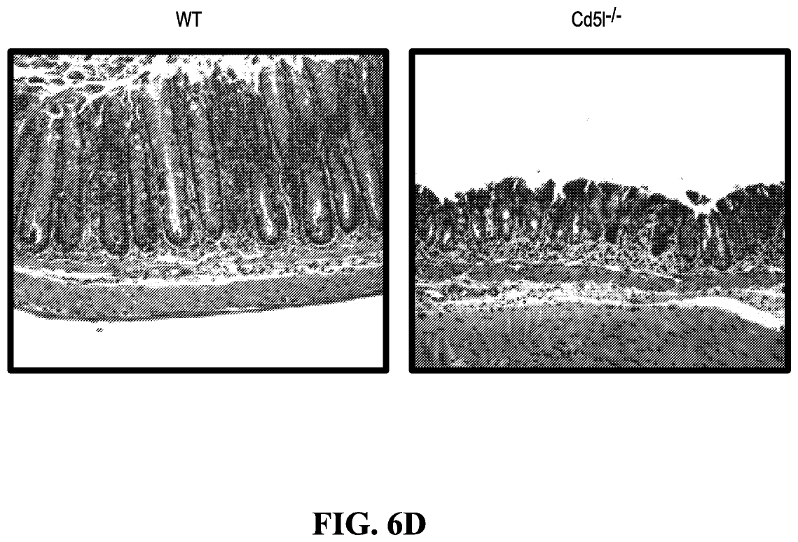

To test the function of CD5L and CD5L:p40 in vivo, we tested several disease models. CD5L^{-/-} mice were treated with 2% DSS in drinking water for 6 days followed by normal water. Weight loss was reported as a percentage of initial weight in FIG. 6A. Colitis score and colon length were determined on day 14, and are shown in FIGS. 6B and C, respectively. Colon histology on day 14 is shown in FIG. 6D. This data demonstrates that CD5L influenced tumor progression in a B16 melanoma model.

### Example 7—CD5L Ameliorates Autoimmune Diseases (Including MS), Acute Colitis, and Cancer

To show that CD5L:p40 can ameliorate disease, we therapeutically treat mouse models of multiple sclerosis (EAE), colitis (e.g., DSS-induced injury model which is a mouse model for ulcerative colitis and T-cell dependent colitis model) or cancer (e.g., mice with inflammation-induced cancers, or human cancer xenografted onto mice) with recombinant CD5L:p40, or antibodies or antigen-binding fragments thereof or that bind to the heterodimers.

### Example 8—Recombinant CD5L Binds to T Cells and Suppresses EAE and DSS-Induced Colitis

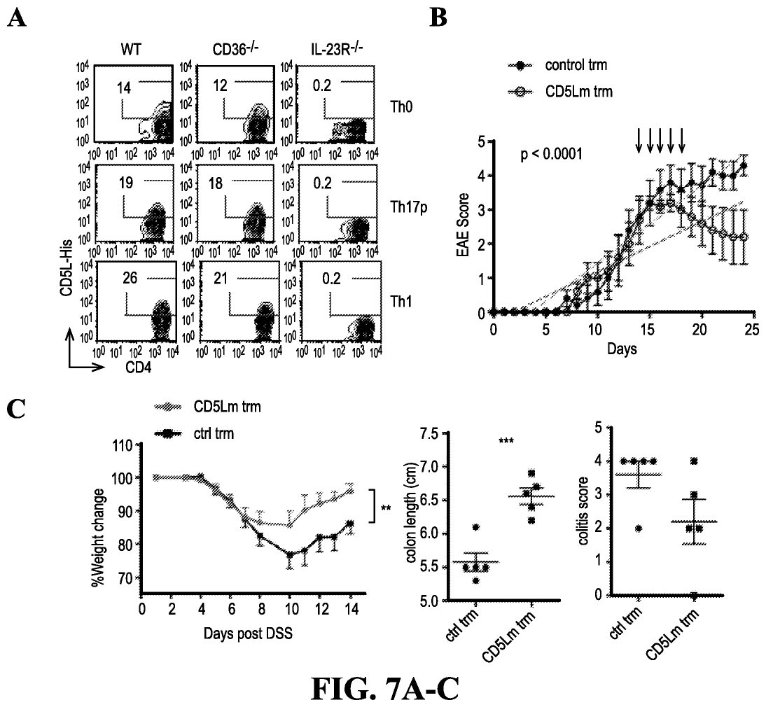

Experiments were conducted to assess whether soluble CD5L could regulate effector T cells. In particular, soluble CD5L was directly evaluated using recombinant CD5L with a His-tag. Th0, Th1 (IL-12), and TH17p (IL-1b, IL-6, IL-23) cells were differentiated from naïve CD4 T cells in vitro for 4 days, and cells were harvested for staining with recombinant CD5L followed by anti-His APC antibodies and flow cytometry analysis. Flow cytometry data showed that CD5L can bind to both Th1 and pathogenic Th17 cells (Th17p) and to a lesser extent Th0 cells (FIG. 7A). The binding of CD5L on T cells was shown to not require CD36, but to be dependent on IL-23R (e.g., loss of IL-23R abrogated CD5L binding to T cells).

In vivo therapeutic experiments were conducted by immunizing wildtype mice with MOG/CFA following by PT injection to induce EAE. Mice at peak of disease (score=3 in FIG. 7B) were injected with either PBS (solid circles) or recombinant CD5L (empty circles) intraperitoneally daily for 5 consecutive days and mice were measured for disease progression. As shown in FIG. 7B, soluble CD5L was shown to have a therapeutic effect on EAE.

In a separate experiment, wildtype mice were induced with colitis via 2.5% DS in drinking water for 6 consecutive days, followed by normal water for 8 days. Mice were given either a control (PBS) or recombinant CD5L (CD5Lm) intraperitoneally on day 4, 6, and 8. Colon length and colitis score were recorded on day 14. As shown in FIGS. 7C and 7E, recombinant CD5L was sufficient in alleviating colitis disease severity.

### Example 9—Endogenous CD5L Forms a Heterodimer (CD5L:p40) and is Inducible During an Acute Inflammation

CD5L can bind to p40, the subunit shared by the cytokines IL-12 and IL-23, and form a heterodimer in vitro. This raises the intriguing possibility that CD5L can generate different soluble mediators with potentially distinct functions. To determine whether CD5L:p40 heterodimer can be detected in vivo in biological settings, recombinant CD5L:p40 (FIG. 8A) was generated and used to optimize an ELISA that allowed the detection of endogenous CD5L:p40 heterodimer.

Serum was collected kinetically from wildtype and Cd51^{-/-} mice with DSS-induced colitis (2% DSS in drinking water for 6 days followed by 7 days of normal water) and the level of CD5L:p40 was measured using an ELISA assay. In the ELISA assay anti-IL-12 p40 was used to capture the heterodimer and enzyme linked anti-CD5L was used to detect the heterodimer. Data from this assay showed that natural CD5L:p40 heterodimer was induced during the course of DSS-induced colitis in serum (FIGS. 8B and 8C).

### Example 10—IL-27 and TLR9 Induce CD5L Dimerization

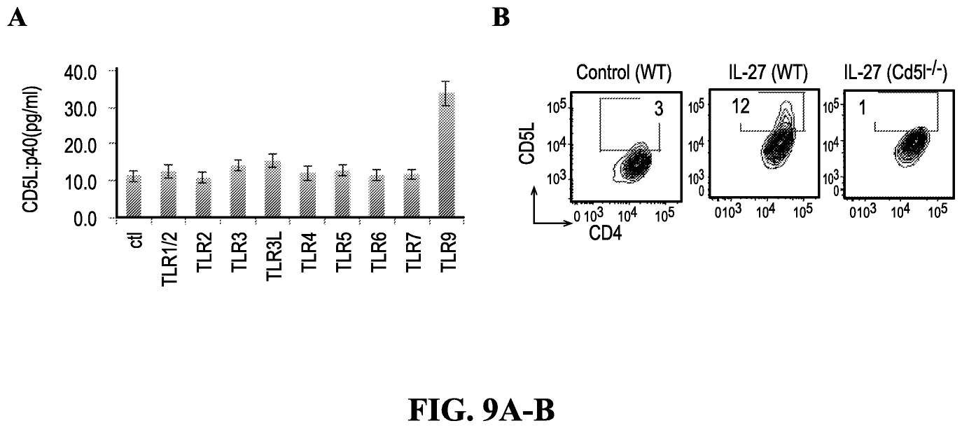

Preliminary screens were conducted to determine what signals could induce CD5L homodimer and CD5L:p40 heterodimer. In particular, bone marrow derived dendritic cells were stimulated with TLR ligands for 24 hours and the supernatant was analyzed for CD5L:p40 secretion by ELISA. The screens showed that TLR9 can induce the secretion of CD5L:p40 (FIG. 9A). To determine the signals that could induce CD5L on T cells, CD5L expression in Th0, Th1, Th2, Th17 and Tr1 cells was analyzed, and the data showed that the immunosuppressive cytokine IL-27 can indeed induce CD5L (FIG. 9B and data not shown).

### Example 11—CD5L Homo/Heterodimer Inhibits IL-17 Production and the Pathogenic Th17 Cell Signature

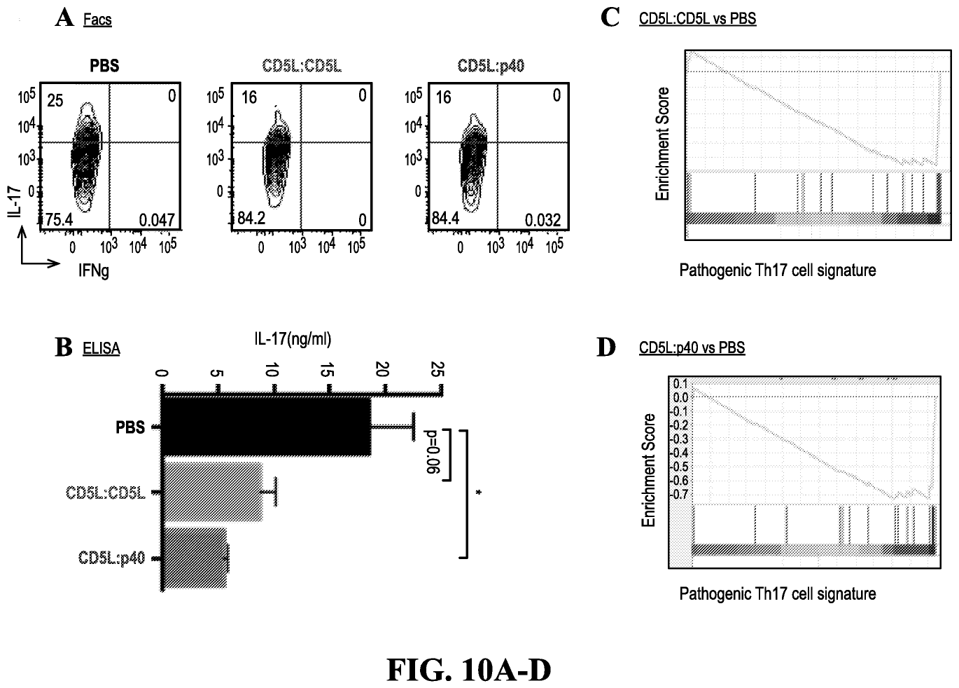

To determine the function of CD5L homo/heterodimers on Th17 cells directly, pathogenic Th17 cells (IL-1b+IL-6+IL-23) were treated with either PBS (control), CD5L homodimers or CD5L:p40 heterodimers. IL-17 expression of T cells was measuring by FACS (FIG. 10A), and IL-17 production in serum was measured by ELISA (FIG. 10B). These experiments showed that both forms of CD5L inhibited IL-17 expression (FIGS. 10A-B).

To test whether recombinant CD5L can regulate the transcriptome of Th17 cells and particularly the pathogenic signature, the RNA expression of control and treated cells was studied with a custom-code set of 337 genes, and analyzed against signature genes of pathogenic Th17 cells (e.g. i123r, il22, il1r1, csf2) with GSEA, using the nanostring platform. The signature of pathogenic Th17 cells was significantly reduced by both CD5L:CD5L and CD5L:p40 as compared to a control (FIG. 10 C (FDR q=0.031, NOM p=0.000, NES=−1.66) and 10D (FDR q=0.031, NOM p=0.000, NES=−1.47), respectively).

### Example 12—CD5L Suppresses IL-17 and IFNg Expression from Pathogenic Th17 Cells and Th1 Cells, Respectively

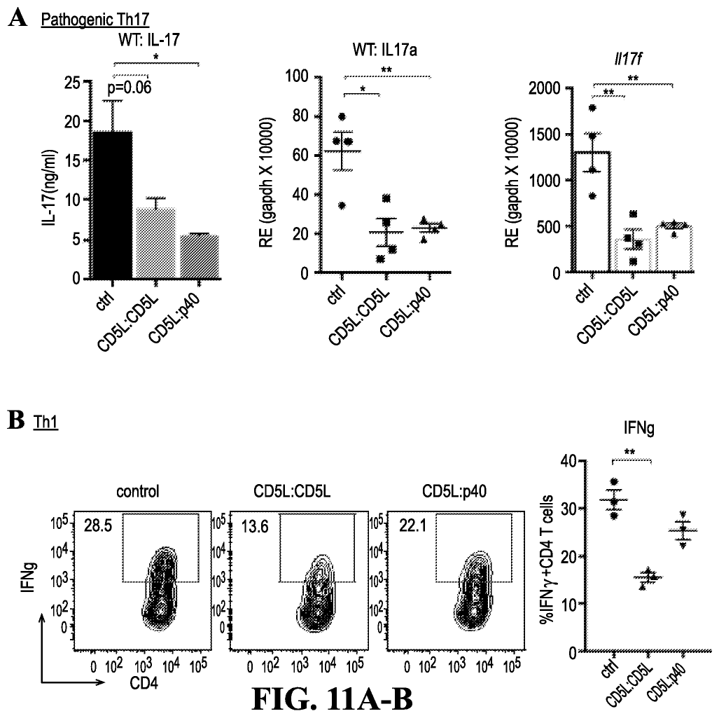

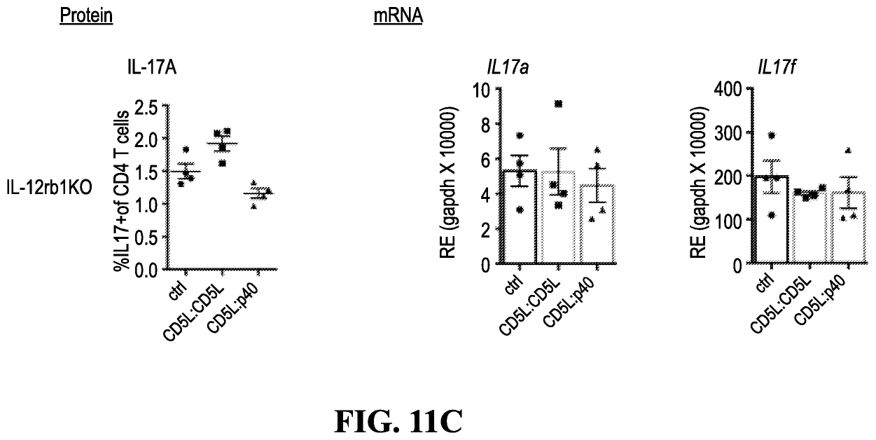

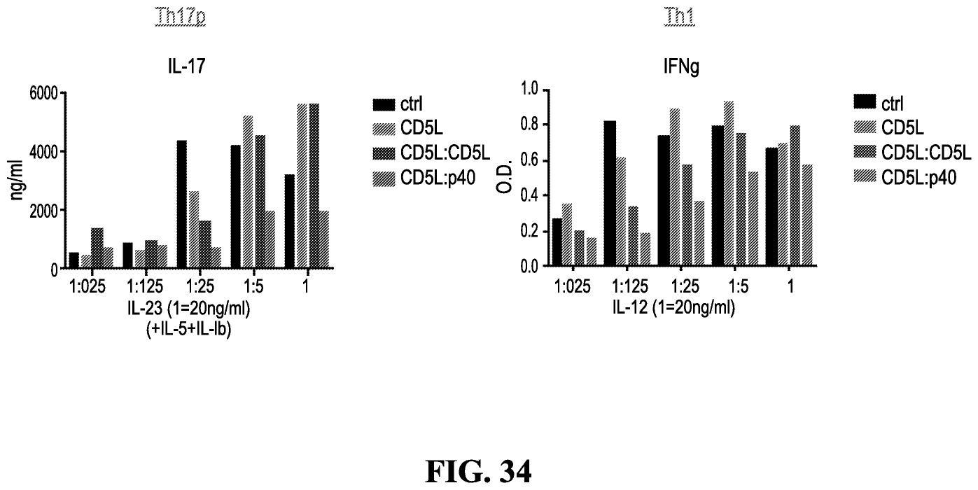

Pathogenic Th17 cells and Th1 cells were differentiated from naïve CD4 cells (CD44^{low}CD62L⁺CD25-CD4⁺) from wildtype mice with IL-1b, IL-6, and IL-23 (Th17) or IL-12 (Th1) in the presence of a control, CD5L homodimer, or CD5L:p40 heterodimer for 48 hrs (Th17) or 72 hours (Th1). IL-27 expression in Th17 cells was measured by ELISA in supernatant (FIG. 11A, left side) and by qPCR from RNA purified from cells (FIG. 11A, right side). A reversal of this effect is shown in IL12rblknockout mice subjected to the same protocol (FIG. 11C), demonstrating that the effects of CD5L:p40 heterodimer and CD5L:CD5L homodimer on Th17 cells are IL12rb1 dependent. IFNg expression in Th1 cells was measured by intracellular staining followed by flow cytometry analysis (FIG. 11B). A similar protocol was repeated for all three CD5L entities, including the CD5L monomer. (FIG. 34). The results showed that CD5L suppresses IL-17 and IFNg production in pathogenic T cells.

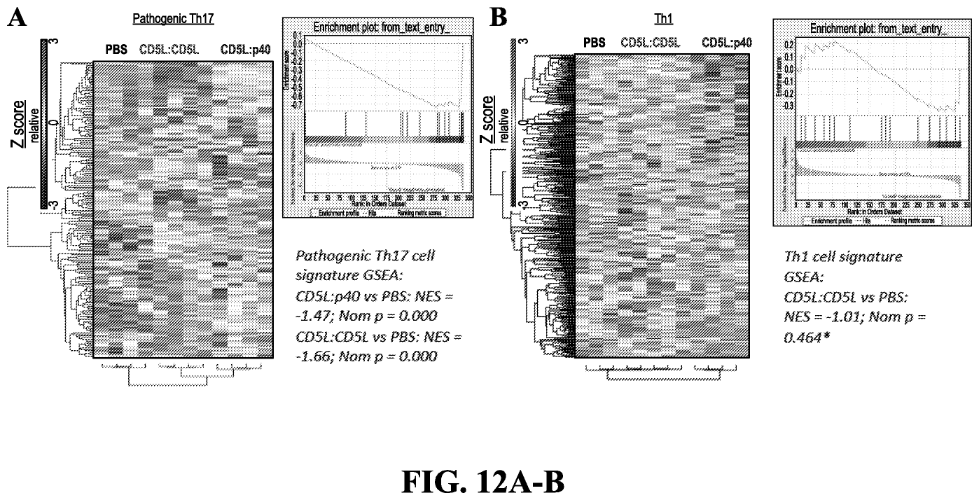

To assess pathogenic T cell signatures, RNA was extracted from both Th17 and Th1 cells after 48 hours of differentiation. Extracted RNA was analyzed with a custom codeset of 337 genes using the nanostring platform (four replicates for each conditions were measured). The Spearman coefficient was used for clustering. A heat map of differentially expressed genes as compared to control (defined by p<0.05) is shown in FIG. 12A for Th17 cells and FIG. 12B for Th1 cells (left panels). GSEA analysis against the pathogenic signatures are shown in the right panels of FIGS. 12A and B.

### Example 13—Endogenous CD5L Promotes EAE Resolution and is Expressed by Both Non-Pathogenic Th17 Cells and CD11b+ Cells During EAE Development

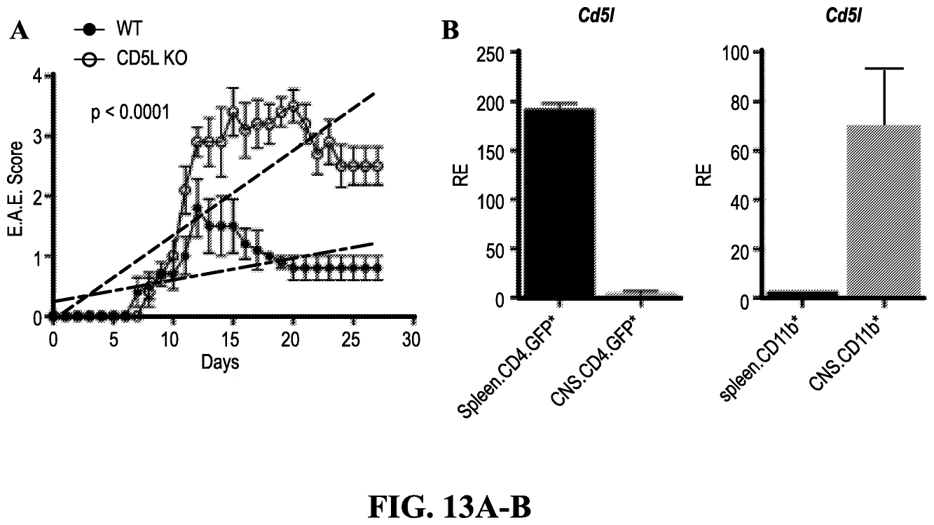

To determine which cells express CD5L during EAE, CD5L^{-/-} mice were immunized with MOG/CFA to induce EAE and followed for clinical scores. Th17 cells (IL-17.GFP+CD4+) and CD11b+ myeloid cells were sorted from both spleen and CNS of mice at peak disease (score=3). Mice with global CD5L deficiency showed more severe and sustained EAE compared to controls (FIG. 13A), indicated that CD5L contributes to EAE resolution.

To assess CD5L expression in EAE, IL-17 GFP reported mice were immunized with MOG/CFA to induce EAE. Mice were sacrificed at peak of disease (score=3). Th17 cells were sorted based on CD4+GFP+ and macrophage were sorted based on CD1 lb+ from both the spleen and CNS of the mice. RNA was purified from sorted cells and qPCR was used to measured CD5L expression. The experiments showed that CD5L was preferentially expressed by Th17 cells in the spleen and by macrophage cells in the CNS (FIG. 13B).

### Example 14—Generation of CD5L Conditional Knockout Mouse; Role in Tumor Immunity

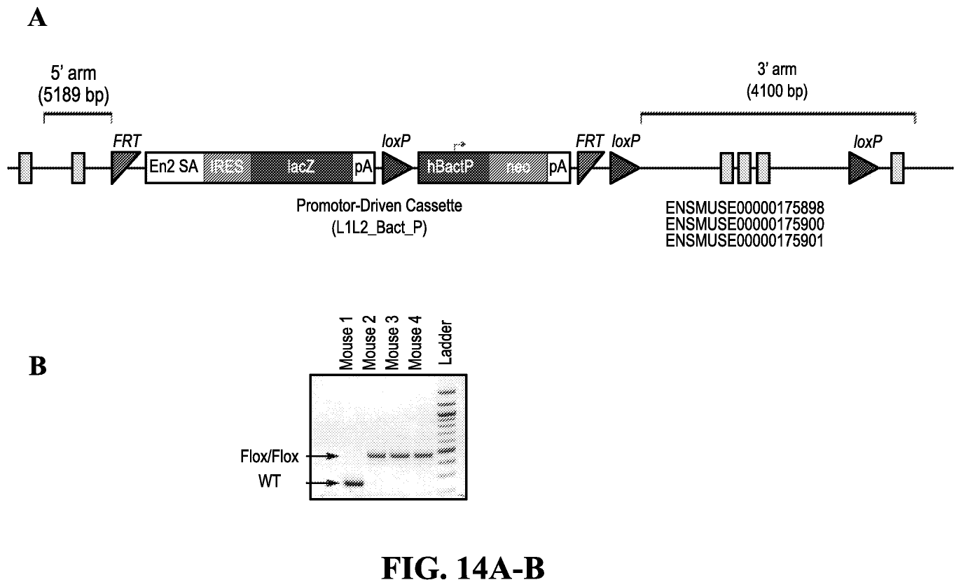

To study the cellular source of CD5L during EAE development, CD5L flox/flox mice (CD5Lf1/f1) were generated by crossing FLPo mice and mice that were heterozygous with the construct shown in FIG. 14A (purchased from EUCOMM/KOMP). The CD5L flox/flox mice were bred to homozygosity and crossed with CD4-Cre, IL-17-Cre and LysM-Cre for conditional deletion of the Cre-loxP system. Representative genotyping results for CD5L flox/flox mice are shown in FIG. 14B. CD5Ln^{fl/fl} mice were successfully crossed with LysMCre, CD4Cre and IL-17Cre mice to specifically delete CD5L in myeloid lineage cells, T cells and IL-17− producing cells respectively.

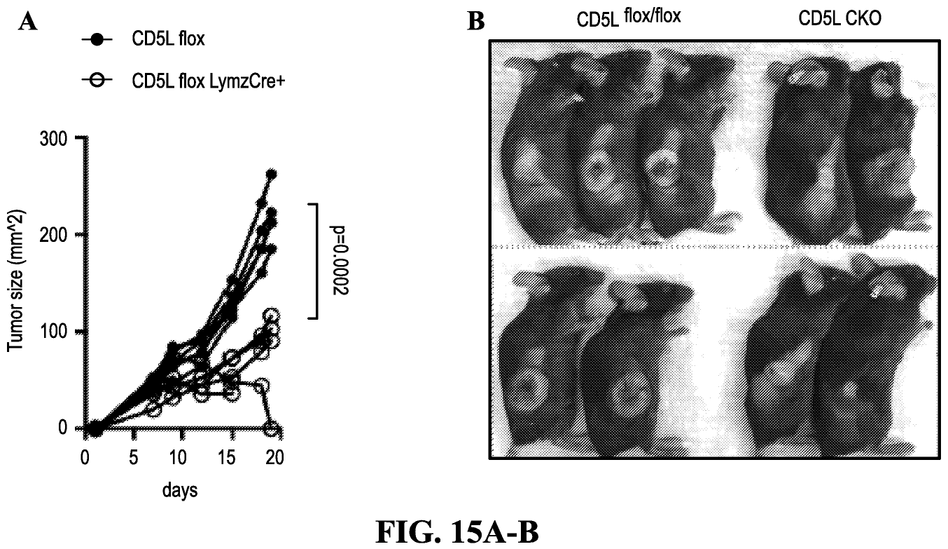

CD5L^{flox/flox}Lymz^{Cre+} (CD5L CKO) and CD5L^{flox/flox} mice were injected with 1×10⁶ MC38 colon carcinoma subcutaneously on the right flank. Tumor size was measured up to 19 days post-injection, and is plotted in FIG. 15A. Pictures of mice sacrificed on day 19 post tumor cell injection are shown in FIG. 15B.

### Example 15—CD5L and IL-23 Alter Lipidome of Th17 Cells in Correlation with T Cell Function and EAE

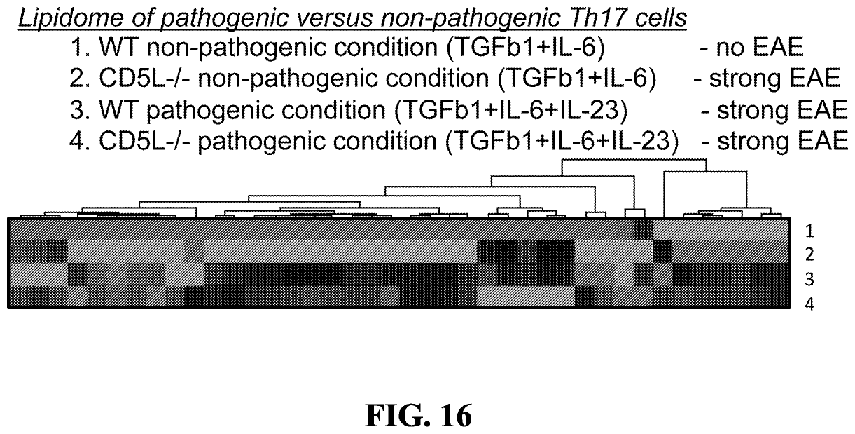

Th17 cells were differentiated from naïve cells under pathogenic and non-pathogenic conditions and harvested for LC/MS at 96 hours. The lipidome of wildtype and Cd51^{-/-} Th17 cells was analyzed. A striking correlation of the lipidome of Th17 cells to their function and ability to induce EAE was found (FIG. 16). In fact, Th17 cell function could be changed based on alterations of the Th17 cell lipidome.

### Example 16—Gene Expression Profile of Metabolic Pathways Correlates with Th17 Cell Pathogenicity

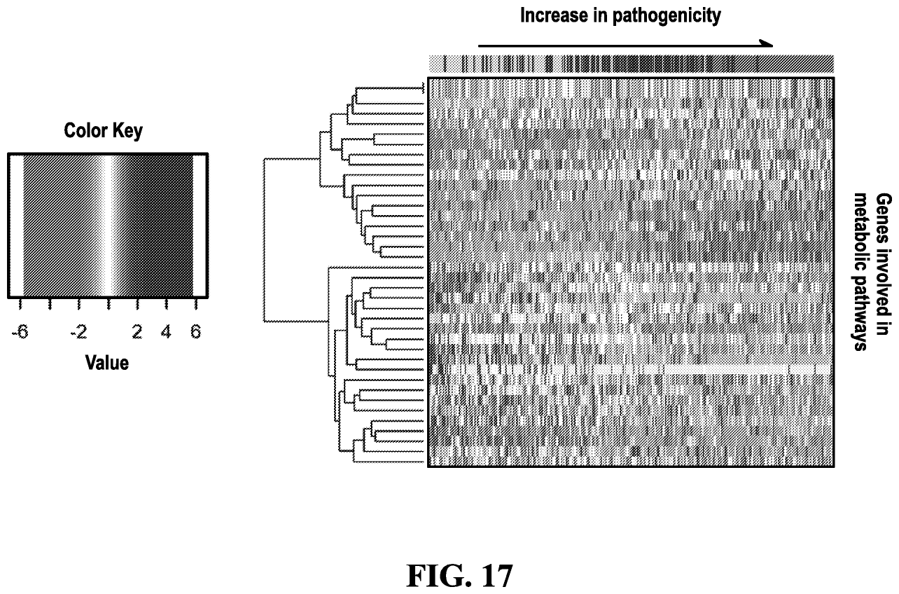

To determine whether metabolic genes are differentially expressed at the transcriptome level in Th17 cells with different functional state, the metabolic transcriptome in single cell RNA-seq data was analyzed. The analysis showed metabolic transcriptome expression covariance with Th17 cell pathogenicity (FIG. 17).

### Example 17—CD5L Plays a Critical Role in Tumor Immunity, Regulating T Cell Exhaustion

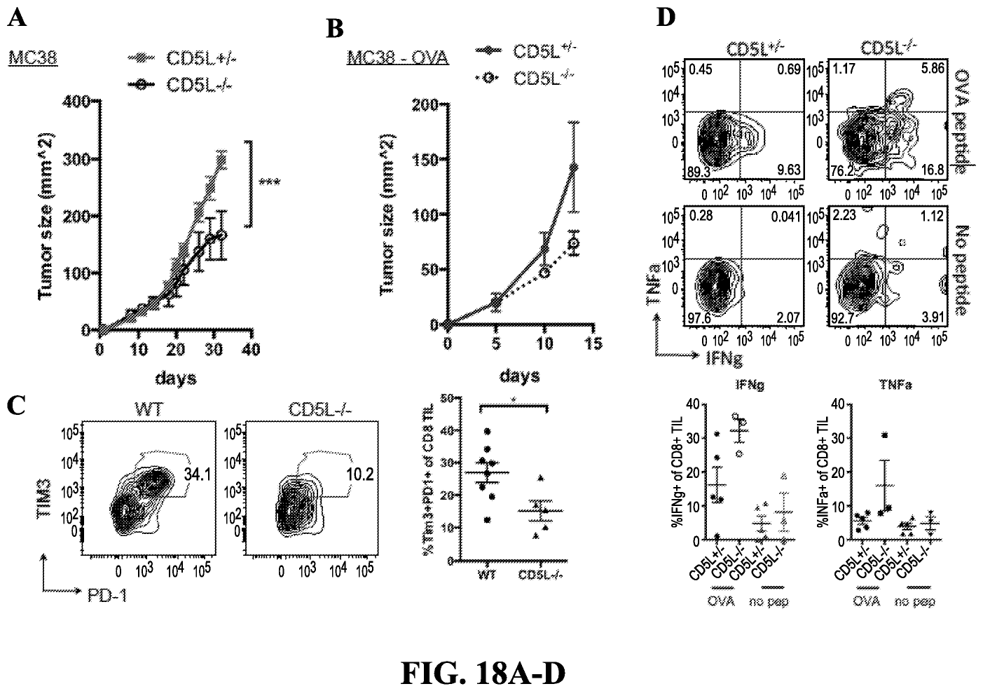

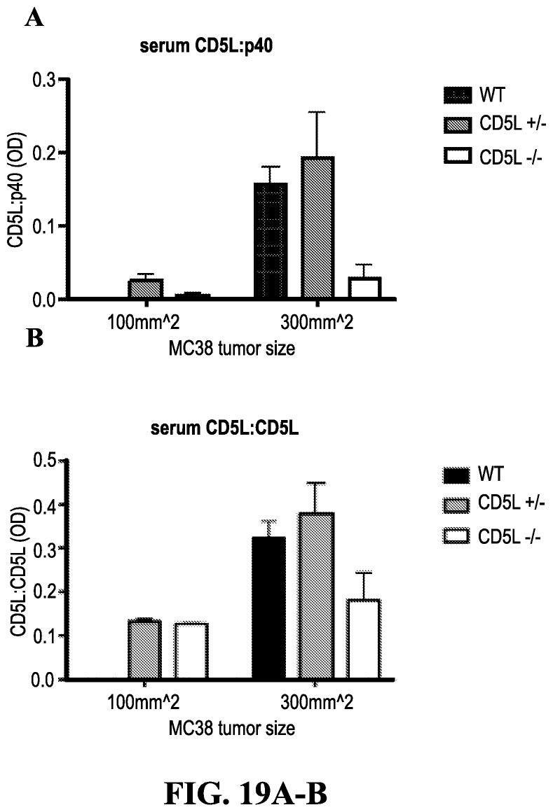

Littermate controls of CD5L^{+/-} and CD5L^{-/-} mice were grafted with 1×10⁶ MC38 or MC38-OVA colon carcinoma subcutaneously on the right flank, and then tumor progression was followed. Tumor size progression for MC38 and MC38-OVA experiments are shown FIGS. 18A and B, respectively. Tumor infiltrating lymphocytes were isolated from MC38 on day 30 and analyzed, and the results are shown in FIG. 19C. Tumor infiltrating lymphocytes were isolated from MC38-OVA on day 14 and inculcated with OVA peptide or no peptide (control) for 20 hours. Brefaldin A and monensin was added in the last 4 hours and cytokines were measured intracellularly by flow cytometry (see FIG. 19D). These results demonstrate that CD5L deficiency inhibits T cell dysfunction and promotes tumor suppression.

### Example 18—Link Between CD5L:p40 Heterodimer and Tumor Progression

Litter mate controls of wildtype, CD5L^{+/+} and CD5L^{-/-} mice were injected with 1×10⁶ MC38 colon carcinoma subcutaneously on the right flank, and CD5L:CD5L and CD5L:p40 were measured in serum during tumor progression. Serum was obtained and measured for (a) CD5L:p40 heterodimer using sandwich ELISA captured by anti-IL-12p40 antibody and detected with biotinylated anti-CD5L antibody and (b) CD5L:CD5L homodimer using sandwich ELISA captured and detected by anti-CD5L antibodies. Results are shown in FIGS. 19A-B.

### Example 19—CD5L Suppresses Pathogenic T Cell Signatures and Induces Unique Transcriptomes

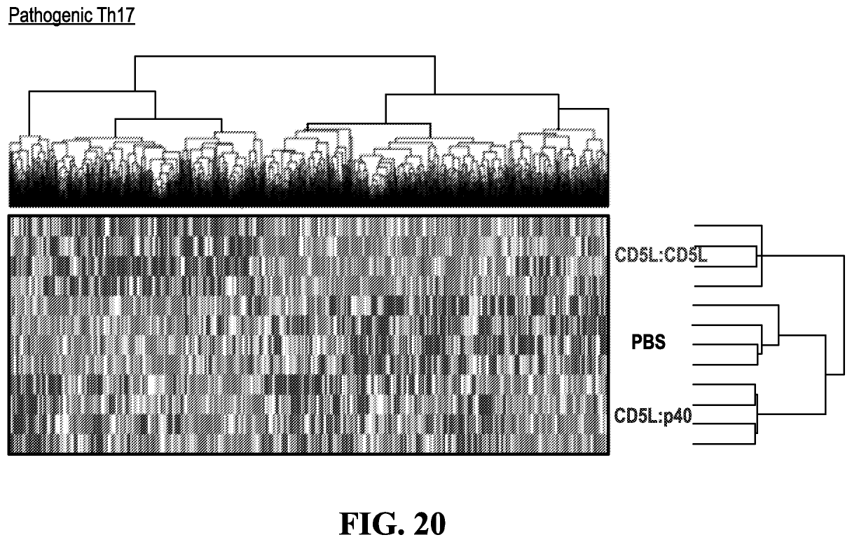

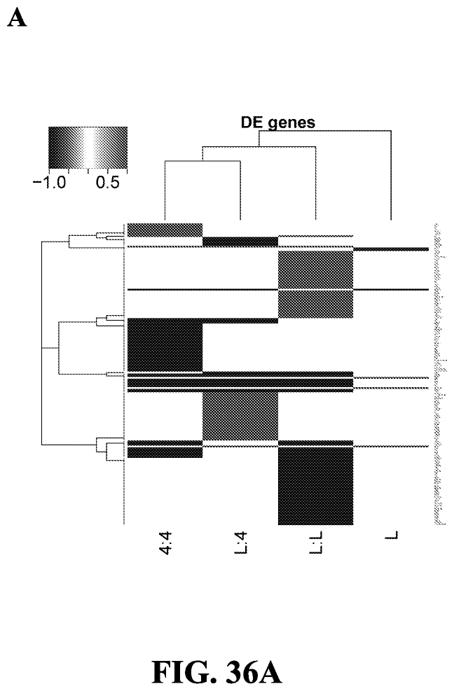

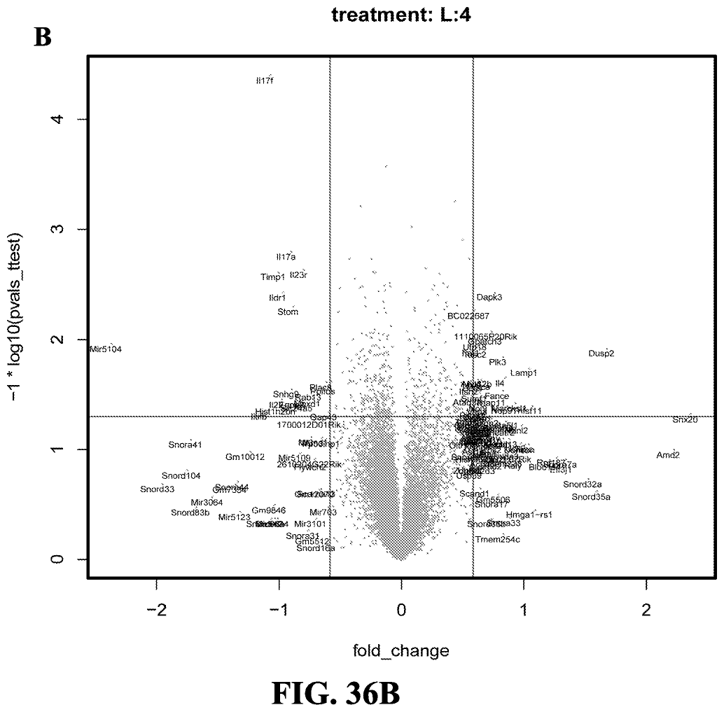

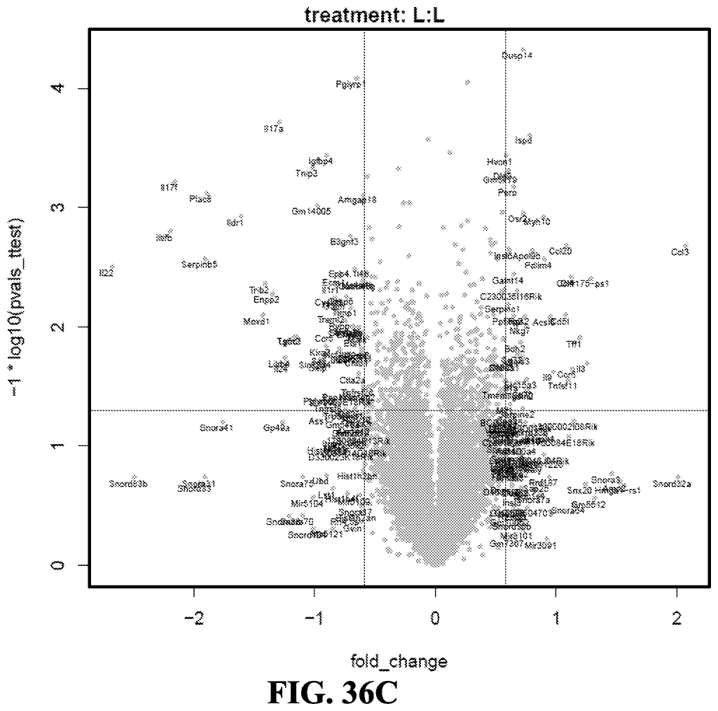

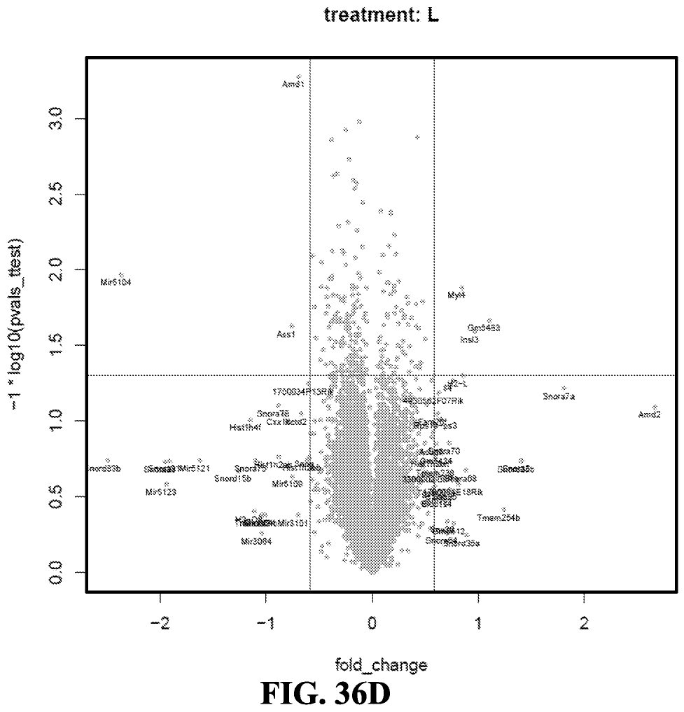

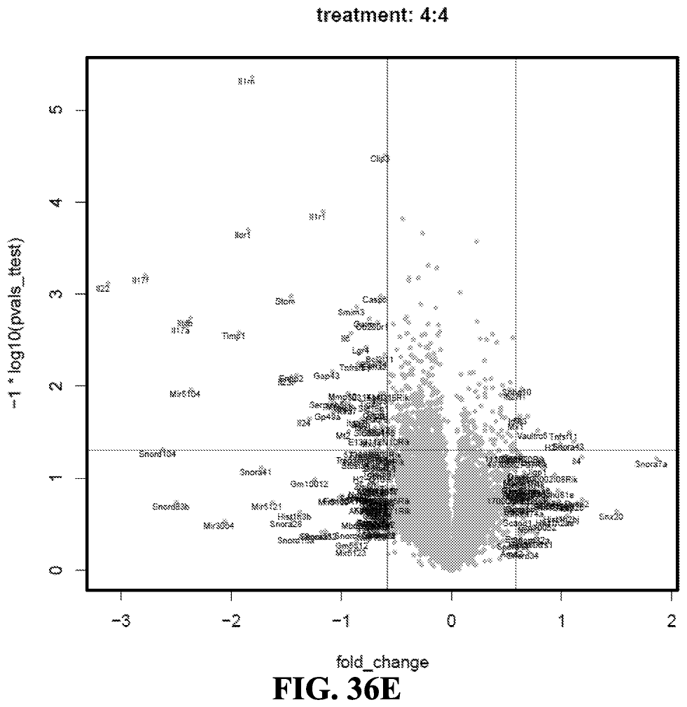

Pathogenic Th17 cells and Th1 cells were differentiated from naïve CD4 T cells (CD44^{low}CD62L⁺CD25-CD4⁺) from wildtype mice with IL-1b, IL-6 and IL-23 (Th17) in the presence of control, CD5L homodimer, or CD5L:p40 heterodimer for 48 hours. RNA were extracted and subjected to RNAseq using NextSeq. A heat map prepared from this data (FIG. 20; four replicates from each condition is shown; spearman coefficient was used for clustering) shows that the presence of CD5L:CD5L results in expression of different signature genes than does the presence of CD5L:p40. The heat map shows differentially expressed genes in the CD5L:CD5L and CD5L:p40 experiments as compared to the control (differentially expressed genes are defined by p<0.5 as compared to control). The expression of DE genes of treatment samples and control were shown in a binary plot (FIG. 36A). A volcano plot shows DE gene expression from CD5L:p40 treatment is illustrated in FIG. 36B. This data demonstrates that both CD5L:CD5L and CD5L:p40 can suppress pathogenic T cell signatures, but that the suppression via CD5L:CD5L and CD5L:p40 is associated with expression of distinct cell signatures.

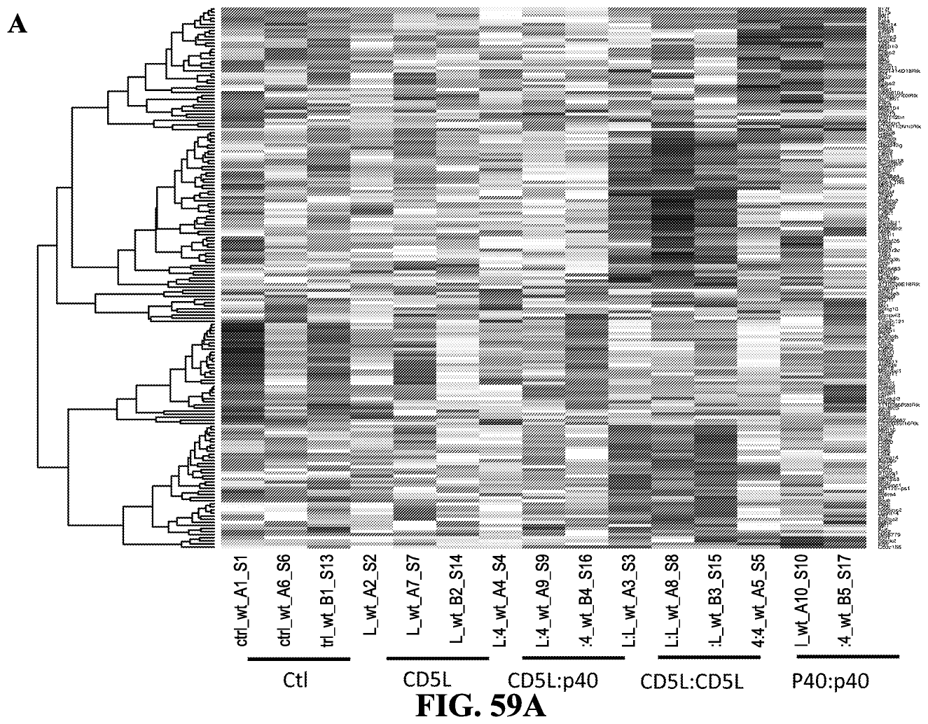

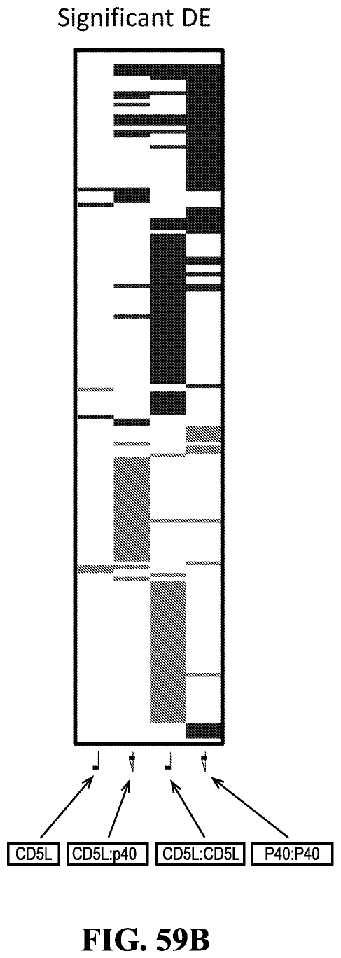

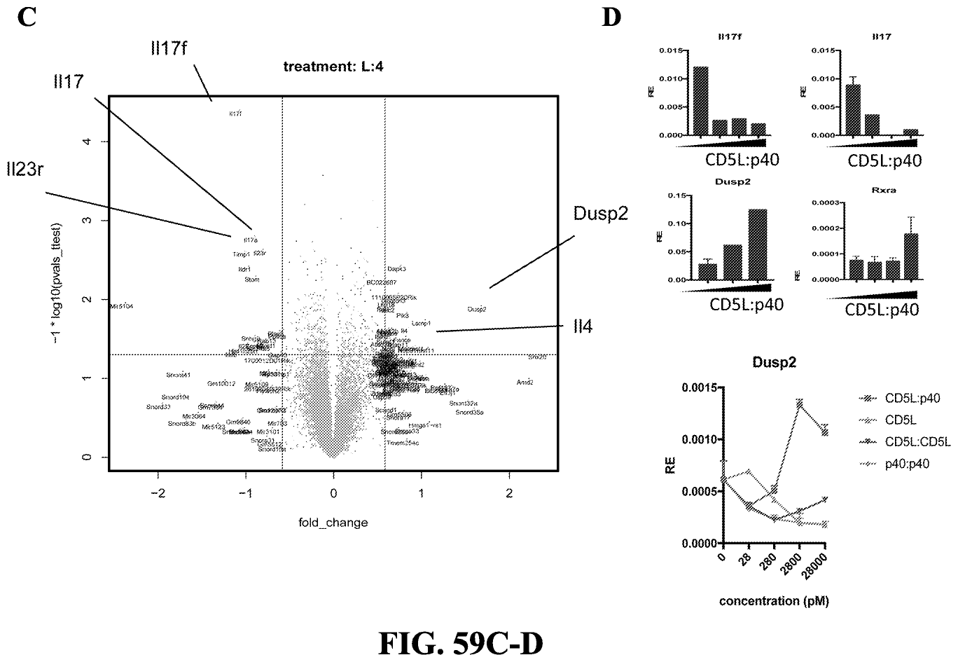

Further, FIG. 59 shows that recombinant CD5L:p40 induces a unique transcriptome in Th17 cells. FIG. 59A,B show heatmaps illustrating differentially expressed genes in Th17 cells treated with control, CD5L, CD5L:p40, CD5L:CD5L and p40:p40. The genes in the heatmap may be downstream targets of each CD5L molecule. The genes in the heatmap from top to bottom are 1117f, 1117a, Ildr1, Il1r1, Lgr4, Ptpnl4, Paqr8, Timp1, Il1rn, Smim3, Gap43, Tigit, Mmp10, Il22, Enpp2, Iltifb, Ido1, I123r, Stom, Bc12111, 5031414D18Rik, Il24, Itga7, Il6, Epha2, Mt2, Upp1, Snord104, 5730577I03Rik, Slcl8b1, Ptprj, Clip3, Mir5104, Ppifos, Rab13, Hist1h2bn, Ass1, Cd200r1, E130112N10Rik, Mxd4, Casp6, Gatm, Tnfrsf8, Gp49a, Gadd45g, Ccr5, Tgm2, Lilrb4, Ecm1, Arhgap18, Serpinb5, Cysltr1, Enpp1, Selp, Slc38a4, Gm14005, Epb4.1l4b, Moxd1, Klra7, Igfbp4, Tnip3, Gstt1, Pglyrp2, Il12rb2, Ctla2a, Plac8, Ly6c1, Sell, Ncf1, Trp53i11, B3gnt3, Kremen2, Matk, Ltb4e1, Ets1, Tnfrsf26, Cd28, Rybp, Ppp1r3c, Thy1, Trib2, Sema3b, Pros1, 1133, Gm5483, Myh11, Cntd1, Ms4a4b, Treml2, 3110009E18Rik, Pglyrp1, Amd1, Slc24a5, Snhg9, Ifi27l1, Irf7, Mx1, Snhg10, 114, Snora43, H2-L, Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Dusp2, Snx20, Lamp1, Faf1, Gpatch3, Dapk3, 1110065P20Rik, Vaultrc5, Myl4, Insl3, Tgoln2, BC022687, C230035I16Rik, Hvcn1, Myh10, Dhrs3, Acsl6, Rgs2, Cc120, Cc13, Dlg2, Ccr6, Cc14, Dusp14, Apol9b, Cd72, Ispd, Cd70, S100al, Lgals3, Slc15a3, Nkg7, Serpinc1, Olfr175-ps1, 119, Pdlim4, Il3, Insl6, Perp, Cd51, Serpine2, Galnt14, Tff1, Ppfibp2, Bdh2, Mlf1, Il1a, Osr2, Gm5779, Ebf1, Spink2, Egfr and Ccdc155. Specific genes upregulated by CD5L:p40 include Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl, Tnfsf11, Nol9, Itsn2, Sumf1, Dusp2, Snx20, Lamp1, Faf1, Gpatch3, Dapk3, 1110065P20Rik and Vaultrc5. FIG. 59C shows a volcano plot of differentially expressed genes. FIG. 59D shows graphs indicating the effects of increased concentrations of CD5L:p40 on expression of the indicated genes.

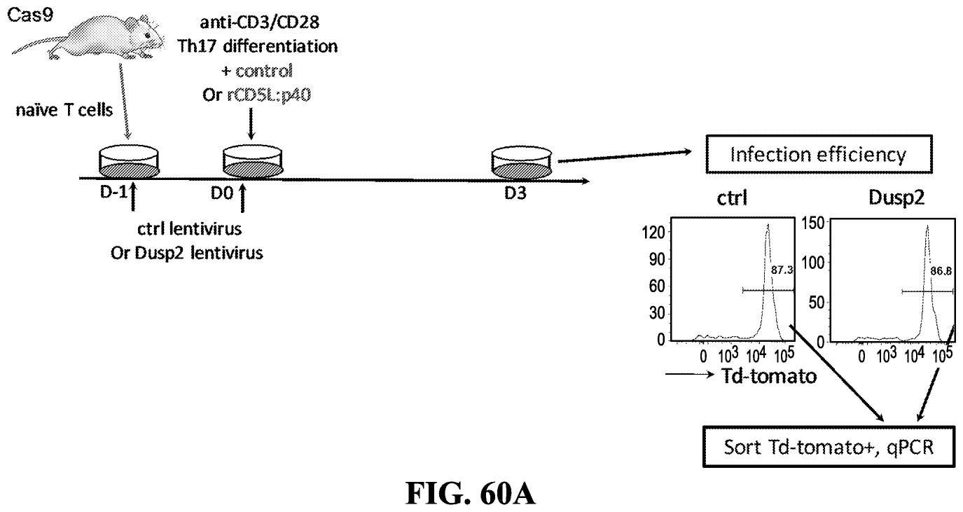

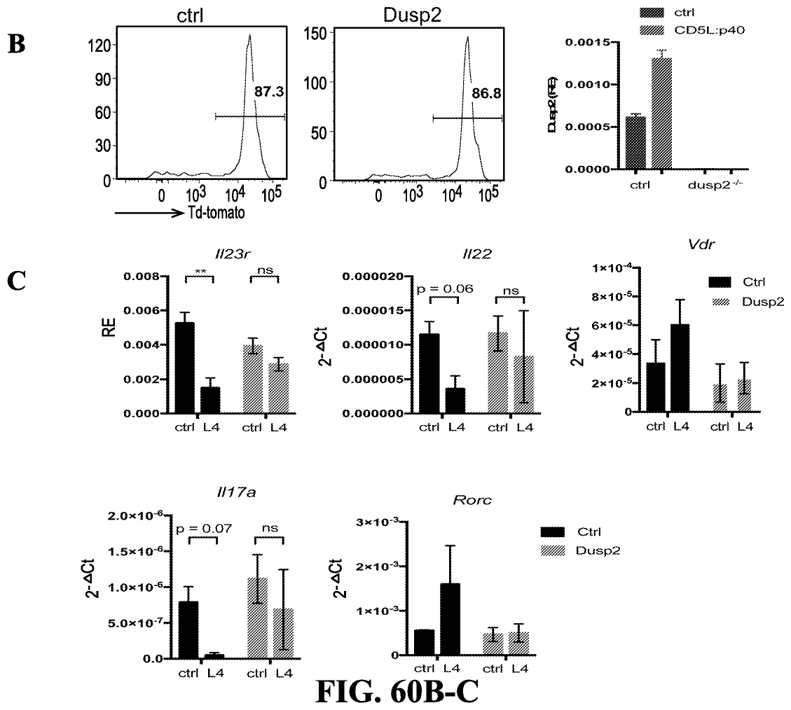

FIG. 60 shows that Dusp2 is a downstream signaling molecule of CD5L:p40 and that deleting Dusp2 rescues the effect of rCD5L:p40. FIG. 60A shows the experimental method used. FIG. 60B shows that Dusp2 was deleted using CRISPR. FIG. 60C shows that repression of I123r, Il17a, and 1122 and induction of Vdr and Rorc by CD5L:p40 is partially inhibited with loss of Dusp2.

### Example 20—In Vivo Effect of CD5L:p40

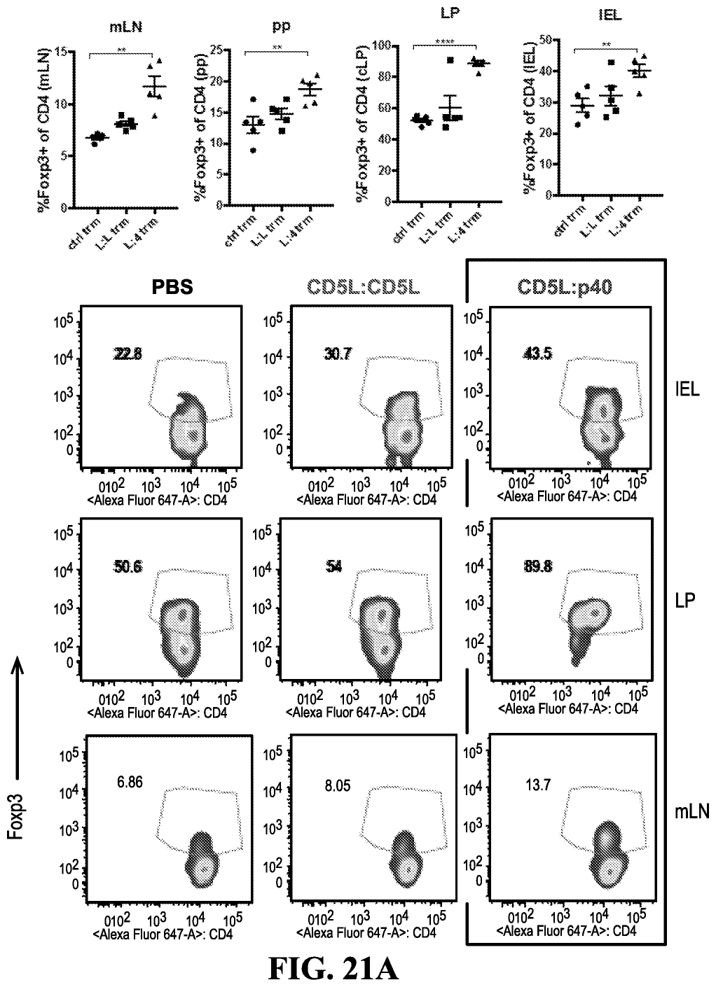

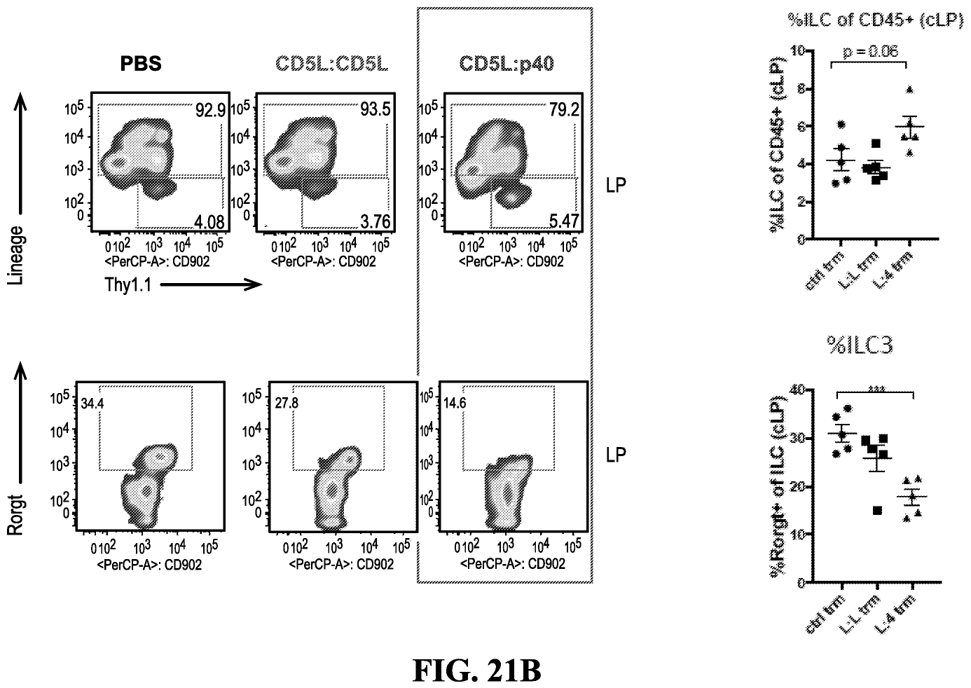

To assess in vivo efficacy of CD5L dimers, wildtype mice were treated with 2% DSS in drinking water for 5 days, followed by normal water for 6 days. Mice were injected with PBS, recombinant CD5L:CD5L, or recombinant CD5L:p40 intraperitoneally on days 4, 6, and 8. Cells from mesenteric lymph nodes (mLN), peyer's patches (pp), lamina propria of colon (LP), and intraepithelial lymphocytes (IEL) were isolated, stained, and analyzed directly with flow cytometry on day 11. The frequency of Foxp3+CD4 T cells in various cell types is shown in FIG. 21A. The frequency of ILC3 as defined by CD45+Lineage-Thy1.2+CD127+Roryt and the percent total of ILC is shown in FIG. 21B. This data demonstrates that CD5L:p40 increased Tregs in vivo in DSS-induced colitis.

### Example 21—Characterizing CD5L Expression on Various Immune and Tumor Cells

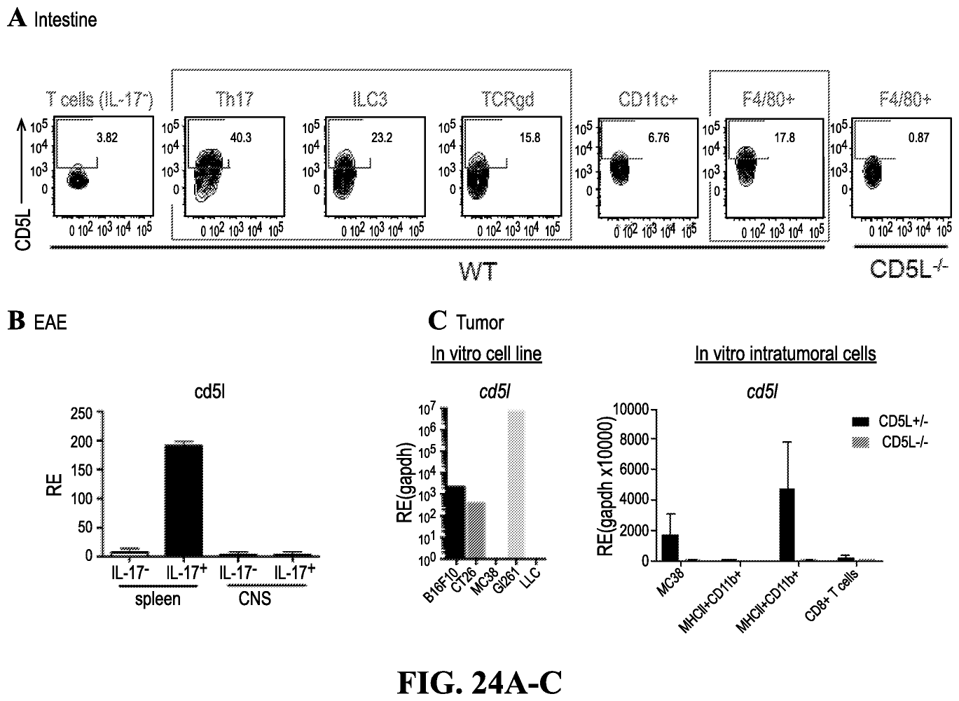

IL-17.GFP mice were induced with DSS colitis. Lamina propria of intestine were isolated on day 9 and stained for intracellular CD5L. Th17, ILC3 and TCRgd were gated on IL-17.GFP+ that were also CD4+(Th17) or TCRgd+(gamma delta T cells) or lineage-CD45+Thy1.1+IL-7R+(ILC3). (FIG. 24A).

IL-17.GFP mice were induced with EAE by MOG immunization. At peak of disease, IL-17.GFP+CD4+ T cells or GFP-CD4+ T cells were isolated and sorted from spleen or CNS and analyzed for mRNA expression of Cd51 by qPCR. (FIG. 24B).

Tumor cells were grown in vitro and mRNA expression of Cd5L in tumor cell lines were assessed by qPCR. (FIG. 24C, left panel).

Model mice were implanted with MC38 tumor on the right flank. Respective populations were sorted from tumor at around size=100 mm\hat{ }2 and analyzed for mRNA expression of Cd5L by qPCR. (FIG. 24C, right panel).

### Example 22—Determining Effects of Soluble CD5L Monomer, CD5L:CD5L Homodimer, and CD5:p40 Heterodimer on Dendritic Cells

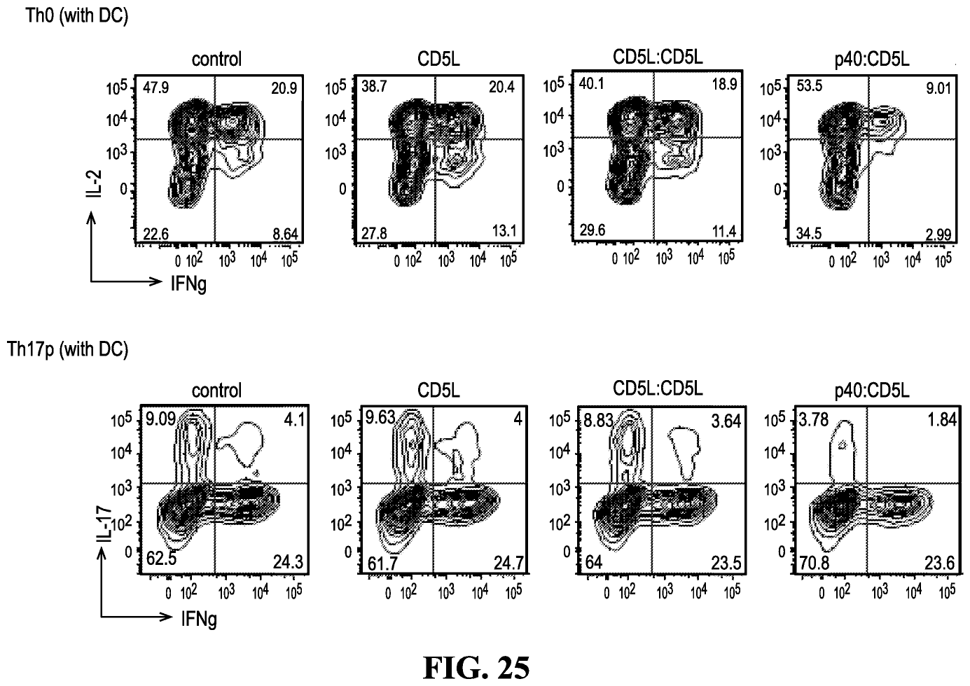

CD11c+ cells were sorted and challenged with LPS for 20 hours; soluble CD5L monomer, CD5L:CD5L homodimer, of CD5L:p40 heterodimer was added to the cells. The cells were washed and naiive CD4+ T cells were added. Anti-CD3 (2.5 μg/mL) and Th0 or TH17p cytokines were added. The results were analyzed by FACS. CD5L:p40 heterodimer demonstrated a regulatory effect on dendritic cells (FIG. 25). Not to be bound by theory, it is believed that CD5L:p40 heterodimer may have a regulatory mechanism that is unique relative to CD5L monomer and CD5L:CD5L homodimer.

### Example 23—Determining Factors Influencing CD5L:CD5L and CD51:p40 Binding

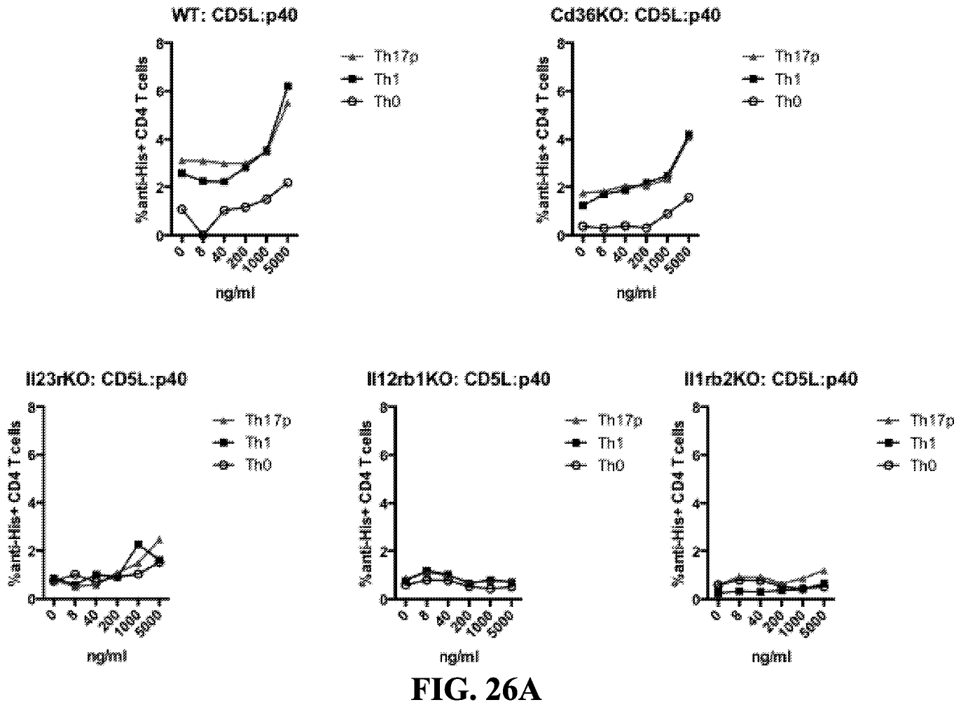

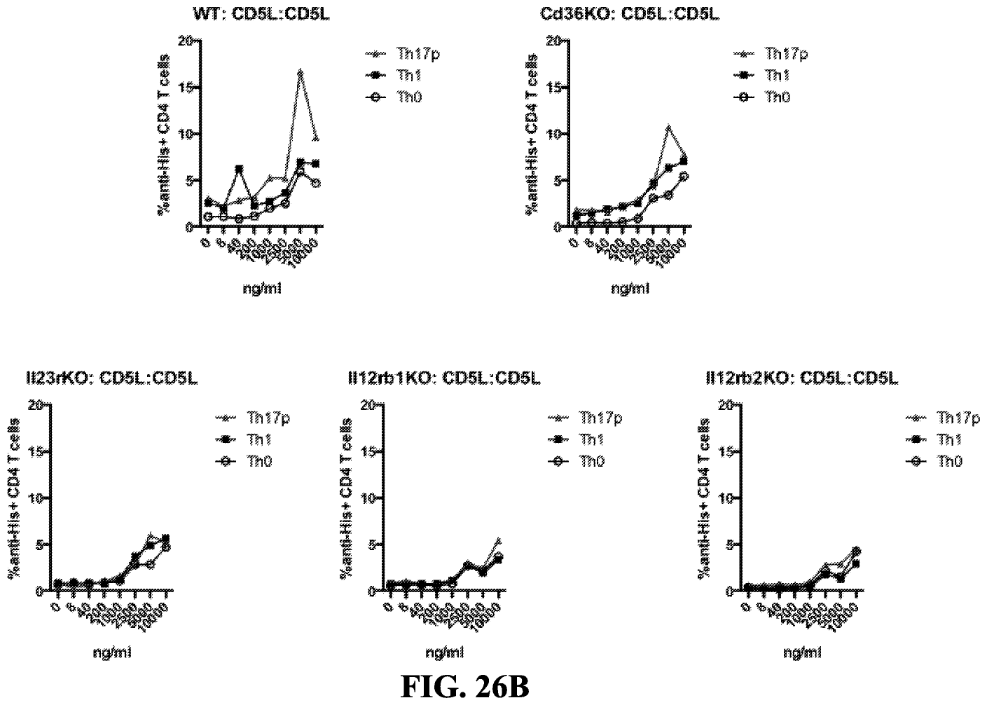

His-tagged CD5L:p40 (FIG. 26A) or his-tagged CD5L:CD5L (FIG. 26B) were used to stain Th17 cells generated from naive cells isolated from the respective wild type and knockout mice. anti-His APC antibody is used as a secondary antibody. Cells were analyzed by flow cytometry.

### Example 24—Assessing Effect of CD5L Deficiency on Antigen Specific CD8 T-Cell Frequency

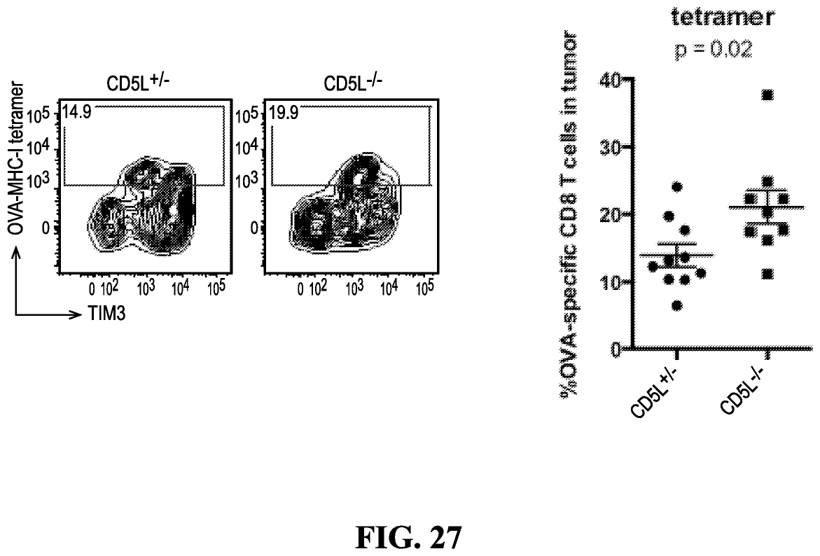

CD5L+/− and CD5L−/− mice were implanted with MC38-OVA tumor cells. Tumor infiltrating lymphocytes (TIL) were isolated on day 14. OVA-specific CD8 T cells in tumor was measured by OVA-MHC class I tetramer staining directly ex vivo. (FIG. 27). It was determined that CD5L deficiency promotes antigen specific CD8 T cell frequencies.

### Example 25—Assessing Effect of CD5L Deficiency on CD4 and CD8 T-Cell Function

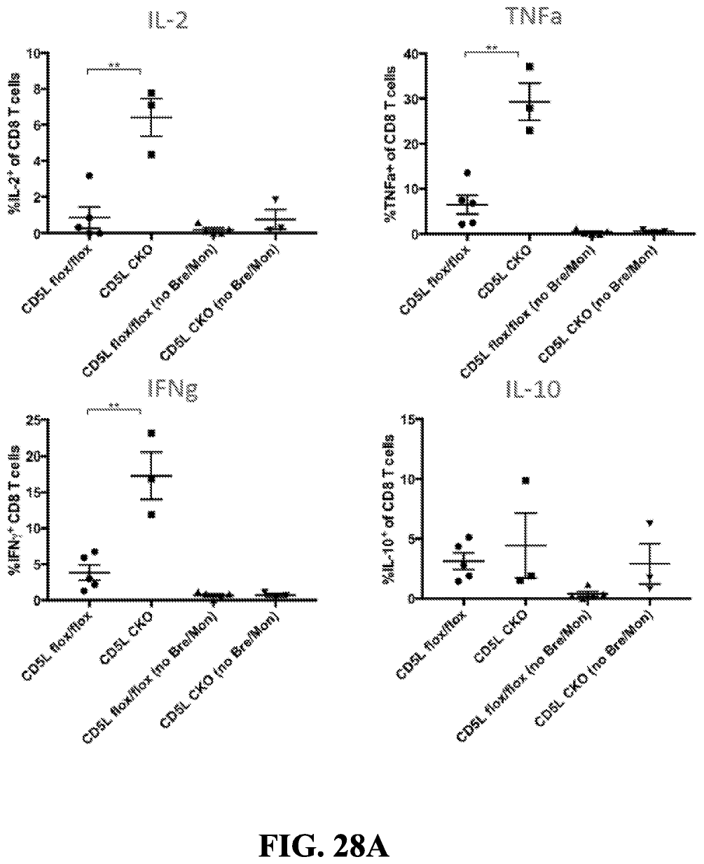

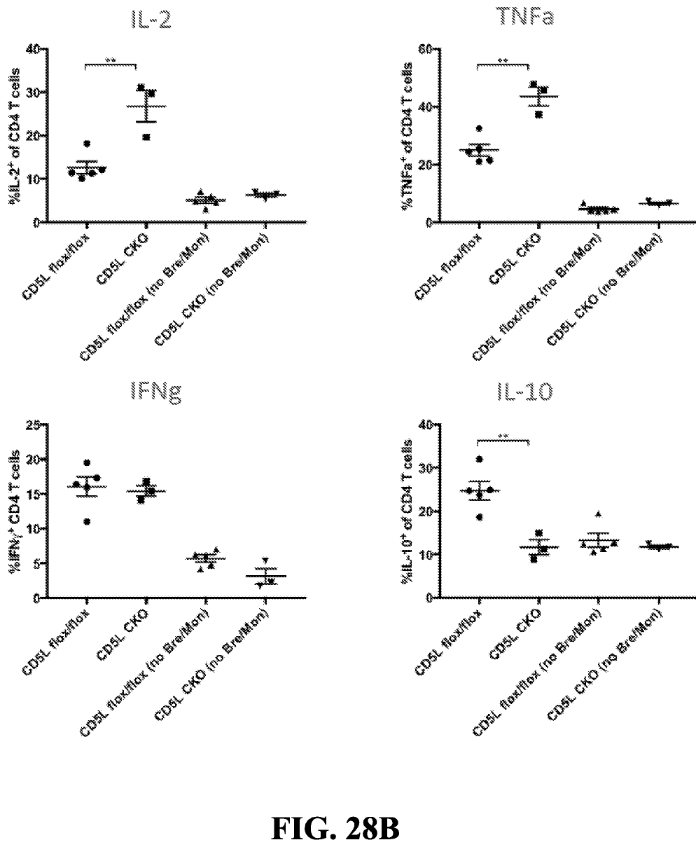

CD5L flox/flox and CD5L flox/flox.Lyz2cre+ mice were implanted with MC38 tumor cells. TIL were isolated on day 14 and are incubated with PMA/ionomycin with or without Golgi plug/stop (labeled no Bre/Mon) as control for 6 hours (FIG. 28A-B) Cells were stained with antibodies against respective surface markers and intracellular cytokine. Cells were analyzed by flow cytometry. CD5L deficiency promoted both CD4 and CD8 T-cell functions.

### Example 26—Assessing Effect of CD5L Deficiency on MDSC and TNFa Production

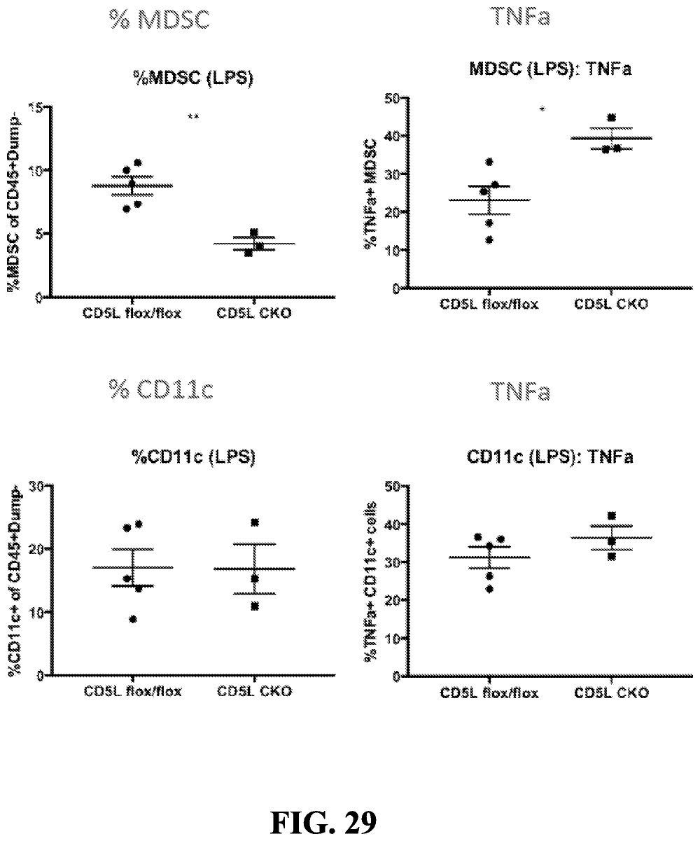

CD5L flox/flox and CD5L flox/flox.Lyz2cre+ mice were implanted with MC38 tumor cells. TIL were isolated on day 14 and are incubated with LPS for 24 hours with golgi stop/plug added in the last 4 hours (FIG. 29). Cells were stained with antibodies against respective surface markers and intracellular cytokine. Cells were analyzed by flow cytometry. CD5L deficiency reduced the number of MDSCs and promoted production of TNFa.

### Example 27—Determining CD5L, p34, p40, and p19 Levels in Bone Marrow Derived Dendritic Cells/Macrophages

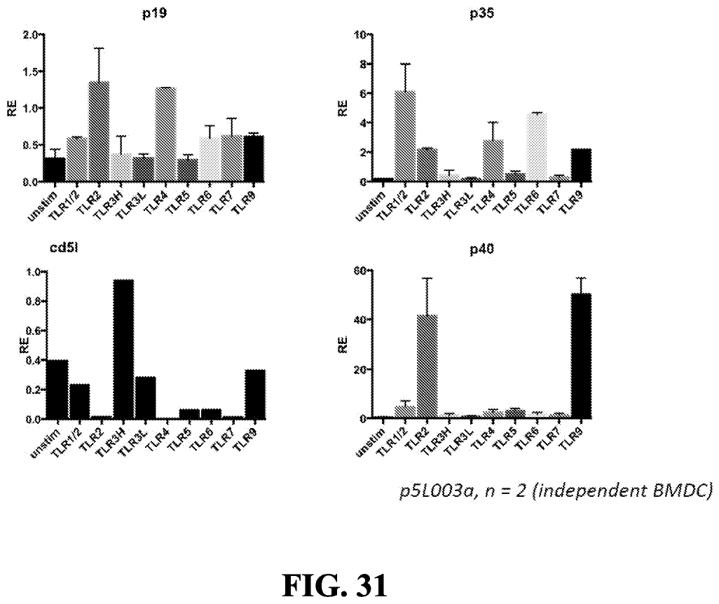

Bone Marrow derived dendritic cells/macrophages were generated by standard protocol with GM-CSF from wild type mice. Cells were stimulated with ligands to the respective TLR. Cells were then lysed; RNA, extracted; and mRNA of Cd51, p35, p40, p19, measured by qPCR. (FIG. 31).

### Example 28—Generation of Anti-CD5L:CD5L Homodimer and Anti-CD5L:p40 Heterodimer Antibodies

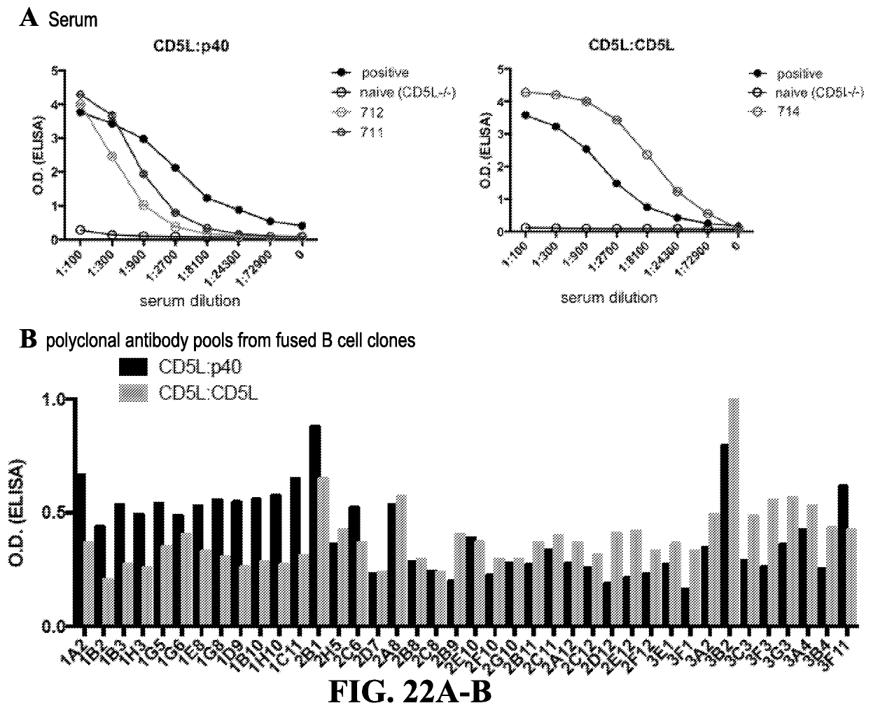

CD5L−/− mice were immunized with either recombinant CD5L:CD5L (labeled “714” in FIG. 22A) or recombinant CD5L:p40 (“711”, “712”) were used as antigen in CFA emulsion, followed by three boosts, for antibody generation. The mutants of recombinant CD5L or CD5L:p40 are also used as immunogen for generation of antibodies with reduced off-target effects. Serum samples were taken from each mouse before spleen infusion and tested for their ability to bind to either CD5L:p40 or CD5L:CD5L, as well as negative binding to IL-12, IL-23, p40:p40 homodimer in a sandwich ELISA assay (FIG. 20A). B cells from the spleen of immunized mice were fused to generate pools of clones that were allowed to expand. Serum from the pools were tested in the same ELISA assay. Polyclonal antibody pools that have preferential specificity to either CD5L:p40 or CD5L:CD5L were observed (FIG. 22B).

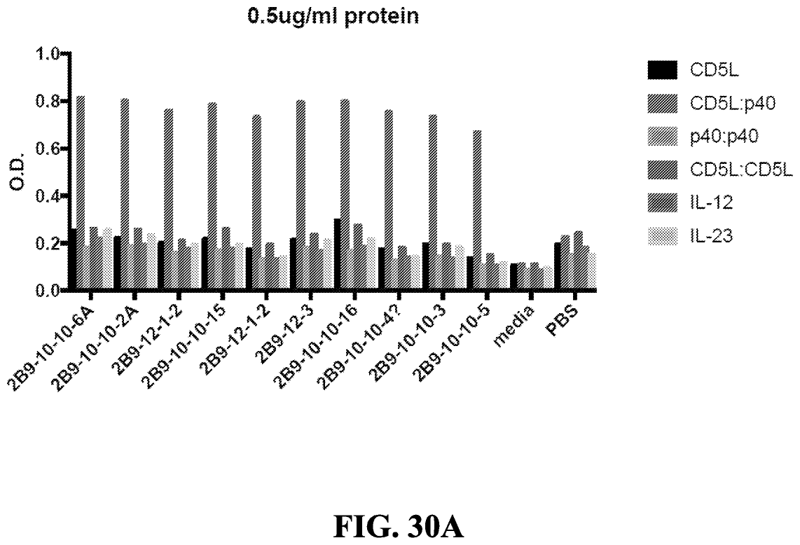

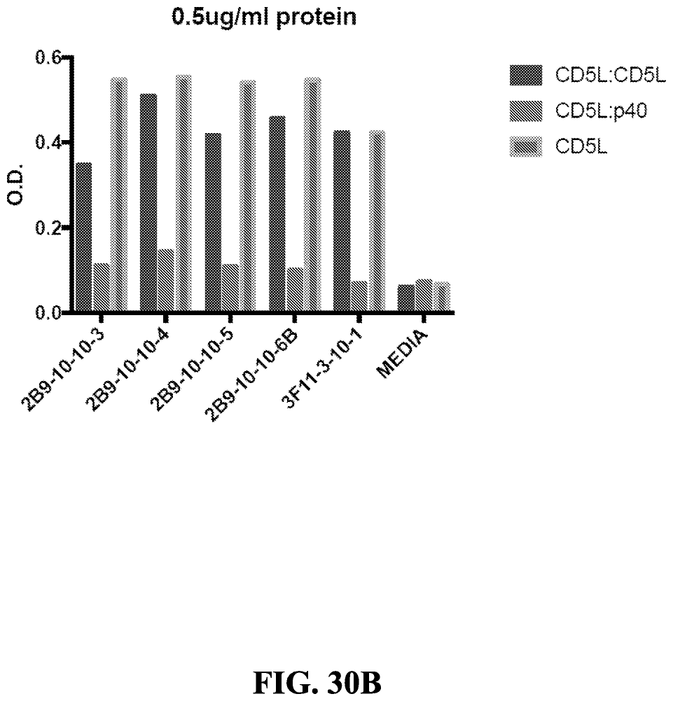

ELISAs were performed with 0.5 micrograms/mL of CD5L, CD5L:p40, p40:p40, CD5L:CD5L, IL-12, and IL-23 to determine suitable candidates that specifically bound to (i) CD5L and/or CD5L:CD5L (Table 1) or CD5L:p40 (Table 2) (FIG. 30 A-B).

Experiments are repeated to identify additional candidate monoclonal antibodies that are specific to one of the CD5L entities and not cross-reactive to the rest of the entities or IL-12, IL-23, or p40:p40 homodimer. Antibodies are also screened for recognition of CD5L:IgM complex vs. soluble CD5L.

These antibodies are screened for antagonistic or agonistic effect on their specific binding partner. The antibodies are sequenced and mapped to understand the binding pocket of each of the antibodies on the CD5L entity. Agonists and antagonists are then further screened against libraries of other molecules, e.g. aptamers, affimers, non-immunoglobulin scaffolds, small molecules, and fragments and derivatives thereof, to identify suitable equivalent candidates.

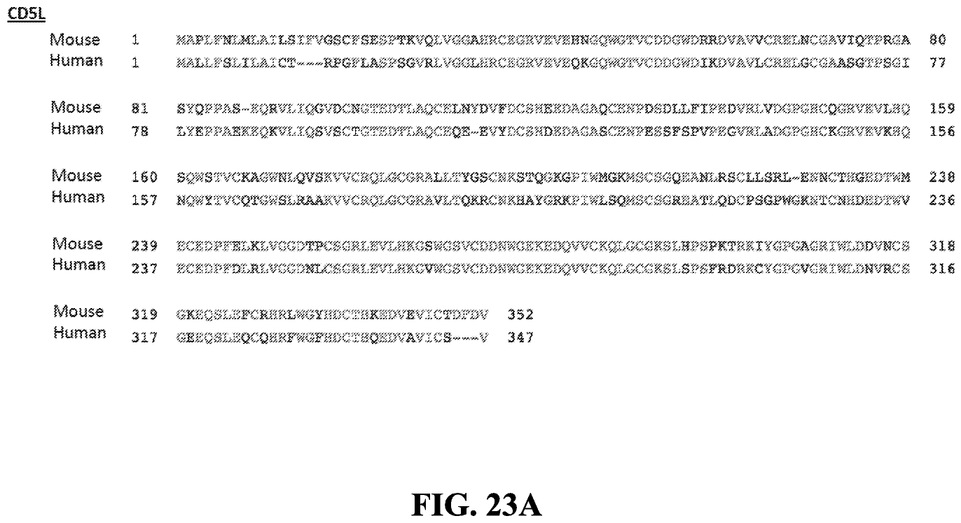

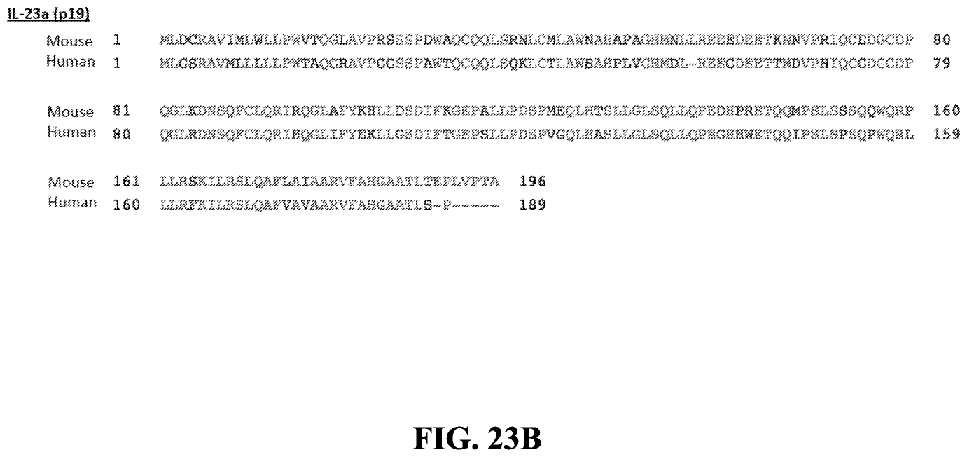

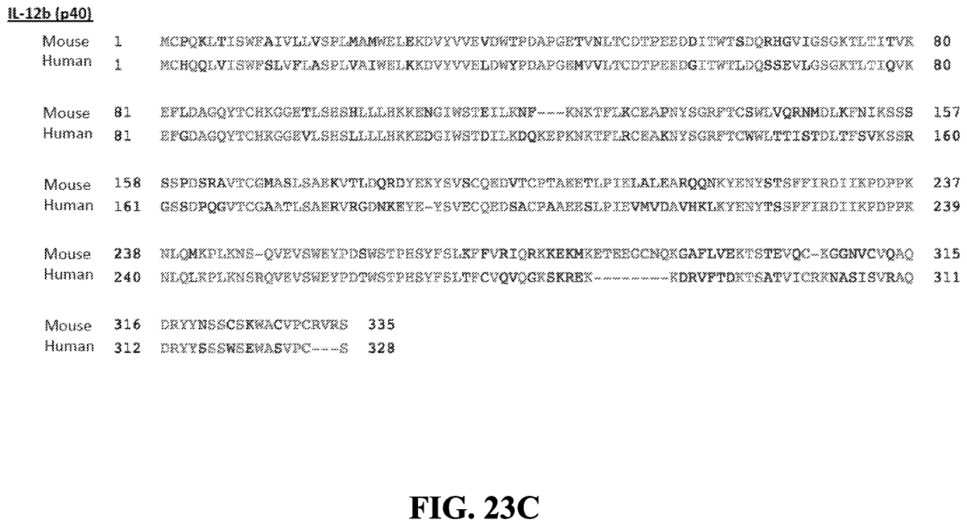

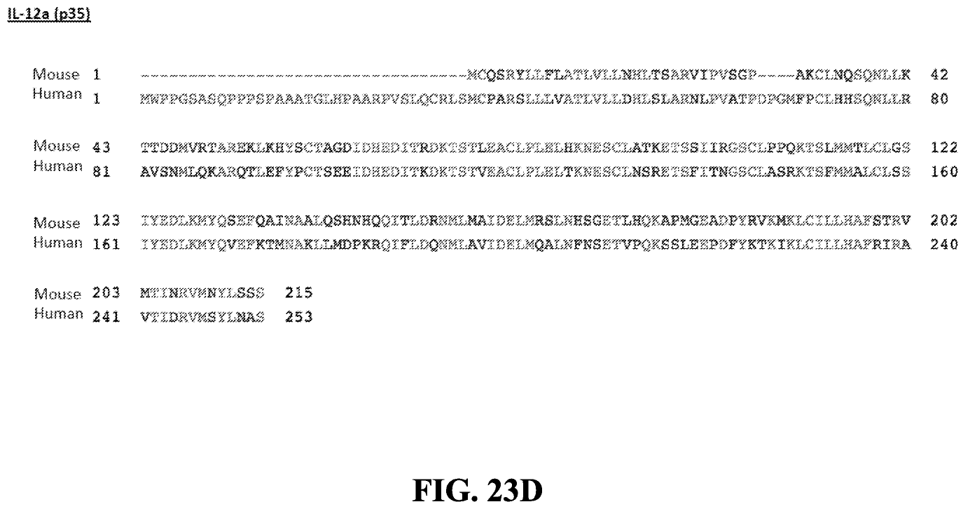

It is contemplated that human antibodies to CD5L:CD5L and CD5L:p40 can be prepared based on the degree of homology between mouse and human CD5L and p40 (FIGS. 23A and C). Also shown are homology between mouse and human protein sequences in p19 and p35 (FIGS. 23B and D), which can form a dimer with p40.

Cross-reactivity of the various antibodies and the equivalent candidates is tested against the respective human CD5L entity. The process of agonist and antagonist antibody identification and screening for equivalent candidates is also repeated for human CD5L entities, i.e. by immunizing a CD5L−/− mice with human recombinant CD5L:CD5L or CD5L:p40 and carrying out the same process steps.

The generated antibodies or equivalent candidates can be altered through humanization or other suitable techniques to be non-immunogenic in humans while retaining specificity to the human CD5L entity.

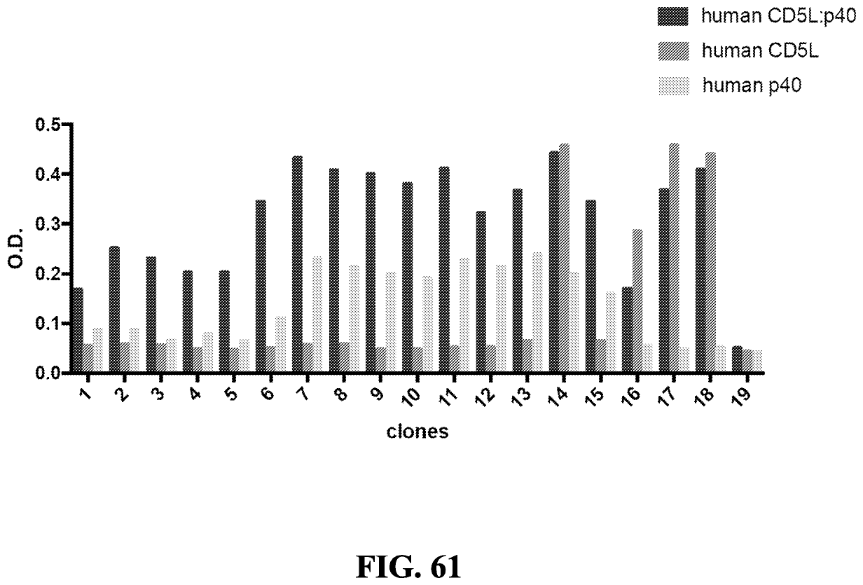

Applicants further generated antibodies specific for human CD5L:p40 (FIG. 61).

### Example 29—Identification of CD5L Associated Cancers

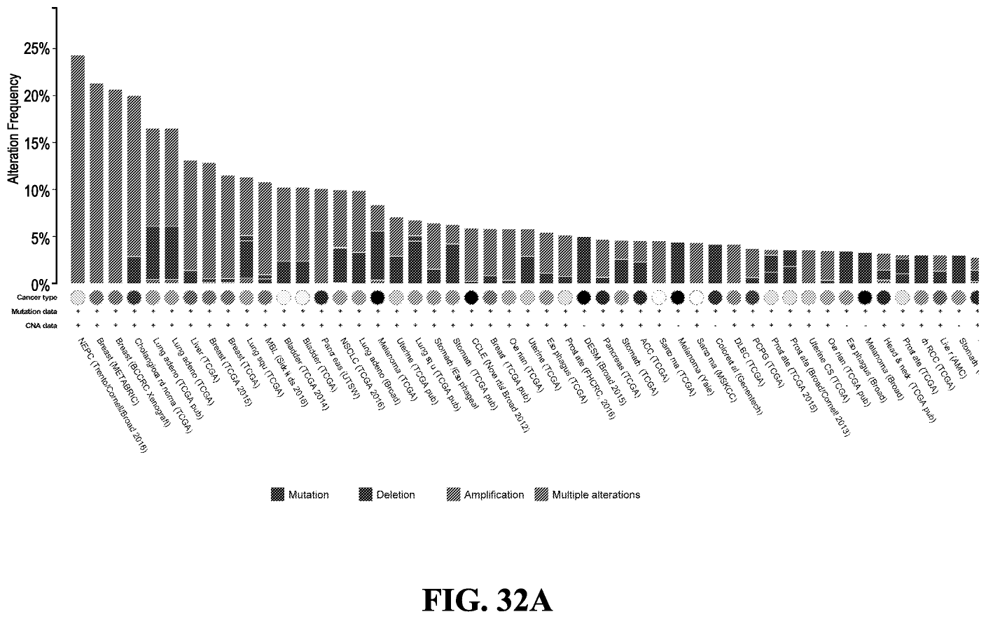

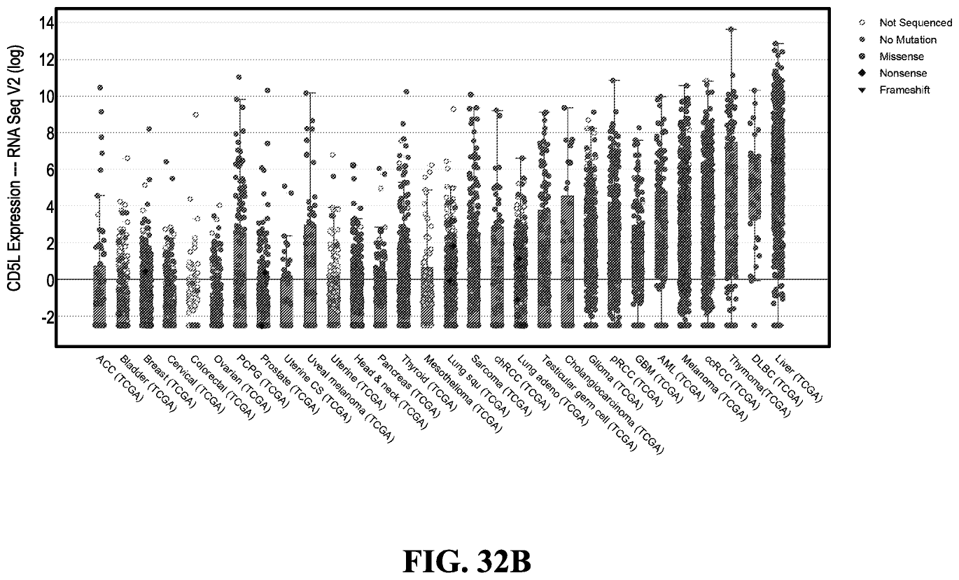

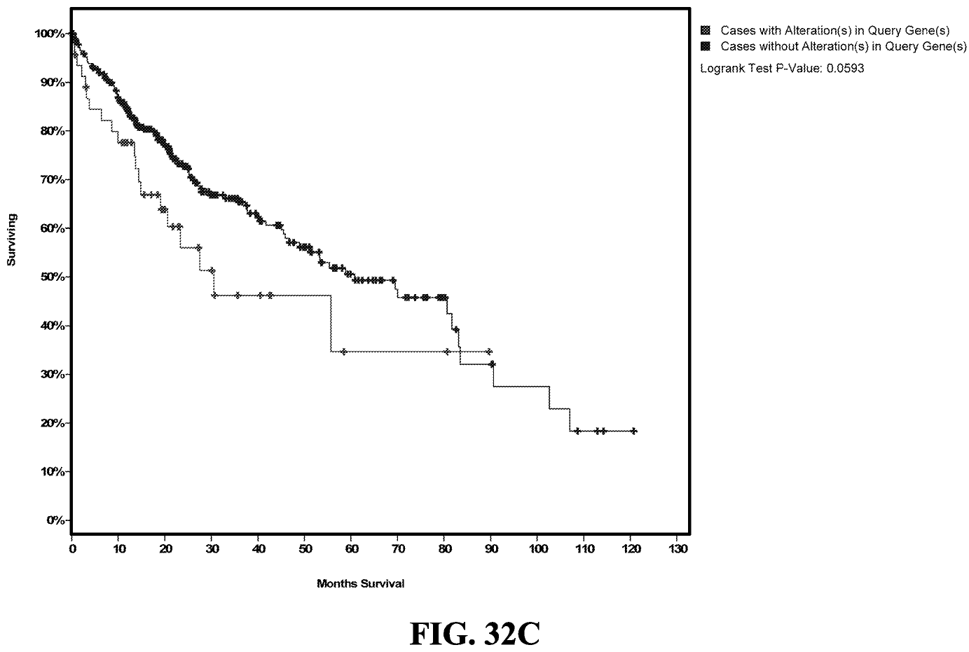

Genetic information was compiled on CD5L alterations in human tumors. Alterations were categorized as mutations, deletions, amplifications, and/or multiple alterations in a variety of cancer cell lines, e.g. neuroendocrine prostate cancer (NEPC), non-small cell lung cancer (NSCLC), stomach and/or esophageal cancer, desmoplastic small-round-cell tumor (DESM), adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), and liver cancer (FIG. 32A). CD5L RNA expression was studied in adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), and liver cancer. Nonsense, missense, and frameshift mutations were categorized (FIG. 32B). Survival rates were compared between liver hepatocellular carcinoma patients having a CD5L alterations and those with wild type. Alterations appear to be linked to overall survival (FIG. 32C).

The identified cancers and others identified through the same or similar methods are screened to determine the CD5L expression profiles and immune correlates of protection and dysfunction.

### Example 30—In Vivo Testing of Anti-CD5L:CD5L Homodimer and Anti-CD5L:p40 Heterodimer Antibody Function

It is contemplated that a suitable animal model for the methods of treatment may be found from a commercially available source or generated through in vivo use of the one or more techniques, e.g. CRISPR-Cas genome editing or perturbation, to edit cells of a suitable organism to present with an autoimmune phenotype. Further contemplated are the use of humanized animal models that suppress the animal host immune system and introduce human or humanized cells to mimic the human immune system. It is appreciated that CRISPR-Cas based methods may also prove useful in the generation of such humanized models.

Varying amounts of one or more agonists discovered through the method of Example 27 are administered to a one or model organisms that has an autoimmune disease or hyperimmune response, e.g. EAE mice. Disease severity, e.g. EAE score, is compared between mice treated with a positive or negative control and those mice treated with agonist. A reduction in severity is observed at certain doses of agonist.

Further replications are run using combination treatments with simultaneous or sequential administration of the agonist and one or more of (1) soluble CD5L monomer, CD5L:CD5L homodimer, and/or CD5L:p40 heterodimer (which may also be used as a positive control), (2) standard treatments for autoimmune diseases (dosed as appropriate based on the model organism), and (3) the antagonists identified above (to determine the agonist's mechanism of action).

Replications are also run using combination treatments with simultaneous or sequential administration of the agonist and a treatment that induces up- and/or down-regulation, and/or knocks out, of one or more genes and/or proteins associated with cancer, autoimmune disease, and/or inflammatory disease, e.g., ALCAM, C-MAF, CCR8, CD83, CAF-expressed complement proteins (e.g., C1R, C3, C4A, CFB, SERPINGI), CYSLTR2, FAS, FOXO1, GATA3, GPR65, HMMR, ILT3, MT1, MT2, PDPN, POU2AF1, PRDM1, PROCR, REE4, SGK1, TNFSF11, NMUR1 and/or ULBP1.

Additional replications of this experiment are performed in organisms in which an autoimmune disease or hyperimmune response is induced after treatment with control, agonist, or a combination treatment to determine if any one of the proposed regiments has a protective effect.

### Example 31—In Vivo Identification of Gene Up- and/or Down-Regulation Associated with Anti-CD5L:CD5L Homodimer and/or Anti-CD5L:p40 Heterodimer Antibodies

Cells or a cell population is exposed to an antibody that is an agonist of one or more of a CD5L monomer, a CD5L:CD5L homodimer, and a CD5L:p40 monomer. CRISPR-Cas9 is used to perturb endogenous genes in the cell(s), and then the cells are assayed for a phenotype indicative of an enhanced or suppressed immune response. A gene or a set of genes that is up and/or down regulated in the cell or cell population with the enhanced or suppressed immune response is identified. Such experiments can be conducted with a variety of mouse models, including EAE models and tumor models (e.g., for melanoma or colon cancer).

Exemplary EAE mice models are discussed above, e.g., at Example 6. Exemplary tumor mice models include C57CL/6 or BalbC mice, as discussed for instance in US 2016/0377631. Other exemplary tumor mice models include C57/BL6 (see, e.g., Nature, 520: 7546 (2015)), Nu/Nu mice (see, e.g., Cell, 160: 1246 (2015)); cre-dependent cas9 trangenic or knock-in mice (see, e.g., PCT/US2015/051815; Cell, 159: 440 (2014)); FVB/NJ mice (see, e.g., Nature, 514: 380 (2014)); and Mus musculus mice (see, e.g., Genes & Development, 28: 1054 (2014)); pmel transgenic mice; and OTI transgenic mice.

### Example 32—Screening for Receptors of CD5L Monomer, a CD5L:CD5L Homodimer, or a CD5L:p40 Heterodimer

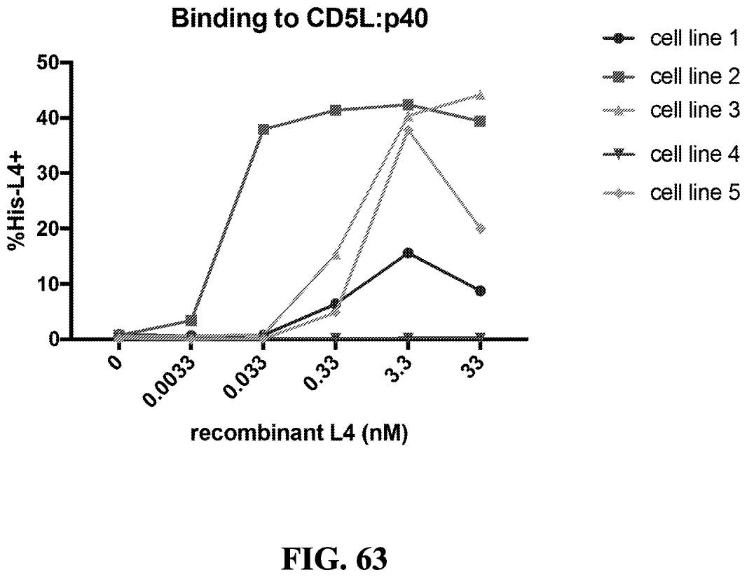

A his-tagged CD5L monomer, CD5L:CD5L homodimer, or CD5L:p40 heterodimer are screened for binding against a library of cell lines. The receptors binding to the respective CD5L entity are identified. The receptors are in some cases common with other cytokines and in others unique to CD5L or the particular CD5L entity. Cell lines were first screened for expression of potential receptor subunits such as Il12rb1 and then used for testing binding to HIS-tagged CD5L:p40. Anti-his APC antibody was used as a secondary antibody and cells were analyzed using flow cytometry. Applicants have identified cell lines differentially stained by HIS-tagged CD5L:p40 (FIG. 63).

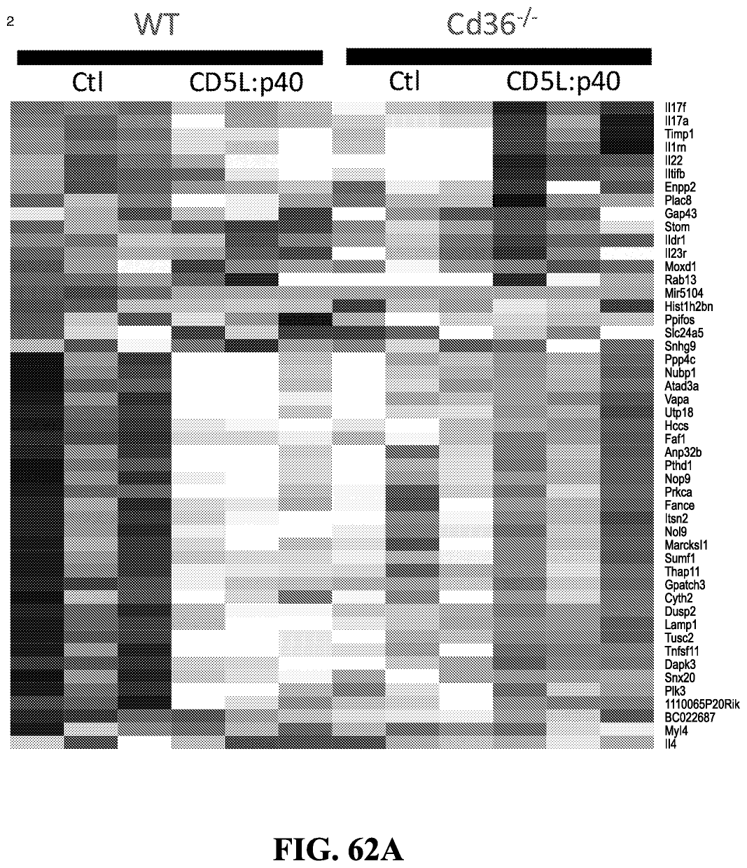

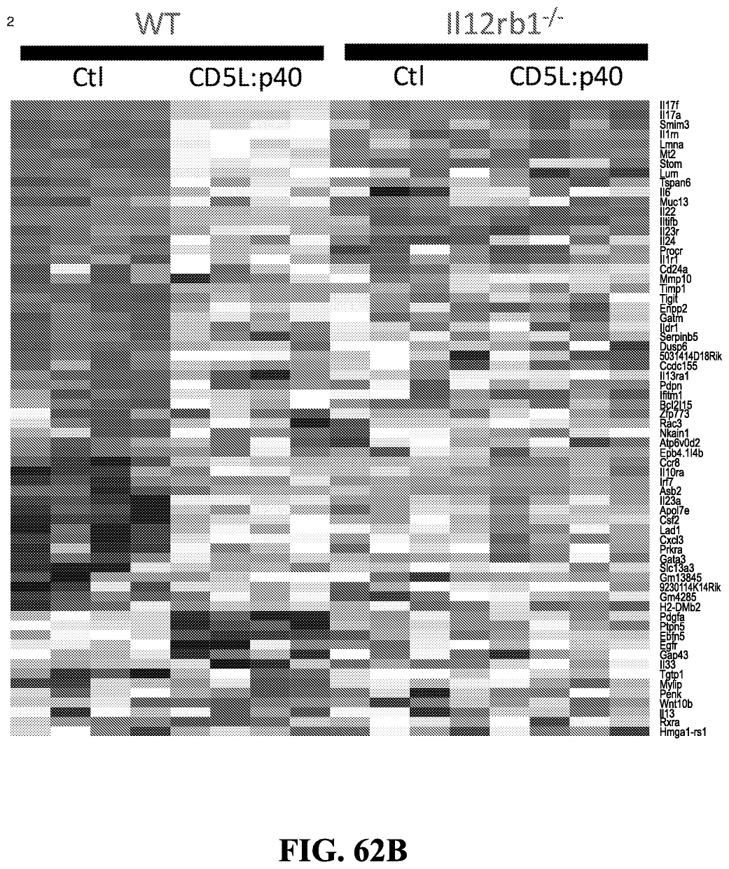

FIG. 62 shows that the effect of CD5L:p40 on Th17p does not depend on CD36, but is dependent on IL-12RB1. There is similar gene expression in the control and treated cells in the Il12rb1−/− cells indicating that the effect of CD5L:p40 on Th17 cells is dependent on the Il12rb1 receptor (FIG. 62B), suggesting that Ilrb1 is the receptor for CD5L:p40.

### Example 33—Characterizing the Effects of CD5L Monomer, a CD5L:CD5L Homodimer, or a CD5L:p40 Heterodimer on Various Immune and Immune-Related or -Mediated Cells

Further iterations and variations of Examples 23-26 and 30 are carried out to elucidate the effect of CD5L entities on T cells, ILCs, DC/myeloid cells, adipocytes and tumor cells. The transcriptome of cells targeted by CD5L is investigated to elucidate the signal pathway regulated by CD5L.

### Example 34—Identification of Specific CD5L:p40 Agonistic and Antagonistic Antibodies

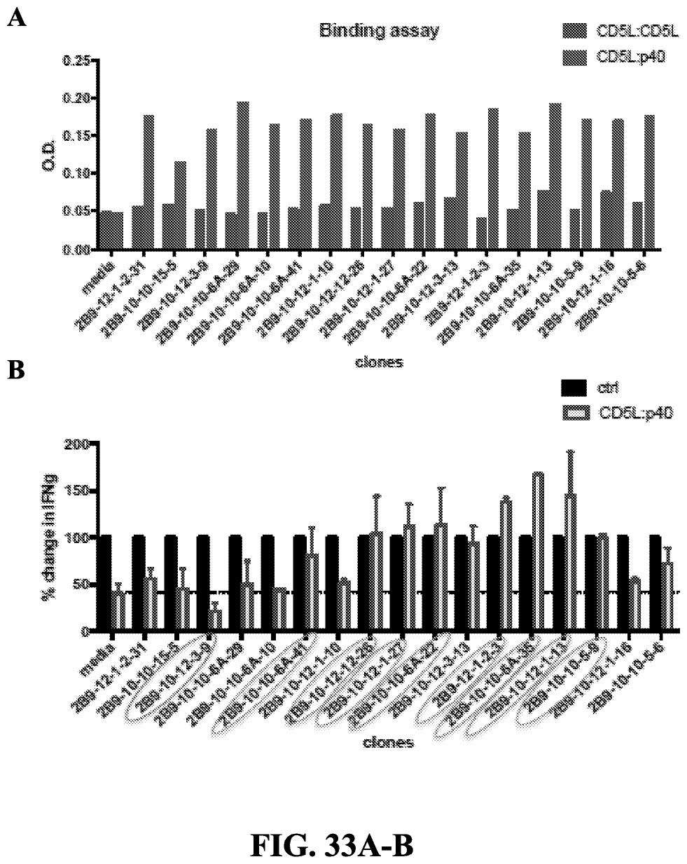

Th1 cells were differentiated as set forth in Example 12, in the presence of recombinant CD5L:p40 heterodimer. Media alone (control) or supernatant from hybridomas previously shown to selectively bind to the recombinant CD5L:p40 heterodimer—but not the CD5L monomer, CD5L:CD5L homodimer, IL-12, IL-23, or p40:p40 homodimer—was added to the culture. The ability of the CD5L:p40 to block IFN-γ production in the presence of the CD5L:p40 antibodies was evaluated. The data in FIG. 33A shows that the clones in FIG. 33B bind specifically to CD5L:p40. The data in FIG. 33B shows that the antibody from clone 2B9-10-12-3-9 is an agonistic antibody. The data in FIG. 33B shows that antibodies from clones 2B9-10-10-6A-41, 2B9-10-12-12-26, 2B910-12-1-27, 2B9-10-10-6A-22, 2B9-12-1-2-3, 2B9-10-10-6A-35, 2B9-10-12-1-13, and 2B9-10-10-5-9 are antagonistic antibodies.

In accordance with the findings in the examples relating to CD5L monomer, CD5L:CD5L homodimer, and CD5L:p40 heterodimer effects on IL-17, TNFa, IL-10, and IL-2, this assay is repeated to use the levels of anyone or more of these cytokines as an endpoint to determine agonistic or antagonistic activity of one or more antibodies specific to the CD5L entity. Further, the immune cells used in the assay are varied, and antibodies are determined related to, e.g., dendritic cells, macrophages, Th1 cells, non-pathogenic Th17 cells, pathogenic Th17 cells, and so forth.

Based on results of transcriptome analysis, genes selectively regulated by CD5L:p40 or CD5L monomer or CD5L:CD5L homodimer are also used as a readout for screening antibodies. For CD5L:p40 agonist screening, cells were treated with CD5L:p40 heterodimer, and a gene or a set of genes up and/or down-regulated in the cell or population of cells are identified. The cells are then treated with a candidate agent, and genes up or down-regulated by the candidate agent are determined. The candidate agent is an agonist if the genes up and/or down-regulated in the cells are the same genes up or down-regulated by CD5L:p40. The selected agonist is analyzed by its effect on whole transcriptome in Th17 cells, Th1 cells, and CD8T cells, as well as in autoimmune disease models. For CD5L monomer agonist screening, cells were treated with CD5L monomer, and a gene or a set of genes up and/or down-regulated in the cell or population of cells are identified. The cells are then treated with a candidate agent, and genes up or down-regulated by the candidate agent are determined. The candidate agent is an agonist if the genes up and/or down-regulated in the cells are the same genes up or down-regulated by CD5L monomer. The selected agonist is analyzed by its effect on whole transcriptome in Th17 cells, Th1 cells, and CD8T cells, as well as in autoimmune disease models. For CD5L:CD5L homodimer agonist screening, cells were treated with CD5L:CD5L homodimer, and a gene or a set of genes up and/or down-regulated in the cell or population of cells are identified. The cells are then treated with a candidate agent, and genes up or down-regulated by the candidate agent are determined. The candidate agent is an agonist if the genes up and/or down-regulated in the cells are the same genes up or down-regulated by CD5L:CD5L homodimer. The selected agonist is analyzed by its effect on whole transcriptome in Th17 cells, Th1 cells, and CD8T cells, as well as in autoimmune disease models. Primarily, Th1 cells are used for screening agonists. Potential cell lines are also screened for this functional assay.

### Example 35—CD5L:p40 Heterodimer has Therapeutic Effects in DSS Colitis and EAE

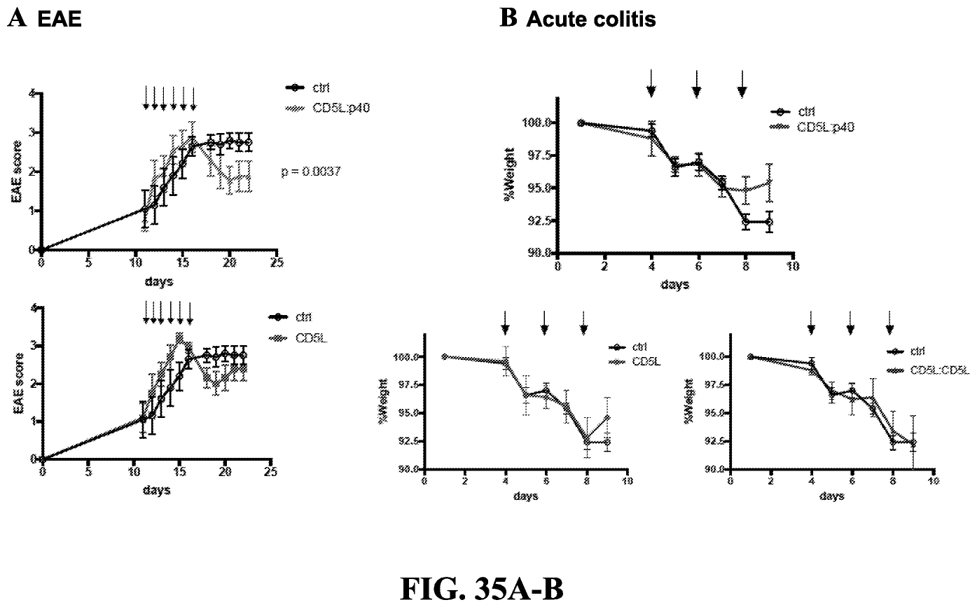

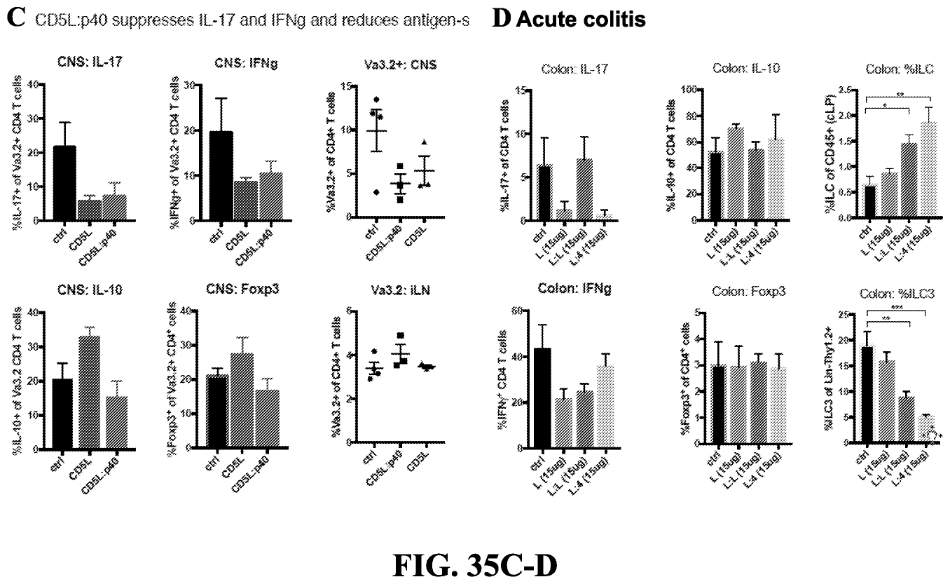

To assess the therapeutic effects of CD5L:p40 heterodimer in DSS colitis and EAE, at day −1, wildtype (WT) mice were injected intravenously with 10,000 naïve 2D2 CD4 T cells for analysis of antigen specific cells. WT mice were immunized with MOG/CFA followed by PT injection to induce EAE. Mice at onset of disease (score=1) were injected intraperitoneally daily with either PBS, recombinant CD5L:p40, or CD5L for six consecutive days and mice were followed for disease progression. As shown in FIG. 35A, CD5L:p40 heterodimer alleviated established neuroinflammation in the EAE model evidenced by a decrease in EAE scores compared to the control. Cell analysis was also conducted on samples of mice from day 23 of the experiment. Va3.2 was used as a surrogate to track 2D2 antigen-specific cells transferred. The results show that both CD5L:p40 and CD5L suppressed IL-17 and IFNg in tissue in EAE model. CD5L:p40 and CD5L also suppressed CNS infiltration of antigen-specific CD4 T cells in EAE (FIG. 35C).

WT mice were also induced with colitis by treating with 2% DSS in drinking water for a consecutive of 7 days followed by normal water. Mice were given either control (PBS), recombinant CD5L:p40, CD5L, or CD5L:CD5L homodimer intraperitoneally on day 4, 6 and 8. As shown in FIG. 35B, CD5L:p40 heterodimer alleviated acute colitis evidenced by an increase in weight compared to the control. Cell analysis was also conducted on samples of mice from day 9 of the experiment. The results show that both CD5L:p40 and CD5L suppressed IL-17 and IFNg in tissue in colitis model. CD5L:p40 and CD5L also increased the frequency of bulk ILC and reduced the frequency of ILC3 in colitis (FIG. 35D).

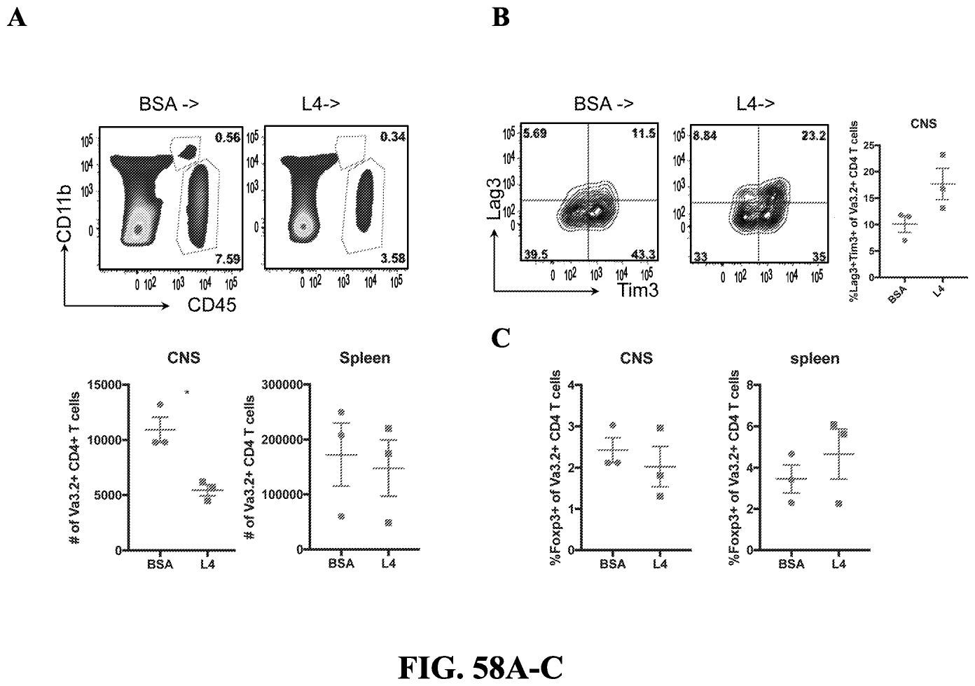

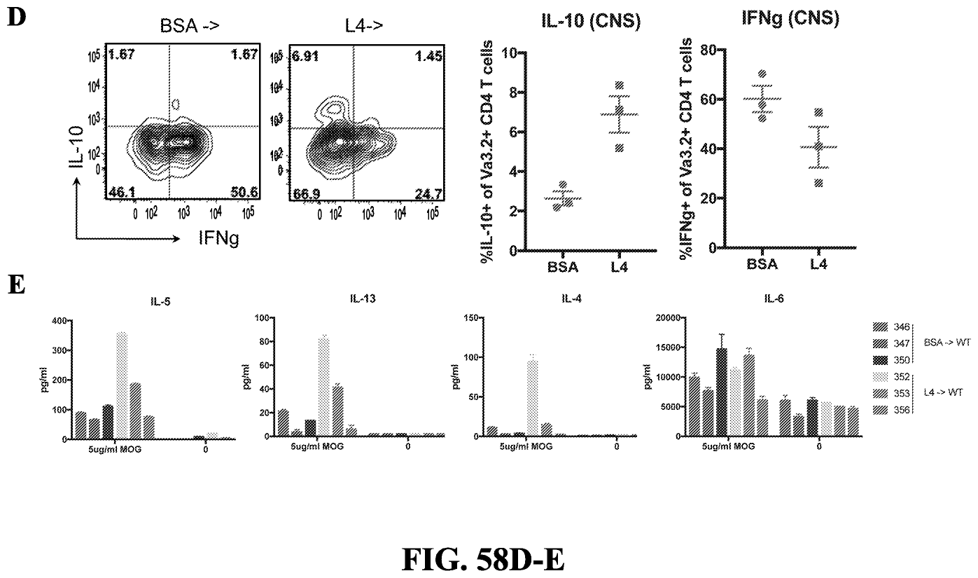

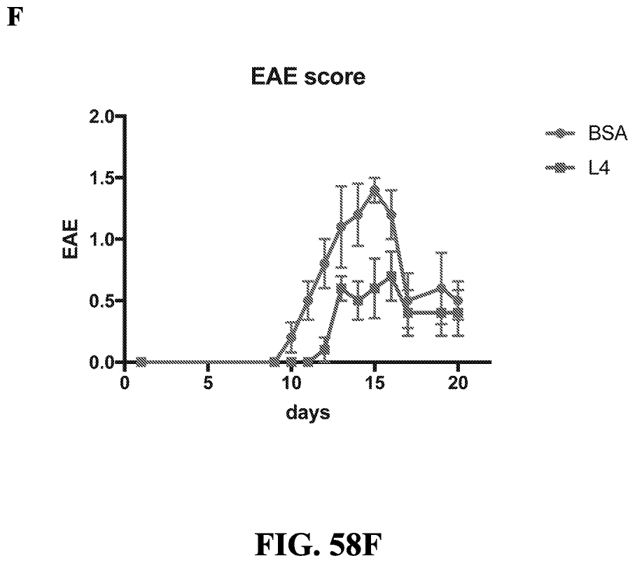

Further, FIG. 58 shows that Th17p cells treated with CD5L:p40 showed reduced pathogenicity in vivo in transfer EAE model. Th17p cells were differentiated (IL-1b+IL-6+IL-23) in the presence of either BSA or CD5L:p40 from naïve T cells isolated from 2D2 transgenic mice. Th17 cells were then transferred into wildtype host and mice were followed for EAE clinical scores and CNS infiltrating cells and splenocytes were analyzed for cell surface markers and cytokine production. FIG. 58A shows that the number of CNS-infiltrating antigen-specific CD4 T cells is reduced in mice transferred with L4 treated Th17 cells. FIG. 58B shows that coinhibitory receptor expression on antigen-specific CD4 T cells is enhanced in mice with L4 treated Th17 cell transfer. FIG. 58C shows that the frequency of induced antigen-specific Treg cells is unchanged. FIG. 58D,E show that antigen-specific T cells make more IL-10 and less IFNg (D, flow cytometry) and make more type2 cytokines in response to antigen (E, legendplex analysis of supernatant from CNS lymphocytes restimulated with MOG peptide or control for 3 days). FIG. 58F shows a decrease in EAE score with CD5L:p40 treatment of Th17 cells.

### Example 36—Elucidating Biochemical Features of p40 in CD5L:p40 Heterodimer

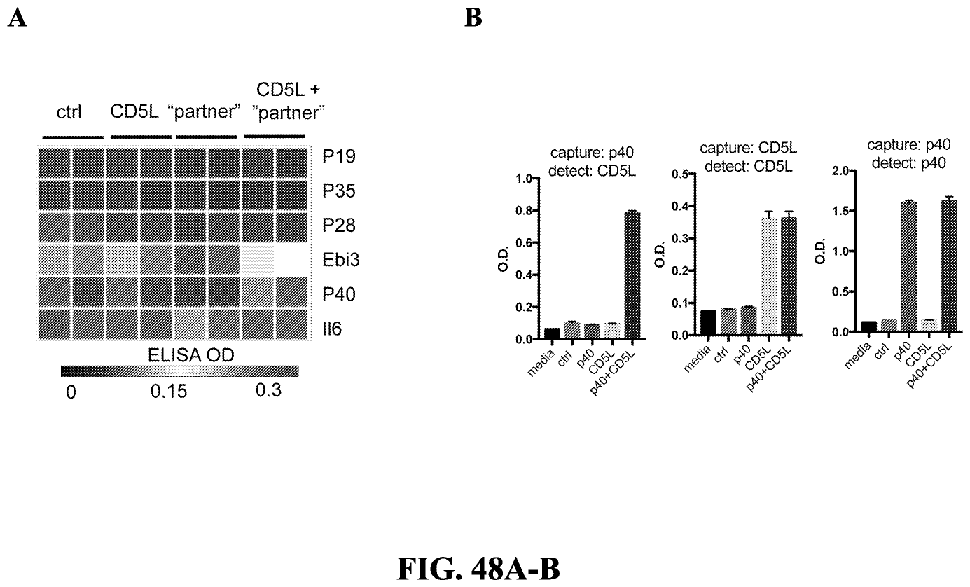

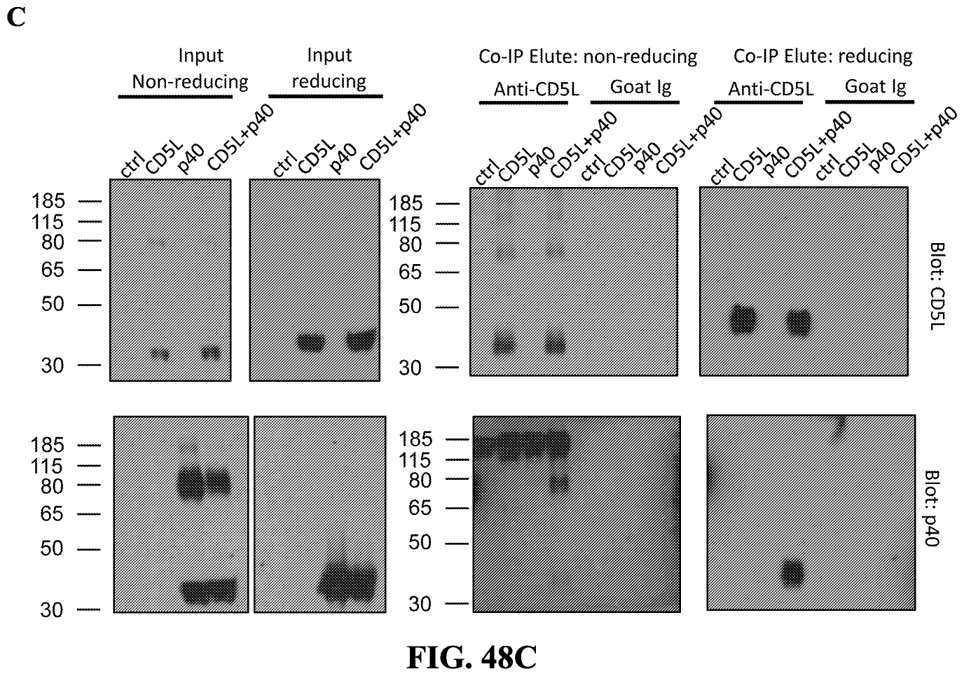

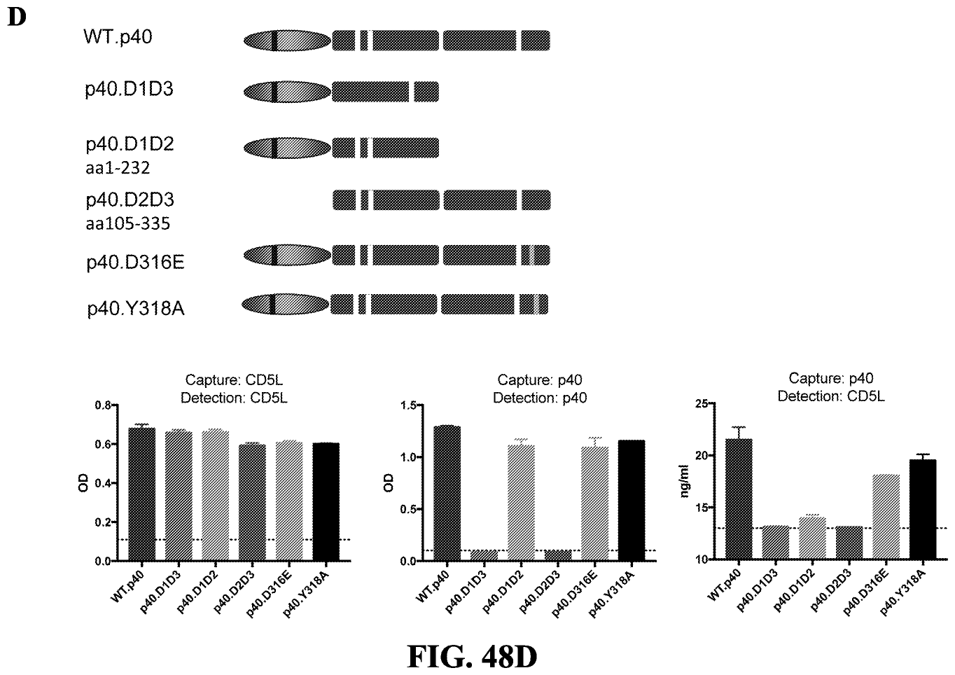

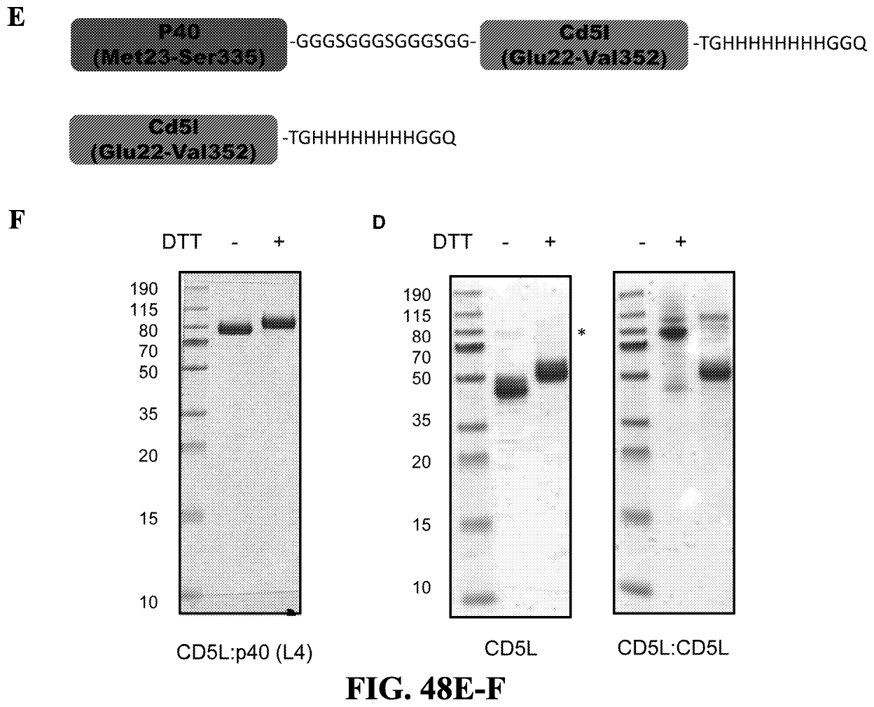

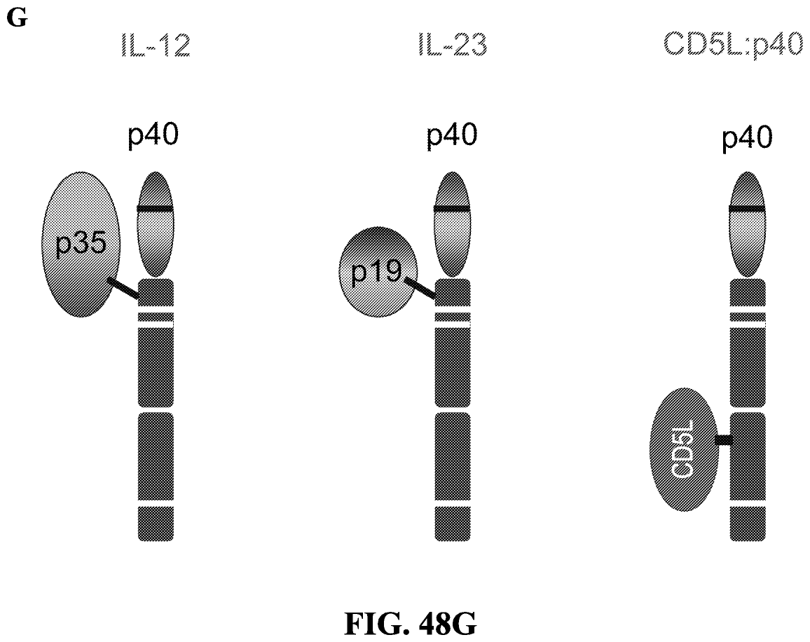

To determine CD5L interacting proteins binding of CD5L to various partners was analyzed by ELISA and co-immunoprecipitation. FIGS. 48A and B shows that CD5L can form a heterodimer with p40. Using a sandwich ELISA, capture of p40 from the supernatant with p40 antibodies followed by detection of CD5L with CD5L antibodies shows that p40 and CD5L can form a heterodimer. Additionally, FIG. 48C shows that immunoprecipitation of CD5L using anti-CD5L antibodies can co-immunoprecipitate p40.

### Example 37—Mutagenesis of CD5L and p40

Mutagenesis is conducted to alter specific sites on p40 critical to binding to p35 and/or p19. The effects on CD5L binding to p40 to form the heterodimer and the biological activity of any resulting heterodimer are observed.

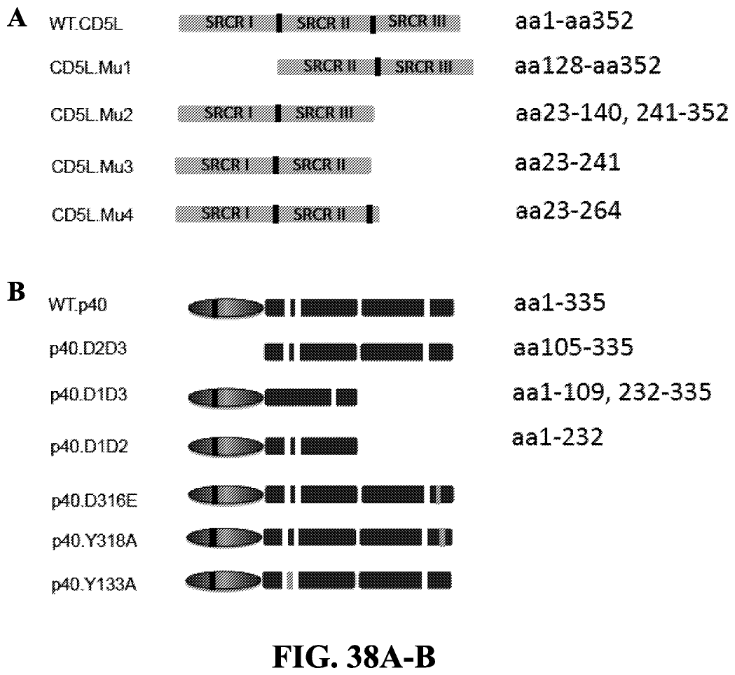

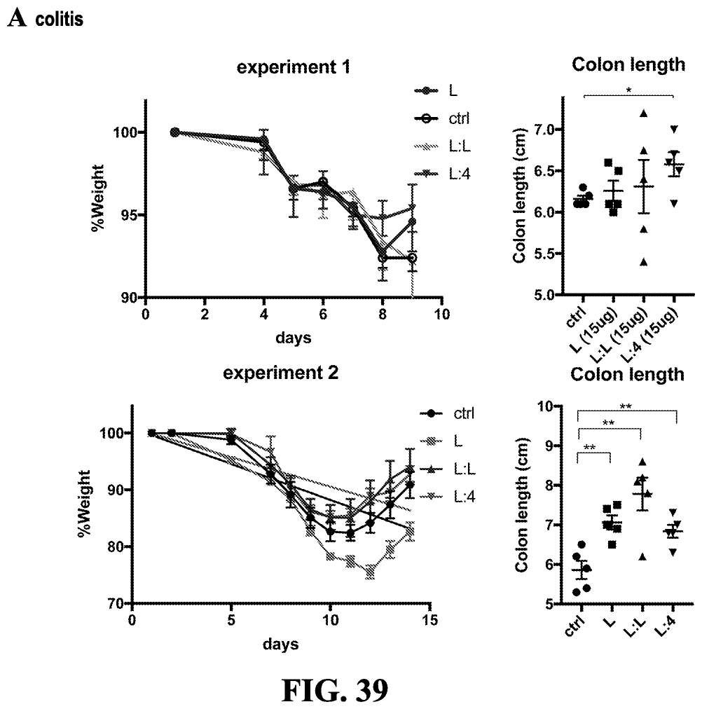

To determine the binding site on CD5L and p40, i.e. the region and/or amino acid residues essential for CD5L and p40 interactions, various CD5L and p40 mutant construct were generated. As shown in FIG. 38, three truncated CD5L were generated with each of SRCRI domain eliminated. Specifically, as shown in FIG. 38A, the SRCR I domain was truncated to generate CD5L.Mu1 mutant. The SRCRII domain was truncated and the SRCRI domain was directly joined to the SRCRIII domain to generate CD5L.Mu2 mutant. The SRCRIII domain was truncated to generate CD5L.Mu3 mutant. As shown in FIGS. 38B and 48D, D1 domain of p40 was truncated to generate p40.D2D3 mutant. D2 domain was truncated and the D1 domain was directly joined to the D3 domain to generate D1D3 mutant. D3 domain was truncated to generate p40.D1D2 mutant. Aspartate at position 316 was mutated to Glutamate to generate p40. D316E mutant. Tyrosine at position 318 was mutated to Alanine to generate p40.Y318A mutant. Binding of each CD5L mutant to p40 and each p40 mutant to CD5L was carried out to determine which domain and/or residue is critical for binding of CD5L to p40.

Determination of the region and/or amino acid residues essential for CD5L and p40 interactions helps in the strategic design of targeting CD5L and CD5L:p40 as therapeutics in the context of the following known interactions for the purpose of limiting off-target effects and/or enhancing desirable effects:

a. critical domain/residues on p40 for p40 and p19 interaction: D3, D316/Y318, Y133 (Lupardus et al., J. Mol. Biol. (2008) 382(4):931-41)

b. critical domain/residues on p40 for p40 and p35 interaction: D3, D316/Y318, Y133 (Yoon et al., EMBO J. (2000) 19(14):3530-41)

c. critical domain/residues on CD5L for CD5L and IgM interaction: 3^{rd} SRCR domain after K264 (Yamazaki et al., Sci. Rep. (2016) 6:38762; Maehara et al., Cell Rep. (2014) 9(1):61-74)

d. critical domain on p40 for p40 and receptor to IL-23 interaction: D1 and D2 domains (Schroder et al., J Biol. Chem. (2015) 290(1):359-70).

The understanding of how CD5L interacts with p40 allows prediction of its biological functions and interactions with its receptor(s) in relevance to known biology about IL12 (p40-p35) and IL-23 (p40-pl9) as well as recent evidence on complement activation by surface bound CD5L enhanced through IgM binding (Maehara et al., Cell Reports (2014) 9:61-74). Results from this experiment allows for the generation of unique recombinant CD5L:p40 proteins that can optimize generation of both agonistic and antagonistic antibodies against CD5L and/or CD5L:p40 for the purpose of limiting off-target effects and/or enhancing desirable effects. The results show that p40.D1D2 fails to bind to CD5L suggesting that the Fibronectin domain 2 (D3) is required for CD5L binding.

FIG. 48D show generation of mutant p40 constructs and analysis of their bindings to CD5L using the same system as in FIG. 48B. Results show that p40.D1D2 fail to bind to CD5L suggest that the fibronectin domain 2 (D3) is required for binding.

FIGS. 48 E and F show that recombinant CD5L:p40 was generated. A CD5L:p40 fusion protein was generated using a Gly-Ser linker (FIG. 48E). The indicated residues from p40 and CD5L are indicated. Purified proteins under reducing and non-reducing conditions are shown (FIG. 48F). FIG. 48G shows the differential binding sites on p40 for p35, p19 and CD5L.

| Protein sequence of mouse CD5L:p40 construct | | | | |
|---|---|---|---|---|
| MCPQKLTISWFAIVLLVSPLMAMWELEKDVYVVEVDWTPDAPGETVNLT | | | | |
| CDTPEEDDITWTSDQRHGVIGSGKTLTITVKEFLDAGQYTCHKGGETLSHSHLLLHK | | | | |
| KENGIWSTEILKNFKNKTFLKCEAPNYSGRFTCSWLVQRNMDLKFNIKSSSSSPDSRA | | | | |
| VTCGMASLSAEKVTLDQRDYEKYSVSCQEDVTCPTAEETLPIELALEARQQNKYEN | | | | |
| YSTSFFIRDIIKPDPPKNLQMKPLKNSQVEVSWEYPDSWSTPHSYFSLKFFVRIQRKKE | | | | |
| KMKETEEGCNQKGAFLVEKTSTEVQCKGGNVCVQAQDRYYNSSCSKWACVPCRV | | | | |
| RSGGGSGGGSGGGSGGESPTKVQLVGGAHRCEGRVEVEHNGQWGTVCDDGWDRR | | | | |
| DVAVVCRELNCGAVIQTPRGASYQPPASEQRVLIQGVDCNGTEDTLAQCELNYDVF | | | | |
| DCSHEEDAGAQCENPDSDLLFIPEDVRLVDGPGHCQGRVEVLHQSQWSTVCKAGW | | | | |
| NLQVSKVVCRQLGCGRALLTYGSCNKSTQGKGPIWMGKMSCSGQEANLRSCLLSRL | | | | |
| ENNCTHGEDTWMECEDPFELKLVGGDTPCSGRLEVLHKGSWGSVCDDNWGEKED | | | | |
| QVVCKQLGCGKSLHPSPKTRKIYGPGAGRIWLDDVNCSGKEQSLEFCRHRLWGYHD | | | | |
| CTHKEDVEVICTDFDVTGHHHHHHHH* | | | | |
| Underline is the signal peptide, which is | | | | |
| removed in the final product | | | | |
| Protein sequence of mCD5L construct | | | | |
| ESPTKVQLVGGAHRCEGRVEVEHNGQWGTVCDDGWDRRDVAVVCRELN | | | | |
| CGAVIQTPRGASYQPPASEQRVLIQGVDCNGTEDTLAQCELNYDVFDCSHEEDAGA | | | | |
| QCENPDSDLLFIPEDVRLVDGPGHCQGRVEVLHQSQWSTVCKAGWNLQVSKVVCR | | | | |
| QLGCGRALLTYGSCNKSTQGKGPIWMGKMSCSGQEANLRSCLLSRLENNCTHGEDT | | | | |
| WMECEDPFELKLVGGDTPCSGRLEVLHKGSWGSVCDDNWGEKEDQVVCKQLGCG | | | | |
| KSLHPSPKTRKIYGPGAGRIWLDDVNCSGKEQSLEFCRHRLWGYHDCTHKEDVEVIC | | | | |
| TDFDVTGHHHHHHH* | | | | |
| Protein sequence of human CD5L:p40 construct | | | | |
| SPSGVRLVGGLHRCEGRVEVEQKGQWGTVCDDGWDIKDVAVLCRELGC | | | | |
| GAASGTPSGILYEPPAEKEQKVLIQSVSCTGTEDTLAQCEQEEVYDCSHDEDAGASC | | | | |
| ENPESSFSPVPEGVRLADGPGHCKGRVEVKHQNQWYTVCQTGWSLRAAKVVCRQL | | | | |
| GCGRAVLTQKRCNKHAYGRKPIWLSQMSCSGREATLQDCPSGPWGKNTCNHDEDT | | | | |
| WVECEDPFDLRLVGGDNLCSGRLEVLHKGVWGSVCDDNWGEKEDQVVCKQLGCG | | | | |
| KSLSPSFRDRKCYGPGVGRIWLDNVRCSGEEQSLEQCQHRFWGFHDCTHQEDVAVI | | | | |
| CSGGGGSGGGSGGGSGGIWELKKDVYVVELDWYPDAPGEMVVLTCDTPEEDGITW | | | | |
| TLDQSSEVLGSGKTLTIQVKEFGDAGQYTCHKGGEVLSHSLLLLHKKEDGIWSTDIL | | | | |
| KDQKEPKNKTFLRCEAKNYSGRFTCWWLTTISTDLTFSVKSSRGSSDPQGVTCGAAT | | | | |
| LSAERVRGDNKEYEYSVECQEDSACPAAEESLPIEVMVDAVHKLKYENYTSSFFIRDI | | | | |
| IKPDPPKNLQLKPLKNSRQVEVSWEYPDTWSTPHSYFSLTFCVQVQGKSKREKKDRV | | | | |
| FTDKTSATVICRKNASISVRAQDRYYSSSWSEWASVPCSTGHHHHHHHHGGQ | | | | |
| Protein sequence of human CD5L | | | | |
| SPSGVRLVGGLHRCEGRVEVEQKGQWGTVCDDGWDIKDVAVLCRELGC | | | | |
| GAASGTPSGILYEPPAEKEQKVLIQSVSCTGTEDTLAQCEQEEVYDCSHDEDAGASC | | | | |
| ENPESSFSPVPEGVRLADGPGHCKGRVEVKHQNQWYTVCQTGWSLRAAKVVCRQL | | | | |
| GCGRAVLTQKRCNKHAYGRKPIWLSQMSCSGREATLQDCPSGPWGKNTCNHDEDT | | | | |
| WVECEDPFDLRLVGGDNLCSGRLEVLHKGVWGSVCDDNWGEKEDQVVCKQLGCG | | | | |
| KSLSPSFRDRKCYGPGVGRIWLDNVRCSGEEQSLEQCQHRFWGFHDCTHQEDVAVI | | | | |
| CSGTGHHHHHHHHGGQ | | | | |
| Nucleotide information of mP40-mCD5L construct | | | | |
| GAATTCGCCACCATGTGTCCTCAGAAGCTAACCATCTCCTGGTTTGCCA | | | | |
| TCGTTTTGCTGGTGTCTCCACTCATGGCCATGTGGGAGCTGGAGAAAGACGTTTA | | | | |
| TGTTGTAGAGGTGGACTGGACTCCCGATGCCCCTGGAGAAACAGTGAACCTCAC | | | | |
| CTGTGACACGCCTGAAGAAGATGACATCACCTGGACCTCAGACCAGAGACATGG | | | | |
| AGTCATAGGCTCTGGAAAGACCCTGACCATCACTGTCAAAGAGTTTCTAGATGCT | | | | |
| GGCCAGTACACCTGCCACAAAGGAGGCGAGACTCTGAGCCACTCACATCTGCTG | | | | |
| CTCCACAAGAAGGAAAATGGAATTTGGTCCACTGAAATTTTAAAAAATTTCAAA | | | | |
| AACAAGACTTTCCTGAAGTGTGAAGCACCAAATTACTCCGGACGGTTCACGTGCT | | | | |
| CATGGCTGGTGCAAAGAAACATGGACTTGAAGTTCAACATCAAGAGCAGTAGCA | | | | |
| GTTCCCCTGACTCTCGGGCAGTGACATGTGGAATGGCGTCTCTGTCTGCAGAGAA | | | | |
| GGTCACACTGGACCAAAGGGACTATGAGAAGTATTCAGTGTCCTGCCAGGAGGA | | | | |
| TGTCACCTGCCCAACTGCCGAGGAGACCCTGCCCATTGAACTGGCGTTGGAAGC | | | | |
| ACGGCAGCAGAATAAATATGAGAACTACAGCACCAGCTTCTTCATCAGGGACAT | | | | |
| CATCAAACCAGACCCGCCCAAGAACTTGCAGATGAAGCCTTTGAAGAACTCACA | | | | |
| GGTGGAGGTCAGCTGGGAGTACCCTGACTCCTGGAGCACTCCCCATTCCTACTTC | | | | |
| TCCCTCAAGTTCTTTGTTCGAATCCAGCGCAAGAAAGAAAAGATGAAGGAGACA | | | | |
| GAGGAGGGGTGTAACCAGAAAGGTGCGTTCCTCGTAGAGAAGACATCTACCGAA | | | | |
| GTCCAATGCAAAGGCGGGAATGTCTGCGTGCAAGCTCAGGATCGCTATTACAAT | | | | |
| TCCTCGTGCAGCAAGTGGGCATGTGTTCCCTGCAGGGTCCGATCCGGAGGAGGA | | | | |
| TCCGGCGGGGGAAGCGGTGGAGGGTCTGGTGGGGAGTCTCCAACCAAAGTGCAG | | | | |
| CTAGTGGGAGGTGCCCACCGCTGTGAAGGGCGAGTGGAGGTGGAACACAATGGC | | | | |
| CAGTGGGGGACTGTGTGTGATGATGGCTGGGACCGGCGTGATGTGGCTGTGGTG | | | | |
| TGCCGAGAGCTCAATTGTGGAGCAGTCATCCAAACCCCGCGTGGCGCATCATATC | | | | |
| AGCCACCAGCATCAGAGCAAAGAGTTCTTATTCAAGGGGTTGACTGCAACGGAA | | | | |
| CGGAAGACACGTTGGCTCAATGTGAGCTAAATTACGATGTTTTTGACTGCTCACA | | | | |
| TGAAGAAGATGCTGGGGCACAGTGTGAGAACCCAGACAGTGACCTCCTCTTCAT | | | | |
| TCCAGAGGATGTGCGTCTAGTAGATGGCCCGGGGCACTGCCAGGGTCGAGTGGA | | | | |
| GGTGCTCCACCAGTCCCAGTGGAGCACTGTGTGTAAAGCAGGCTGGAACTTACA | | | | |
| GGTCTCAAAGGTGGTGTGCAGGCAGCTCGGGTGTGGGCGGGCATTACTGACCTA | | | | |
| CGGAAGCTGCAACAAGAGTACTCAGGGCAAAGGACCCATCTGGATGGGCAAGAT | | | | |
| GTCGTGTTCTGGACAAGAAGCAAACCTTCGGTCTTGCCTTTTGAGTCGTTTGGAG | | | | |
| AACAACTGTACCCATGGCGAGGACACATGGATGGAATGTGAAGATCCTTTTGAG | | | | |
| CTGAAGCTGGTGGGAGGAGACACCCCCTGCTCTGGGAGGTTGGAGGTGCTGCAC | | | | |
| AAGGGTTCCTGGGGCTCCGTCTGTGATGACAACTGGGGAGAAAAGGAGGACCAA | | | | |
| GTGGTCTGCAAGCAACTGGGTTGTGGGAAGTCCCTCCATCCATCCCCCAAAACCC | | | | |
| GGAAAATCTATGGGCCTGGGGCAGGCCGCATCTGGCTGGATGACGTCAACTGCT | | | | |
| CAGGGAAGGAACAGTCTCTGGAGTTCTGCCGGCACAGGTTGTGGGGGTACCACG | | | | |
| ACTGTACCCACAAGGAAGATGTGGAGGTGATCTGCACAGACTTTGATGTGACCG | | | | |
| GTCATCATCACCATCACCATCACCATGGAGGACAGTGA | | | | |
| Nucleotide information for mCD5L construct | | | | |
| GAGTCTCCAACCAAAGTGCAGCTAGTGGGAGGTGCCCACCGCTGTGAA | | | | |
| GGGCGAGTGGAGGTGGAACACAATGGCCAGTGGGGGACTGTGTGTGATGATGGC | | | | |
| TGGGACCGGCGTGATGTGGCTGTGGTGTGCCGAGAGCTCAATTGTGGAGCAGTC | | | | |
| ATCCAAACCCCGCGTGGCGCATCATATCAGCCACCAGCATCAGAGCAAAGAGTT | | | | |
| CTTATTCAAGGGGTTGACTGCAACGGAACGGAAGACACGTTGGCTCAATGTGAG | | | | |
| CTAAATTACGATGTTTTTGACTGCTCACATGAAGAAGATGCTGGGGCACAGTGTG | | | | |
| AGAACCCAGACAGTGACCTCCTCTTCATTCCAGAGGATGTGCGTCTAGTAGATGG | | | | |
| CCCGGGGCACTGCCAGGGTCGAGTGGAGGTGCTCCACCAGTCCCAGTGGAGCAC | | | | |
| TGTGTGTAAAGCAGGCTGGAACTTACAGGTCTCAAAGGTGGTGTGCAGGCAGCT | | | | |
| CGGGTGTGGGCGGGCATTACTGACCTACGGAAGCTGCAACAAGAGTACTCAGGG | | | | |
| CAAAGGACCCATCTGGATGGGCAAGATGTCGTGTTCTGGACAAGAAGCAAACCT | | | | |
| TCGGTCTTGCCTTTTGAGTCGTTTGGAGAACAACTGTACCCATGGCGAGGACACA | | | | |
| TGGATGGAATGTGAAGATCCTTTTGAGCTGAAGCTGGTGGGAGGAGACACCCCC | | | | |
| TGCTCTGGGAGGTTGGAGGTGCTGCACAAGGGTTCCTGGGGCTCCGTCTGTGATG | | | | |
| ACAACTGGGGAGAAAAGGAGGACCAAGTGGTCTGCAAGCAACTGGGTTGTGGG | | | | |
| AAGTCCCTCCATCCATCCCCCAAAACCCGGAAAATCTATGGGCCTGGGGCAGGC | | | | |
| CGCATCTGGCTGGATGACGTCAACTGCTCAGGGAAGGAACAGTCTCTGGAGTTCT | | | | |
| GCCGGCACAGGTTGTGGGGGTACCACGACTGTACCCACAAGGAAGATGTGGAGG | | | | |
| TGATCTGCACAGACTTTGATGTG | | | | |
| Nucleotide information for human CD5L construct | | | | |
| TCTCCATCTG | GAGTGCGGCT | GGTGGGGGGC | CTCCACCGCT | |
| GTGAAGGGCG | GGTGGAGGTG | GAACAGAAAG | GCCAGTGGGG | CACCGTGTGT |
| GATGACGGCT | GGGACATTAA | GGACGTGGCT | GTGTTGTGCC | GGGAGCTGGG |
| CTGTGGAGCT | GCCAGCGGAA | CCCCTAGTGG | TATTTTGTAT | GAGCCACCAG |
| CAGAAAAAGA | GCAAAAGGTC | CTCATCCAAT | CAGTCAGTTG | CACAGGAACA |
| GAAGATACAT | TGGCTCAGTG | TGAGCAAGAA | GAAGTTTATG | ATTGTTCACA |
| TGATGAAGAT | GCTGGGGCAT | CGTGTGAGAA | CCCAGAGAGC | TCTTTCTCCC |
| CAGTCCCAGA | GGGTGTCAGG | CTGGCTGACG | GCCCTGGGCA | TTGCAAGGGA |
| CGCGTGGAAG | TGAAGCACCA | GAACCAGTGG | TATACCGTGT | GCCAGACAGG |
| CTGGAGCCTC | CGGGCCGCAA | AGGTGGTGTG | CCGGCAGCTG | GGATGTGGGA |
| GGGCTGTACT | GACTCAAAAA | CGCTGCAACA | AGCATGCCTA | TGGCCGAAAA |
| CCCATCTGGC | TGAGCCAGAT | GTCATGCTCA | GGACGAGAAG | CAACCCTTCA |
| GGATTGCCCT | TCTGGGCCTT | GGGGGAAGAA | CACCTGCAAC | CATGATGAAG |
| ACACGTGGGT | CGAATGTGAA | GATCCCTTTG | ACTTGAGACT | AGTAGGAGGA |
| GACAACCTCT | GCTCTGGGCG | ACTGGAGGTG | CTGCACAAGG | GCGTATGGGG |
| CTCTGTCTGT | GATGACAACT | GGGGAGAAAA | GGAGGACCAG | GTGGTATGCA |
| AGCAACTGGG | CTGTGGGAAG | TCCCTCTCTC | CCTCCTTCAG | AGACCGGAAA |
| TGCTATGGCC | CTGGGGTTGG | CCGCATCTGG | CTGGATAATG | TTCGTTGCTC |
| AGGGGAGGAG | CAGTCCCTGG | AGCAGTGCCA | GCACAGATTT | TGGG |
| ACCGGTCATCATCACCATCACCATCACCATGGAGGACAGTGA | | | | |
| Nucleotide information for human CD5L:p40 construct | | | | |
| TCTCCATCTGGAGTGCGGCTGGTGGGGGGCCTCCACCGCTGTGAAGGG | | | | |
| CGGGTGGAGGTGGAACAGAAAGGCCAGTGGGGCACCGTGTGTGATGACGGCTG | | | | |
| GGACATTAAGGACGTGGCTGTGTTGTGCCGGGAGCTGGGCTGTGGAGCT | | | | |
| GCCAGCGGAACCCCTAGTGGTATTTTGTATGAGCCACCAG | | | | CAGAAAAAGA |
| GCAAAAGGTC | CTCATCCAAT | CAGTCAGTTG | CACAGGAACA | GAAGATACAT |
| TGGCTCAGTG | TGAGCAAGAA | GAAGTTTATG | ATTGTTCACA | TGATGAAGAT |
| GCTGGGGCAT | CGTGTGAGAA | CCCAGAGAGC | TCTTTCTCCC | CAGTCCCAGA |
| GGGTGTCAGG | CTGGCTGACG | GCCCTGGGCA | TTGCAAGGGA | CGCGTGGAAG |
| TGAAGCACCA | GAACCAGTGG | TATACCGTGT | GCCAGACAGG | CTGGAGCCTC |
| CGGGCCGCAA | AGGTGGTGTG | CCGGCAGCTG | GGATGTGGGA | GGGCTGTACT |
| GACTCAAAAA | CGCTGCAACA | AGCATGCCTA | TGGCCGAAAA | CCCATCTGGC |
| TGAGCCAGAT | GTCATGCTCA | GGACGAGAAG | CAACCCTTCA | GGATTGCCCT |
| TCTGGGCCTT | GGGGGAAGAA | CACCTGCAAC | CATGATGAAG | ACACGTGGGT |
| CGAATGTGAA | GATCCCTTTG | ACTTGAGACT | AGTAGGAGGA | GACAACCTCT |
| GCTCTGGGCG | ACTGGAGGTG | CTGCACAAGG | GCGTATGGGG | CTCTGTCTGT |
| GATGACAACT | GGGGAGAAAA | GGAGGACCAG | GTGGTATGCA | AGCAACTGGG |
| CTGTGGGAAG | TCCCTCTCTC | CCTCCTTCAG | AGACCGGAAA | TGCTATGGCC |
| CTGGGGTTGG | CCGCATCTGG | CTGGATAATG | TTCGTTGCTC | AGGGGAGGAG |
| CAGTCCCTGGAGCAGTGCCAGCACAGATTTTGGGGGAGGAGGATCCGGCGGGGG | | | | |
| AAGCGGTGGAGGGTCTGGTGGGATATGGGAACTGAAGAAAGATGTTTATGTCGT | | | | |
| AGAATTGGAT | TGGTATCCGG | ATGCCCCTGG | AGAAATGGTG | GTCCTCACCT |
| GTGACACCCC | TGAAGAAGAT | GGTATCACCT | GGACCTTGGA | CCAGAGCAGT |
| GAGGTCTTAG | GCTCTGGCAA | AACCCTGACC | ATCCAAGTCA | AAGAGTTTGG |
| AGATGCTGGC | CAGTACACCT | GTCACAAAGG | AGGCGAGGTT | CTAAGCCATT |
| CGCTCCTGCT | GCTTCACAAA | AAGGAAGATG | GAATTTGGTC | CACTGATATT |
| TTAAAGGACC | AGAAAGAACC | CAAAAATAAG | ACCTTTCTAA | GATGCGAGGC |
| CAAGAATTAT | TCTGGACGTT | TCACCTGCTG | GTGGCTGACG | ACAATCAGTA |
| CTGATTTGAC | ATTCAGTGTC | AAAAGCAGCA | GAGGCTCTTC | TGACCCCCAA |
| GGGGTGACGT | GCGGAGCTGC | TACACTCTCT | GCAGAGAGAG | TCAGAGGGGA |
| CAACAAGGAG | TATGAGTACT | CAGTGGAGTG | CCAGGAGGAC | AGTGCCTGCC |
| CAGCTGCTGA | GGAGAGTCTG | CCCATTGAGG | TCATGGTGGA | TGCCGTTCAC |
| AAGCTCAAGT | ATGAAAACTA | CACCAGCAGC | TTCTTCATCA | GGGACATCAT |
| CAAACCTGAC | CCACCCAAGA | ACTTGCAGCT | GAAGCCATTA | AAGAATTCTC |
| GGCAGGTGGA | GGTCAGCTGG | GAGTACCCTG | ACACCTGGAG | TACTCCACAT |
| TCCTACTTCT | CCCTGACATT | CTGCGTTCAG | GTCCAGGGCA | AGAGCAAGAG |
| AGAAAAGAAA | GATAGAGTCT | TCACGGACAA | GACCTCAGCC | ACGGTCATCT |

GCCGCAAAAATGCCAGCATTAGCGTGCGGGCCCAGGACCGCTACTATAGCTCAT
CTTGGAGCGAATGGGCATCTGTGCCCTGCAGTACCGGTCATCATCACCATCACCA
TCACCATGGAGGACAGTGA

### Example 38—CD5L:p40 Heterodimer Rescues CD5L Deficiency in Myeloid Cells in DSS Colitis

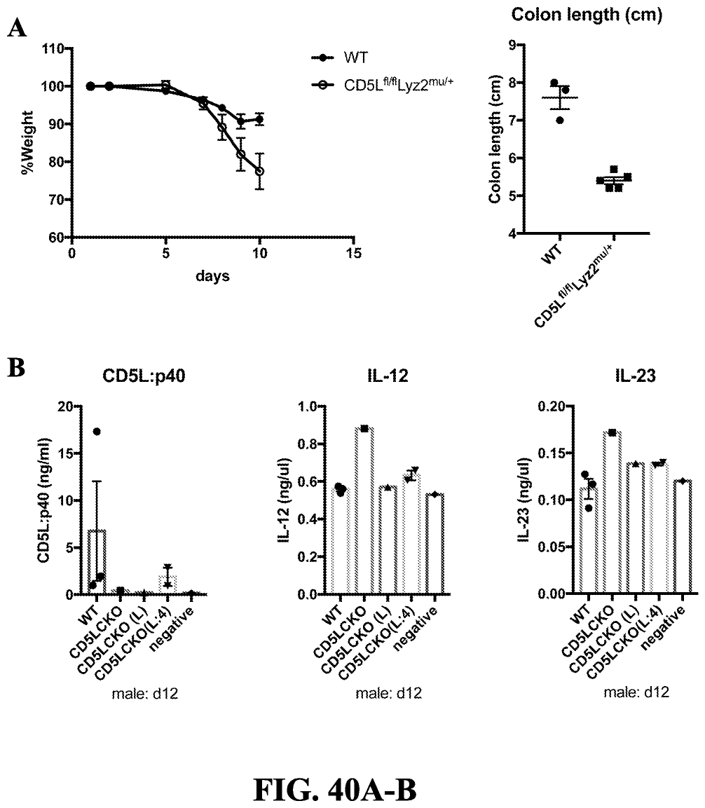

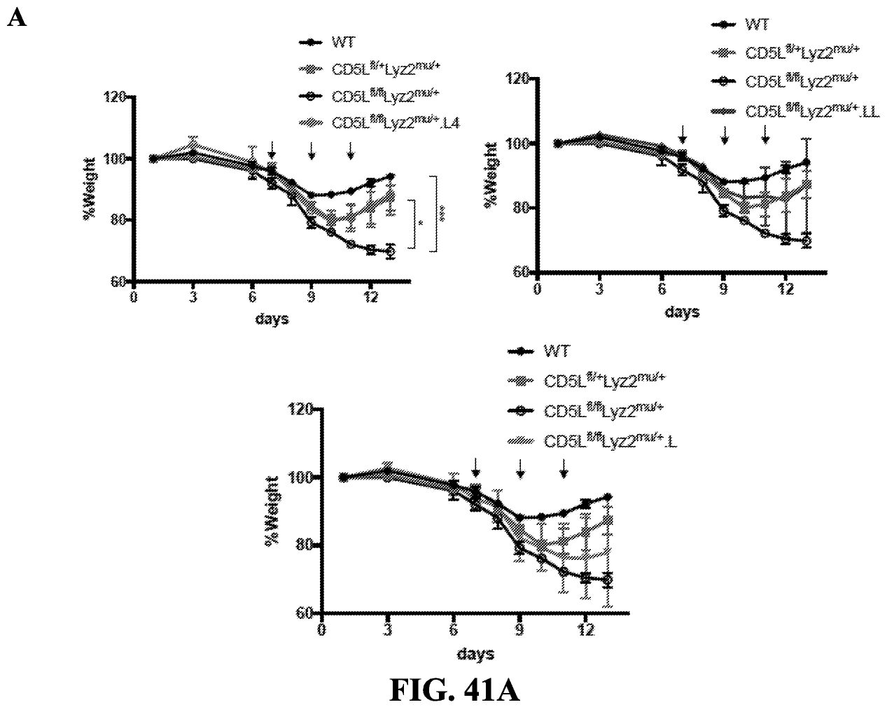

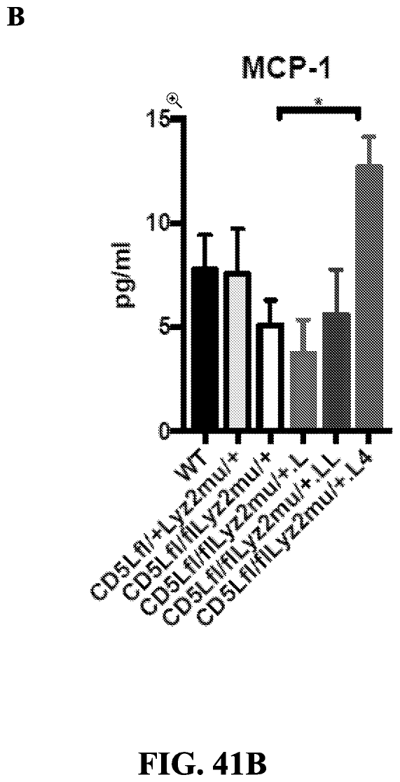

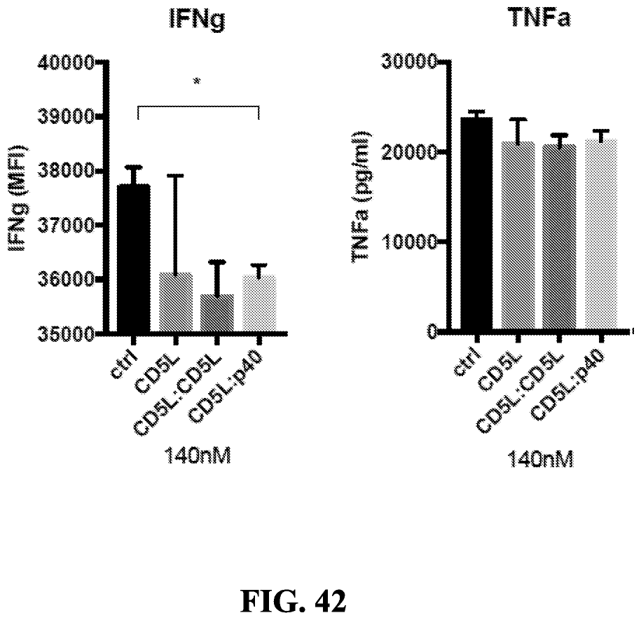

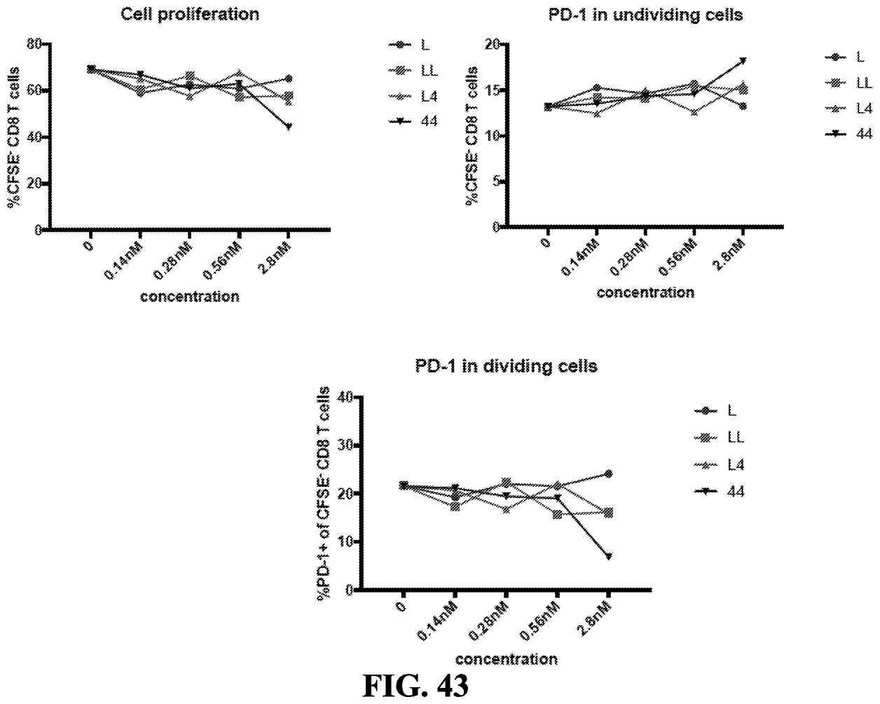

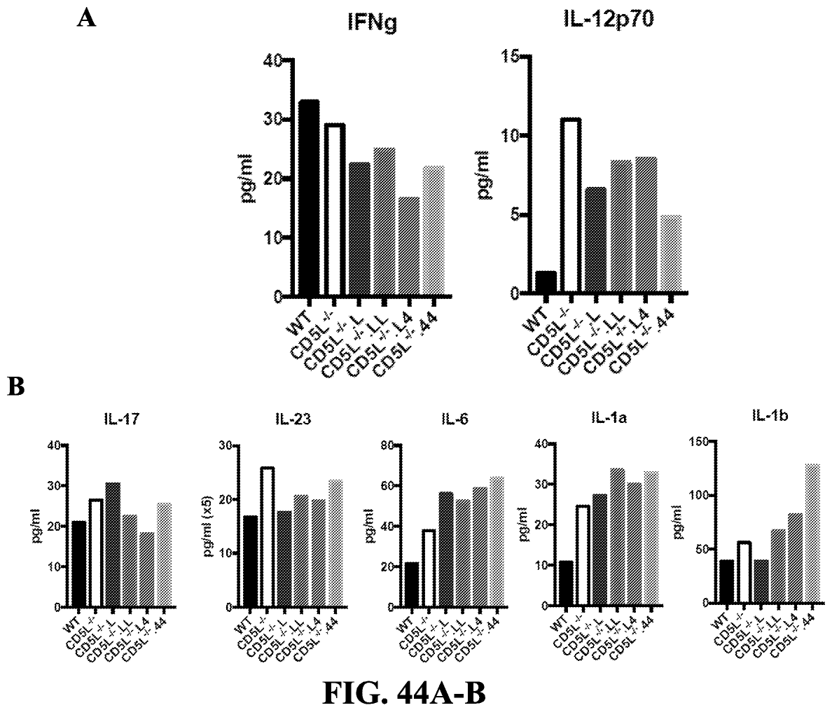

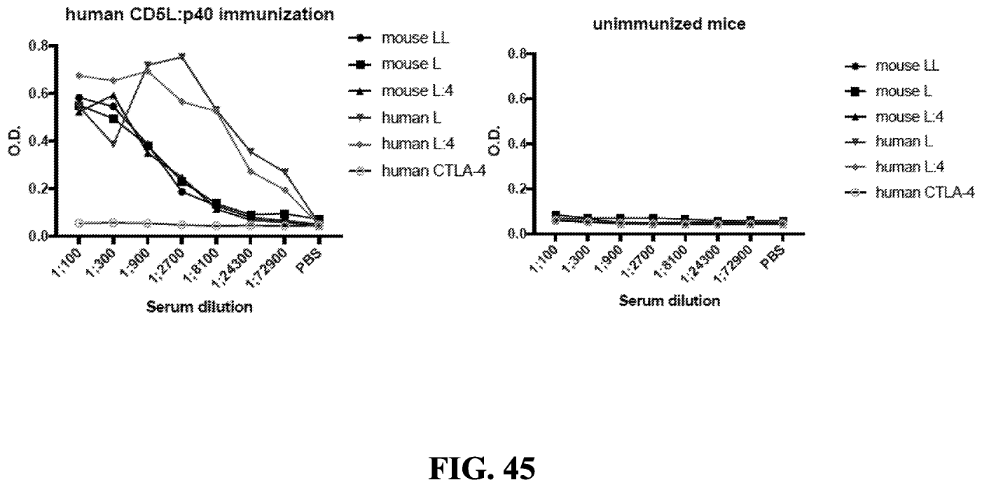

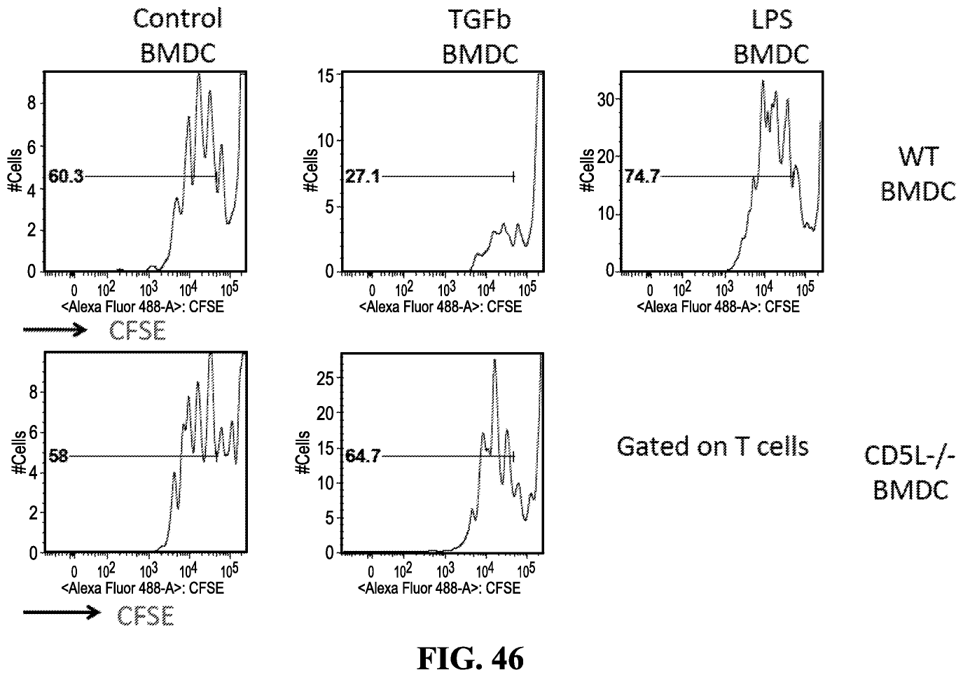

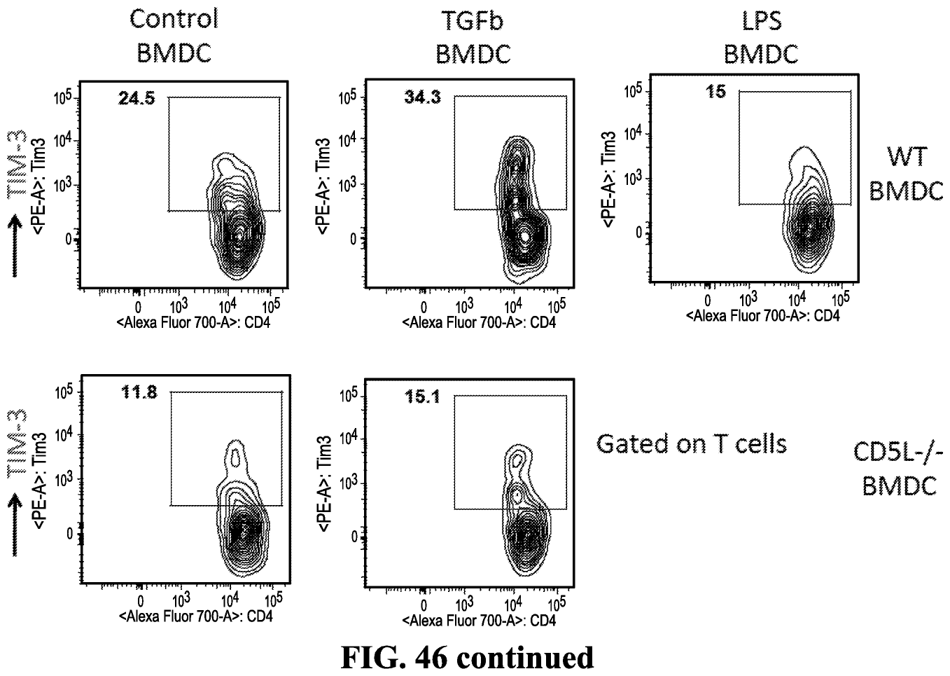

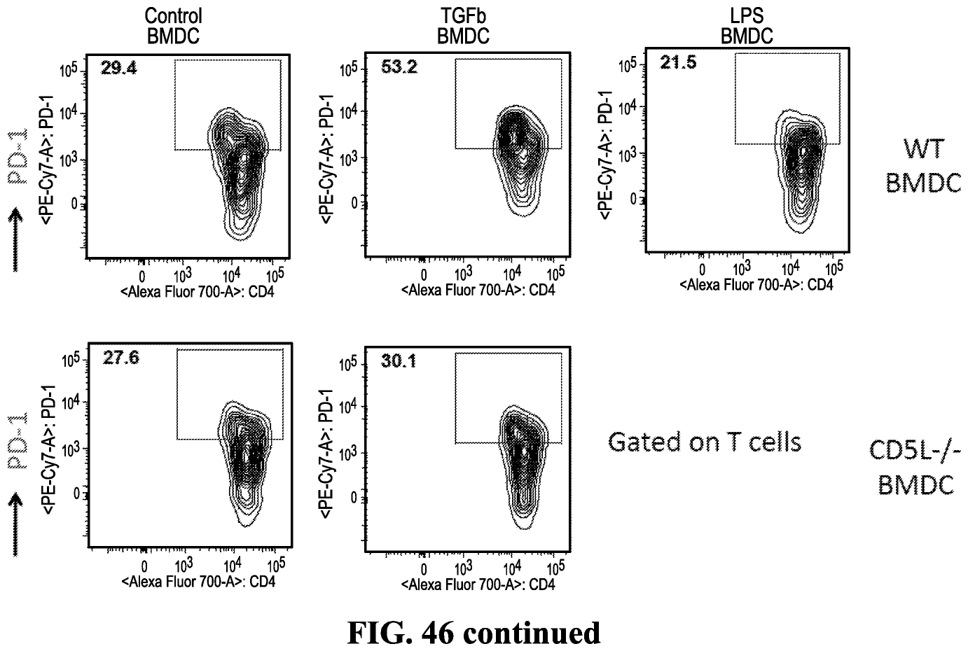

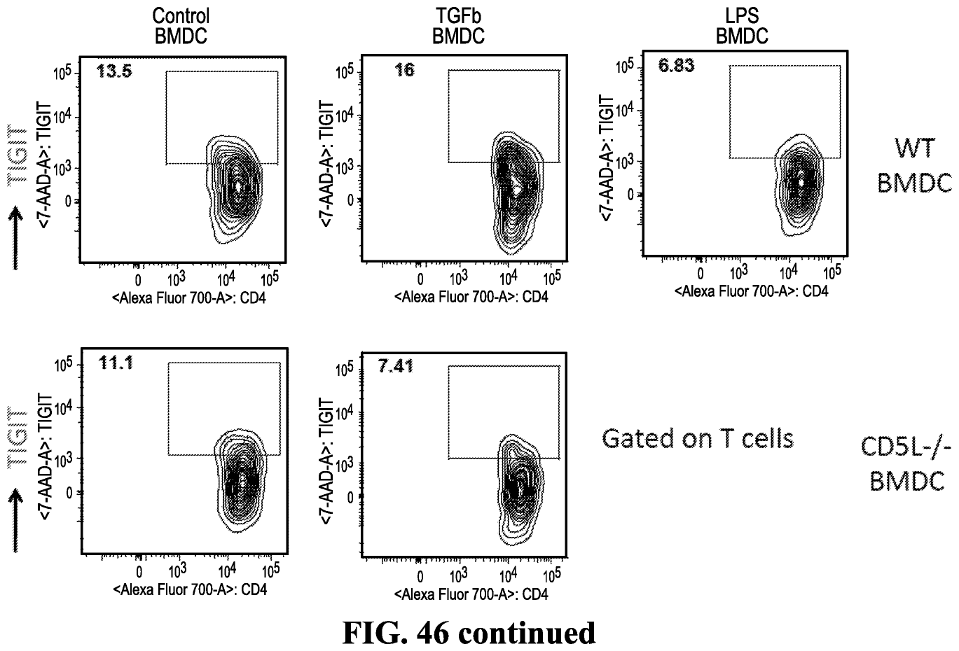

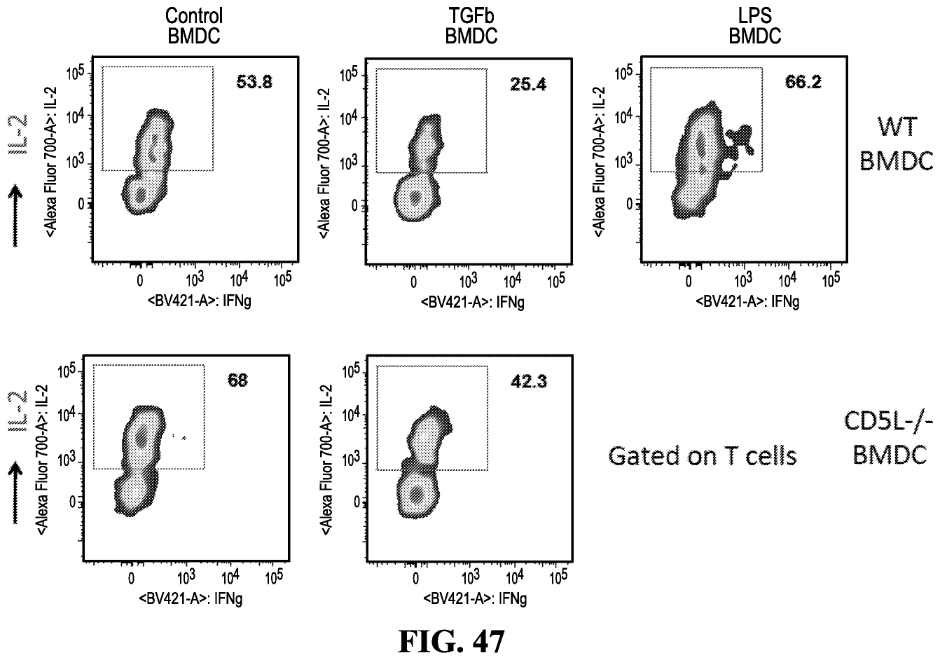

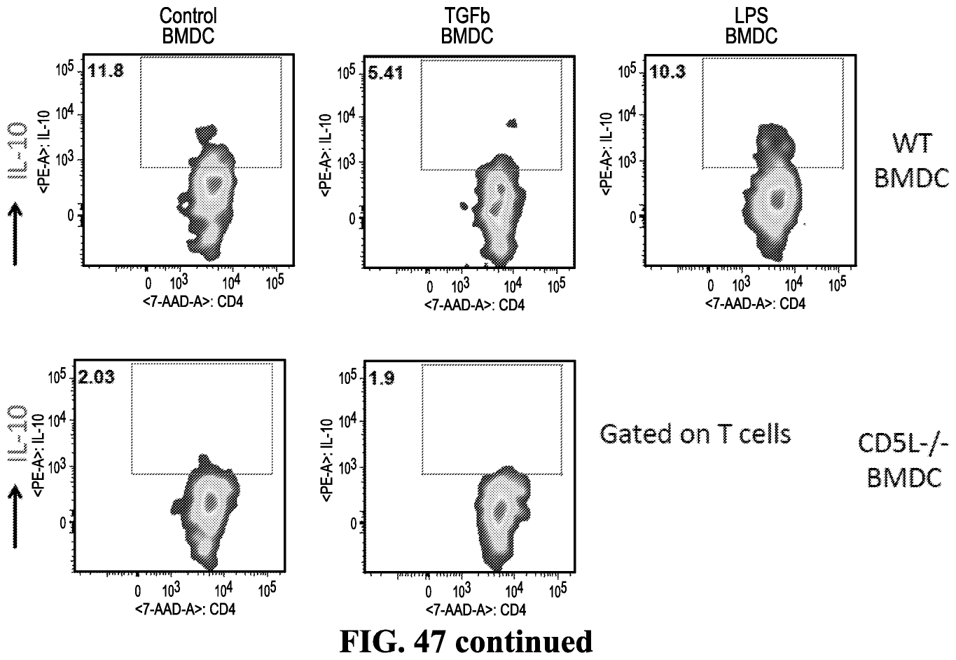

Conditional CD5L knockout female mice (CD5L^{fl/+}Lyz2^{mu/+} and CD5L^{fl/fl}Lyz2^{mu/+}) and global CD5L knockout male mice (CD5L^{-/-}) were generated. Wild-type mice and the knockout mice were induced with colitis by treating with 2% DSS in drinking water for a consecutive of 7 days followed by normal water. As shown in FIG. 40, myeloid cells are the major generator of CD5L:p40 heterodimer in DSS colitis setting in vivo, and in the absence of which IL-23 and IL-12 expression goes up in serum. To assess whether CD5L:p40 can rescue CD5L deficiency in DSS colitis, the female CD5L knockout mice were given either control (PBS), recombinant CD5L:p40, CD5L, or CD5L:CD5L homodimer intraperitoneally on day 7, 9 and 11, and the male global CD5L knockout mice were given either control (PBS), recombinant CD5L:p40, CD5L, or CD5L:CD5L homodimer intraperitoneally on day 7 and 9. As shown in FIG. 41A, CD5L:p40 but not CD5L:CD5L homodimer or CD5L monomer can rescue CD5L deficiency in myeloid cells in female mice undergoing DSS-colitis. No rescue was observed in male mice that are CD5L global knockout.

Recombinant CD5L:p40 was also shown to promote MCP-1 during recovery phase of DSS-colitis (FIG. 41B). Splenocytes from respective mice were isolated from day 12 and incubated ex vivo for 4 hours in the presence of Monensin and Brefeldin A. Supernatant was harvested for analysis of MCP-1. MCP-1 was shown to contribute to gut homeostasis and is important in recruiting M2 macrophase (Takada et al., Journal of Immunology (2010) 184(5):2671-2676). MCP-1 drives TH2 differentiation (Gu et al., Nature (2000) 404 (6776):407-411) and its expressin is significantly correlated with infiltration of tumor-associated macrophase, angiogenesis and poor survival in breast cancer patients (reviewed in Lim et al., Oncotarget (2016) 7(19):28697-710); and Deshmane et al., J Interferon Cytokine Res. (2009) 29(6):313-326). Whether CD5L:p40 uniquely (as compared to CD5L monomer, homodimer and p40:p40 domodimer) induces MCP-1 and drives Th2 response and M2 macrophage recruitment is also tested.

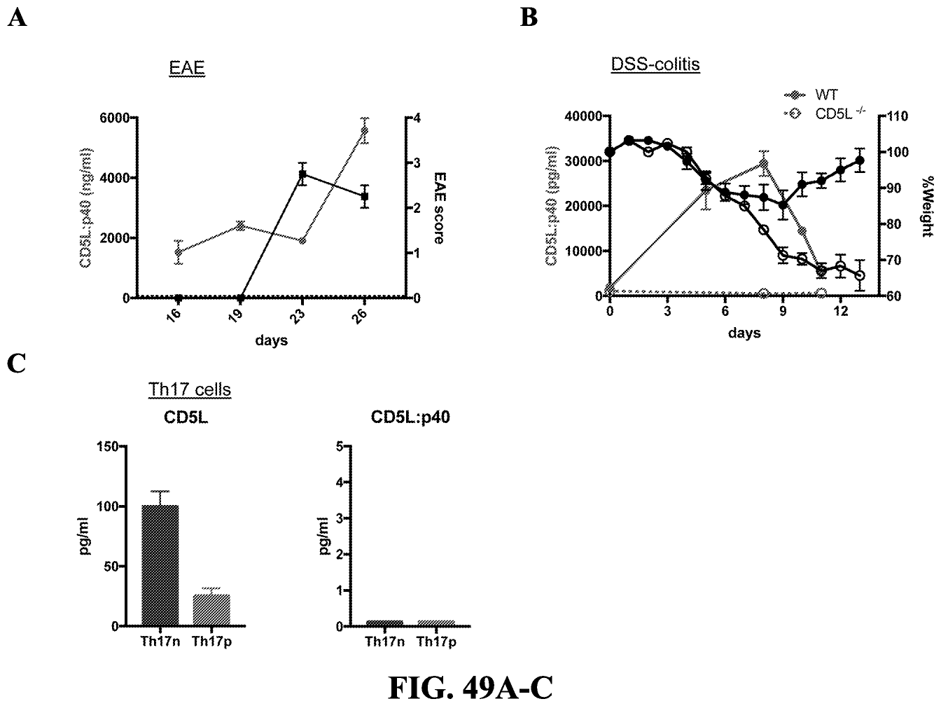

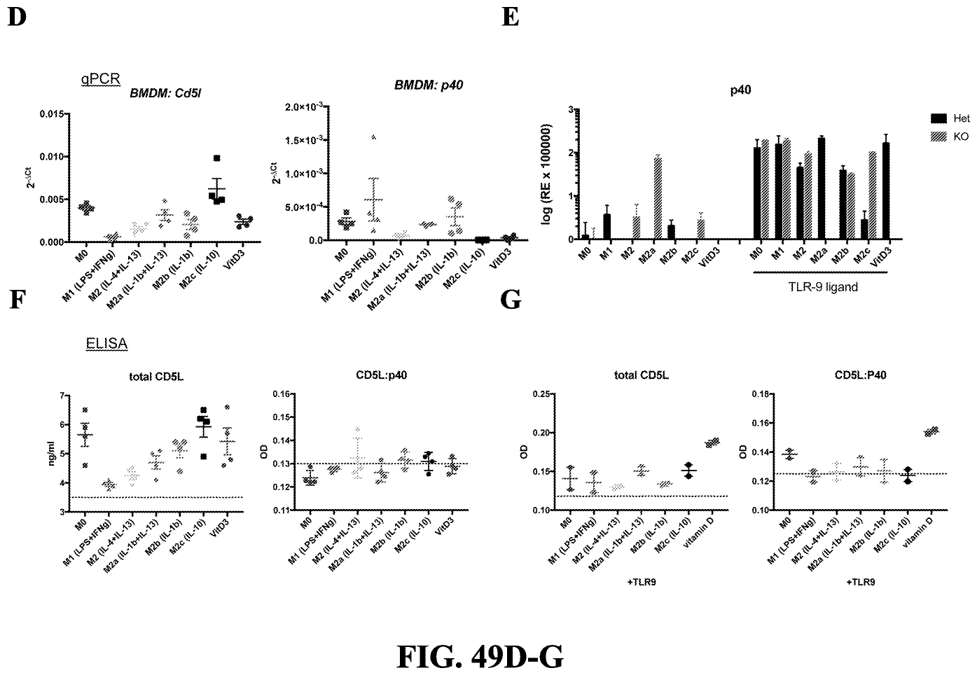

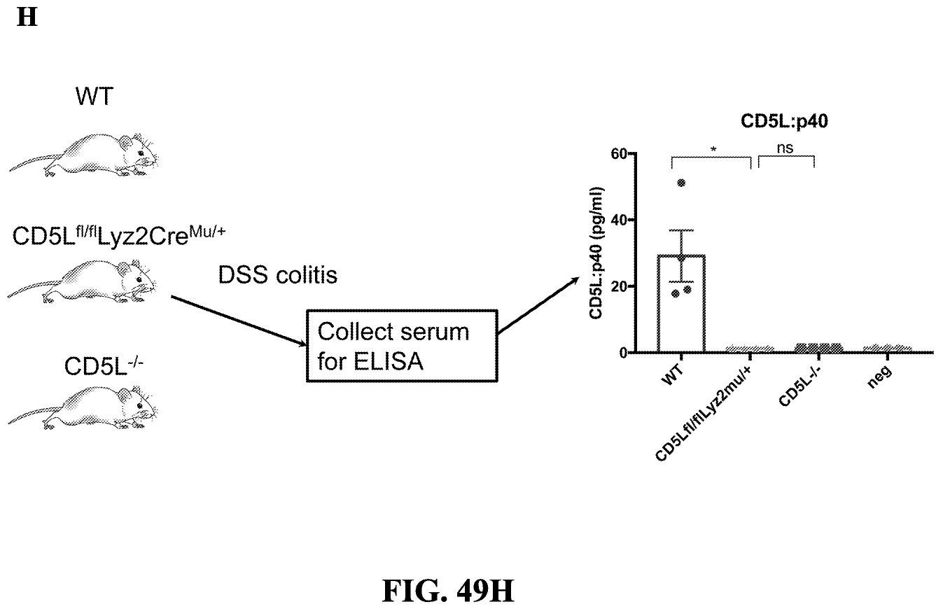

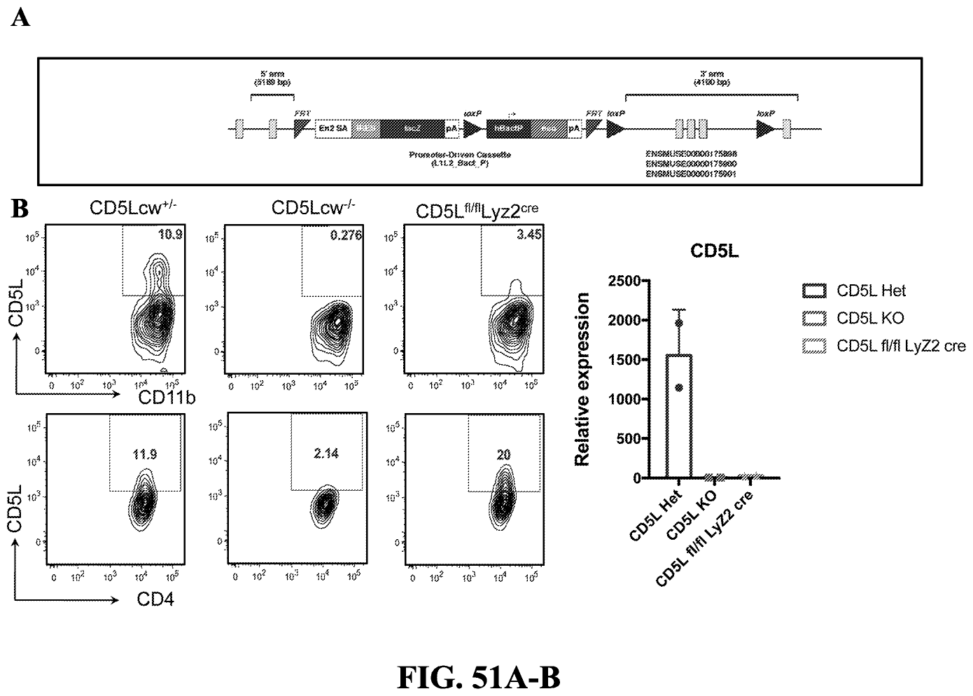

FIGS. 49A and B show CD5L:p40 secretion during disease progression in mouse models. For EAE, CD5L:p40 is secreted at the peak of EAE disease and the EAE score decreases. For DSS induced colitis, CD5L:p40 is secreted in response to weight loss in the wild type mouse followed by an increase in weight. In the CD5L−/− mouse, weight loss continues to decrease. These results suggest that CD5L:p40 is secreted during inflammation to reverse or ameliorate disease. FIG. 49C shows that Th17 cells secrete CD5L mostly during differentiation under pathogenic conditions. However, the CD5L secreted by Th17 cells is not CD5L:p40, as Th17 cells differentiation under pathogenic and non-pathogenic conditions does not result in any secretion of CD5L:p40. Thus, CD5L:p40 is secreted by a different cell type. FIGS. 49 D-E show mRNA expression of CD5L and p40 in BMDM macrophages (CD5L−/− and CD5L+/−) under the indicated conditions. TLR9 stimulation resulted in p40 expression. FIGS. 49 F-G show ELISA results for total CD5L and CD5L:p40 in BMDM macrophages under the indicated conditions. TLR9 stimulation resulted in p40 expression. FIG. 49 H shows that CD5L:p40 is secreted by myeloid cells. Wild type mice induced with DSS secrete CD5L:p40, however, when CD5L is knocked out in myeloid cells in the conditional knockout mouse, CD5L:p40 secretion is not detected. FIG. 51 shows the generation and validation of conditional CD5L knockout mice in myeloid cells (see, also, Example 14).

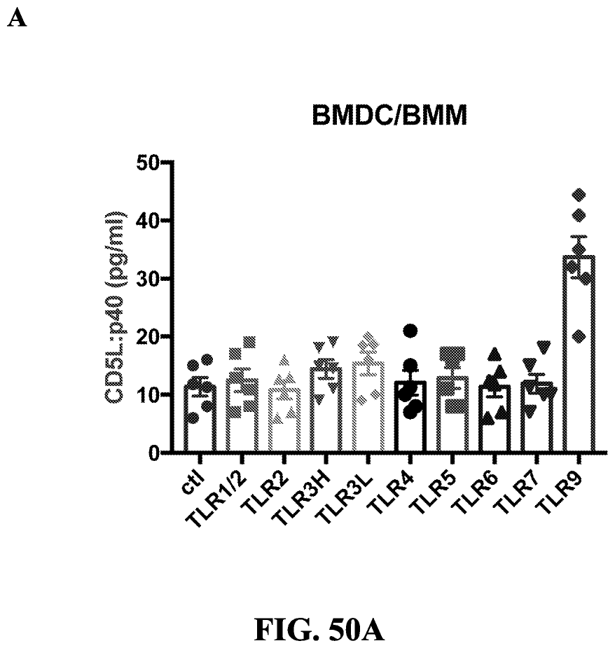

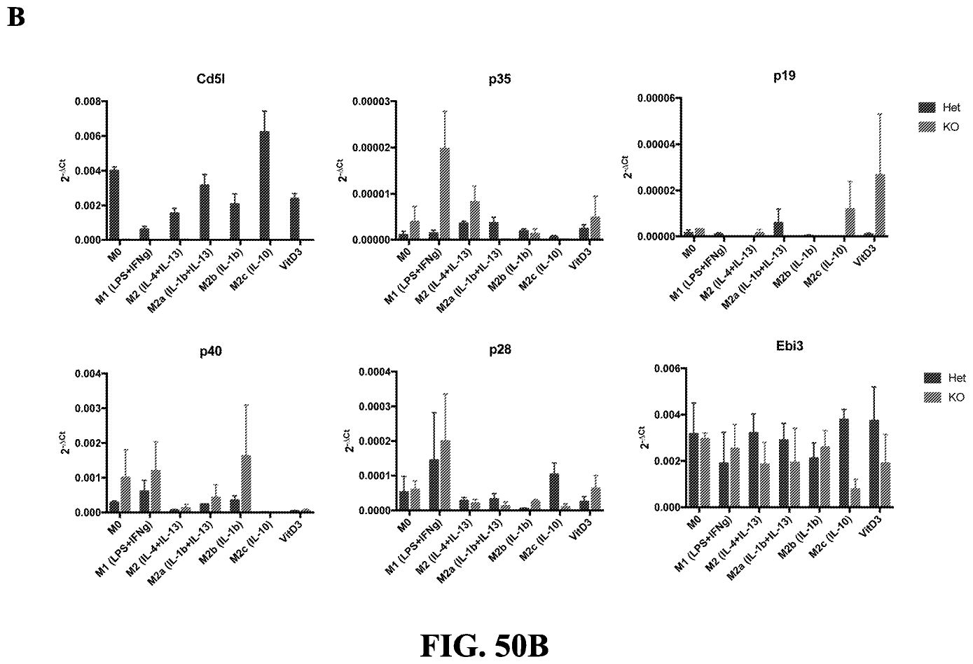

FIG. 50A shows that CD5L:p40 secretion is highest in mixed BMDC/BMM mixed cultures when stimulated with TLR9. FIG. 50B shows expression of p19 and p35 in myeloid cells (CD5L−/− and CD5L+/−) and their regulation by Cd51. Expression was determined under the indicated conditions.

### Example 39—Effects of Recombinant CD5L Monomers, Dimers and Heterodimers

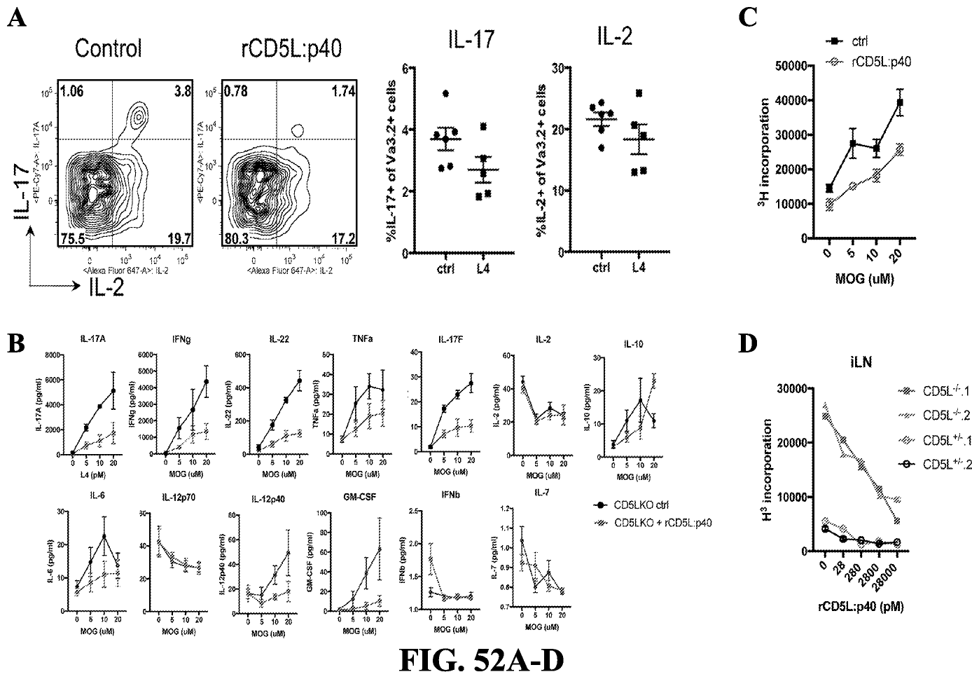

FIG. 52 further shows that recombinant CD5L:p40 alters antigen specific responses. Wildtype B6 mice (A) were immunized with MOG/CFA and recombinant CD5L:p40 were given at 1 pmol/g of body weight on day 2, 4 and 7 post immunization by intraperitoneal injection. FIG. 52 A shows cytokine production from antigen-specific T cells from a similar experiment where naïve 2D2 T cells were transferred 2 days prior to immunization. 1117 was decreased after treatment with CD5L:p40. FIG. 52B shows an ex vivo MOG recall response for the indicated cytokines. CD5L:p40 causes decreased inflammatory cytokines (e.g., IL-17A) and increased suppressive cytokines (e.g., IL-10). FIG. 52C shows a thymidine incorporation assay from same condition as in B). CD5L:p40 causes decreased incorporation of ³H. FIG. 52D shows CD5L+/− or CD5L−/− mice immunized by MOG/CFA. Inguinal lymph nodes were isolated for the MOG recall assay in the presence of control or recombinant CD5L:p40 followed by thymidine incorporation assay as in C. CD5L:p40 decreased incorporation of ³H and rescued CD5L loss.

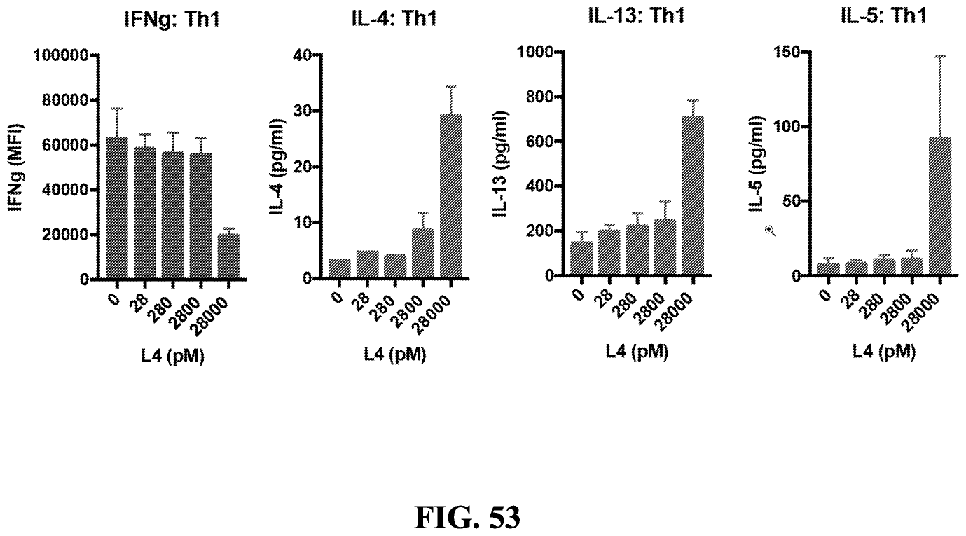

FIG. 53 shows that recombinant CD5L:p40 suppresses IFNg production but promotes Th2 cytokines from Th1 cells in vitro. Naïve T cells were differentiated under Th1 condition in the presence of different doses of CD5L:p40. IFNg, IL-4, IL-5 and IL-13 were measured using legendplex using a flow-based assay on day 3 of T cell culture. CD5L:p40 caused a decrease in IFNg and an increase in IL-4, IL-13 and IL-5.

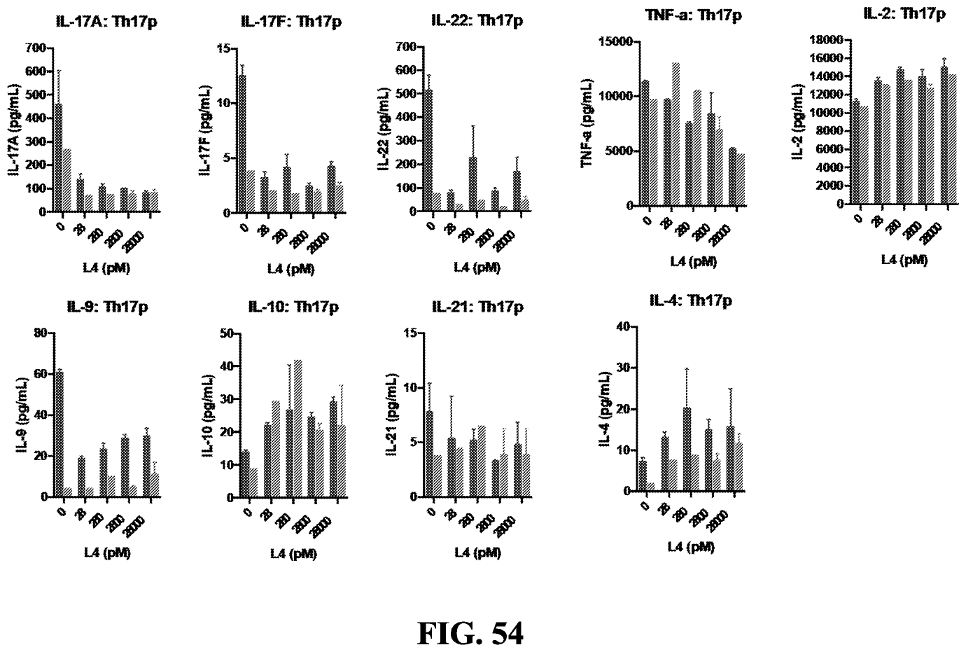

FIG. 54 shows the effect of recombinant CD5L:p40 on Th17 cells. CD5L−/− and CD5L+/−Th17 cells were treated in the presence of different doses of CD5L:p40.

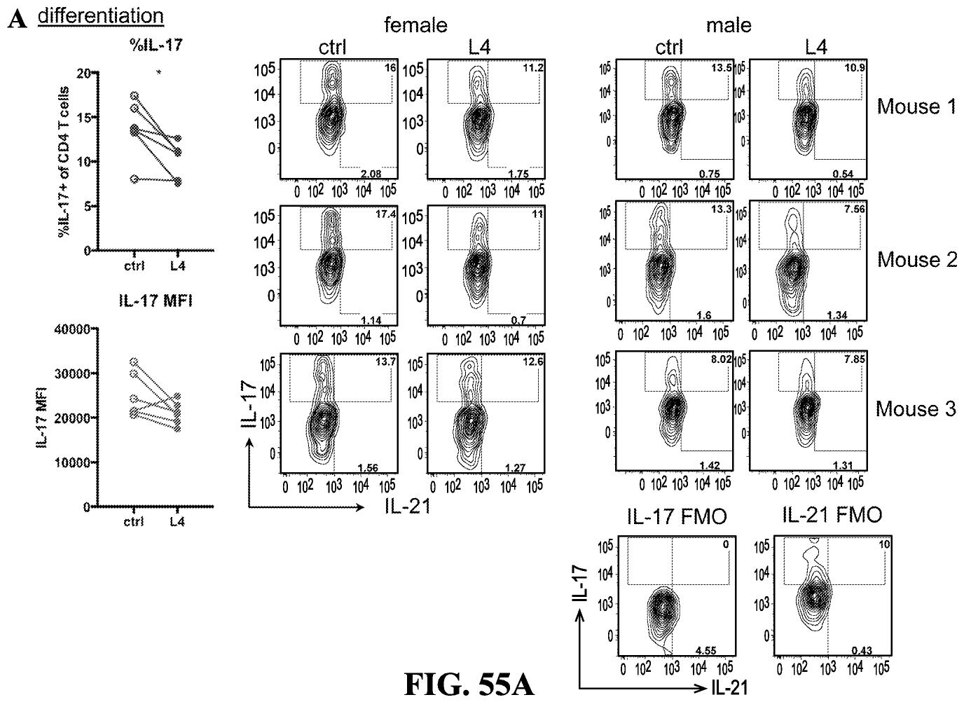

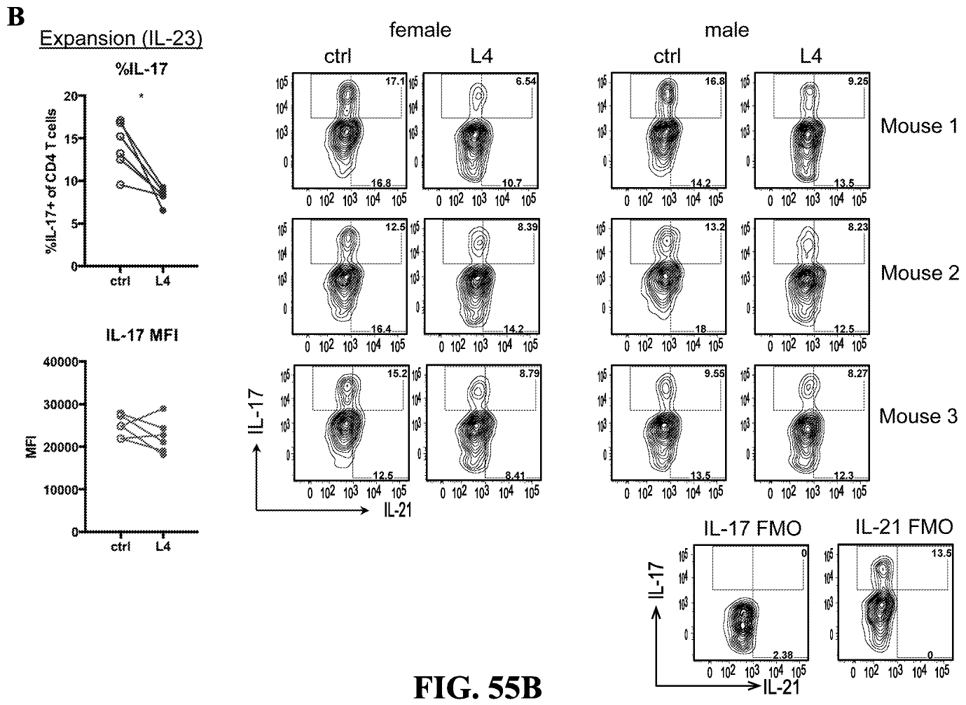

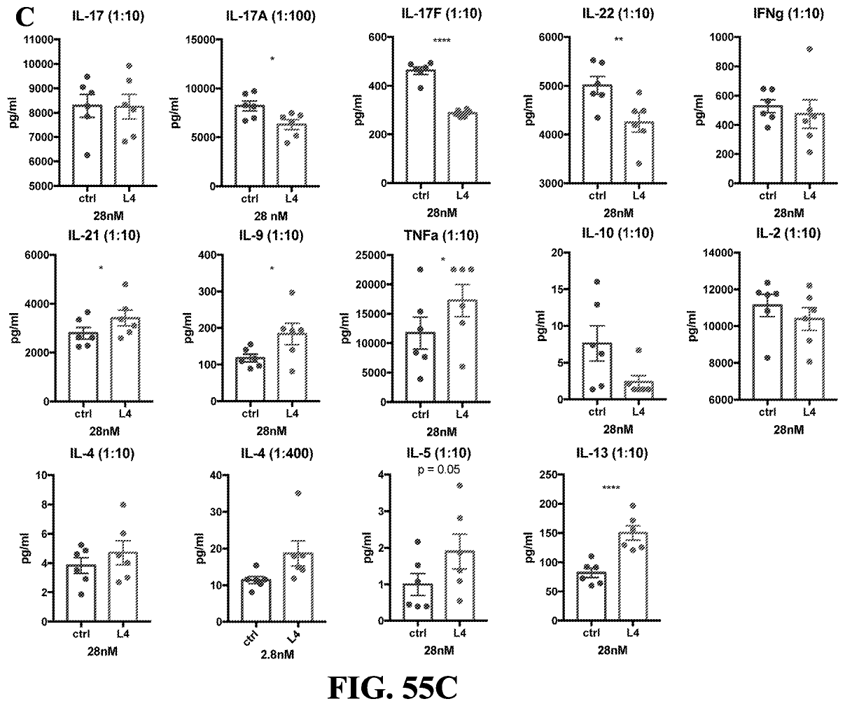

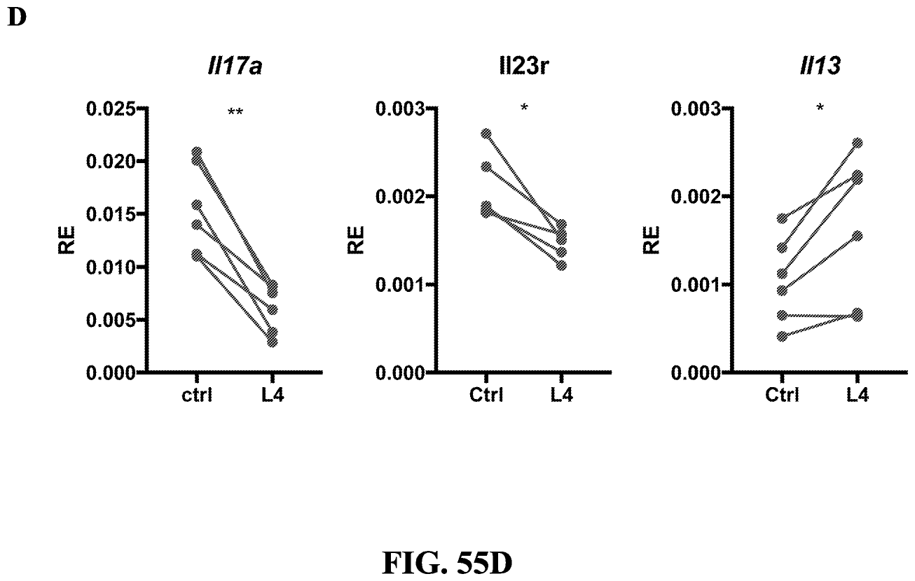

FIG. 55 further shows that recombinant CD5L:p40 suppresses Th17 responses and promotes type 2 responses directly in vitro. FIG. 55A shows decreased intracellular IL-17 production in naïve T cells differentiated under the pathogenic Th17 condition (IL-1b+IL-6+IL-23) in the presence of CD5L:p40 (L4). FIG. 55B shows decreased intracellular IL-17 production in Th17p cells differentiated as in A), and further expanded in IL-23 without addition of other cytokines (e.g. L4). FIG. 55C shows changes in cytokine secretion detected in the supernatant of Th17p differentiation culture as in A). FIG. 55D shows a decrease in 1117a and 1123r and an increase in 1113 with recombinant CD5L:p40 treatment.

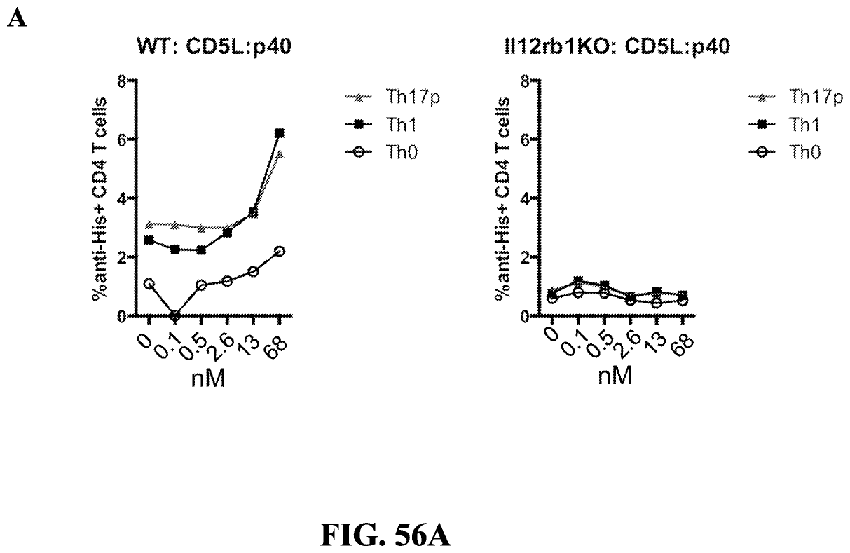

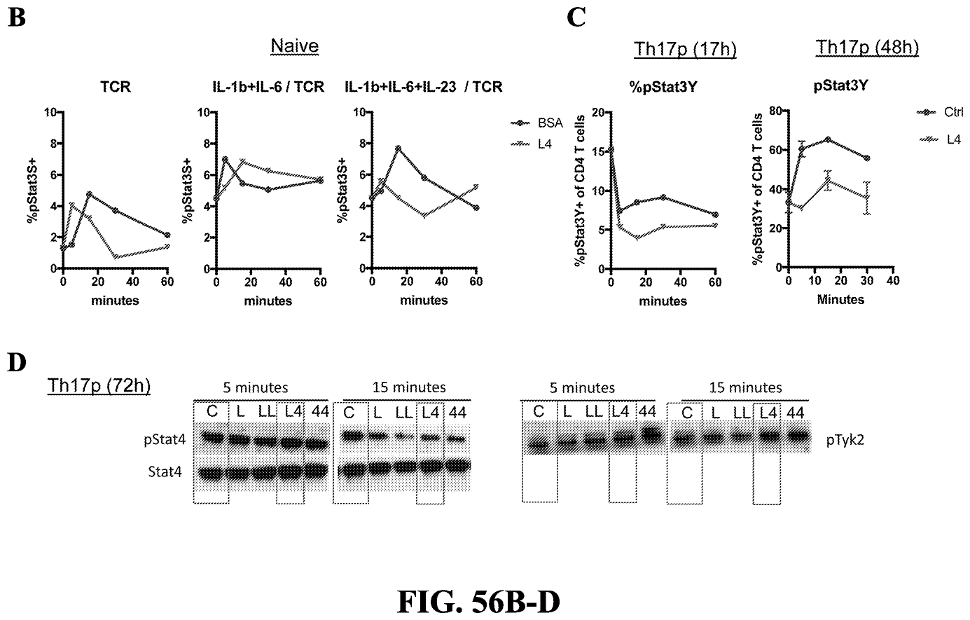

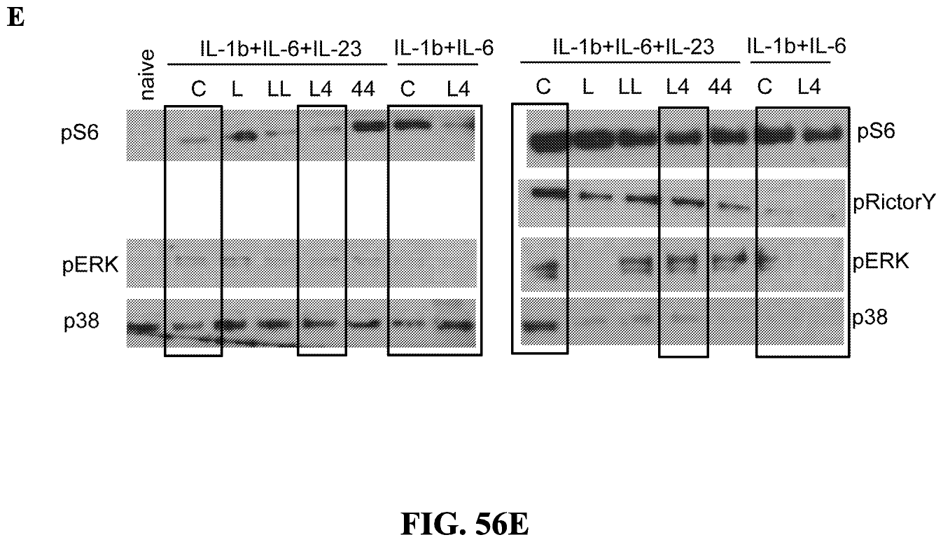

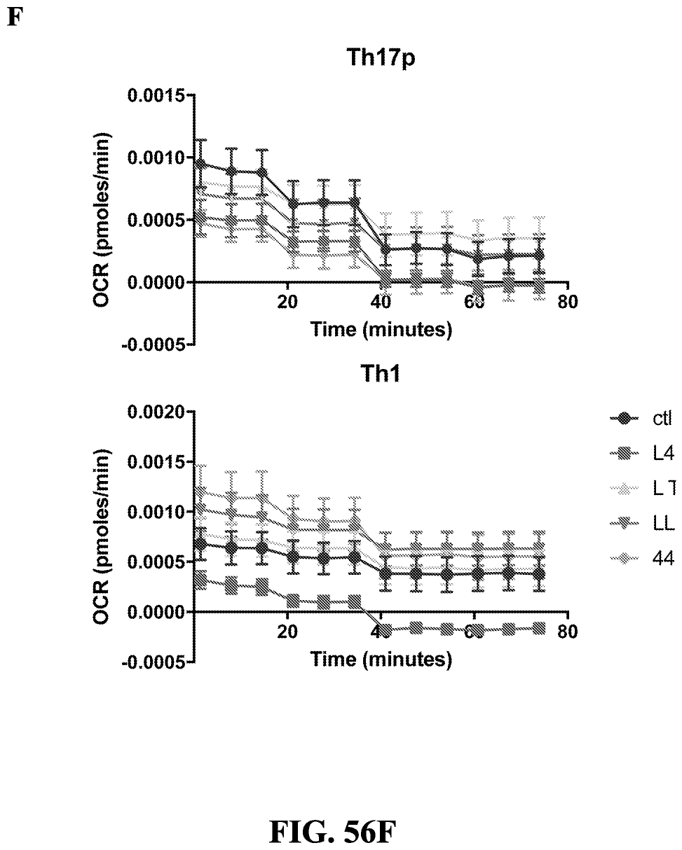

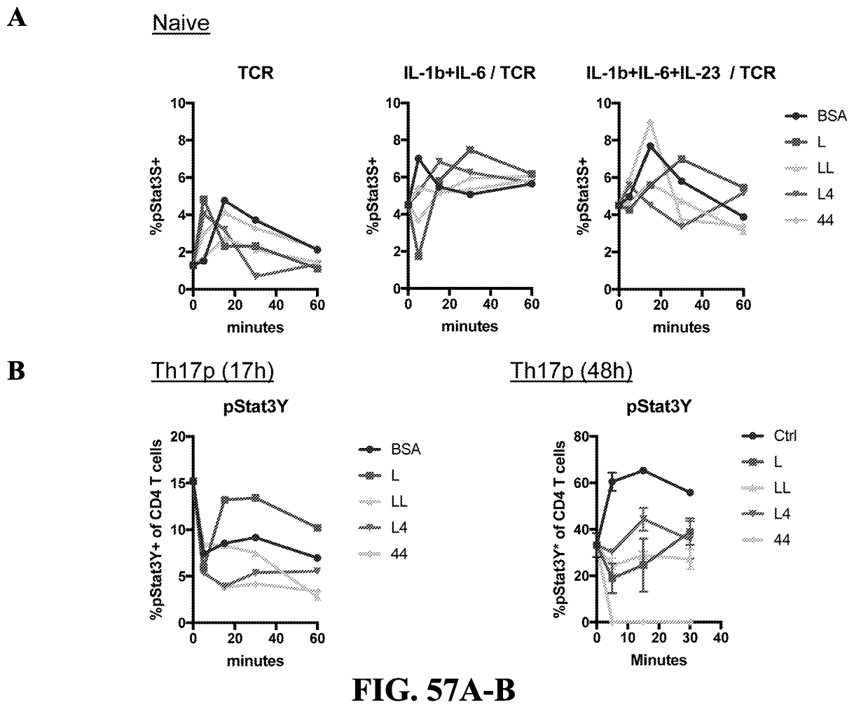

FIG. 56 further shows that recombinant CD5L:p40 can bind to Th17 cells directly and alters T cell signaling pathways and metabolism. FIG. 56A shows that Th17, Th1 and Th0 cells can be stained with recombinant CD5L:p40, but the staining is lost when Il12rb1 is knocked out. Thus, suggesting Il12rb1 is the receptor for CD5L:p40. FIGS. 56B-C and 57A-B show that CD5L:p40 suppresses phosphorylation of Stat3 (pStat3). The effect is stronger in Th17 cells. FIG. 56D shows CD5L:p40 suppresses pStat4 but not pTyk2 in Th17p cells. FIG. 56E shows that CD5L:p40 suppresses the phospho-proteins pRictorY, pS6 and p38 to study whether CD5L:p40 influence other signaling pathways. FIG. 56F shows that CD5L:p40 alters T cell metabolism in response to glutamate.

### Example 40—CD5L Deficiency has Additive or Synergistic Effect with PD-1 Blockade in Mice Implanted with B16-F10 Melanoma

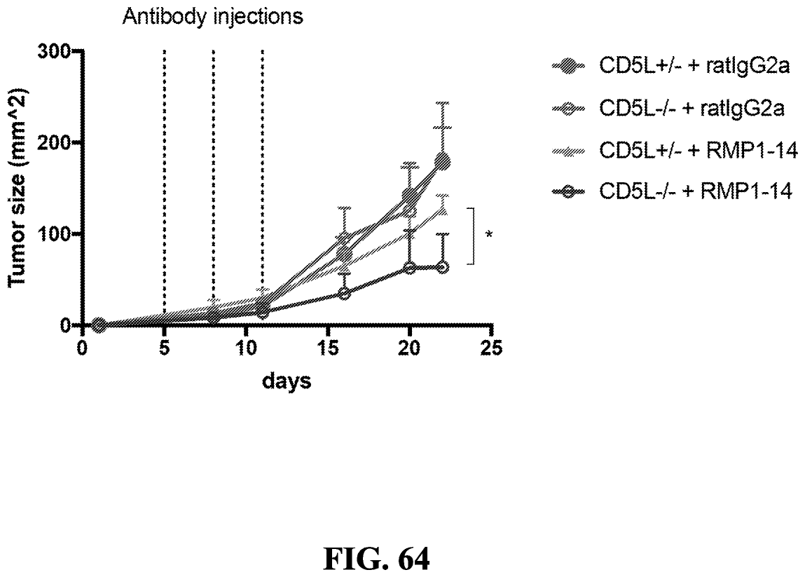

Control or CD5L−/− mice were implanted with B16-F10 melanoma subcutaneously. PD-1 blocking antibody (RMP1-14) or isotype control antibodies were given intraperitoneally to control or CD5L−/− mice at 200 ug/mice on day 5, 8 and 11. Whereas PD-1 blockade or CD5L deficiency alone did not show significant effect on b16 tumor growth under the tested condition, combining PD-1 blockade and CD5L deficiency resulted in enhance tumor control (FIG. 64).

### REFERENCES

Abdi et al., (2014). Free IL-12p40 monomer is a polyfunctional adaptor for generating novel IL-12-like heterodimers extracellularly. Journal of immunology 192, 6028-6036.
Burkett et al., (2015). Pouring fuel on the fire: Th17 cells, the environment, and autoimmunity. J Clin Invest 125, 2211-2219.
Cho et al., (2015). Heterogeneity of autoimmune diseases: pathophysiologic insights from genetics and implications for new therapies. Nature medicine 21, 730-738.
Didonna, A. and J. R. Oksenberg, (2015) Genetic determinants of risk and progression in multiple sclerosis. Clin Chim Acta, 449: p. 16-22.
Floss, D. M., et al. (2015) Insights into IL-23 biology: From structure to function. Cytokine Growth Factor Rev. 26(5): p. 569-78.
Gaublomme, J. T., et al., Single-Cell Genomics Unveils Critical Regulators of Th17 Cell Pathogenicity. Cell, 2015. 163(6): p. 1400-12.
Hill et al., (2008). Retinoic acid enhances Foxp3 induction indirectly by relieving inhibition from CD4+CD44hi Cells. Immunity 29, 758-770.
Jeoung, N. H. and R. A. Harris (2008) Pyruvate dehydrogenase kinase-4 deficiency lowers blood glucose and improves glucose tolerance in diet-induced obese mice. Am J Physiol Endocrinol Metab. 295(1): p. E46-54.
Kurokawa et al., (2010). Macrophage-derived AIM is endocytosed into adipocytes and decreases lipid droplets via inhibition of fatty acid synthase activity. Cell metabolism 11, 479-492.
Langrish, C. L., et al. (2005) IL-23 drives a pathogenic T cell population that induces autoimmune inflammation. J Exp Med. 201(2): p. 233-40.
Lee, Y., et al. (2012) Induction and molecular signature of pathogenic TH17 cells. Nat Immunol. 13(10): p. 991-9.
Lock, C., et al. (2002) Gene-microarray analysis of multiple sclerosis lesions yields new targets validated in autoimmune encephalomyelitis. Nat Med. 8(5): p. 500-8.
Mascanfroni, I. D., et al. (2013) IL-27 acts on DCs to suppress the T cell response and autoimmunity by inducing expression of the immunoregulatory molecule CD39. Nat Immunol. 14(10): p. 1054-63.
Miyazaki et al., (1999). Increased susceptibility of thymocytes to apoptosis in mice lacking AIM, a novel murine macrophage-derived soluble factor belonging to the scavenger receptor cysteine-rich domain superfamily. The Journal of experimental medicine 189, 413-422.
Parnas, O., et al. (2015) A Genome-wide CRISPR Screen in Primary Immune Cells to Dissect Regulatory Networks. Cell. 162(3): p. 675-86.
Sanjurjo et al., (2015). The human CD5L/AIM-CD36 axis: A novel autophagy inducer in macrophages that modulates inflammatory responses. Autophagy 11, 487-502.
Stumhofer, J. S. et al. (2007) Interleukins 27 and 6 induce STAT3-mediated T cell production of interleukin 10. Nat Immunol. 8(12): p. 1363-71.
Teng et al., (2015). IL-12 and IL-23 cytokines: from discovery to targeted therapies for immune-mediated inflammatory diseases. Nature medicine 21, 719-729.
Wang, C., et al. (2015) CD5L/AIM Regulates Lipid Biosynthesis and Restrains Th17 Cell Pathogenicity. Cell. 163(6): p. 1413-27.
Wang and Karin, (2015). The IL-23 to IL-17 cascade inflammation-related cancers. Clin Exp Rheumatol 33, 87-90.
Yosef, N., et al. (2013) Dynamic regulatory network controlling TH17 cell differentiation. Nature. 496(7446): p. 461-8.
Yoshida, H. and C. A. Hunter (2015) The immunobiology of interleukin-27. Annu Rev Immunol. 33: p. 417-43.
Zhu, C., et al. (2015) An IL-27/NFIL3 signalling axis drives Tim-3 and IL-10 expression and T-cell dysfunction. Nat Commun. 6: p. 6072.

The invention is further described by the following numbered paragraphs:
1. An antagonist against one or more of a CD5L monomer, a CD5L:CD5L homodimer, and a CD5L:p40 heterodimer.
2. The antagonist of paragraph 1, wherein the antagonist is an antibody, or an antigen binding fragment or equivalent thereof, that interacts with (e.g., specifically binds with) one or more of the CD5L monomer, the CD5L:CD5L homodimer, and the CD5L:p40 heterodimer.
3. The antagonist of paragraph 2, wherein the antibody is a polyclonal antibody, a monoclonal antibody, a chimeric antibody, a human antibody, a veneered antibody, a diabody, a humanized antibody, an antibody derivative, a recombinant humanized antibody.
4. The antagonist of paragraph 2, wherein the equivalent is an aptamer, affimer, non-immunoglobulin scaffold, small molecule, or fragment or derivative thereof.
5. The antagonist of paragraph 2, wherein the antibody specifically binds the CD5L monomer.
6. The antagonist of paragraph 2, wherein the antibody specifically binds the CD5L:CD5L homodimer.
7. The antagonist of paragraph 6, wherein the antibody is produced by a cell line selected from the group of cell lines listed in Table 1.
8. The antagonist of paragraph 2, wherein the antibody specifically binds a CD5L:p40 heterodimer.
9. The antagonist of paragraph 8, wherein the antibody is produced by a cell line selected from the group of cell lines in Table 2.
10. A composition comprising the antagonist of any one of paragraphs 1 to 7 and a pharmaceutically acceptable carrier.
11. The composition of paragraph 10, further comprising an additional active agent used to treat a cancer that is not inflammation related.
12. The composition of paragraph 9, wherein the additional active agent is one or more checkpoint inhibitors, PD-1/PDL-1, anti-cancer vaccines, adoptive T cell therapy, and/or inhibitory nucleic acids that target CD5L and/or p40.
13. The composition of paragraph 12, wherein inhibitory nucleic acids are small interfering RNAs (e.g., shRNA), antisense oligonucleotides, and/or CRISPR-Cas.
14. A method of treating a cancer that is not inflammation related in a subject comprising administering to the subject a therapeutically effective amount of an antagonist of any one of paragraphs 1 to 9 or a composition of any one of paragraphs 10 to 13.
15. The method of paragraph 14, further comprising sequentially or simultaneously administering an additional active agent used to treat the cancer.
16. The method of paragraph 15, wherein the additional active agent is a standard treatment for the cancer.
17. The method of any one of paragraphs 14 to 16, wherein the cancer is adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer.
18. A method for enhancing an immune response in a subject, comprising administering to the subject a therapeutically effective amount of an antagonist of any one of paragraphs 1 to 9 or a composition of any one of paragraphs 10 to 13.
19. The method of paragraph 18, wherein the subject has an immune deficiency.
20. The method of paragraph 19, wherein the immune deficiency is a primary or secondary immune deficiency.
21. The method of paragraph 18, wherein the subject has an infection with a pathogen.
22. The method of paragraph 21, wherein the pathogen is a viral, bacterial, or fungal pathogen.
23. A method of modulating CD8⁺ T cell exhaustion in a subject in need thereof, the method comprising administering to the subject a therapeutically effective amount of an antagonist antibody to one or more of a CD5L monomer, a CD5L:CD5L homodimer, and a CD5L:p40 heterodimer.
24. An antagonistic antibody that associates with an epitope of one or more of a CD5L monomer, a CD5L:CD5L homodimer, and a CD5L:p40 heterodimer.
25. A method of identifying a gene or a set of genes up and/or downregulated in response to an antagonistic antibody, the method comprising:
   exposing a cell or population of cells to the antagonist of any one of paragraphs 1 to 9, and introducing one or more guide RNAs that target one or more endogenous genes into the cell or population of cells, wherein the cell or population of cells express a CRISPR-Cas9 protein or a CRISPR-Cas9 protein or a nucleic acid encoding the CRISPR-Cas9 protein has been introduced into the cell or population of cells simultaneously or sequentially with the guide RNAs, assaying for a phenotype indicative of enhanced or suppressed immune response, and identifying a gene or set of genes up and/or down regulated in the cell or population of cells with the enhanced or suppressed immune response.
26. The method of paragraph 25, wherein the cell or population of cells are cancer cell(s).
27. The method of paragraph 26, wherein the cancer is adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer.
28. The method of paragraph 26 or paragraph 27, wherein the cancer cell(s) are human cells.
29. The method of paragraph 28, wherein the human cancer cell(s) have been transplanted into a mouse.
30. A method of treating a cancer that is not inflammation related comprising administering to a subject in need thereof (i) the antagonist of any one of paragraphs 1 to 9 and (ii) an agent that targets a gene or set of genes identified according to paragraph 25.
31. A method of screening for an antagonist of one or more of a CD5L monomer, a CDSL:CDSL homodimer, and a CD5L:p40 heterodimer, the method comprising:
   exposing a cell or a population of cells to an agent that interacts with one or more of a CD5L monomer, a CDSL:CDSL homodimer, and a CD5L:p40 heterodimer;
   identifying a gene or set of genes up and/or down-regulated in the cell or population of cells; and
   determining that the agent is an antagonist based on the gene or set of genes up and/or down-regulated in the cell or population of cells.
32. The method of paragraph 31, wherein the antagonist is an antibody.
33. The method of paragraph 31, further comprising comparing the identified gene or set of genes to a previously-identified gene or set of genes up and/or down-regulated upon exposure to an antagonist of one or more of a CD5L monomer, a CDSL:CDSL homodimer, and a CD5L:p40 heterodimer.
34. A method of screening for an antagonistic agent comprising:
   identifying an epitope on one or more of a CD5L monomer, a CDSL:CDSL homodimer, and a CD5L:p40 heterodimer that interacts with an antagonist of one or more of a CD5L monomer, a CDSL:CDSL homodimer, and a CD5L:p40 heterodimer; and
   screening against a library of candidate antagonistic agents for an antagonistic agent that interacts with the epitope.
35. The method of paragraph 34, wherein the antagonist is an antibody.
36. The method of paragraph 34, wherein the antagonistic agent is an antibody, a small molecule, a peptide, an aptamer, an affimer, a non-immunoglobulin scaffold, or fragment or derivative thereof.
37. The method of paragraph 34, wherein the library comprises a computer database and the screening comprises a virtual screening.
38. The method of paragraph 34, wherein the screening comprises evaluating the three-dimensional structure of one or more of the CD5L monomer, the CD5L:CD5L homodimer, and the CD5L:p40 heterodimer.
39. A method of identifying an agent for treating a cancer that is not inflammation related in a subject, comprising contacting the agent with a T cell, wherein decreased expression of CD5L monomer, CD5L:CD5L homodimer, and/or CD5L:p40 heterodimer indicates that the agent is effective for treating the cancer that is not inflammation related in the subject.
40. A method of identifying an agent for enhancing an immune response in a subject, comprising contacting the agent with a T cell, wherein decreased expression of CD5L monomer, CD5L:CD5L homodimer, and/or CD5L:p40 heterodimer indicates that the agent is effective for enhancing the immune response in the subject.
41. The method of paragraph 40, wherein the subject has an immune deficiency.
42. The method of paragraph 41, wherein the immune deficiency is a primary or secondary immune deficiency.
43. The method of paragraph 40, wherein the subject has an infection with a pathogen.
44. The method of paragraph 40, wherein the pathogen is a viral, bacterial, or fungal pathogen.
45. A method of treating cancer in a subject, comprising administering to the subject a therapeutically effective amount of an antagonist of any one of paragraphs 1-9 or a composition of any one of paragraphs 10 to 12, wherein the cancer is promoted by complement.
46. The method of paragraph 45, wherein the antagonist is an antibody.
47. The method of paragraph 46, wherein the antibody specifically binds CD5L monomer.
48. The method of paragraph 46, wherein the antibody specifically binds CD5L:CD5L homodimer.
49. The method of paragraph 46, wherein the antibody specifically binds CD5L:p40 heterodimer.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An antagonist against the function or signaling of one or more of a CD5L monomer, a CD5L:CD5L homodimer, and a CD5L:p40 heterodimer.

2. The antagonist of claim 1, wherein the antagonist is an antibody, or an antigen binding fragment or equivalent thereof, that interacts with (e.g., specifically binds with) one or more of the CD5L monomer, the CD5L:CD5L homodimer, and the CD5L:p40 heterodimer.

3. The antagonist of claim 1, wherein the antagonist is an antibody, or an antigen binding fragment or equivalent thereof, that interacts with (e.g., specifically binds with) Il12rb1.

4. The antagonist of claim 2 or 3, wherein the antibody is a polyclonal antibody, a monoclonal antibody, a chimeric antibody, a human antibody, a veneered antibody, a diabody, a humanized antibody, an antibody derivative, a recombinant humanized antibody.

5. The antagonist of claim 2 or 3, wherein the equivalent is an aptamer, affimer, non-immunoglobulin scaffold, small molecule, or fragment or derivative thereof.

6. The antagonist of claim 2, wherein the antibody specifically binds the CD5L monomer.

7. The antagonist of claim 2, wherein the antibody specifically binds the CD5L:CD5L homodimer.

8. The antagonist of claim 6 or 7, wherein the antibody is produced by a cell line selected from the group of cell lines listed in Table 1.

9. The antagonist of claim 2, wherein the antibody specifically binds a CD5L:p40 heterodimer.

10. The antagonist of claim 9, wherein the antibody is produced by a cell line selected from the group of cell lines in Table 2.

11. The antagonist of claim 1, wherein the antagonist is an antibody, an antigen binding fragment or equivalent thereof, small molecule, or genetic modifying agent, said antagonist targeting a downstream target of a CD5L:p40 heterodimer, a CD5L monomer, or a CD5L:CD5L homodimer.

12. The method of claim 11, wherein the downstream target is selected from the group consisting of Dusp2, Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Snx20, Lamp1, Faf1, Gpatch3, Dapk3, 1110065P20Rik, Vaultrc5, 1Il17f, I1Il17a, Ildr1, Il1r1, Lgr4, Ptpnl4, Paqr8, Timp1, Il1rn, Smim3, Gap43, Tigit, Mmp10, Il22, Enpp2, Iltifb, Ido1, 1123r, Stom, Bcl2l11, 5031414D18Rik, Il24, Itga7, Il6, Epha2, Mt2, Upp1, Snord104, 5730577I03Rik, Slcl8b1, Ptprj, Clip3, Mir5104, Ppifos, Rab13, Hist1h2bn, Ass1, Cd200r1, E130112N10Rik, Mxd4, Casp6, Gatm, Tnfrsf8, Gp49a, Gadd45g, Ccr5, Tgm2, Lilrb4, Ecm1, Arhgap18, Serpinb5, Cysltr1, Enpp1, Selp, Slc38a4, Gm14005, Epb4.1l4b, Moxd1, Klra7, Igfbp4, Tnip3, Gstt1, Pglyrp2, Il12rb2, Ctla2a, Plac8, Ly6c1, Sell, Ncf1, Trp53i11, B3gnt3, Kremen2, Matk, Ltb4r1, Ets1, Tnfrsf26, Cd28, Rybp, Ppp1r3c, Thy1, Trib2, Sema3b, Pros1, Il33, Gm5483, Myh11, Cntd1, Ms4a4b, Treml2, 3110009E18Rik, Pglyrp1, Amd1, Slc24a5, Snhg9, Ifi27l1, Irf7, Mx1, Snhg10, 114, Snora43, H2-L, Myl4, Insl3, Tgoln2, BC022687, C230035I16Rik, Hvcn1, Myh10, Dhrs3, Acsl6, Rgs2, Ccl20, Ccl3, Dlg2, Ccr6, Ccl4, Dusp14, Apol9b, Cd72, Ispd, Cd70, S100al, Lgals3, Slc15a3, Nkg7, Serpinc1, Olfr175-ps1, Il9, Pdlim4, Il3, Insl6, Perp, Cd51, Serpine2, Galnt14, Tff1, Ppfibp2, Bdh2, Mlf1, Il1a, Osr2, Gm5779, Ebf1, Spink2, Egfr and Ccdc155.

13. A composition comprising the antagonist of claim 1 and a pharmaceutically acceptable carrier.

14. The composition of claim 13, further comprising an additional active agent used to treat a cancer.

15. The composition of claim 13, wherein the cancer is not inflammation related.

16. The composition of claim 14 or 15, wherein the additional active agent is one or more checkpoint inhibitors, anti-PD-1, anti-PDL-1, anti-CTLA4, anti-cancer vaccines, adoptive T cell therapy, and/or inhibitory nucleic acids that target CD5L and/or p40.

17. The composition of claim 16, wherein inhibitory nucleic acids are genetic modifying agents, small interfering RNAs (e.g., shRNA), antisense oligonucleotides, and/or CRISPR system.

18. A method of treating a cancer in a subject in need thereof comprising administering to the subject a therapeutically effective amount of an antagonist of claim 1 or a composition of claim 13.

19. The method of claim 18, further comprising sequentially or simultaneously administering an additional active agent used to treat the cancer.

20. The method of claim 19, wherein the additional active agent is a standard treatment for the cancer.

21. The method of claim 20, wherein the cancer treatment is an immunotherapy treatment.

22. The method of claim 21, wherein the immunotherapy treatment is checkpoint blockade therapy.

23. The method of claim 22, wherein the checkpoint blockade therapy comprises anti-CTLA4, anti-PD1, anti-PDL1 or combination thereof.

24. The method of any one of claims 18 to 23, wherein the cancer is adenoid cystic carcinoma (ACC), bladder cancer, breast cancer, cervical cancer, colorectal cancer cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer.

25. A method for enhancing an immune response in a subject, comprising administering to the subject a therapeutically effective amount of an antagonist of claim 1 or a composition of claim 13.

26. The method of claim 25, wherein the subject has an immune deficiency.

27. The method of claim 26, wherein the immune deficiency is a primary or secondary immune deficiency.

28. The method of claim 25, wherein the subject has an infection with a pathogen.

29. The method of claim 28, wherein the pathogen is a viral, bacterial, or fungal pathogen.

30. A method of modulating CD8⁺ T cell exhaustion in a subject in need thereof, the method comprising administering to the subject a therapeutically effective amount of an antagonist antibody to one or more of a CD5L monomer, a CD5L:CD5L homodimer, and a CD5L:p40 heterodimer.

31. An antagonistic antibody that associates with an epitope of one or more of a CD5L monomer, a CD5L:CD5L homodimer, and a CD5L:p40 heterodimer.

32. A method of screening for an antagonist of one or more of a CD5L monomer, a CD5L:CD5L homodimer, and a CD5L:p40 heterodimer, the method comprising:
exposing a cell or a population of cells to an agent that interacts with one or more of a CD5L monomer, a CD5L:CD5L homodimer, and a CD5L:p40 heterodimer;
determining expression of a gene or set of genes up and/or down-regulated upon exposure to one or more of a CD5L monomer, a CD5L:CD5L homodimer, a CD5L:p40 heterodimer or antagonist thereof in the cell or population of cells; and
determining that the agent is an antagonist based on the gene or set of genes up and/or down-regulated in the cell or population of cells.

33. The method of claim 32, wherein the antagonist is an antibody.

34. A method of screening for an antagonistic agent comprising:
identifying an epitope on one or more of a CD5L monomer, a CD5L:CD5L homodimer, and a CD5L:p40 heterodimer that interacts with an antagonist of one or more of a CD5L monomer, a CD5L:CD5L homodimer, and a CD5L:p40 heterodimer; and
screening against a library of candidate antagonistic agents for an antagonistic agent that interacts with the epitope.

35. The method of claim 34, wherein the antagonist is an antibody.

36. The method of claim 34, wherein the antagonistic agent is an antibody, a small molecule, a peptide, an aptamer, an affimer, a non-immunoglobulin scaffold, or fragment or derivative thereof.

37. The method of claim 34, wherein the library comprises a computer database and the screening comprises a virtual screening.

38. The method of claim 34, wherein the screening comprises evaluating the three-dimensional structure of one or more of the CD5L monomer, the CD5L:CD5L homodimer, and the CD5L:p40 heterodimer.

39. A method of identifying an agent for treating a cancer that is not inflammation related in a subject, comprising contacting the agent with a T cell, wherein decreased expression of CD5L monomer, CD5L:CD5L homodimer, and/or CD5L:p40 heterodimer indicates that the agent is effective for treating the cancer that is not inflammation related in the subject.

40. A method of identifying an agent for enhancing an immune response in a subject, comprising contacting a myeloid cell with the agent, wherein decreased expression of CD5L monomer, CD5L:CD5L homodimer, and/or CD5L:p40 heterodimer indicates that the agent is effective for enhancing the immune response in the subject.

41. The method of claim 40, wherein the subject has an immune deficiency.

42. The method of claim 41, wherein the immune deficiency is a primary or secondary immune deficiency.

43. The method of claim 40, wherein the subject has an infection with a pathogen.

44. The method of claim 43, wherein the pathogen is a viral, bacterial, or fungal pathogen.

45. A method of treating cancer in a subject, comprising administering to the subject a therapeutically effective amount of an antagonist of claim 1 or a composition of claim 13, wherein the cancer is promoted by complement.

46. The method of claim 45, wherein the antagonist is an antibody.

47. The method of claim 46, wherein the antibody specifically binds CD5L monomer.

48. The method of claim 46, wherein the antibody specifically binds CD5L:CD5L homodimer.

49. The method of claim 46, wherein the antibody specifically binds CD5L:p40 heterodimer.

50. The antagonist of claim 1, wherein the antagonist is an antibody that binds to the fibronectin domain 2 of p40.


SEQUENCE LISTING (24 sequences)

SEQ ID NO: 1 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 347 aa, Homo sapiens
MALLFSLILAICTRPGFLASPSGVRLVGGLHRCEGRVEVEQKGQWGTVCDDGWDIKDVAVLCRELGCGAASGTPSGILYEPPAEKEQKVLIQSVSCTGTEDTLAQCEQEEVYDCSHDEDAGASCENPESSFSPVPEGVRLADGPGHCKGRVEVKHQNQWYTVCQTGWSLRAAKVVCRQLGCGRAVLTQKRCNKHAYGRKPIWLSQMSCSGREATLQDCPSGPWGKNTCNHDEDTWVECEDPFDLRLVGGDNLCSGRLEVLHKGVWGSVCDDNWGEKEDQVVCKQLGCGKSLSPSFRDRKCYGPGVGRIWLDNVRCSGEEQSLEQCQHRFWGFHDCTHQEDVAVICSG

SEQ ID NO: 3 - protein, 352 aa, Mus musculus
MAPLFNLMLAILSIFVGSCFSESPTKVQLVGGAHRCEGRVEVEBNGQWGTVCDDGWRDRDVAVVCRELNCGAVIQTPRGASYQPPASEQRVLIQGVDCNGTEDTLAQCELNYDVFDCSHEEDAGAQCENPDSDLLFIPEDVRLVDGPGBCQGRVEVLBQSQWSTVCKAGWNLQVSKVVCRQLGCGRALLTYGSCNKSTQGKGPIWMGKMSCSGQEANLRSCLLSRLENNCTHGEDTWMECEDPFELKLVGGDTPCSGRLEVLHKGSWGSVCDDNWGEKEDQVVCKQLGCGKHSPLSPKTRKIYGPGAGRIWLDDVNCSGKEQSLEFCRHRLWGYHDCTHKEDVEVICTDFDV

SEQ ID NO: 4 - protein, 347 aa, Homo sapiens
MALLFSLILAICTRPGFLASPSGVRLVGGLBRCEGRVEVEQKGQWGTVCDDGWIDKDVAVLCRELGCGAASGTPSGILYEPPAEKEQKVLIQSVSCTGTEDTLAQCEQEEVYDCSHDEDAGASCENPESSFSPVPEGVRLADGPGBCKGRVEVKBQNQWYTVCQTGWSLRAAKVVCRQLGCGRAVLTQKRCNKBAYGRKPIWLSQMSCSGREATLQDCPSGPWGKNTCNHDEDTWVECEDPFDLRLVGGDNLCSGRLEVLHKGVWGSVCDDNWGEKEDQVVCKQLGCGKSSPLSFRDRKCYGPGVGRIWLDNVRCSGEEQSLEQCQBRFWGFHDCTHQEDVAVICSV

SEQ ID NO: 5 - protein, 195 aa, Mus musculus
MLDCRAVIMLWLLPWVTQGLAVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSQAFLAIAARVFAHGAATLTEPLVPTA

SEQ ID NO: 6 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 7 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 8 - protein, 214 aa, Mus musculus
MCQSRYLLFLATLVLLNHLTSARVIPVSGPAKCLNQSQNLLKTTDDMVRTAREKLKHSCTAGDIDHEDITRDKTSTLEACLPLELHKNESCLATKETSSIIRGSCLPPQKTSLMMTLCLGSIYEDLKMYQSEFQAINAALQSHNHQQITLDRNMLMAIDELMRSLNHSGETLHQKAPMGEADPYRVKMKLCILLHAFSTRVMTINRVMNYLSSS

SEQ ID NO: 9 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLALTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLEILLHAFRIRAVTIDRVMSYLNAS

SEQ ID NO: 10 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 11 - protein, 330 aa, Homo sapiens [sequence omitted]

SEQ ID NO: 12 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 13 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 15 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 17 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 18 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 20 - protein, 9 aa, Homo sapiens
IEVMYPPPY

SEQ ID NO: 21 - protein, 288 aa, Artificial Sequence
  Synthetic Peptide
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 22 - protein, 183 aa, Artificial Sequence
  Synthetic Peptide
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 23 - protein, 4 aa, Artificial Sequence
  Synthetic Peptide
SGGG

SEQ ID NO: 24 - protein, 1015 aa, Artificial Sequence
  Synthetic Peptide
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGSGGGSGGGSGGESPTKVQLVGGAHRCEGRVEVEHNGQWGTVCDDGWDRRDVAVVCRELNCGAVIQTPRGASYQPPASEQRVLIQGVDCNGTEDTLAQCELNYDVFDCSHEEDAGAQCENPDSDLLFIPEDVRLVDGPGHCQGRVEVLHQSQWSTVCKAGWNLQVSKVVCRQLGCGRALLTYGSCNKSTQGKGPIWMGKMSCSGQEANLRSCLLSRLENNCTHGEDTWMECEDPFELKLVGGDTPCSGRLEVLHKGSWGSVCDDNWGEKEDQVVCKQLGCGKSLHPSPKTRKIYGPGAGRIWLDDVNCSGKEQSLEFCRHRLWGYHDCTHKEDVEVICTDFDVTGHHHHHHHHGGQESPTKVQLVGGAHRCEGRVEVEHNGQWGTVCDDGWDRRDVAVVCRELNCGAVIQTPRGASYQPPASEQRVLIQGVDCNGTEDTLAQCELNYDVFDCSHEEDAGAQCENPDSDLLFIPEDVRLVDGPGHCQGRVEVLHQSQWSTVCKAGWNLQVSKVVCRQLGCGRALLTYGSCNKSTQGKGPIWMGKMSCSGQEANLRSCLLSRLENNCTHGEDTWMECEDPFELKLVGGDTPCSGRLEVLHKGSWGSVCDDNWGEKEDQVVCKQLGCGKSLHPSPKTRKIYGPGAGRIWLDDVNCSGKEQSLEFCRHRLWGYHDCTHKEDVEVICTDFDVTGHHHHHHHHGGQ